SEQUENCE LISTING (241 sequences)

SEQ ID NO: 1 - DNA, 330 bp, Artificial Sequence
  BLV1H12 Light chain DNA
caggccgtcctgaaccagccaagcagcgtctccgggtctctggggcagcgggtctcaatcacctgtagcgggtcttcctccaatgtcggcaacggctacgtgtcttggtatcagctgatccctggcagtgccccacgaaccctgatctacggcgacacatccagagcttctggggtccccgatcggttctcagggagcagatccggaaacacagctactctgaccatcagctccctccaggctgaggacgaagcagattatttctgcgcgtctgccgaggactctagttcaaatgccgtgtttggaagcggcaccacactgacagtccta

SEQ ID NO: 2 - protein, 110 aa, Artificial Sequence
  BLV1H12 variable Light chain
QAVLNQPSSVSGSLGQRVSITCSGSSSNVGNGYVSWYQLIPGSAPRTLIYGDTSRASGVPDRFSGSRSGNTATLTISSLQAEDEADYFCASAEDSSSNAVFGSGTTLTVL

SEQ ID NO: 3 - DNA, 25 bp, Artificial Sequence
  igGCDNA2REV
ccgctcttcagggcacccgagttcc

SEQ ID NO: 4 - DNA, 25 bp, Artificial Sequence
  igMCDNA2REV
ctgactgtgctgttgttgaacttcc

SEQ ID NO: 5 - DNA, 25 bp, Artificial Sequence
  igACDNA2REV
gacacgctgtcgccattctggttcc

SEQ ID NO: 6 - DNA, 32 bp, Artificial Sequence
  igGCDNA1.7REV
cgggcacggtcaccatgctgctgagagagtag

SEQ ID NO: 7 - DNA, 56 bp, Artificial Sequence
  BOVVHFR4REV
ttacctgcggccgctgaggagacggtgaccaggagtccaactggagctccatcaag

SEQ ID NO: 8 - DNA, 40 bp, Artificial Sequence
  BOVSTALKFOR1
cagccggccatggccacatactacagtactactgtacacc

SEQ ID NO: 9 - DNA, 40 bp, Artificial Sequence
  BOVSTALKFOR2
cagccggccatggccacatactacagtactactgtatacc

SEQ ID NO: 10 - DNA, 40 bp, Artificial Sequence
  BOVSTALKFOR3
cagccggccatggccacatactacagtactactgtgctcc

SEQ ID NO: 11 - DNA, 40 bp, Artificial Sequence
  BOVSTALKFOR4
cagccggccatggccacatactacagtggtactgtgcacc

SEQ ID NO: 12 - DNA, 37 bp, Artificial Sequence
  BOVVHNCOFOR2
aaaaagccatggtgcaggtgcagctgcgggagtcggg

SEQ ID NO: 13 - DNA, 36 bp, Artificial Sequence
  BOVVHFR4XHOREV
ttacctctcgagtgaggagacggtgaccaggagtcc

SEQ ID NO: 14 - DNA, 507 bp, Artificial Sequence
  R2G3
caggtgcagctgcgggagtcgggccccagcctggtgaagccctcacagaccctctcgctcacctgcgcggcctctggattctcattgagcgacaaggctgtaggctgggtccgccgggctccagggaaggcgctggagtggctcggtagtatagacactggtggaagcacaggctataacccaggcctgaaatcccggctcagcatcaccaaggacaactccaagagccaagtctccctgtcaattagcagcgtaacgtctgaggactcggccacatactactgtgcaactgtacaccagaaaacagctgaaggagacaaaacgtgtcctgatggttacgagcatacttgtggttgcattgggggttgtggttgcaaaaggtctgcctgtataggtgcactttgttgccaagcgtcgttgggtggttggcttagtgacggtgaaacctacacttacgagttccacgtcgatacctggggccaaggactcgtggtcaccgtctcctca

SEQ ID NO: 15 - DNA, 507 bp, Artificial Sequence
  SR3A3
caggtgcagctgcgggagtcgggccccagcctggtgaagccctcacagaccctctccctcacctgcacaatctctggattctcattgagtagctatgctgtactctgggtccgccaggctccagggaagccgctggagtggctcggtagtatagacactgcggaaaacacaggctataacccaggcctgaaatcccggctcagcatcaccaaggacaactccaagagccaagtctctctgtcagtgagcagcgtgacaaccgaggactcggccacatactactgtgctactgtacaccagaaaacgcgaaaagaaaaaaattgtcctgatggctatatctatagttctaatatcactagcggttttgattgtggtgtctggatttgtcgtcgcgtcggtagtgccttctgtagtcgtactggtgattatactagtcctactgaacttgacatttacgagttctacgtcgaagggtggggccagggagtcccggtcaccgtctcctca

SEQ ID NO: 16 - DNA, 498 bp, Artificial Sequence
  R2F12
caggtgcagctgcgggagtcgggccccagcctggtgaagccctcacagaccctctccctcacctgcacggtctctggattctcattgagcgacaaggctgtaggctgggtccgccgggctccagggaaggcgctggagtggctcggtagtatagacactggtggaatgacaggctataacccaggcctgaaatcccggctcagcatcaccaaggacaactccaaaagccaagtctctctatcagtgaatagcgtgacaactgaggactcggccacgtactactgtgccactgtagaccagaaaacgaaaaatgcttgccctgatgatttcgattatcgttgttcgtgtatcggtggttgtggctgcgcccgtaaaggatgcgttggtcctctttgttgtcgttctgatttgggtggctatcttactgatagtcctgcttacatttacgaatggtatattgatctttggggccaaggactcctggtcaccgtctcctca

SEQ ID NO: 17 - DNA, 405 bp, Artificial Sequence
  R5A3
caggtgcagctgcgggagtcgggccccagcctggtgaagccgtcacagaccctctcgctcacctgcacggcctctggattctcattgagcgacaaggctgtaggctgggtccgccaggctccagggaaggcgctggagtggctcggtagtatagacactggtggaagcacaggctataacccaggcctgaaatcccggctcagcatcaccaaggacaactccaagagccaagtctctctgtcagtgagcagcgtgacaactgaggactcggccacatactactgtactactgtgcactgtagtgatggtggttatgttgaggcgggttttggttgttggccttgggattatggttatccttacgtcgatgcctggggccaaggactcctggtcaccgtctcctca

SEQ ID NO: 18 - DNA, 396 bp, Artificial Sequence
  R4G11
caggtgcagctgcgggagtcgggccccagcctggtgaagccctcacagaccctctccctcacctgcacggtctctggattctcattgagcagctatggtataacctgggtccgccaggctccagggaaggcgctggagtgcctcggtagtataagcagtggtggaaccacagactacaacccagccctgaaatcccggctcagcatcaccaaggacaactccaagagccaagtctctctgtcagtgagcagcgtgacacctgaggacacggccacatactactgttcgaagtggaatttagaatatacttggggtggtgttggttgcgctagttttgctgatgaggacacccacgttgatgcctggggccaaggactcctggtcaccgtctcctca

SEQ ID NO: 19 - DNA, 417 bp, Artificial Sequence
  R4G3
caggtgcagctgcgggagtcgggccccagcctgatgaagccctcacagaccctctccctcacctgcacggtctctgggttctcattgagcgactatgctgtaggctgggtccgccaggccccagggaaggcgctggagtggctcggtggtatagacactggtggaagcacaggctataacccaggcctggaatcccggctcagcatcaccaaggacaactccaagagccaagtctctctgtcagtgagcagcgtgacaactgaggactcggccacatactactgtactactgtggtcctttgttattttaattatgttgttcgtcgttataattgtggtggtcttggttatgggcatggctttaatagtttctacgtcgatgcctggggccaaggactcctggtcaccgtctcctca

SEQ ID NO: 20 - DNA, 417 bp, Artificial Sequence
  R4E5
caggtgcagctgcgggagtcgggccccagcctggtgaagccctcacagaccctctccctcacctgcacgacctctggattctcactgagaaactatgctgtaggctgggtccgccaggctccggggaaggcgctggagtggctcggtggtatagacactggtggaagcacaggctataacccaggcctggaatcccggctcagcatcaccaaggacaactccaagagccaagtctctctgtcagtgagcagcgtgacaactgaggactcggccacatactactgtactactgtggtcctttgttattttaattatgttgttcgtcgttataattgtggtggtcttggttatgggcatggctttaatagtttctacgtcgatgcctggggccaaggactcctggtcaccgtctcctca

SEQ ID NO: 21 - DNA, 507 bp, Artificial Sequence
  R4C1
caggtgcagctgcgggagtcgggccccagcctggtgaagccgtcacagaccctctcgctcacctgcacggcctctggattctcattgagcgataaggctgtaggctgggtccgccaggctccagggaagccgctggagtggctcggtagtatagacactgcggaaaacacaggctataacccaggcctgaaatctcggctcagcatcaccaaggacaactccaagagccaagtctctctgtcagtgagcagcgtgacaactgaggactcggccacatactactgtgctactgtacaccagaaaacgcgaaaagaaaaaaattgtcctgatggctatatctatagttctaataccgccagcggttatgattgtggtgtctggatttgtcgtcgcgtcggtagtgccttctgtagtcgtactggtgattatactagtcctagtgaatttgacatttacgagttctacgtcgaagggtggggccagggactcctggtcaccgtctcctca

SEQ ID NO: 22 - DNA, 417 bp, Artificial Sequence
  R4A10
caggtgcagctgcgggagtcgggccccagcctggtgaagccctcacagaccctctccctcacctgcacgacctctggattctcattgagcgactatgctgtaggctgggtccgccaggctccagggaaggcgctggagtggctcggtggtatagacactggtggaagcacaggctataacccaggcctgaaatcccggctcagcatcaccaaggacaactccaagagtcaagtctctctgtcagtgagcagcgtgacaactgaggattcggccacatactactgtactgccgtggtcctctgttattacaatcgggttgtgcgtcgtaataattgtggtgggcttggttatgattatggttttgatcatttctacgtcgatgcctggggccaaggactcctggtcaccgtctcctca

SEQ ID NO: 23 - DNA, 384 bp, Artificial Sequence
  R2G1
caggtgcagctgcgggagtcgggccccagcctggtgaagccctcacagaccctctccctcacctgcacggtctctggattctcattgagcaactatgctgtaggctgggtccgccaggctccagggaaggcgctggagtgcctcggtgatgtagacagtagtggaggcacagcctataacccagccctgaaatcccggttcatcatcgccaaggacaactccaagaaccaagtctctctgtcagtccgcagcgtgacacctgaggacacggccacatactactgtgcgaagtttgctaagggtactacgagtgctggtgcttgtgattattcagaaagctacgtcgatgcctggggccagggactcctggtcaccgtctcctca

SEQ ID NO: 24 - DNA, 399 bp, Artificial Sequence
  R2D6
caggtgcagctgcgggagtcgggccccagcctggtgaagccctcacagaccctctccctcacctgcacgacctctggattctcactgagcagctatgctgtaggctgggtccgccaggctccggggaaggcgctggagtgggttggtgatatagattatgtcggaaacacagactataacccagccctgaaatcccggctcagcatcaccaaggacaactccaagagccaagtctctctggtagtgagcagcgtgacagctgaggacgcggccacatactactgtgcgaaatattccggtgcttatgcttatgctgcttgcaattattatggttggcgttgtgcttgggaaagctacatcgatgcctggggccaaggactcctggtcaccgtctcctca

SEQ ID NO: 25 - DNA, 399 bp, Artificial Sequence
  R2B1
caggtgcagctgcgggagtcgggccccagcctggtgaagccctcacagaccctctccctcacctgcacggtctctggattttcattaagcgataataatgtaggctgggtccgccaggctccaggaaaggcgctggagtggctcggtgtaatgcataatgatgggaacaaaggctataacccagccctgaaatcccggctcagcatcaccaaggacagctccaagagccaagtctctctatcactaagcagcgtgacaagtgaggacacggccacatactactgtacaagagacaatgcacgttgtgatagttggacgtatgacagctgtgatacttggtatcgcaattcgtggcacgttgatgcctggggccaaggactcctggtcaccgtctcctca

SEQ ID NO: 26 - DNA, 504 bp, Artificial Sequence
  SKM-BLV1H12
caggtgcagctgcgcgagtcgggccccagcctggtgaagccgtcacagaccctctcgctcacctgcacggcctctggattctcattgagcgacaaggctgtaggctgggtccgccaggctccagggaaggcgctggagtggctcggtagtatagacactggtggaaacacaggctataacccaggcctgaaatcccggctcagcatcaccaaggacaactccaagagtcaagtctctctgtcagtgagcagcgtgacaactgaggactcggccacatactactgtactactgtgcaccaagagaccttacgtagttgtcctgatggttatattgataattctggatgcacggctgattggggttgtgcagctcttgattgttggcggcgtcgttttggttaccacagcactgatccttctcattatactggtgcgacgtatatttacacgtacagcttgcacatcgatgcctggggccaaggactcctggtcaccgtctcctca

SEQ ID NO: 27 - DNA, 507 bp, Artificial Sequence
  SKD-BLV1H12
caggtgcagctgcgcgagtcgggccccagcctggtgaagccgtcacagaccctctcgctcacctgcacggcctctggattctcattgagcgacaaggctgtaggctgggtccgccaggctccagggaaggcgctggagtggctcggtagtatagacactggtggaaacacaggctataacccaggcctgaaatcccggctcagcatcaccaaggacaactccaagagtcaagtctctctgtcagtgagcagcgtgacaactgaggactcggccacatactactgtactactgtgcaccagcgtacaagcgaaaaaagaagttgtcctggcggtagtagtagacgttatcctagtggcgccagttgtgacgttagtgggggcgcttgtgcgtgttatgtttctaattgtagaggcgttttgtgtcctactcttaacgaaatcgttgcttatacctacgaatggcacgtcgacgcctggggccaaggactcctggtcaccgtctcctca

SEQ ID NO: 28 - DNA, 378 bp, Artificial Sequence
  RBD F4
caggtgcagctgcgggagtcgggccccagcctggtgaagccctcacagaccctctccctcacctgcacggtctctggattctcattgagcagcaatggtgtggtctgggtccgccaggctccagggaaggcgctggagtggctcggtgatatatgcagtactggaggcacaagctttaacccagccctgaaatcccggctcagcatcgccaaggacaactccaagagccaagtctctctgtcagtgagaagcgtgacacctgaggacacggccacatattactgtgcaagaagtcgtggttatgattgttatgctaatgtggatgctttggactacgtcgatgcctggggccaaggactcctggtcaccgtctcctca

SEQ ID NO: 29 - DNA, 378 bp, Artificial Sequence
  RBD C6
caggtgcagctgcgggagtcgggccccagcctggtgaagccctcacagaccctctccctcacctgcacggtctctggattctcattgagcagcaatggtgtagtctgggtccgccaggctccagggagaccactggagtggctcggtgatatatgcagtaatggaggcacaagctttaacccagccctgaaatcccggctcagcatcgccaaggacaactccgagagccaagtctctctgaccgtgagaagcgtgacacctgaggacacagccacatattactgtgcaagaagtcgtggttatgattgttatgcttatgtttatgctttggacaccgtcgatgcctggggccaaggactcctggtcaccgtctcctca

SEQ ID NO: 30 - DNA, 378 bp, Artificial Sequence
  RBD A2
caggtgcagctgcgggagtcgggccccagcctggtgaagcccctacagatcctctccctcacctgcacggtctctggattctcattgagcagcaatggtgtggtctgggtccgccaggctccagggaaggcgctggagtggctcggtgatatatgcagtactggaggcacaagctttaacccagccctgaaatcccggctcagcatcgccaaggacaactccaagagccaagtctctctgtcagtgagaagcgtgacacctgaggacacggccacatattactgtgcaagaagtcgtggttatgattgttatgctaatgtggatgctttggactacgtcgatgcctggggccaaggactcctggtcaccgtctcctca

SEQ ID NO: 31 - DNA, 507 bp, Artificial Sequence
  SA-R2C3
caggtgcagctgcgggagtcgggccccagcctggtgaagccgtcacagaccctctcgctcacctgcacggcctctggattctcattgagcgataagcctgtaggctgggtccgccaggctccagggaagccactggagtggctcggtagtatagacactgcggaaaacacaggctataacccaggcctgaaatctcggctcagcatcaccaaggacaactccaagagccaagtctctctgtcactgagcagcgtgacgactgaggactcggccacatactactgtgctactgtacaccagaaaacgcggaaggaaaaaagttgtcctgatggctatctctatagttctaataccggccgcggttatgattgtggtgtctggacttgtcgtcgcgtcggtggtgaattctgtagtgctactggtgattggactagtcctagtgaagaagacttttacgaattctacgtcgatacgtggggccagggagccccggtcaccgtctcctca

SEQ ID NO: 32 - DNA, 501 bp, Artificial Sequence
  SA-R2D9
caggtgcagctgcgggagtcgggccccagcctggtgaagccgtcacagaccctctcgctcacctgcacggcctctggattctcattaagcgacaaggctattggctgggtccgccaggctccagggaaggcgctggagtggctcggtagtatagacacccgtggaaacacaggctataacccaggcctgaaatcccgactcagcatcaccaaggacagctccaagagccaagtctctctgtcagtgaacagcgtgacaactgaagactcggccacgtacctctgtgctattgtgcagcagatcacacacaaaacttgtcctaatggttacaattggtttgatcgttgttgttcttgggatggtacctgtggtgatggttgttgcagtaatcgtgcttggcctagtggtaatggtagagccgacagtagtattggtgaaacttatggttacgaatttcacgtggctgcctggggccaaggactcctggtcaccgtctcctca

SEQ ID NO: 33 - protein, 169 aa, Artificial Sequence
  R2G3
QVQLRESGPSLVKPSQTLSLTCAASGFSLSDKAVGWVRRAPGKALEWLGSIDTGGSTGYNPGLKSRLSITKDNSKSQVSLSISSVTSEDSATYYCATVHQKTAEGDKTCPDGYEHTCGCIGGCGCKRSACIGALCCQASLGGWLSDGETYTYEFHVDTWGQGLVVTVSS

SEQ ID NO: 34 - protein, 169 aa, Artificial Sequence
  SR3A3
QVQLRESGPSLVKPSQTLSLTCTISGFSLSSYAVLWVRQAPGKPLEWLGSIDTAENTGYNPGLKSRLSITKDNSKSQVSLSVSSVTTEDSATYYCATVHQKTRKEKNCPDGYIYSSNITSGFDCGVWICRRVGSAFCSRTGDYTSPTELDIYEFYVEGWGQGVPVTVSS

SEQ ID NO: 35 - protein, 166 aa, Artificial Sequence
  R2F12
QVQLRESGPSLVKPSQTLSLTCTVSGFSLSDKAVGWVRRAPGKALEWLGSIDTGGMTGYNPGLKSRLSITKDNSKSQVSLSVNSVTTEDSATYYCATVDQKTKNACPDDFDYRCSCIGGCGCARKGCVGPLCCRSDLGGYLTDSPAYIYEWYIDLWGQGLLVTVSS

SEQ ID NO: 36 - protein, 135 aa, Artificial Sequence
  R5A3
QVQLRESGPSLVKPSQTLSLTCTASGFSLSDKAVGWVRQAPGKALEWLGSIDTGGSTGYNPGLKSRLSITKDNSKSQVSLSVSSVTTEDSATYYCTTVHCSDGGYVEAGFGCWPWDYGYPYVDAWGQGLLVTVSS

SEQ ID NO: 37 - protein, 132 aa, Artificial Sequence
  R4G11
QVQLRESGPSLVKPSQTLSLTCTVSGFSLSSYGITWVRQAPGKALECLGSISSGGTTDYNPALKSRLSITKDNSKSQVSLSVSSVTPEDTATYYCSKWNLEYTWGGVGCASFADEDTHVDAWGQGLLVTVSS

SEQ ID NO: 38 - protein, 139 aa, Artificial Sequence
  R4G3
QVQLRESGPSLMKPSQTLSLTCTVSGFSLSDYAVGWVRQAPGKALEWLGGIDTGGSTGYNPGLESRLSITKDNSKSQVSLSVSSVTTEDSATYYCTTVVLCYFNYVVRRYNCGGLGYGHGFNSFYVDAWGQGLLVTVSS

SEQ ID NO: 39 - protein, 139 aa, Artificial Sequence
  R4E5
QVQLRESGPSLVKPSQTLSLTCTTSGFSLRNYAVGWVRQAPGKALEWLGGIDTGGSTGYNPGLESRLSITKDNSKSQVSLSVSSVTTEDSATYYCTTVVLCYFNYVVRRYNCGGLGYGHGFNSFYVDAWGQGLLVTVSS

SEQ ID NO: 40 - protein, 169 aa, Artificial Sequence
  R4C1
QVQLRESGPSLVKPSQTLSLTCTASGFSLSDKAVGWVRQAPGKPLEWLGSIDTAENTGYNPGLKSRLSITKDNSKSQVSLSVSSVTTEDSATYYCATVHQKTRKEKNCPDGYIYSSNTASGYDCGVWICRRVGSAFCSRTGDYTSPSEFDIYEFYVEGWGQGLLVTVSS

SEQ ID NO: 41 - protein, 139 aa, Artificial Sequence
  R4A10
QVQLRESGPSLVKPSQTLSLTCTTSGFSLSDYAVGWVRQAPGKALEWLGGIDTGGSTGYNPGLKSRLSITKDNSKSQVSLSVSSVTTEDSATYYCTAVVLCYYNRVVRRNNCGGLGYDYGFDHFYVDAWGQGLLVTVSS

SEQ ID NO: 42 - protein, 128 aa, Artificial Sequence
  R2G1
QVQLRESGPSLVKPSQTLSLTCTVSGFSLSNYAVGWVRQAPGKALECLGDVDSSGGTAYNPALKSRFIIAKDNSKNQVSLSVRSVTPEDTATYYCAKFAKGTTSAGACDYSESYVDAWGQGLLVTVSS

SEQ ID NO: 43 - protein, 133 aa, Artificial Sequence
  R2D6
QVQLRESGPSLVKPSQTLSLTCTTSGFSLSSYAVGWVRQAPGKALEWVGDIDYVGNTDYNPALKSRLSITKDNSKSQVSLVVSSVTAEDAATYYCAKYSGAYAYAACNYYGWRCAWESYIDAWGQGLLVTVSS

SEQ ID NO: 44 - protein, 133 aa, Artificial Sequence
  R2B1
QVQLRESGPSLVKPSQTLSLTCTVSGFSLSDNNVGWVRQAPGKALEWLGVMHNDGNKGYNPALKSRLSITKDSSKSQVSLSLSSVTSEDTATYYCTRDNARCDSWTYDSCDTWYRNSWHVDAWGQGLLVTVSS

SEQ ID NO: 45 - protein, 168 aa, Artificial Sequence
  SKM-BLV1H12
QVQLRESGPSLVKPSQTLSLTCTASGFSLSDKAVGWVRQAPGKALEWLGSIDTGGNTGYNPGLKSRLSITKDNSKSQVSLSVSSVTTEDSATYYCTTVHQETLRSCPDGYIDNSGCTADWGCAALDCWRRRFGYHSTDPSHYTGATYIYTYSLHIDAWGQGLLVTVSS

SEQ ID NO: 46 - protein, 169 aa, Artificial Sequence
  SKD-BLV1H12
QVQLRESGPSLVKPSQTLSLTCTASGFSLSDKAVGWVRQAPGKALEWLGSIDTGGNTGYNPGLKSRLSITKDNSKSQVSLSVSSVTTEDSATYYCTTVHQRTSEKRSCPGGSSRRYPSGASCDVSGGACACYVSNCRGVLCPTLNEIVAYTYEWHVDAWGQGLLVTVSS

SEQ ID NO: 47 - protein, 126 aa, Artificial Sequence
  RBD F4
QVQLRESGPSLVKPSQTLSLTCTVSGFSLSSNGVVWVRQAPGKALEWLGDICSTGGTSFNPALKSRLSIAKDNSKSQVSLSVRSVTPEDTATYYCARSRGYDCYANVDALDYVDAWGQGLLVTVSS

SEQ ID NO: 48 - protein, 126 aa, Artificial Sequence
  RBD C6
QVQLRESGPSLVKPSQTLSLTCTVSGFSLSSNGVVWVRQAPGRPLEWLGDICSNGGTSFNPALKSRLSIAKDNSESQVSLTVRSVTPEDTATYYCARSRGYDCYAYVYALDTVDAWGQGLLVTVSS

SEQ ID NO: 49 - protein, 126 aa, Artificial Sequence
  RBD A2
QVQLRESGPSLVKPLQILSLTCTVSGFSLSSNGVVWVRQAPGKALEWLGDICSTGGTSFNPALKSRLSIAKDNSKSQVSLSVRSVTPEDTATYYCARSRGYDCYANVDALDYVDAWGQGLLVTVSS

SEQ ID NO: 50 - protein, 169 aa, Artificial Sequence
  SA-R2C3
QVQLRESGPSLVKPSQTLSLTCTASGFSLSDKPVGWVRQAPGKPLEWLGSIDTAENTGYNPGLKSRLSITKDNSKSQVSLSLSSVTTEDSATYYCATVHQKTRKEKSCPDGYLYSSNTGRGYDCGVWTCRRVGGEFCSATGDWTSPSEEDFYEFYVDTWGQGAPVTVSS

SEQ ID NO: 51 - protein, 167 aa, Artificial Sequence
  SA-R2D9
QVQLRESGPSLVKPSQTLSLTCTASGFSLSDKAIGWVRQAPGKALEWLGSIDTRGNTGYNPGLKSRLSITKDSSKSQVSLSVNSVTTEDSATYLCAIVQQITHKTCPNGYNWFDRCCSWDGTCGDGCCSNRAWPSGNGRADSSIGETYGYEFHVAAWGQGLLVTVSS

SEQ ID NO: 52 - DNA, 144 bp, Artificial Sequence
  R2G3
gaaggagacaaaacgtgtcctgatggttacgagcatacttgtggttgcattgggggttgtggttgcaaaaggtctgcctgtataggtgcactttgttgccaagcgtcgttgggtggttggcttagtgacggtgaaacctacact

SEQ ID NO: 53 - DNA, 153 bp, Artificial Sequence
  SR3A3
aaagaaaaaaattgtcctgatggctatatctatagttctaatatcactagcggttttgattgtggtgtctggatttgtcgtcgcgtcggtagtgccttctgtagtcgtactggtgattatactagtcctactgaacttgacatttacgagttc

SEQ ID NO: 54 - DNA, 150 bp, Artificial Sequence
  R2F12
aaaacgaaaaatgcttgccctgatgatttcgattatcgttgttcgtgtatcggtggttgtggctgcgcccgtaaaggatgcgttggtcctctttgttgtcgttctgatttgggtggctatcttactgatagtcctgcttacatttacgaa

SEQ ID NO: 55 - DNA, 147 bp, Artificial Sequence
  R4C1
aaagaaaaaaattgtcctgatggctatatctatagttctaataccgccagcggttatgattgtggtgtctggatttgtcgtcgcgtcggtagtgccttctgtagtcgtactggtgattatactagtcctagtgaatttgacatttac

SEQ ID NO: 56 - DNA, 147 bp, Artificial Sequence
  SKM-BLV1H12
ctgcgtagttgtcctgatggttatattgataattctggatgcacggctgattggggttgtgcagctcttgattgttggcggcgtcgttttggttaccacagcactgatccttctcattatactggtgcgacgtatatttacacgtac

SEQ ID NO: 57 - DNA, 150 bp, Artificial Sequence
  SKD-BLV1H12
agcgaaaaaagaagttgtcctggcggtagtagtagacgttatcctagtggcgccagttgtgacgttagtgggggcgcttgtgcgtgttatgtttctaattgtagaggcgttttgtgtcctactcttaacgaaatcgttgcttatacctac

SEQ ID NO: 58 - DNA, 156 bp, Artificial Sequence
  SA-R2C3
cggaaggaaaaaagttgtcctgatggctatctctatagttctaataccggccgcggttatgattgtggtgtctggacttgtcgtcgcgtcggtggtgaattctgtagtgctactggtgattggactagtcctagtgaagaagacttttacgaattc

SEQ ID NO: 59 - DNA, 156 bp, Artificial Sequence
  SA-R2D9
atcacacacaaaacttgtcctaatggttacaattggtttgatcgttgttgttcttgggatggtacctgtggtgatggttgttgcagtaatcgtgcttggcctagtggtaatggtagagccgacagtagtattggtgaaacttatggttacgaattt

SEQ ID NO: 60 - protein, 51 aa, Artificial Sequence
  R2G3
EGDKTCPDGYEHTCGCIGGCGCKRSACIGALCCQASLGGWLSDGETYTYEF

SEQ ID NO: 61 - protein, 51 aa, Artificial Sequence
  SR3A3
KEKNCPDGYIYSSNITSGFDCGVWICRRVGSAFCSRTGDYTSPTELDIYEF

SEQ ID NO: 62 - protein, 50 aa, Artificial Sequence
  R2F12
KTKNACPDDFDYRCSCIGGCGCARKGCVGPLCCRSDLGGYLTDSPAYIYE

SEQ ID NO: 63 - protein, 50 aa, Artificial Sequence
  R4C1
RKEKNCPDGYIYSSNTASGYDCGVWICRRVGSAFCSRTGDYTSPSEFDIY

SEQ ID NO: 64 - protein, 49 aa, Artificial Sequence
  SKM-BLV1H12
LRSCPDGYIDNSGCTADWGCAALDCWRRRFGYHSTDPSHYTGATYIYTY

SEQ ID NO: 65 - protein, 50 aa, Artificial Sequence
  SKD-BLV1H12
SEKRSCPGGSSRRYPSGASCDVSGGACACYVSNCRGVLCPTLNEIVAYTY

SEQ ID NO: 66 - protein, 52 aa, Artificial Sequence
  SA-R2C3
RKEKSCPDGYLYSSNTGRGYDCGVWTCRRVGGEFCSATGDWTSPSEEDFYEF

SEQ ID NO: 67 - protein, 52 aa, Artificial Sequence
  SA-R2D9
ITHKTCPNGYNWFDRCCSWDGTCGDGCCSNRAWPSGNGRADSSIGETYGYEF

SEQ ID NO: 68 - protein, 75 aa, Artificial Sequence
  SKD
CTTVHQRTSEKRSCPGGSSRRYPSGASCDVSGGACACYVSNCRGVLCPTLNEIVAYTYEWHVDAWGQGLLVTVSS

SEQ ID NO: 69 - protein, 74 aa, Artificial Sequence
  SKM
CTTVHQETLRSCPDGYIDNSGCTADWGCAALDCWRRRFGYHSTDPSHYTGATYIYTYSLHIDAWGQGLLVTVSS

SEQ ID NO: 70 - protein, 75 aa, Artificial Sequence
  R4C1
CATVHQKTRKEKNCPDGYIYSSNTASGYDCGVWICRRVGSAFCSRTGDYTSPSEFDIYEFYVEGWGQGLLVTVSS

SEQ ID NO: 71 - protein, 75 aa, Artificial Sequence
  R5C1
CATVHQKTRKEKSCPDGYLYSSNTGRGYDCGVWTCRRVGGEFCSATGDWTSPSEEDFYEFYVDTWGQGLLVTVSS

SEQ ID NO: 72 - protein, 75 aa, Artificial Sequence
  SR3A3
CATVHQKTRKEKNCPDGYIYSSNITSGFDCGVWICRRVGSAFCSRTGDYTSPTELDIYEFYVEGWGQGVPVTVSS

SEQ ID NO: 73 - protein, 72 aa, Artificial Sequence
  RR2F12
CATVDQKTKNACPDDFDYRCSCIGGCGCARKGCVGPLCCRSDLGGYLTDSPAYIYEWYIDLWGQGLLVTVSS

SEQ ID NO: 74 - protein, 75 aa, Artificial Sequence
  RR2G3
CATVHQKTAEGDKTCPDGYEHTCGCIGGCGCKRSACIGALCCQASLGGWLSDGETYTYEFHVDTWGQGLVVTVSS

SEQ ID NO: 75 - protein, 69 aa, Artificial Sequence
  Germ
CTTVHQSCPDGYSYGYGCGYGYGCSGYDCYGYGGYGGYGGYGYSSYSYSYTYEYYVDAWGQGLLVTVSS

SEQ ID NO: 76 - protein, 1272 aa, Artificial Sequence
  WT Wuhan-Hu-1 S protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELKYEQYIKWPWYIWLGFIAGLIAIVMVTIMLCCMTSCCSCLKGCCSCGSCCKFDEDDSEPVLKGVKLHYT

SEQ ID NO: 77 - protein, 223 aa, Artificial Sequence
  Wuhan-Hu-1 S protein
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 78 - protein, 1229 aa, Artificial Sequence
  Wuhan-Hu-1 S protein, no furin
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELKYEQYIKWPWYIWLGFIAGLIAIVMVTI

SEQ ID NO: 79 - protein, 1288 aa, Artificial Sequence
  S-GSAS-B.1.351
MFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLLALHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGHHHHHHHHSAWSHPQFEKGGGSGGGGSGGSAWSHPQFEK

SEQ ID NO: 80 - protein, 1288 aa, Artificial Sequence
  S-GSAS-B.1.351
MFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLLALHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGHHHHHHHHSAWSHPQFEKGGGSGGGGSGGSAWSHPQFEK

SEQ ID NO: 81 - protein, 100 aa, Artificial Sequence
  IgHV1-7 V gene
QVQLRESGPSLVKPSQTLSLTCTVSGFSLSDKAVGWVRQAPGKALEWLGGIDTGGSTGYNPGLKSRLSITKDNSKSQVSLSVSSVTTEDSATYYCTTVHQ

SEQ ID NO: 82 - protein, 48 aa, Artificial Sequence
  IGHD8-2
SCPDGYSYGYGCGYGYGCSGYDCYGYGGYGGYGGYGYSSYSYSYTYEY

SEQ ID NO: 83 - protein, 15 aa, Artificial Sequence
  IGHJ2-4
YVDAWGQGLLVTVSS

SEQ ID NO: 84 - protein, 5 aa, Artificial Sequence
  CDR H1
SNGVV

SEQ ID NO: 85 - protein, 16 aa, Artificial Sequence
  CDRH2
DICSTGGTSFNPALKS

SEQ ID NO: 86 - protein, 18 aa, Artificial Sequence
  CDR H3
SRGYDCYANVDALDYVDA

SEQ ID NO: 87 - protein, 16 aa, Artificial Sequence
  CDR H2
DICSNGGTSFNPALKS

SEQ ID NO: 88 - protein, 18 aa, Artificial Sequence
  CDR H3
SRGYDCYAYVYALDTVDA

SEQ ID NO: 89 - protein, 18 aa, Artificial Sequence
  CDRH3
SRGYDCYANVDALDYVDA

SEQ ID NO: 90 - protein, 5 aa, Artificial Sequence
  CDR H1
DNNVG

SEQ ID NO: 91 - protein, 16 aa, Artificial Sequence
  CDR H2
VMHNDGNKGYNPALKS

SEQ ID NO: 92 - protein, 25 aa, Artificial Sequence
  CDR H3
DNARCDSWTYDSCDTWYRNSWHVDA

SEQ ID NO: 93 - protein, 5 aa, Artificial Sequence
  CDR H1
SYAVG

SEQ ID NO: 94 - protein, 16 aa, Artificial Sequence
  CDR H2
DIDYVGNTDYNPALKS

SEQ ID NO: 95 - protein, 25 aa, Artificial Sequence
  CDR H3
YSGAYAYAACNYYGWRCAWESYIDA

SEQ ID NO: 96 - protein, 5 aa, Artificial Sequence
  CDR H1
NYAVG

SEQ ID NO: 97 - protein, 16 aa, Artificial Sequence
  CDR H2
DVDSSGGTAYNPALKS

SEQ ID NO: 98 - protein, 20 aa, Artificial Sequence
  CDR H3
FAKGTTSAGACDYSESYVDA

SEQ ID NO: 99 - protein, 5 aa, Artificial Sequence
  CDR H1
DYAVG

SEQ ID NO: 100 - protein, 16 aa, Artificial Sequence
  CDR H2
GIDTGGSTGYNPGLKS

SEQ ID NO: 101 - protein, 31 aa, Artificial Sequence
  CDR H3
VVLCYYNRVVRRNNCGGLGYDYGFDHFYVDA

SEQ ID NO: 102 - protein, 16 aa, Artificial Sequence
  CDR H2
GIDTGGSTGYNPGLES

SEQ ID NO: 103 - protein, 31 aa, Artificial Sequence
  CDR H3
VVLCYFNYVVRRYNCGGLGYGHGFNSFYVDA

SEQ ID NO: 104 - protein, 31 aa, Artificial Sequence; identical to SEQ ID NO: 103
  CDR H3

SEQ ID NO: 105 - protein, 5 aa, Artificial Sequence
  CDR H1
SYGIT

SEQ ID NO: 106 - protein, 16 aa, Artificial Sequence
  CDR H2
SISSGGTTDYNPALKS

SEQ ID NO: 107 - protein, 24 aa, Artificial Sequence
  CDR H3
WNLEYTWGGVGCASFADEDTHVDA

SEQ ID NO: 108 - protein, 5 aa, Artificial Sequence
  CDR H1
DKAVG

SEQ ID NO: 109 - protein, 16 aa, Artificial Sequence
  CDR H2
SIDTGGSTGYNPGLKS

SEQ ID NO: 110 - protein, 11 aa, Artificial Sequence
  CDR H3
WGQGLLVTVSS

SEQ ID NO: 111 - protein, 16 aa, Artificial Sequence
  CRD H2
SIDTAENTGYNPGLKS

SEQ ID NO: 112 - protein, 63 aa, Artificial Sequence
  CDR H3
ATVHQKTRKEKNCPDGYIYSSNTASGYDCGVWICRRVGSAFCSRTGDYTSPSEFDIYEFYVEG

SEQ ID NO: 113 - protein, 5 aa, Artificial Sequence
  CDR H1
DKPVG

SEQ ID NO: 114 - protein, 63 aa, Artificial Sequence
  CDR H3
ATVHQKTRKEKSCPDGYLYSSNTGRGYDCGVWTCRRVGGEFCSATGDWTSPSEEDFYEFYVDT

SEQ ID NO: 115 - protein, 16 aa, Artificial Sequence
  CDR H2
SIDTGGNTGYNPGLKS

SEQ ID NO: 116 - protein, 63 aa, Artificial Sequence
  CDR H3
TTVHQRTSEKRSCPGGSSRRYPSGASCDVSGGACACYVSNCRGVLCPTLNEIVAYTYEWHVDA

SEQ ID NO: 117 - protein, 63 aa, Artificial Sequence
  CDR H3
TTVHQETLRSCPDGYIDNSGCTADWGCAALDCWRRRFGYHSTDPSHYTGATYIYTYSLHIDAW

SEQ ID NO: 118 - protein, 16 aa, Artificial Sequence
  CDR H2
SIDTGGSTGYNPGLKS

SEQ ID NO: 119 - protein, 63 aa, Artificial Sequence
  CDR H3
ATVHQKTAEGDKTCPDGYEHTCGCIGGCGCKRSACIGALCCQASLGGWLSDGETYTYEFHVDT

SEQ ID NO: 120 - protein, 16 aa, Artificial Sequence
  CDR H2
SIDTGGMTGYNPGLKS

SEQ ID NO: 121 - protein, 60 aa, Artificial Sequence
  CDR H3
ATVDQKTKNACPDDFDYRCSCIGGCGCARKGCVGPLCCRSDLGGYLTDSPAYIYEWYIDL

SEQ ID NO: 122 - protein, 5 aa, Artificial Sequence
  CDR H1
SYAVL

SEQ ID NO: 123 - protein, 63 aa, Artificial Sequence
  CDR H3
ATVHQKTRKEKNCPDGYIYSSNITSGFDCGVWICRRVGSAFCSRTGDYTSPTELDIYEFYVEG

SEQ ID NO: 124 - protein, 5 aa, Artificial Sequence
  CDR H1
DKAIG

SEQ ID NO: 125 - protein, 16 aa, Artificial Sequence
  CDR H2
SIDTRGNTGYNPGLKS

SEQ ID NO: 126 - protein, 61 aa, Artificial Sequence
  CDR H3
AIVQQITHKTCPNGYNWFDRCCSWDGTCGDGCCSNRAWPSGNGRADSSIGETYGYEFHVAA

SEQ ID NO: 127 - protein, 6 aa, Artificial Sequence
  Stalk A
CTTVHQ

SEQ ID NO: 128 - protein, 6 aa, Artificial Sequence
  Stalk A
CATVHQ

SEQ ID NO: 129 - protein, 6 aa, Artificial Sequence
  Stalk A
CAIVQQ

SEQ ID NO: 130 - protein, 6 aa, Artificial Sequence
  Stalk A
CATVDQ

SEQ ID NO: 131 - protein, 5 aa, Artificial Sequence
  Tyrosine motif
  VARIANT 2: Xaa = any amino acid
  VARIANT 4: Xaa = any amino acid
YXYXY

SEQ ID NO: 132 - protein, 99 aa, Artificial Sequence
  VH4-39
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCAR

SEQ ID NO: 133 - protein, 96 aa, Artificial Sequence
  4-59*03
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCA

SEQ ID NO: 134 - protein, 97 aa, Artificial Sequence
  4-34*02
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCAR

SEQ ID NO: 135 - protein, 97 aa, Artificial Sequence
  4-34*09
QVQLQESGPGLVKPSQTLSLTCAVYGGSFSGYYWSWIRQPPGKGLEWIGEINHSGSTNYNPSLKSRVTISVDTSKNQFSLKLSSVTAADTAVYYCAR

SEQ ID NO: 136 - DNA, 311 bp, Artificial Sequence
  sequence 4-39
cagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctattatagtgggagcacctactacaacccgtccctcaagagtcgagtcaccatatccgtagacacgtccaagaaccagttctccctgaagctgagctctgtgaccgccgcagacacggctgtgtattactgtgcgagacacacagtgagggg

SEQ ID NO: 137 - DNA, 288 bp, Artificial Sequence
  sequence 4-59*03
caggtgcagctgcaggagtcgggcccaggactggtgaagccttcggagaccctgtccctcacctgcactgtctctggtggctccatcagtagttactactggagctggatccggcagcccccagggaagggactggagtggattgggtatatctattacagtgggagcaccaactacaacccctccctcaagagtcgagtcaccatatcagtagacacgtccaagaaccaattctccctgaagctgagctctgtgaccgctgcggacacggccgtgtattactgtgcg

SEQ ID NO: 138 - DNA, 291 bp, Artificial Sequence
  sequence 4-34*09
caggtgcagctgcaggagtcgggcccaggactggtgaagccttcacagaccctgtccctcacctgcgctgtctatggtgggtccttcagtggttactactggagctggatccgccagcccccagggaagggactggagtggattggggaaatcaatcatagtggaagcaccaactacaacccgtccctcaagagtcgagttaccatatcagtagacacgtctaagaaccagttctccctgaagctgagctctgtgactgccgcggacacggccgtgtattactgtgcgaga

SEQ ID NO: 139 - DNA, 290 bp, Artificial Sequence
  sequence 4-34*02
caggtgcagctacaacagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgtgtattactgtgcgag

SEQ ID NO: 140 - protein, 94 aa, Artificial Sequence
  BLV1H12 V1 Region
QVQLRESGPSLVKPSQTLSLTCTASGFSLSDKAVGWVRQAPGKALEWLGSIDTGGNTGYNPGLKSRLSITKDNSKSQVSLSVSSVTTEDSATYY

SEQ ID NO: 141 - protein, 94 aa, Artificial Sequence
  Humanized V region
QVQLREWGAGLLKPSETLSLTCAVYGGSFSDKYWSWIRQPPGKGLEWIGSINHSGSTNYNPSLKSRVTISVDTSKNQFSLKLSSVTAADTAVYY

SEQ ID NO: 142 - protein, 11 aa, Artificial Sequence
  V2 alternative sequence
WGHGTAVTVSS

SEQ ID NO: 143 - protein, 11 aa, Artificial Sequence
  V2 alternative sequence
WGKGTTVTVSS

SEQ ID NO: 144 - protein, 11 aa, Artificial Sequence
  V2 alternative sequence
WGKGTTVTVSS

SEQ ID NO: 145 - protein, 11 aa, Artificial Sequence
  V2 alternative sequence
WGRGTLVTVSS

SEQ ID NO: 146 - protein, 11 aa, Artificial Sequence
  V2 alternative sequence
WGKGTTVTVSS

SEQ ID NO: 147 - protein, 11 aa, Artificial Sequence
  V2 alternative sequence
WGQGLLVTVSS

SEQ ID NO: 148 - protein, 330 aa, Artificial Sequence [sequence omitted]

SEQ ID NO: 149 - protein, 216 aa, Artificial Sequence
  BLV1H12 Light Chain
QAVLNQPSSVSGSLGQRVSITCSGSSSNVGNGYVSWYQLIPGSAPRTLIYGDTSRASGVPDRFSGSRSGNTATLTISSLQAEDEADYFCASAEDSSSNAVFGSGTTLTVLGQPKSPPSVTLFPPSTEELNGNKATLVCLISDFYPGSVTVVWKADGSTITRNVETTRASKQSNSKYAASSYLSLTSSDWKSKGSYSCEVTHEGSTVTKTVKPSECS

SEQ ID NO: 150 - protein, 269 aa, Artificial Sequence
  BLV5B8 heavy chain
QVQLRESGPSLVQPSQTLSLTCTASGFSLSDKAVGWVRQAPGKALEWLGSIDTGGSTGYNPGLKSRLSITKDNSKSQVSLSVSSVTTEDSATYYCTTVHQETRKTCSDGYIAVDSCGRGQSDGCVNDCNSCYYGWRNCRRQPAIHSYEFHVDAWGRGLLVTVSSASTTAPKVYPLSSCCGDKSSSTVTLGCLVSSYMPEPVTVTWNSGALKSGVHTFPAVLQSSGLYSLSSMVTVPGSTSGQTFTCNVAHPASSTKVDKAVEPKSCDGS

SEQ ID NO: 151 - protein, 13 aa, Artificial Sequence
  CDR L1
SGSSSNVGNGYVS

SEQ ID NO: 152 - protein, 7 aa, Artificial Sequence
  CDR L2
GDTSRAS

SEQ ID NO: 153 - protein, 11 aa, Artificial Sequence
  CDR L3
ASAEDSSSNAV

SEQ ID NO: 154 - protein, 216 aa, Artificial Sequence
  Human VL 1-51
QSVLTQPPSVSAAPGQKVTISCSGSSSNIGNNYVSWYQQLPGTAPKLLIYDNNKRPSGIPDRFSGSKSGTSATLGITGLQTGDEADYYCASAEDSSSNAVFGSGTTLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 155 - protein, 98 aa, Artificial Sequence
  VL1-47
QSVLTQPPSASGTPGQRVTISCSGSSSNIGSNYVYWYQQLPGTAPKLLIYRNNQRPSGVPDRFSGSKSGTSASLAISGLRSEDEADYYCAAWDDSLSG

SEQ ID NO: 156 - protein, 99 aa, Artificial Sequence
  VL1-40*1
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSG

SEQ ID NO: 157 - protein, 98 aa, Artificial Sequence
  VL1-51*01
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSA

SEQ ID NO: 158 - protein, 99 aa, Artificial Sequence
  VL2-18*02
QSALTQPPSVSGSPGQSVTISCTGTSSDVGSYNRVSWYQQPPGTAPKLMIYEVSNRPSGVPDRFSGSKSGNTASLTISGLQAEDEADYYCSSYTSSSTF

SEQ ID NO: 159 - protein, 53 aa, Artificial Sequence
  R4C1 knob
RKEKNCPDGYIYSSNTASGYDCGVWICRRVGSAFCSRTGDYTSPSEFDIYEFY

SEQ ID NO: 160 - protein, 43 aa, Artificial Sequence
  R4C1 knob
RKEKNCPDGYIYSSNTASGYDCGVWICRRVGSAFCSRTGDYTS

SEQ ID NO: 161 - protein, 44 aa, Artificial Sequence
  R4C1 knob
RKEKNCPDGYIYSSNTASGYDCGVWICRRVGSAFCSRTGDYTSP

SEQ ID NO: 162 - protein, 45 aa, Artificial Sequence
  R4C1 knob
RKEKNCPDGYIYSSNTASGYDCGVWICRRVGSAFCSRTGDYTSPS

SEQ ID NO: 163 - protein, 46 aa, Artificial Sequence
  R4C1 knob
RKEKNCPDGYIYSSNTASGYDCGVWICRRVGSAFCSRTGDYTSPSE

SEQ ID NO: 164 - protein, 47 aa, Artificial Sequence
  R4C1 knob
RKEKNCPDGYIYSSNTASGYDCGVWICRRVGSAFCSRTGDYTSPSEF

SEQ ID NO: 165 - protein, 48 aa, Artificial Sequence
  R4C1 knob
RKEKNCPDGYIYSSNTASGYDCGVWICRRVGSAFCSRTGDYTSPSEFD

SEQ ID NO: 166 - protein, 49 aa, Artificial Sequence
  R4C1 knob
RKEKNCPDGYIYSSNTASGYDCGVWICRRVGSAFCSRTGDYTSPSEFDI

SEQ ID NO: 167 - protein, 51 aa, Artificial Sequence
  R4C1 knob
RKEKNCPDGYIYSSNTASGYDCGVWICRRVGSAFCSRTGDYTSPSEFDIYE

SEQ ID NO: 168 - protein, 52 aa, Artificial Sequence
  R4C1 knob
RKEKNCPDGYIYSSNTASGYDCGVWICRRVGSAFCSRTGDYTSPSEFDIYEF

SEQ ID NO: 169 - protein, 43 aa, Artificial Sequence
  R2C3 knob
RKEKSCPDGYLYSSNTGRGYDCGVWTCRRVGGEFCSATGDWTS

SEQ ID NO: 170 - protein, 44 aa, Artificial Sequence
  R2C3 knob
RKEKSCPDGYLYSSNTGRGYDCGVWTCRRVGGEFCSATGDWTSP

SEQ ID NO: 171 - protein, 45 aa, Artificial Sequence
  R2C3 knob
RKEKSCPDGYLYSSNTGRGYDCGVWTCRRVGGEFCSATGDWTSPS

SEQ ID NO: 172 - protein, 46 aa, Artificial Sequence
  R2C3 knob
RKEKSCPDGYLYSSNTGRGYDCGVWTCRRVGGEFCSATGDWTSPSE

SEQ ID NO: 173 - protein, 47 aa, Artificial Sequence
  R2C3 knob
RKEKSCPDGYLYSSNTGRGYDCGVWTCRRVGGEFCSATGDWTSPSEE

SEQ ID NO: 174 - protein, 48 aa, Artificial Sequence
  R2C3 knob
RKEKSCPDGYLYSSNTGRGYDCGVWTCRRVGGEFCSATGDWTSPSEED

SEQ ID NO: 175 - protein, 49 aa, Artificial Sequence
  R2C3 knob
RKEKSCPDGYLYSSNTGRGYDCGVWTCRRVGGEFCSATGDWTSPSEEDF

SEQ ID NO: 176 - protein, 50 aa, Artificial Sequence
  R2C3 knob
RKEKSCPDGYLYSSNTGRGYDCGVWTCRRVGGEFCSATGDWTSPSEEDFY

SEQ ID NO: 177 - protein, 51 aa, Artificial Sequence
  R2C3 knob
RKEKSCPDGYLYSSNTGRGYDCGVWTCRRVGGEFCSATGDWTSPSEEDFYE

SEQ ID NO: 178 - protein, 55 aa, Artificial Sequence
  SKD knob
SEKRSCPGGSSRRYPSGASCDVSGGACACYVSNCRGVLCPTLNEIVAYTYEWHVD

SEQ ID NO: 179 - protein, 45 aa, Artificial Sequence
  SKD knob
SEKRSCPGGSSRRYPSGASCDVSGGACACYVSNCRGVLCPTLNEI

SEQ ID NO: 180 - protein, 46 aa, Artificial Sequence
  SKD knob
SEKRSCPGGSSRRYPSGASCDVSGGACACYVSNCRGVLCPTLNEIV

SEQ ID NO: 181 - protein, 47 aa, Artificial Sequence
  SKD knob
SEKRSCPGGSSRRYPSGASCDVSGGACACYVSNCRGVLCPTLNEIVA

SEQ ID NO: 182 - protein, 48 aa, Artificial Sequence
  SKD knob
SEKRSCPGGSSRRYPSGASCDVSGGACACYVSNCRGVLCPTLNEIVAY

SEQ ID NO: 183 - protein, 49 aa, Artificial Sequence
  SKD knob
SEKRSCPGGSSRRYPSGASCDVSGGACACYVSNCRGVLCPTLNEIVAYT

SEQ ID NO: 184 - protein, 51 aa, Artificial Sequence
  SKD knob
SEKRSCPGGSSRRYPSGASCDVSGGACACYVSNCRGVLCPTLNEIVAYTYE

SEQ ID NO: 185 - protein, 52 aa, Artificial Sequence
  SKD knob
SEKRSCPGGSSRRYPSGASCDVSGGACACYVSNCRGVLCPTLNEIVAYTYEW

SEQ ID NO: 186 - protein, 53 aa, Artificial Sequence
  SKD knob
SEKRSCPGGSSRRYPSGASCDVSGGACACYVSNCRGVLCPTLNEIVAYTYEWH

SEQ ID NO: 187 - protein, 54 aa, Artificial Sequence
  SKD knob
SEKRSCPGGSSRRYPSGASCDVSGGACACYVSNCRGVLCPTLNEIVAYTYEWHV

SEQ ID NO: 188 - protein, 53 aa, Artificial Sequence
  SKM knob
LRSCPDGYIDNSGCTADWGCAALDCWRRRFGYHSTDPSHYTGATYIYTYSHID

SEQ ID NO: 189 - protein, 44 aa, Artificial Sequence
  SKM knob
LRSCPDGYIDNSGCTADWGCAALDCWRRRFGYHSTDPSHYTGAT

SEQ ID NO: 190 - protein, 45 aa, Artificial Sequence
  SKM knob
LRSCPDGYIDNSGCTADWGCAALDCWRRRFGYHSTDPSHYTGATY

SEQ ID NO: 191 - protein, 46 aa, Artificial Sequence
  SKM knob
LRSCPDGYIDNSGCTADWGCAALDCWRRRFGYHSTDPSHYTGATYI

SEQ ID NO: 192 - protein, 47 aa, Artificial Sequence
  SKM knob
LRSCPDGYIDNSGCTADWGCAALDCWRRRFGYHSTDPSHYTGATYIY

SEQ ID NO: 193 - protein, 48 aa, Artificial Sequence
  SKM knob
LRSCPDGYIDNSGCTADWGCAALDCWRRRFGYHSTDPSHYTGATYIYT

SEQ ID NO: 194 - protein, 50 aa, Artificial Sequence
  SKM knob
LRSCPDGYIDNSGCTADWGCAALDCWRRRFGYHSTDPSHYTGATYIYTYS

SEQ ID NO: 195 - protein, 50 aa, Artificial Sequence; identical to SEQ ID NO: 194
  SKM knob

SEQ ID NO: 196 - protein, 51 aa, Artificial Sequence
  SKM knob
LRSCPDGYIDNSGCTADWGCAALDCWRRRFGYHSTDPSHYTGATYIYTYSH

SEQ ID NO: 197 - protein, 52 aa, Artificial Sequence
  SKM knob
LRSCPDGYIDNSGCTADWGCAALDCWRRRFGYHSTDPSHYTGATYIYTYSHI

SEQ ID NO: 198 - protein, 42 aa, Artificial Sequence
  R2G3 knob
EGDKTCPDGYEHTCGCIGGCGCKRSACIGALCCQASLGGWLS

SEQ ID NO: 199 - protein, 43 aa, Artificial Sequence
  R2G3 knob
EGDKTCPDGYEHTCGCIGGCGCKRSACIGALCCQASLGGWLSD

SEQ ID NO: 200 - protein, 44 aa, Artificial Sequence
  R2G3 knob
EGDKTCPDGYEHTCGCIGGCGCKRSACIGALCCQASLGGWLSDG

SEQ ID NO: 201 - protein, 45 aa, Artificial Sequence
  R2G3 knob
EGDKTCPDGYEHTCGCIGGCGCKRSACIGALCCQASLGGWLSDGE

SEQ ID NO: 202 - protein, 46 aa, Artificial Sequence
  R2G3 knob
EGDKTCPDGYEHTCGCIGGCGCKRSACIGALCCQASLGGWLSDGET

SEQ ID NO: 203 - protein, 47 aa, Artificial Sequence
  R2G3 knob
EGDKTCPDGYEHTCGCIGGCGCKRSACIGALCCQASLGGWLSDGETY

SEQ ID NO: 204 - protein, 48 aa, Artificial Sequence
  R2G3 knob
EGDKTCPDGYEHTCGCIGGCGCKRSACIGALCCQASLGGWLSDGETYT

SEQ ID NO: 205 - protein, 49 aa, Artificial Sequence
  R2G3 knob
EGDKTCPDGYEHTCGCIGGCGCKRSACIGALCCQASLGGWLSDGETYTY

SEQ ID NO: 206 - protein, 50 aa, Artificial Sequence
  R2G3 knob
EGDKTCPDGYEHTCGCIGGCGCKRSACIGALCCQASLGGWLSDGETYTYE

SEQ ID NO: 207 - protein, 52 aa, Artificial Sequence
  R2F12 knob
KTKNACPDDFDYRCSCIGGCGCARKGCVGPLCCRSDLGGYLTDSPAYIYEWY

SEQ ID NO: 208 - protein, 44 aa, Artificial Sequence
  R2F12 knob
KTKNACPDDFDYRCSCIGGCGCARKGCVGPLCCRSDLGGYLTDS

SEQ ID NO: 209 - protein, 45 aa, Artificial Sequence
  R2F12 knob
KTKNACPDDFDYRCSCIGGCGCARKGCVGPLCCRSDLGGYLTDSP

SEQ ID NO: 210 - protein, 46 aa, Artificial Sequence
  R2F12 knob
KTKNACPDDFDYRCSCIGGCGCARKGCVGPLCCRSDLGGYLTDSPA

SEQ ID NO: 211 - protein, 47 aa, Artificial Sequence
  R2F12 knob
KTKNACPDDFDYRCSCIGGCGCARKGCVGPLCCRSDLGGYLTDSPAY

SEQ ID NO: 212 - protein, 48 aa, Artificial Sequence
  R2F12 knob
KTKNACPDDFDYRCSCIGGCGCARKGCVGPLCCRSDLGGYLTDSPAYI

SEQ ID NO: 213 - protein, 49 aa, Artificial Sequence
  R2F12 knob
KTKNACPDDFDYRCSCIGGCGCARKGCVGPLCCRSDLGGYLTDSPAYIY

SEQ ID NO: 214 - protein, 51 aa, Artificial Sequence
  R2F12 knob
KTKNACPDDFDYRCSCIGGCGCARKGCVGPLCCRSDLGGYLTDSPAYIYEW

SEQ ID NO: 215 - protein, 42 aa, Artificial Sequence
  SR3A3 knob
KEKNCPDGYIYSSNITSGFDCGVWICRRVGSAFCSRTGDYTS

SEQ ID NO: 216 - protein, 43 aa, Artificial Sequence
  SR3A3 knob
KEKNCPDGYIYSSNITSGFDCGVWICRRVGSAFCSRTGDYTSP

SEQ ID NO: 217 - protein, 44 aa, Artificial Sequence
  SR3A3 knob
KEKNCPDGYIYSSNITSGFDCGVWICRRVGSAFCSRTGDYTSPT

SEQ ID NO: 218 - protein, 45 aa, Artificial Sequence
  SR3A3 knob
KEKNCPDGYIYSSNITSGFDCGVWICRRVGSAFCSRTGDYTSPTE

SEQ ID NO: 219 - protein, 46 aa, Artificial Sequence
  SR3A3 knob
KEKNCPDGYIYSSNITSGFDCGVWICRRVGSAFCSRTGDYTSPTEL

SEQ ID NO: 220 - protein, 47 aa, Artificial Sequence
  SR3A3 knob
KEKNCPDGYIYSSNITSGFDCGVWICRRVGSAFCSRTGDYTSPTELD

SEQ ID NO: 221 - protein, 48 aa, Artificial Sequence
  SR3A3 knob
KEKNCPDGYIYSSNITSGFDCGVWICRRVGSAFCSRTGDYTSPTELDI

SEQ ID NO: 222 - protein, 49 aa, Artificial Sequence
  SR3A3 knob
KEKNCPDGYIYSSNITSGFDCGVWICRRVGSAFCSRTGDYTSPTELDIY

SEQ ID NO: 223 - protein, 50 aa, Artificial Sequence
  SR3A3 knob
KEKNCPDGYIYSSNITSGFDCGVWICRRVGSAFCSRTGDYTSPTELDIYE

SEQ ID NO: 224 - protein, 43 aa, Artificial Sequence
  R2D9 knob
ITHKTCPNGYNWFDRCCSWDGTCGDGCCSNRAWPSGNGRADSS

SEQ ID NO: 225 - protein, 44 aa, Artificial Sequence
  R2D9 knob
ITHKTCPNGYNWFDRCCSWDGTCGDGCCSNRAWPSGNGRADSSI

SEQ ID NO: 226 - protein, 45 aa, Artificial Sequence
  R2D9 knob
ITHKTCPNGYNWFDRCCSWDGTCGDGCCSNRAWPSGNGRADSSIG

SEQ ID NO: 227 - protein, 46 aa, Artificial Sequence
  R2D9 knob
ITHKTCPNGYNWFDRCCSWDGTCGDGCCSNRAWPSGNGRADSSIGE

SEQ ID NO: 228 - protein, 47 aa, Artificial Sequence
  R2D9 knob
ITHKTCPNGYNWFDRCCSWDGTCGDGCCSNRAWPSGNGRADSSIGET

SEQ ID NO: 229 - protein, 48 aa, Artificial Sequence
  R2D9 knob
ITHKTCPNGYNWFDRCCSWDGTCGDGCCSNRAWPSGNGRADSSIGETY

SEQ ID NO: 230 - protein, 49 aa, Artificial Sequence
  R2D9 knob
ITHKTCPNGYNWFDRCCSWDGTCGDGCCSNRAWPSGNGRADSSIGETYG

SEQ ID NO: 231 - protein, 50 aa, Artificial Sequence
  R2D9 knob
ITHKTCPNGYNWFDRCCSWDGTCGDGCCSNRAWPSGNGRADSSIGETYGY

SEQ ID NO: 232 - protein, 51 aa, Artificial Sequence
  R2D9 knob
ITHKTCPNGYNWFDRCCSWDGTCGDGCCSNRAWPSGNGRADSSIGETYGYE

SEQ ID NO: 233 - protein, 1273 aa, Artificial Sequence
  Spike protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 234 - protein, 8 aa, Artificial Sequence
  Linker
GGGGAMGS

SEQ ID NO: 235 - protein, 5 aa, Artificial Sequence
  Cleavable linker
DDDDK

SEQ ID NO: 236 - protein, 46 aa, Artificial Sequence
  G3 Parental
GGSEGDKTCPDGYEHTCGCIGGCGCKRSACIGALCCQASLGGWLSD

SEQ ID NO: 237 - protein, 45 aa, Artificial Sequence
  G3 NTRUNC1
GGSGDKTCPDGYEHTCGCIGGCGCKRSACIGALCCQASLGGWLSD

SEQ ID NO: 238 - protein, 44 aa, Artificial Sequence
  G3 NTRUNC2
GGSDKTCPDGYEHTCGCIGGCGCKRSACIGALCCQASLGGWLSD

SEQ ID NO: 239 - protein, 43 aa, Artificial Sequence
  G3 NTRUNC3
GGSKTCPDGYEHTCGCIGGCGCKRSACIGALCCQASLGGWLSD

SEQ ID NO: 240 - protein, 42 aa, Artificial Sequence
  G3 NTRUNC4
GGSTCPDGYEHTCGCIGGCGCKRSACIGALCCQASLGGWLSD

SEQ ID NO: 241 - protein, 41 aa, Artificial Sequence
  G3 NTRUNC5
GGSCPDGYEHTCGCIGGCGCKRSACIGALCCQASLGGWLSD

(19) United States
(12) Patent Application Publication
(10) Pub. No.: US 2024/0262893 A1
(43) Pub. Date: Aug. 8, 2024

(54) BINDING POLYPEPTIDES AGAINST SARS COV-2 AND USES THEREOF

(71) Applicants: Applied Biomedical Science Institute, San Diego, CA (US); Kansas State University Research Foundation, Manhattan, KS (US)

(72) Inventors: Duncan MCGREGOR, San Diego, CA (US); Ruiqi HUANG, San Diego, CA (US); Gabrielle WARNER JENKINS, San Diego, CA (US); Vaughn SMIDER, San Diego, CA (US); Kyeong-Ok CHANG, Manhattan, KS (US); Waithaka MWANGI, Manhattan, KS (US)

(73) Assignees: Applied Biomedical Science Institute, San Diego, CA (US); Kansas State University Research Foundation, Manhattan, KS (US)

(21) Appl. No.: 18/560,371

(22) PCT Filed: May 11, 2022
(86) PCT No.: PCT/US2022/028863

§ 371 Date: Nov. 10, 2023

Related U.S. Application Data
(60) Provisional application No. 63/289,013, filed on Dec. 13, 2021.
(60) Provisional application No. 63/187,929, filed on May 12, 2021.

(51) Int. Cl. C07K 16/10; A61K 9/00; A61K 39/00; A61P 31/14

(52) U.S. Cl. CPC C07K 16/1003; A61K 9/0019; A61K 9/0073; A61P 31/14; A61K 2039/505; C07K 2317/24; C07K 2317/31; C07K 2317/33; C07K 2317/55; C07K 2317/565; C07K 2317/622; C07K 2317/76; C07K 2317/92

(57) ABSTRACT

Provided herein are binding polypeptides, such as binding peptides and antibodies, against a SARS CoV-2. The binding polypeptides relate to or are based on bovine antibodies, particularly those containing an ultralong CDR3. Also provided herein are methods of treating a virus infection, such as a coronavirus infection, by delivering to a subject in need a provided binding polypeptide.

### CROSS REFERENCE TO RELATED APPLICATIONS

This application claims priority to U.S. provisional application No. 63/187,929, filed May 12, 2021, entitled “Binding Polypeptides Against SARS CoV-2 and Uses Thereof”, and to U.S. provisional application No. 63/289,013, filed Dec. 13, 2021, entitled “Binding Polypeptides Against SARS CoV-2 and Uses Thereof”, the contents of which are incorporated by reference in their entirety for all purposes.

### STATEMENT OF GOVERNMENT INTEREST

This invention was made with government support under R01 GM105826 and R01 HD088400 awarded by the National Institutes of Health. The government has certain rights in the invention.

### INCORPORATION BY REFERENCE OF SEQUENCE LISTING

The present application is being filed along with a Sequence Listing in electronic format. The Sequence Listing is provided as a filed entitled “16577 2000240 SEQLIST”, created May 11, 2022, which is 184,304 bytes in size. The information in the electronic format of the Sequence Listing is incorporated by reference in its entirety.

### FIELD

The present disclosure relates to binding polypeptides, such as binding peptides and antibodies, against a SARS CoV-2. The binding polypeptides relate to or are based on bovine antibodies, particularly those containing an ultralong CDR3. The present disclosure also relates to methods of treating a virus infection, such as a coronavirus infection, by delivering to a subject in need a provided binding polypeptide.

### BACKGROUND

Coronaviruses(CoV) comprise a large group of diverse viruses belonging to the order Nidovirales, the family Coronaviridae, and the genus Coronavirus. Coronaviruses are single-stranded positive sense RNA viruses that replicate efficiently in most mammals, including humans. While infection with human coronaviruses generally results in mild disease, Severe Acute Respiratory Syndrome (SARS) CoV-2 was first isolated in 2019 and is now known as the causative agent of COVID-19.

There are currently no approved therapies specific for SARS CoV-2 on the market, therefore vaccination efforts are still considered the most effective means for controlling the ongoing pandemic. Antibodies to SARS CoV-2 as a result of natural infection or vaccination have been identified, however these are known to display sub-optimal features with respect to viral neutralization and further many candidates remain to be validated in vitro and/or in vivo. They also have limited routes of administration that may not be amenable for respiratory infections. There remains a need for improved neutralizing therapeutic molecules against SARS-CoV-2 and their use for treating or preventing viral infection.

### SUMMARY

Provided herein is an isolated knob peptide of over 30 amino acids in length directed against SARS-CoV-2, wherein the knob peptide is isolated or derived from an ultralong CDR3 of a bovine antibody.

In some of any embodiments, the provided knob peptide has an amino acid sequence length K, in which the sequence begins at position X1- and ends at X+K; and K=L−2X; and wherein L is the number of amino acids in an amino acid sequence of an antibody starting at the conserved cysteine in framework 3 and ending at the conserved tryptophan in framework 4, and X is the number of amino acids from the first cysteine in framework 3 to the first conserved cysteine encoded by the D_{H} region in CDR H3. In some embodiments, the antibody sequence is a bovine antibody. In some embodiments, the knob sequence has a sequence that is further extended by one, two, three, four, or five amino acids at the N and/or C termini.

In some of any of the provided embodiments, the knob peptide comprises a contiguous sequence of amino acids within the sequence set forth in any one of SEQ ID NOS: 112, 114, 116, 117, 119, 121, 123, or 126, wherein the knob peptide is up to 70 amino acids in length and contains 2-6 disulfide bonds.

Provided herein is a knob peptide directed against SARS-CoV-2 spike protein, comprising a contiguous sequence of amino acids of at least 40 amino acids within the sequence set forth in any one of SEQ ID NOS: 112, 114, 116, 117, 119, 121, 123, or 126, wherein the knob peptide is up to 70 amino acids in length and contains 2-6 disulfide bonds. In some of any of the provided embodiments, the knob peptide comprises 6-9 C-terminal amino acids after the last cysteine (Cys) residue of the respective sequence of amino acids set forth in any one of SEQ ID NOS: 112, 114, 116, 117, 119, 121, 123, or 126, optionally at or about 9 C-terminal amino acids after the last cysteine (Cys) residue of the respective sequence of amino acids set forth in any one of SEQ ID NOS: 112, 114, 116, 117, 119, 121, 123, or 126. In some of any of the provided embodiments, the sequence of the knob peptide begins 3-6 amino acids before the N-terminal Cys residue of the respective sequence of amino acids set forth in any one of SEQ ID NOS: 112, 114, 116, 117, 119, 121, 123, or 126. In some of any of the provided embodiments, the knob peptide comprises at or about 9 C-terminal amino acids after the last cysteine (Cys) residue of the respective sequence of amino acids set forth in any one of SEQ ID NOS: 112, 114, 116, 117, 119, 121, 123, or 126.

In some of any of the provided embodiments, the knob peptide is 40 to 60 amino acids in length. In some embodiments, the knob peptide is at least 42 amino acids in length. In some of any of the provided embodiments, the knob peptide is 42 amino acids, 43 amino acids, 44 amino acids, 43 amino acids, 46 amino acids, 47 amino acids, 48 amino acids, 49 amino acids, 50 amino acids, 51 amino acids, 52 amino acids, 33 amino acids, 54 amino acids, 55 amino acids, 56 amino acids, 57 amino acids, 58 amino acids, 59 amino acids or 60 amino acids in length.

In some of any of the provided embodiments, the knob peptide comprises at least 4 Cys residues. In some embodiments, the knob peptide contains 4 Cys residues. In some of any of the provided embodiments, the knob peptide contains 6, 8, 10 or 12 Cys residues. In some of any of the provided embodiments, the knob peptide has at least 2 disulfide bonds. In some of any of the provided embodiments, the knob peptide has 2 disulfide bonds. In some of any of the provided embodiments, the knob peptide has 3, 4 or 5 disulfide bonds.

In some of any of the provided embodiments, the knob peptide comprises an amino acid sequence set forth in any one of SEQ ID NOS: 160, 169, 179, 189, 198, 208, 215 or 224. In some of any of the provided embodiments, the knob peptide is at or about 42 amino acids, 43 amino acids, 44 amino acids, 45 amino acids, 46 amino acids, 47 amino acids, 48 amino acids, 49 amino acids, 50 amino acids, 51 amino acids, 52 amino acids, 53 amino acids, 54 amino acids, or 55 amino acids. In some of any of the provided embodiments, the knob peptide comprises the amino acid sequence set forth in SEQ ID NO: 160. In some of any of the provided embodiments, the knob peptide is set forth in any one of SEQ ID NOS: 63 or 159-168. In some of any of the provided embodiments, the knob peptide comprises the amino acid sequence set forth in SEQ ID NO: 169. In some of any of the provided embodiments, the knob peptide is set forth in any one of SEQ ID NOS: 66 or 169-177. In some of any of the provided embodiments, the knob peptide comprises the amino acid sequence set forth in SEQ ID NO:179. In some of any of the provided embodiments, the knob peptide is set forth in any one of SEQ ID NOS: 65 or 178-187. In some of any of the provided embodiments, the knob peptide comprises the amino acid sequence set forth in SEQ ID NO:189. In some of any of the provided embodiments, the knob peptide is set forth in any one of SEQ ID NOS: 64 or 188-197. In some of any of the provided embodiments, the knob peptide comprise the amino acid sequence set forth in SEQ ID NO:198. In some of any of the provided embodiments, the knob peptide is set forth in any one of SEQ ID NOS: 60 or 198-206. In some of any of the provided embodiments, the knob peptide comprises the amino acid sequence set forth in SEQ U) NO:208. In some of any of the provided embodiments, the knob peptide is set forth in any one of SEQ ID NOS: 62 or 207-214. In some of any of the provided embodiments, the knob peptide comprises the amino acid sequence set forth in SEQ ID NO:215. In some of any of the provided embodiments, the knob peptide is set forth in any of SEQ ID NOS: 61 and 215-223. In some of any of the provided embodiments, the knob peptide comprises the amino acid sequence set forth in SEQ ID NO:224. In some of any of the provided embodiments, the knob peptide is set forth in any one of SEQ ID NOS: 68 or 224-232.

In some of any of the provided embodiments, the knob peptide further comprises an N-terminal or C-terminal linker. In some embodiments, the linker is a GS linker. In some of any of the provided embodiments, the knob peptide further comprises a linker for cyclization of the peptide. In some of any of the provided embodiments, the linker is GGGGAMGS (SEQ ID NO: 234). In some of any of the provided embodiments, the peptide is cyclized.

In some of any of the provided embodiments, the knob peptide binds to the spike protein of SARS-CoV2 or a binding portion or epitope thereof with a binding affinity dissociation constant of less than at or about 10⁻⁹ M, less than at or about 10⁻¹⁰ M, less than at or about 10⁻¹¹ M or less than at or about 10⁻¹² M. In some of any of the provided embodiments, the knob peptide neutralizes SARS-CoV2 infection of a human cell. In some of any of the provided embodiments, the knob peptide neutralizes SARS-CoV2 with an EC50 of less than about 100 ng/mL, 10 ng/mL, 1 ng/mL, 0.1 ng/mL, 0.01 ng/mL, 0.001 ng/mL. In some of any of the provided embodiments, the knob peptide neutralizes SARS-COV2 with an EC50 of less than about 100 nM, 10 nM, 1 nM, 0.1 nM, 0.01 nM or 0.001 nM In some of any of the provided embodiments, the knob peptide neutralizes SARS-COV2 with an EC50 of less than at or about 500 pM, 250 pM, 100 pM, 50 pM, 25 pM, 10 pM, 5 pM, 2.5 pM, 1 pM, 0.5 pM or less.

In some of any of the provided embodiments, the SARS CoV-2 is selected from Wuhan-Hu-1 strain, UK (B.1.1.7) strain or South African (B.1.351) strain. In some of any of the provided embodiments, the SARS CoV-2 is Wuhan-Hu-1 strain. In some of any of the provided embodiments, the SARS CoV-2 is Wuhan-Hu-1 strain.

In some of any of the provided embodiments, the knob peptide is recombinantly produced. In some of any of the provided embodiments, the knob peptide is purified.

Provided herein is a multispecific binding protein, comprising a plurality of knob peptides of any of the provided. In some of any of the provided embodiments, the plurality of knob peptides are paratopes. In some of any of the provided embodiments, the plurality of knob peptides are 2, 3, or 4 peptides.

Provided herein is a composition comprising any of the provided knob peptides. Provided herein is a composition comprising a plurality of any of the provided knob peptides. In some of any of the provided embodiments, the plurality of knob peptides are paratopes. In some of any of the provided embodiments, the plurality of knob peptides are 2, 3 or 4 peptides.

In some of any of the provided embodiments, any of the provided compositions is pharmaceutical composition. In some embodiments, the pharmaceutical composition comprises a pharmaceutical carrier. Provided herein are pharmaceutical compositions comprising any of the provided knob peptides and a pharmaceutical carrier. Also provided herein are pharmaceutical compositions comprising a plurality of any of the provided knob peptides and a pharmaceutical carrier. In some of any of the provided embodiments, the composition is formulated for inhalation.

Provided herein is an antibody or antigen binding fragment directed against the spike protein of SARS-CoV2, wherein the antibody or antigen binding fragment comprises a heavy chain variable region (VH) and a light chain variable region (VL), wherein the VH comprises a CDR3 H3 set forth in any one of SEQ D NOS: 86, 88, 89, 92, 95, 98, 101, 103, 104, 107, 110, 112, 114, 116, 117, 119, 121, 123, or 126. In some of any of the provided embodiments, the VH further comprises a CDR-H1 set forth in any one of SEQ ID NOS: 84, 90, 93, 96, 99, 105, 108, 113, 122 or 124 and a CDR-H2 set forth in any one of SEQ ID NOS: 85, 87, 91, 94, 97, 100, 102, 106, 109, 111, 115, 118, 120 or 125.

In some of any of the provided embodiments, the VH comprises: (a) a CDR-H1, a CDR-H2 and CDR-H3 set forth in SEQ ID NOS: 84, 85 and 86, respectively; (b) a CDR-H1, a CDR-H2 and CDR-13 set forth in SEQ ID NOS: 84, 87 and 88, respectively; (c) a CDR-H1, a CDR-H2 and CDR-13 set forth in SEQ ID NOS: 84, 85 and 89, respectively; (d) a CDR-H1, a CDR-H2 and CDR-13 set forth in SEQ ID NOS: 90, 91 and 92, respectively; (e) a CDR-H1, a CDR-H2 and CDR-H3 set forth in SEQ ID NOS: 93, 94 and 95, respectively; (f) a CDR-H1, a CDR-H2 and CDR-H3 set forth in SEQ ID NOS: 96, 97 and 98, respectively; (g) a CDR-H1, a CDR-H2 and CDR-H3 set forth in SEQ ID NOS: 99, 100 and 101, respectively; (h) a CDR-H1, a CDR-H2 and CDR-H3 set forth in SEQ ID NOS: 96, 102 and 103, respectively; (i) a CDR-H1, a CDR-H2 and CDR-H3 set forth in SEQ ID NOS: 99, 102 and 104, respectively; (j) a CDR-H1, a CDR-H2 and CDR-H3 set forth in SEQ ID NOS: 105, 106 and 107, respectively; (k) a CDR-H1, a CDR-H2 and CDR-H3 set forth in SEQ ID NOS: 108, 109 and 110, respectively; (l) a CDR-H1, a CDR-H2 and CDR-H3 set forth in SEQ ID NOS: 108, 111 and 112, respectively; (n) a CDR-H1, a CDR-H2 and CDR-H3 set forth in SEQ ID NOS: 113, 111 and 114, respectively; (n) a CDR-H1, a CDR-H2 and CDR-H3 set forth in SEQ ID NOS: 108, 115 and 116, respectively; (o) a CDR-H1, a CDR-H2 and CDR-H3 set forth in SEQ ID NOS: 108, 115 and 117, respectively; (p) a CDR-H1, a CDR-H2 and CDR-H3 set forth in SEQ ID NOS: 108, 118 and 119, respectively; (q) a CDR-H1, a CDR-H2 and CDR-H3 set forth in SEQ ID NOS: 108, 120 and 121, respectively; (r) a CDR-H1, a CDR-H2 and CDR-H3 set forth in SEQ ID NOS: 122, 111 and 123, respectively; or(s) a CDR-H1, a CDR-H2 and CDR-H3 set forth in SEQ ID NOS: 124, 125 and 126, respectively.

In some of any of the provided embodiments, the VH comprises the sequence set forth in any one of SEQ ID NOS: 33, 34, 35, 40, 45, 46, 50 or 51. In some of any of the provided embodiments, the VH is humanized. In some of any of the provided embodiments, the VH has the formula V1-X-V2, wherein the V1 region is a humanized variant of the V region of a bovine antibody, optionally wherein the bovine antibody is BLV1H12, the X is the CDR3 and X2 is a portion of the heavy chain including FR-4 of a human J gene segment or a humanized bovine J gene. In some of any of the provided embodiments, the V1 is set forth in SEQ ID NO:141, X is set forth in any one of SEQ ID NOS: 112, 114, 116, 117, 119, 121, 123, or 126, and V2 is set forth in any of SEQ ID NOS: 142-147.

In some of any of the provided embodiments, the VH further comprises a constant region. In some of any of the provided embodiments, the constant region is a constant region of an immunoglobulin IgG1.

In some of any of the provided embodiments, the VL is a bovine light chain variable region or is a humanized variant thereof. In some of any of the provided embodiments, the VL is a light chain variable region of BLVH12, BLV5D3, BLVSC11, BFIH1, BLV5B8 or F18. In some of any of the provided embodiments, the VL comprises a CDR-L1, CDR-L2 and CDR-L3 set forth in SEQ ID NO: 151, 152 and 153, respectively. In some of any of the provided embodiments, the VL is set forth in SEQ ID NO:2. In some of any of the provided embodiments, the VL is a humanized light chain. In some of any of the provided embodiments, the VL is a human light chain variable region. In some of any of the provided embodiments, the VL is a light chain variable region of VL1-47, VL1-40, VL1-51, and VL2-18, or a variant thereof comprising substitution of amino acids to residues at corresponding positions in a bovine light chain sequence.

In some of any of the provided embodiments, the amino acid substitutions are selected from S2A, T5N, P8S, A12G, A13S, and P14L and/or substitution of DNN to GDT, optionally substitution DNNKRP to GDTSRA. In some of any of the provided embodiments, the VL comprises the sequence set forth in any of SEQ ID NOS: 155-158.

In some of any of the provided embodiments, the light chain further comprises a constant region. In some of any of the provided embodiments, the light chain is set forth in SEQ ID NO: 154.

In some of any of the provided embodiments, the antibody or antigen binding fragment is a full length antibody. In some of any of the provided embodiments, an antigen binding fragment selected from a Fab or a single chain variable fragment (scFv). In some of any of the provided embodiments, the antibody or antigen binding fragment is isolated or recombinant. In some of any of the provided embodiments, the antibody or antigen binding fragment is a monoclonal antibody.

Provided herein is a composition comprising any of the provided antibody or antigen binding fragment. In some of any of the provided embodiments, the composition is a pharmaceutical composition. In some embodiments, the composition comprises a pharmaceutical carrier. Provided herein is a pharmaceutical composition comprising any of the provided antibody or antigen binding fragment and a pharmaceutical carrier.

Provided herein are polynucleotides encoding any of the provided knob peptides. Also provided herein are polynucleotides encoding any of the provided multispecific polypeptides. Provided herein are polynucleotides encoding any of the provided antibody or antigen binding fragments.

In some of any of the provided embodiments, the provided polynucleotide is a synthetic nucleic acid. In some of any of the provided embodiments, the provided polynucleotide is cDNA.

Provided herein is a vector, comprising any of the provided polynucleotides. In some of any of the provided embodiments, the vector is an expression vector.

Provided herein is a method of treating a coronavirus infection in a subject in need, the method comprising administering to a subject any of the provided knob peptide. Provided herein is a method of treating a coronavirus infection in a subject in need, the method comprising administering to a subject. Provided herein is a method of treating a coronavirus infection in a subject in need, the method comprising administering to a subject any of the provided the multispecific polypeptide or any of the provided antibody or antigen binding fragment.

Provided herein is a method of prophylactically treating a subject at risk for developing a coronavirus infection, the method comprising administering to a subject any of the provided knob peptide. Provided herein is a method of prophylactically treating a subject at risk for developing a coronavirus infection, the method comprising administering to a subject any of the provided the multispecific polypeptide. Provided herein is a method of prophylactically treating a subject at risk for developing a coronavirus infection, the method comprising administering to a subject any of the provided antibody or antigen binding fragment.

Provided herein is a method of preventing a coronavirus in a subject at risk for developing a coronavirus infection, the method comprising administering to a subject any of the provided knob peptide. Provided herein is a method of preventing a coronavirus in aa subject at risk for developing a coronavirus infection, the method comprising administering to a subject any of the provided knob peptide any of the provided the multispecific polypeptide. Provided herein is a method of preventing a coronavirus in aa subject at risk for developing a coronavirus infection, the method comprising administering to a subject any of the provided knob peptide any of the provided antibody or antigen binding fragment.

Provided herein is use of any of the provided knob peptides in the manufacture of a medicament for use in treating a coronavirus infection in a subject in need. Provided herein is use of any of the provided multispecific polypeptides in the manufacture of a medicament for treating a coronavirus infection in a subject in need. Provided herein is use of any of the provided antibodies or antigen binding fragments in the manufacture of a medicament for treating a coronavirus infection in a subject in need.

Provided herein is a pharmaceutical composition comprising any of the provided knob peptides for use in treating a coronavirus infection in a subject in need. Provided herein is a pharmaceutical composition comprising any of the provided multispecific polypeptides for use in treating a coronavirus infection in a subject in need. Provided herein is a pharmaceutical composition comprising any of the provided antibodies or antigen binding fragments for use in treating a coronavirus infection in a subject in need.

In some of any embodiments, the use or the pharmaceutical composition for use for use in administering any of the knob peptides, muiltispecific polypeptides or antibodies or antigen binding fragments in accord with any of the provided methods for administering the same to a subject.

In some embodiments, the subject is a subject that is known or suspected of having been infected with coronavirus. In some embodiments, the use can be for prophylactic treatment of a subject at risk for developing coronavirus infection.

In some of any of the provided embodiments, the coronavirus infection is caused by SARS-CoV, SARS-CoV-2, or MERS-CoV. In some of any of the provided embodiments, the coronavirus infection is COVID-19.

In some of any of the provided embodiments, the administration is a parenteral administration. In some of any of the provided embodiments, the administration is a subcutaneous or intravenous administration. In some of any of the provided embodiments, the administration is by inhalation. In some of any of the provided embodiments, the administration is by use of a nebulizer, inhaler, atomizer, aerosolizser, mister, dry powdered inhaler, metered dose inhaler, metered dose sprayer, metered dose mister, or metered dose atomizer.

### BRIEF DESCRIPTION OF THE DRAWINGS

FIG. 1 depicts a schematic of an exemplary Ultralong CDR3 cow antibody, including the “knob” peptide having a size between 4 and 6 KDa.

FIG. 2A depicts binding of immunized calf serum against the RBD domain of the SARS CoV-2 S protein via ELISA. Neutralization activity of the sera IgG against SARS-CoV-2 pseudovirus is shown in FIG. 2B.

FIG. 3A depicts the pill phage fusion constructs in each display library (i.e., scFv and “knob” display).

FIG. 3B displays a schematic of pTAU1 phage vector multiple cloning site, used for direct cloning of bovine CDR3 knob DNA fragments as NeoI-NotI fragments. A schematic of pTAU1-BLV1H12(-VH) phage scFv vector multiple cloning site used for cloning of bovine VH DNA fragments as NcoI-XhoI fragments in-frame with BLV1H12 V-lambda DNA is shown in FIG. 3C. FIG. 3D depicts the separation between Ultralong VH fragments and shorter VH fragments without the Ultralong CDR3 region on an agarose gel.

Sequences alignments for exemplary Ultralong antibodies R2C1 (SKD), R2C3 (SKM), R4C1, R5C1, SR3A3, RR2F12, and RR2G3 are shown in FIG. 4.

FIG. 5A depicts binding of exemplary chimeric bovine-human IgG1 antibodies to spike protein, binding to the RBD is also shown in FIG. 5B. FIG. 5C shows ELISA binding of TgG antibodies to recombinant stabilized spike proteins derived from several SARS CoV strains. 5D shows ELISA binding curves of select IgG antibodies against the omicron variant RBD (left) or recombinant stabilized spike trimer (right).

FIG. 5E reflects exemplary ELISA data of R4C1 and R2D9 on SARS-CoV-2 compared to SARS-CoV-1 FIG. 5F shows ELISA binding activity (top) for three different exemplary antibody knob candidates against WT (Wuhan) SARS CoV-2 spike protein. FIG. 5G depicts a modified western blot using SDS and detected with biotinylated RBD.

FIG. 6A displays a schematic of the pET32b vector cloning site used for trxA-CDR3-knob fusion and CDR3-knob expression. A schematic of purification process from bacterial lysate is shown in FIG. 6B. FIG. 6C depicts CDR3-knob SDS-PAGE showing efficient purification of soluble CDR3-KNOB from E. coli lysate. FIG. 6D depicts an exemplary SDS-PAGE gel of several purified ultralong CDR H3 knob peptides

FIG. 7A shows the results of a Wuhan-Hu-1 spike protein capture ELISA, using serial dilutions of IMAC purified trxA-fusions. Binding for the TrxA-R2G3 fusion protein is also shown in FIG. 7B.

FIG. 8A depicts a background-subtracted ELISA of soluble biotinylated RBD binding to exemplary purified R2-G3 CDR3-knob. Soluble R2G3 knob binding relative to a reference anti-spike antibody (CR3022) is shown in FIG. 8B.

Amino acid sequences of exemplary truncated R2G3 mutants are shown in FIG. 8C. FIG. 8D. depicts a SDS-PAGE of R2G3 truncations after bacterial expression and purification. Results of an ELISA binding of biotinylated RBD by coated CDR3-knob truncation as shown in FIG. 8E.

FIG. 9A depicts a size exclusion chromatograph for purified R4C1 knobs. A gel electrophoresis gel of two fractions (A4 and A7) are shown in FIG. 9B.

FIG. 9C depicts a size exclusion chromatograph for purified R2G3 knobs. A gel electrophoresis gel of a fraction (A6) are shown in FIG. 9D.

Results of a pseudoviral luciferase assay are shown in FIG. 10 for four exemplary Ultralong CDR3 antibodies (F12, G3, SKD, and SKM) against wild-type (FIG. 10A), “UK” variant (FIG. 10B), “484K” variant (FIG. 10C), and “SA” variant (FIG. 10D) SARS CoV-2 spike protein expressing viruses.

FIG. 11A shows the IC50 values of different IgG antibodies against pseudoviruses from various coronavirus strains. FIG. 11B shows a comparison of the R2G3 IgG, Fab, and knob in neutralization of wild-type SARS-CoV-2 pseudovirus.

FIG. 12 is a depiction of multispecific knob peptide compositions and formats. A plurality of paratope knob peptides can be attached to an immunoglobulin, including as a homodimer or heterodimer, to provide a multispecific binding polypeptide. A plurality of paratope knob peptides also may be linked directly in tandem, such as via a linker. A plurality of knob peptides also may be combined as a mixture or cocktail to provide a combined polyclonal composition.

FIG. 13A depicts the crystal structure of BLVIH12 Fab (PDB 4k3d), an enlarged view of stalk and knob region, with framework 3 cysteine, knob position 1 cysteines, and the framework 4 tryptophan side chains is shown in FIG. 13B.

A sequence alignment of the stalk and knob regions for 12 exemplary antibodies is shown in FIG. 14, the knob regions are flanked by the ascending and descending stalk regions which are shown with white letters highlighted in black.

FIG. 15 is a schematic representation of the stalk and knob domain (L), containing the CDR H3 plus three residues on the N-terminal end.

Binding of biotinylated RBD by coated CDR3-knob truncations as assessed via ELISA are shown in FIG. 16A. An exemplary SDS-PAGE of R2G3 truncations after bacterial expression and purification is shown in FIG. 16B.

FIG. 17A shows ELISA binding of biotinylated RBD by coated CDR3-knob N-terminal truncations, and an exemplary SDS-PAGE of R2G3 N-terminal truncations after bacterial expression and purification is shown in FIG. 17B

### DETAILED DESCRIPTION

Provided herein are binding polypeptides, including bovine-derived antibodies or antigen-binding fragments and knob peptides, directed against a coronavirus, such as a Severe Acute Respiratory Syndrome (SARS) virus, including SARS CoV-2. The antibodies or antigen-binding fragments include bovine or bovine-derived antibodies or antigen binding fragments. In particular, among the provided antibodies and antigen binding fragments are those having an ultralong CDR-H3 in the variable heavy chain. Also provided are unique knob peptides based from or derived from such ultralong CDR-H3. The provided binding polypeptides, including the knob peptides are found to exhibit picomolar binding to the spike (s) protein of SARS-CoV2 and neutralize SARS-CoV2. In some embodiments, provided binding polypeptides are broadly neutralizing antibodies.

Coronaviruses are single-stranded positive sense RNA viruses belongs to the order Nidovirales, the family Coronaviridae, and the genus Coronavirus. The taxonomic classification further divides coronaviruses into three genera: alpha, beta, and gamma; of which the novel SARS CoV-2 that is the causative agent of COVID-19 belongs to the group beta-coronavirus.

Coronaviruses are enveloped with an observed diameter range from 60-220 nm. Coronaviruses are a diverse group of viruses that can be found among a wide range of mammals including humans and domestic animals, and are generally associated with mild infection. Among the coronaviruses associated with significant human disease are SARS CoV-1, SARS CoV-2 (also known as the novel coronavirus, or 2019-nCoV), and Middle East Respiratory Syndrome (MERS). As of 2021, there have also been identified several Variant of Concerns, or VOCs, representing significant alterations to the antigenic S protein. Table 1 provides a description of exemplary known variants.

TABLE 1
SARS-CoV-2 Variants

| Name/Designation | | Distribution | Notable μ | Impact | Sequence |
|---|---|---|---|---|---|
| D614G | | Worldwide | D614G | Increased infectivity, | P0DTC2 |
| | | | | Dominant circulating | |
| | | | | since June 2020 | |
| B.1.1.7 | 501Y.V1 | UK/Worldwide | D614G, | Increased | B.1.1.7 |
| | | (nearly dominant | N501Y, | infectivity | Lineages |
| | | in US) | P681H | | |
| B.1.351 | 501.V2, or | South | N501Y, | Increased | B.1.351 |
| | N501Y.V2 | Africa | E484K*, | infectivity, | Lineages |
| | | | K417N | *escape | |
| | | | | mutation* | |
| B.1.1.248 | P1 | Brazil | N501Y, | Increased | P1 |
| | | | E484K*, | infectivity, | Lineages |
| | | | K417T | *escape | |
| | | | | mutation* | |

SARS CoV-2 is primarily transmitted through human-to-human contact via respiratory droplets or mucosal contact, but is now also demonstrated to efficiently transmit through aerosolized droplets (i.e., airborne transmission). The basic regeneration number (Ro) is estimated to be roughly 2.2 (with a 90% high density interval range of 1.4-3.8). The latency period is generally between 3 and 14 days, with some reports of as many as 24 days prior to symptomatic presentation. The most current data supports that latent individuals are capable of transmission virus and disease, despite lack of traditional indications of respiratory distress. The most common symptoms associated with clinical infection are non-specific and include “flu-like” indications of fever, cough, myalgia, malaise, and fatigue.

As of May 2021, roughly 33 million Americas were confirmed to have been infected with SARS CoV-2, including almost 600,000 deaths as a result of the novel virus. While research is ongoing to determine underlying mechanisms regarding disease severity, SARS CoV-2 is a virus to which no humans prior to 2019 had existing immunity. As a result, individuals with competent and robust immune systems are also susceptible to negative disease outcomes, including pneumonia that can become life threatening. Additionally associated with infection is acute respiratory distress syndrome (ARDS), cytokine dysregulation resulting in a cytokine storm, toxic and septic shock, metabolic acidosis, and blood coagulation/clotting dysregulation. In individuals with and without underlying health conditions, symptoms have also been observed in patients previously infected by currently COVID negative via molecular based assay. These symptoms can cause significant disruption to quality of life, including lasting viral sequelae sometimes known as “post COVID syndrome” or “long COVID”.

Detection of SARS CoV-2 infection is predominately carried out via molecular assay for virus-specific gene sequences or a serological assay to assess a patient's antibodies to virus-specific protein. Molecular assays are generally based on PCR or nucleic acid sequencing of conserved viral genes, such as the E gene or the gene encoding the RNA-dependent RNA polymerase (RdRp) in SARS CoV-2. Serological tests, also known as antigen tests, have been designed to assay for specific patient antibodies directed to SARS CoV-2 antigen (such as S protein antigen) in order to determine whether a person has been infected, and roughly the timing of that infection.

At present there is no specific pharmacological treatment for COVID. Methods of mitigating SARS CoV-2 were limited to a series of measures such as isolation, personal protective equipment, limitation of travel, work and school stoppages etc. in order to limit movement and spread of virus. Therefore, vaccination efforts are considered the most effective means of controlling pandemic virus and immunity at the population scale. Vaccine development around the world has yielded more than 115 candidates, including those from Pfizer, BioNTech/NIH, Astra Zeneca, and Johnson and Johnson.

Antibodies targeting SARS CoV-2 have been identified in response to infection or immunization with SARS CoV-2 (or antigen thereof) and characterized. The majority of these are directed to the spike, or S protein, which is displayed on the viral surface. The S protein is responsible for mediating interaction between the virus and host cell at its cellular receptor, ultimately resulting in internalization. The cellular receptor with which the S protein is known to interact is ACE-2, which is expressed in cells of the upper respiratory track but also throughout the body. The SARS-CoV-2-Spike protein is a 1273 amino acid type I membrane glycoprotein which assembles into trimers that constitute the spikes or peplomers on the surface of the enveloped coronavirus particle. The protein has two essential functions, host receptor binding and membrane fusion, which are attributed to the N-terminal (S1) and C-terminal (S2) halves of the S protein. CoV-S binds to its cognate receptor via a receptor binding domain (RBD) present in the S1 subunit. The amino acid sequence of full-length SARS-CoV-2 spike protein is exemplified by the amino acid sequence provided in SEQ ID NO: 233.

Unfortunately antibodies against SARS CoV-2 thus far characterized display sub-optimal features with respect to viral neutralization (i.e., IC₅₀, protective concentrations etc.). Further, testing of live SARS CoV-2 virus requires dedicated BSL-3 facilities for both in vitro and also in vivo lab work; making validation of these antibodies extremely challenging. While binding ELISAs and work with S protein pseudotyped virus are critical data points, neutralization of live SARS CoV-2 virus represents a standard often not met by current antibodies of the art. There is a need for neutralizing therapeutic anti-SARS-CoV-2-Spike protein (SARS-CoV-2-S) antibodies and their use for treating or preventing viral infection.

Provided binding polypeptide, including antibodies or antigen binding fragments and knob peptides, address one or more of these needs and provide improved binding polypeptides directed to SARS CoV-2. In some embodiments, the binding polypeptides, including antibodies or antigen binding fragments and knob peptides, bind with high affinity to SARS-CoV-2 spike protein. Because this virus uses its spike glycoprotein for interaction with the cellular receptor ACE2 and the serine protease TMPRSS2 for entry into a target cell, this spike protein represents an attractive target for antibody therapeutics. An anti-SARS-CoV-2-Spike protein binding polypeptide with high affinity and that inhibits virus infectivity could be important in the prevention and treatment of COVID-19 and other infections caused by coronaviruses.

The present disclosure provides neutralizing human antigen-binding proteins that specifically bind to SARS-CoV-2-Spike protein, for example, antibodies or antigen-binding fragments thereof or knob peptides. In one aspect, the present disclosure provides an isolated recombinant antibody or antigen-binding fragment thereof that specifically binds to a coronavirus spike protein (CoV-S), wherein the antibody binds to CoV Spike proteins with an EC50 of less than about 10-9 M. In particular embodiments, the provided binding polypeptides, including antibodies or antigen binding fragments and knob peptides, exhibit subnanomolar binding affinity such as picomolar binding affinity.

In particular, among the provided embodiments are ultralong CDR3 antibodies derived from bovine that exhibit picomolar potency for neutralizing SARS-CoV2. From an immunization and screening strategy of bovine antibodies, observations provided herein demonstrate that ultralong CDR3 antibodies were the most potent of all the identified antibodies. Further, the picomolar potency is observed not only for parental Wuhan-Hu-1 strain but also for common variants such as the UK (B.1.1.7) and South African (B.1.351) variants. Results also indicate potency against other variants, such as Delta and Omicron SARS CoV-2 viruses. This is in contrast to many published antibodies neutralizing monoclonal antibodies against SAR-CoV2 that exhibit far lower neutralizing potency (Yu et al. 2020 Signal Transduction, 5:212).

Available methods of analysis and exploitation of the unique ultralong CDR H3 structure are not entirely satisfactory. In many cases, methods require excision and purification of the isolated knob domain (Macpherson et al. 2020 PLOS Biology, 18(9): e30000821). Such methods are not easily amenable to good manufacturing practices for generating therapeutic molecules and also are inefficient in terms of the amount of knob protein that can be produced. Further the use of enzymes for excision of the knob may also compromise the integrity of the isolated protein.

Remarkably, it is found herein that a disulfide bonded knob peptide derived from the ultralong CDR-H3 of the identified antibodies can be independently expressed and produced as an independent binding unit and retains picomolar binding affinity and neutralizing activity against SARS-CoV2. This knob peptide is only roughly 4-5 kDa in size, e.g. about 4.4 kDa, and represents the smallest independent antigen binding domain. It exhibits high affinity and epitope coverage, similar to a larger antibody. Its small size approaches the size of small molecules and thereby opens up the utility of the antigen binding domain as a new and novel therapeutic. For instance, its small size allows for better tissue penetration and also permits alveolar delivery. Further, the provided knob peptides are stable by virtue of their rigid disulfide-bonded small domain. This stable structure avoids aggregates seen in nanobodies and other immunoglobulin domain-based fragments. Findings also demonstrate that it can be produced in high yield in E. coli making it highly developable as a therapeutic molecule.

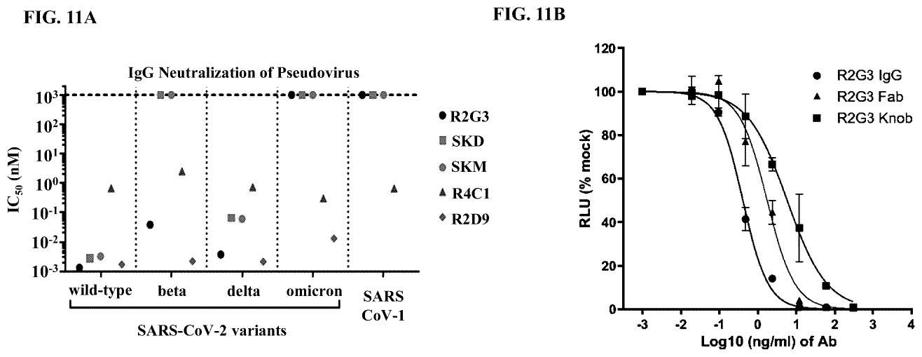

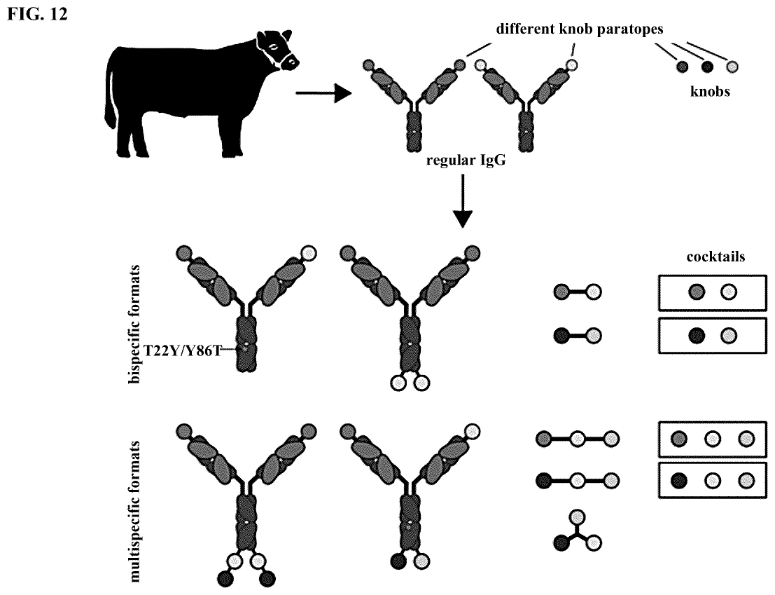

Also provided herein are compositions containing any of the provided knob peptides. In some embodiments, the compositions can be monoclonal providing a single knob peptide to provide a single paratope for binding a desired antigen, such as SARS-CoV2. In other embodiments, provided compositions are polyclonal and contain a mixture or cocktail of different knob peptides directed against different epitopes of an antigen or different antigens (FIG. 12).

Further, also provided herein are multispecific binding formats that exploit the small and unique size of the knob peptides (FIG. 12). For instance, different knob paratopes can be engineered into the backbone of a human or humanized ultralong CDR-H3 full length antibody in which dimerization of the Fc provides a bivalent or multivalent format. In some cases, “knobs into” hole Fc engineering strategy can be used to produce a heterodimeric bispecific or multispecific format containing two, three, four or more different knob peptides each providing a different paratope for binding to a desired antigen, such as spike protein of SARS-CoV2.

Also provided herein are methods of treatment and uses of the provided binding polypeptides, including antibodies or antigen-binding fragments or knob polypeptides, and compositions thereof, for the treatment of coronavirus infection, such as caused by SARS-CoV-2.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the articles “a” and “an” refer to one or to more than one (i.e. to at least one) of the grammatical object of the article. By way of example, “an element” means one element or more than one element.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20% or ±10%, more preferably ±5%, even more preferably ±1%, and still more preferably ±0.10% from the specified value, as such variations are appropriate to perform the disclosed methods.

An “ultralong CDR3” or an “ultralong CDR3 sequence”, used interchangeably herein, comprises a CDR3 or CDR3 sequence that is not derived from a human antibody sequence. An ultralong CDR3 may be 35 amino acids in length or longer, for example, 40 amino acids in length or longer, 45 amino acids in length or longer, 50 amino acids in length or longer, 55 amino acids in length or longer, or 60 amino acids in length or longer. In some embodiments, the ultralong CDR3 is 25-70 amino acids in length, such as 40-70 amino acids in length. Typically, the ultralong CDR3 is a heavy chain CDR3 (CDR-H3 or CDRH3). An ultralong CDR3H3 exhibits features of a CDRH3 of a ruminant (e.g., bovine) sequence. The structure of an ultralong CDR3 includes a “stalk”, composed of ascending and descending strands (e.g. each about 12 amino acids in length), and a disulfide-rich “knob” that sits atop the stalk. The unique “stalk and knob” structure of the ultralong CDR3 results in the two antiparallel β-strands (an ascending and descending stalk strand) supporting a disulfide bonded knob protruding out of the antibody surface to form a mini antigen binding domain. In some embodiments, the ultralong CDR3 antibodies comprise, in order, an ascending stalk region, a knob region, and a descending stalk region.

As used herein, a “CDR3-knob” or “knob,” which are used interchangeably refers to a portion of an ultralong CDR3 that is a peptide sequence of 40-70 amino acids in length, where said CDR3-knob has at least 4 non-canonical Cys residues, such as 6, 8, 10 or up to 12 non-canonical cysteine residues, and forms 2-6 disulfide bonds. Typically a knob contains an initial cysteine residue with the amino acid motif cysteine-proline (CP). In some cases, a CDR3-knob may be positioned between an ascending stalk (Stalk A) or a descending stalk (Stalk B) in an antibody or antigen-binding fragment containing the ultralong CDR3, in which the CDR3-knob protrudes out of the antibody interface to form an antigen binding site with an antigen. In other cases, a CDR3-knob may be independently produced as a “knob” peptide as described herein.

As used herein, a “knob peptide”, “CDR3-knob peptide” or “knob-only peptide,” which are terms used interchangeably, refers to an independently produced linear disulfide-bonded peptide that is 40-70 amino acids in length, and contains 2-6 disulfide bonds formed by at least 4 non-canonical Cys residues, such as 6, 8, 10 or up to 12 non-canonical cysteine residues. A knob peptide may be derived from an ultralong CDR3 or can be produced synthetically. Typically, the first cysteine of the peptide sequences contains an initial cysteine residue with the amino acid motif cysteine-proline (CP). A knob peptide is a linear molecular that is not able to undergo cyclization to form a cyclic molecule.

“Substantially similar,” or “substantially the same”, refers to a sufficiently high degree of similarity between two numeric values (generally one associated with an antibody disclosed herein and the other associated with a reference/comparator antibody) such that one of skill in the art would consider the difference between the two values to be of little or no biological and/or statistical significance within the context of the biological characteristic measured by said values (e.g., Kd values). The difference between said two values is preferably less than about 50%, preferably less than about 40%, preferably less than about 30%, preferably less than about 20%, preferably less than about 10% as a function of the value for the reference/comparator antibody.

“Binding affinity” generally refers to the strength of the sum total of noncovalent interactions between a single binding site of a molecule (e.g., an antibody) and its binding partner (e.g., an antigen). Unless indicated otherwise, “binding affinity” refers to intrinsic binding affinity which reflects a 1:1 interaction between members of a binding pair (e.g., antibody and antigen). The affinity of a molecule X for its partner Y can generally be represented by the dissociation constant. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure.

“Percent (%) amino acid sequence identity” with respect to a peptide or polypeptide sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MegAlign (DNASTAR) software. Those skilled in the art can determine appropriate parameters for measuring alignment, including any algorithms needed to achieve maximal alignment over the full length of the sequences being compared.

“Polypeptide,” “peptide,” “protein,” and “protein fragment” may be used interchangeably to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers.

“Amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refers to compounds that have the same basic chemical structure as a naturally occurring amino acid, e.g., an alpha carbon that is bound to a hydrogen, a carboxyl group, an amino group, and an R group, e.g., homoserine, norleucine, methionine sulfoxide, methionine methyl sulfonium. Such analogs can have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

“Conservatively modified variants” applies to both amino acid and nucleic acid sequences. “Amino acid variants” refers to amino acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated (e.g., naturally contiguous) sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, silent variations of a nucleic acid which encodes a polypeptide is implicit in a described sequence with respect to the expression product, but not with respect to actual probe sequences. As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” including where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles disclosed herein. Typically conservative substitutions include: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

“Humanized” or “Human engineered” forms of non-human (e.g., bovine) antibodies are chimeric antibodies that contain amino acids represented in human immunoglobulin sequences, including, for example, wherein minimal sequence is derived from non-human immunoglobulin. For example, humanized or human engineered antibodies may be non-human (e.g., bovine) antibodies in which some residues are substituted by residues from analogous sites in human antibodies (see, e.g., U.S. Pat. No. 5,766,886). A humanized antibody optionally may also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Cuff. Op. Struct. Biol. 2:593-596 (1992). See also the following review articles and references cited therein: Vaswani and Hamilton, Ann. Allergy, Asthma & Immunol. 1: 105-115 (1998); Harris, Biochem. Soc. Transactions 23:1035-1038 (1995); Hurle and Gross, Curr. Op. Biotech. 5:428-433 (1994).

A “variable domain” with reference to an antibody refers to a specific Ig domain of an antibody heavy or light chain that contains a sequence of amino acids that varies among different antibodies. Each light chain and each heavy chain has one variable region domain (VL, and, VH). The variable domains provide antigen specificity, and thus are responsible for antigen recognition. Each variable region contains CDRs that are part of the antigen binding site domain and framework regions (FRs).

A “constant region domain” refers to a domain in an antibody heavy or light chain that contains a sequence of amino acids that is comparatively more conserved among antibodies than the variable region domain. Each light chain has a single light chain constant region (CL) domain and each heavy chain contains one or more heavy chain constant region (CH) domains, which include, CH1, CH2, CH3 and, in some cases, CH4. Full-length IgA, IgD and IgG isotypes contain CH1, CH2 CH3 and a hinge region, while IgE and IgM contain CH1, CH2 CH3 and CH4. CH1 and CL domains extend the Fab arm of the antibody molecule, thus contributing to the interaction with antigen and rotation of the antibody arms. Antibody constant regions can serve effector functions, such as, but not limited to, clearance of antigens, pathogens and toxins to which the antibody specifically binds, e.g. through interactions with various cells, biomolecules and tissues.

The terms “complementarity determining region,” and “CDR,” synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 Jan. 27(1):55-77 (“IMGT” numbering scheme); Honegger A and Pluckthun A, “Yet another numbering scheme for immunoglobulin variable domains: an automatic modeling and analysis tool,” J Mol Biol, 2001 Jun. 8; 309(3):657-70, (“Aho” numbering scheme); and Martin et al., “Modeling antibody hypervariable loops: a combined algorithm,” PNAS, 1989, 86(23):9268-9272, (“AbM” numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software.

Table 2, below, lists exemplary position boundaries of CDR-L1, CDR-L2, CDR-L3 and CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-L1 located before CDR-L1, FR-L2 located between CDR-L1 and CDR-L2, FR-L3 located between CDR-L2 and CDR-L3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 2
Boundaries of CDRs according to various numbering schemes.

| CDR | Kabat | Chothia | AbM | Contact |
|---|---|---|---|---|
| CDR-L1 | L24--L34 | L24--L34 | L24--L34 | L30--L36 |
| CDR-L2 | L50--L56 | L50--L56 | L50--L56 | L46--L55 |
| CDR-L3 | L89--L97 | L89--L97 | L89--L97 | L89--L96 |
| CDR-H1 | H31--H35B | H26--H32 . . . 34 | H26--H35B | H30--H35B |
| (Kabat Numbering¹) | | | | |
| CDR-H1 | H31--H35 | H26--H32 | H26--H35 | H30--H35 |
| (Chothia Numbering²) | | | | |
| CDR-H2 | H50--H65 | H52--H56 | H50--H58 | H47--H58 |
| CDR-H3 | H95--H102 | H95--H102 | H95--H102 | H93--H101 |

¹Kabat et al. (1991), “Sequences of Proteins of Immunological Interest,” 5th Ed. Public Health Service, National Institutes of Health, Bethesda, MD
²Al-Lazikani et al., (1997) JMB 273, 927-948

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes. For example, where it is stated that a particular CDR (e.g., a CDR-H3) contains the amino acid sequence of a corresponding CDR in a given VH or VL region amino acid sequence, it is understood that such a CDR has a sequence of the corresponding CDR (e.g., CDR-H3) within the variable region, as defined by any of the aforementioned schemes. In some embodiments, specific CDR sequences are specified. Exemplary CDR sequences of provided antibodies are described using various numbering schemes (see e.g. Table 2 and Table 3), although it is understood that a provided antibody can include CDRs as described according to any of the other aforementioned numbering schemes or other numbering schemes known to a skilled artisan

Likewise, unless otherwise specified, a FR or individual specified FR(s) (e.g., FR-H1, FR-H2, FR-H3, FR-H4), of a given antibody or region thereof, such as a variable region thereof, should be understood to encompass a (or the specific) framework region as defined by any of the known schemes. In some instances, the scheme for identification of a particular CDR, FR, or FRs or CDRs is specified, such as the CDR as defined by the Kabat, Chothia, AbM or Contact method. In other cases, the particular amino acid sequence of a CDR or FR is given.

An antibody containing an ultralong CDR3 is an antibody that contains a variable heavy (VH) chain with an ultralong CDR3. An antibody may further include pairing of the VH chain with a variable light (VL) chain. In some embodiments, the antibodies or antigen-binding fragments include a heavy chain variable region and a light chain variable region. Thus, the term antibody include full-length antibodies and portions thereof including antibody fragments, wherein such contain a heavy chain or portion thereof and/or a light chain or portion thereof. An antibody can contain two heavy chains (which can be denoted H and H′) and two light chains (which can be denoted L and L′), in which each L chain is linked to an H chain by a covalent disulfide bond and the two H chains are linked to each other by disulfide bonds. The terms “full-length antibody,” or “intact antibody” are used interchangeably to refer to an antibody in its substantially intact form, as opposed to an antibody fragment. A full-length antibody is an antibody typically having two full-length heavy chains (e.g., VH-CH1-CH2-CH3 or VH-CH1-CH2-CH3-CH4) and two full-length light chains (VL-CL) and hinge regions.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, heavy chain variable (VH) regions capable of specifically binding, and single chain variable fragments (scFv).

An “antibody fragment” comprises a portion of an intact antibody, the antigen binding and/or the variable region of the intact antibody. Antibody fragments, include, but are not limited to, Fab fragments, Fab′ fragments, F(ab′)₂ fragments, Fv fragments, disulfide-linked Fvs (dsFv), Fd fragments, Fd′ fragments; single-chain antibody molecules, including single-chain Fvs (scFv) or single-chain Fabs (scFab); antigen-binding fragments of any of the above and multispecific antibodies from antibody fragments.

A “Fab fragment” is an antibody fragment that results from digestion of a full-length immunoglobulin with papain, or a fragment having the same structure that is produced synthetically, e.g., by recombinant methods. A Fab fragment contains a light chain (containing a V_{L} and C_{L}) and another chain containing a variable domain of a heavy chain (V_{H}) and one constant region domain of the heavy chain (C_{H}1).

An “scFv fragment” refers to an antibody fragment that contains a variable light chain (V_{L}) and variable heavy chain (V_{H}), covalently connected by a polypeptide linker in any order. The linker is of a length such that the two variable domains are bridged without substantial interference. Exemplary linkers are (Gly-Ser)ₙ residues with some Glu or Lys residues dispersed throughout to increase solubility.

The term “isolated” with reference to binding polypeptide, such as antibodies or antigen-binding fragments thereof or knob peptides, or polynucleotides and vectors encoding same, are at least partially free of other biological molecules from the cells or cell culture from which they are produced. Such biological molecules include nucleic acids, proteins, other antibodies or antigen-binding fragments, lipids, carbohydrates, or other material such as cellular debris and growth medium. An isolated antibody or antigen-binding fragment may further be at least partially free of expression system components such as biological molecules from a host cell or of the growth medium thereof. Generally, the term “isolated” is not intended to refer to a complete absence of such biological molecules or to an absence of water, buffers, or salts or to components of a pharmaceutical formulation that includes the antibodies or fragments.

The term, “corresponding to” with reference to positions of a protein, such as recitation that nucleotides or amino acid positions “correspond to” nucleotides or amino acid positions in a disclosed sequence, such as set forth in the Sequence listing, refers to nucleotides or amino acid positions identified upon alignment with the disclosed sequence based on structural sequence alignment or using a standard alignment algorithm, such as the GAP algorithm. For example, corresponding residues of a similar sequence (e.g. fragment or species variant) can be determined by alignment to a reference sequence by structural alignment methods. By aligning the sequences, one skilled in the art can identify corresponding residues, for example, using conserved and identical amino acid residues as guides.

The term “effective amount” or “therapeutically effective amount” as used herein means an amount of a pharmaceutical composition which is sufficient enough to significantly and positively modify the symptoms and/or conditions to be treated (e.g., provide a positive clinical response). The effective amount of an active ingredient for use in a pharmaceutical composition will vary with the particular condition being treated, the severity of the condition, the duration of treatment, the nature of concurrent therapy, the particular active ingredient(s) being employed, the particular pharmaceutically-acceptable excipient(s) and/or carrier(s) utilized, and like factors with the knowledge and expertise of the attending physician.

As used herein, the term “pharmaceutically acceptable” refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

As used herein, a composition refers to any mixture of two or more products, substances, or compounds, including cells. It may be a solution, a suspension, liquid, powder, a paste, aqueous, non-aqueous or any combination thereof.

As used herein, the term “pharmaceutical composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

As used herein, “disease or disorder” refers to a pathological condition in an organism resulting from cause or condition including, but not limited to, infections, acquired conditions, genetic conditions, and characterized by identifiable symptoms.

As used herein, the terms “treat,” “treating,” or “treatment” refer to ameliorating a disease or disorder, e.g., slowing or arresting or reducing the development of the disease or disorder, e.g., a root cause of the disorder or at least one of the clinical symptoms thereof.

As used herein, the term “subject” refers to an animal, including a mammal (e.g., rat, mouse, cat, dog, cow, pig, sheep, horse, goat, rabbit), such as a human being. Typically the subject is a human subject. The term subject and patient can be used interchangeably.

As used herein, “optional” or “optionally” means that the subsequently described event or circumstance does or does not occur, and that the description includes instances where said event or circumstance occurs and instances where it does not. For example, an optionally substituted group means that the group is unsubstituted or is substituted.

### II. Bovine-Derived Binding Polypeptides Against Sars-Cov-2 and Related Coronaviruses

Provided herein are binding polypeptides directed against SARS-CoV-2. In particular, among provided binding polypeptides are polypeptides that exhibit neutralizing activity against SARS-CoV-2. The binding polypeptides include bovine or bovine-derived (e.g. humanized or chimeric) antibodies or antigen binding fragments, including bovine ultralong CDR-H3 antibodies and antigen binding fragments. The binding polypeptides provided herein also include unique knob peptides derived from the ultralong CDR-H3 of any of the provided ultralong CDR-H3 antibodies. Among the provided herein binding polypeptides are broad spectrum neutralizing binding polypeptides, including antibodies or antigen binding fragments or knob peptides, to SARS and related coronaviruses.

Also provided are nucleic acids, e.g., polynucleotides, encoding any of the provided binding polypeptides, such as knob peptides or antibodies and/or portions, e.g., chains, thereof. Among the provided nucleic acids are those encoding the anti-SARS-CoV2 spike protein antibodies (e.g., antigen-binding fragment) described herein. Also provided are nucleic acids, e.g., polynucleotides, encoding one or more knob peptides described herein including any encoding an anti-SARS-CoV2 spike protein knob peptide. The nucleic acids may include those encompassing natural and/or non-naturally occurring nucleotides and bases, e.g., including those with backbone modifications. The terms “nucleic acid molecule”, “nucleic acid” and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide.

Also provided are vectors containing the nucleic acids, e.g., polynucleotides, and host cells containing the vectors, e.g., for producing the antibodies or antigen-binding fragments thereof. Also provided are methods for producing the binding polypeptides, including antibodies or antigen-binding fragments thereof or knob peptides.

In a further embodiment, one or more vectors (e.g., expression vectors) comprising such nucleic acids are provided. In a further embodiment, a host cell comprising such nucleic acids is provided. In some embodiments, a host cell comprises (e.g., has been transformed with) one or more vectors comprising one or more nucleic acid that encodes one or more an amino acid sequence comprising one or more antibodies and/or portions thereof, e.g., antigen-binding fragments thereof. In some embodiments, one or more such host cells are provided. In some embodiments, a composition containing one or more such host cells are provided. In some embodiments, the one or more host cells can express different binding polypeptides (e.g. antibodies or knob peptides), or the same binding polypeptide.

Also provided are methods of making the provided binding polypeptides, including antibodies or antigen-binding fragments or knob peptides. For recombinant production of the binding polypeptide, a nucleic acid sequence or a polynucleotide encoding a binding polypeptide, e.g., as described above, may be isolated and inserted into one or more vectors for further cloning and/or expression in a host cell. Such nucleic acid sequences may be readily isolated and sequenced using conventional procedures (e.g., by using oligonucleotide probes that are capable of binding specifically to genes encoding the heavy and light chains of the antibody or by synthetic methods). In some embodiments, a method of making the binding polypeptide is provided, wherein the method comprises culturing a host cell comprising a nucleic acid sequence encoding the binding polypeptide, any as described herein, under conditions suitable for expression of the binding polypeptide, and optionally recovering the binding polypeptide from the host cell (or host cell culture medium). Methods for producing binding polypeptides are described. The provided embodiments further include vectors and host cells and other expression systems for expressing and producing the antibodies and other antigen-binding proteins, including eukaryotic and prokaryotic host cells, including bacteria, filamentous fungi, and yeast, as well as mammalian cells such as human cells, as well as cell-free expression systems.

### A. Bovine and Bovine-Derived Antibodies and Antigen Binding Fragments

Provided herein are antibodies or antigen binding fragments that exhibit binding to and neutralizing activity against coronaviruses. In some embodiments, the antibodies or antigen binding fragments exhibit binding activity to and neutralizing activity against SARS-CoV2. In particular embodiments, the provided antibodies or antigen-binding fragments bind to SARS-CoV-2 spike protein.

In some embodiments, the antibody or antigen binding fragment comprises a variable heavy chain that has a CDR-H3 of an antibody RBDA2, RBD C6, RBD F4, R2B1, R2D6, R2G1, R4A10, R4E5, R4G3, R4G11, R5A3, R4C1, R2C3, SKD, SKM, R2G3, R2F12, R2F12, SR3A3, or R2D9. Unlike antibodies from other species, such as human and mouse, the CDR regions L1, L2, L3, H1 and H2 of a bovine or bovine-derived antibody exhibit less sequence diversity as most of their sequence diversity is in CDR H3 (Stanfield et al. 2016 Sci. Immunol, 1(1): doi:10.1126/sciimmunol.aaf7962). Thus, for bovine or bovine-derived antibodies, antigen binding is mainly or only through CDR H3 and the other CDRs do not contribute to antigen binding.

In some embodiments, the antibody or antigen binding fragment comprises a variable heavy chain that has a CDR-H1, CDR-H2, and CDR-H3 of an antibody RBDA2, RBD C6, RBD F4, R2B1, R2D6, R2G1, R4A10, R4E5, R4G3, R4G11, R5A3, R4C1, R2C3, SKD, SKM, R2G3, R2F12, R2F12, SR3A3, or R2D9.

The precise amino acid sequence boundaries of a given CDR or FR can be readily determined using any of a number of well-known schemes (Stanfield et al. 2016; Stanfield et al. 2018 Adv Immunol, 137:135-164, doi:10.1016/bs.ai2017.12.004; Wang).

In some cases, the numbering scheme for numbering of CDRs in antibody variable regions can include any of those described by Kabat et al. (1991), “Sequences of Proteins of Immunological Interest,” 5th Ed. Public Health Service, National Institutes of Health, Bethesda, MD (“Kabat” numbering scheme); A1-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 Jan. 27(1):55-77 (“IMGT” numbering scheme); Honegger A and Pluckthun A, “Yet another numbering scheme for immunoglobulin variable domains: an automatic modeling and analysis tool,” J Mol Biol, 2001 Jun. 8:309(3):657-70, (“Aho” numbering scheme); and Martin et al., “Modeling antibody hypervariable loops; a combined algorithm,” PNAS, 1989, 86(23):9268-9272, (“AbM” numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software.

Table 3, below, lists exemplary position boundaries of CDR-L1, CDR-L2, CDR-L3 and CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-L1 located before CDR-L1, FR-L2 located between CDR-L1 and CDR-L2, FR-L3 located between CDR-L2 and CDR-L3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 3
Boundaries of CDRs according to various numbering schemes.

| CDR | Kabat | Chothia | AbM | Contact |
|---|---|---|---|---|
| CDR-L1 | L24--L34 | L24--L34 | L24--L34 | L30--L36 |
| CDR-L2 | L50--L56 | L50--L56 | L50--L56 | L46--L55 |
| CDR-L3 | L89--L97 | L89--L97 | L89--L97 | L89--L96 |
| CDR-H1 | H31--H35B | H26--H32 . . . 34 | H26--H35B | H30--H35B |
| (Kabat Numbering¹) | | | | |
| CDR-H1 | H31--H35 | H26--H32 | H26--H35 | H30--H35 |
| (Chothia Numbering²) | | | | |
| CDR-H2 | H50--H65 | H52--H56 | H50--H58 | H47--H58 |
| CDR-H3 | H95--H102 | H95--H102 | H95--H102 | H93--H101 |

¹Kabat et al. (1991), “Sequences of Proteins of Immunological Interest,” 5th Ed. Public Health Service, National Institutes of Health, Bethesda, MD
²Al-Lazikani et al., (1997) JMB 273, 927-948

In some embodiments, the ultralong (UL)-CDR H3 is too long to be accommodated by a numbering scheme, such as described in Table 3. Thus, in some cases, a CDR numbering scheme, such as any described in Table 3, is used for the light chain (L), and for heavy chain (H) residues 1-100, and 101-128, but residues in CDR H3 encoded by the DH2 and JH1 genes will be numbered sequentially as described in Stanfield et al. 2016 or Wang et al. 2013, Cell 153:1379-1393. For instance, the DH2 encoded region begins with a high conserved CPD motif at residues 2-4 compared to the germline-encoded DH2 sequence; therefore in the numbering scheme for an UL-CDR3 the conserved Cys residue is “D2” followed by D3, D4, etc., and then the JH1-encoded residues J1 and J2 followed by H101. The cysteine DH2 and tryptophan H105 (corresponding to J5) define the boundaries of an UL-CDR H3 (see FIG. 4)

TABLE 4
sets forth exemplary CDRs of a variable heavy chain of a provided antibody.
Exemplary variable heavy complementarity determining region (CDR H)
(SEQ ID NO)

| Antibody | CDR-H1 | CDR-H2 | CDR-H3 |
|---|---|---|---|
| RBD | SNGVV | DICSTGGTSFNPALKS | SRGYDCYANVDALDYVDA |
| | (84) | (85) | (86) |
| RBD C6 | SNGVV | DICSNGGTSFNPALKS | SRGYDCYAYVYALDTVDA |
| | (84) | (87) | (88) |
| RBD F4 | SNGVV | DICSTGGTSFNPALKS | SRGYDCYANVDALDYVDA |
| | (84) | (85) | (89) |
| R2B1 | DNNVG | VMHNDGNKGYNPALKS | DNARCDSWTYDSCDTWYRNSWHVDA |
| | (90) | (91) | (92) |
| R2D6 | SYAVG | DIDYVGNTDYNPALKS | YSGAYAYAACNYYGWRCAWESYIDA |
| | (93) | (94) | (95) |
| R2G1 | NYAVG | DVDSSGGTAYNPALKS | FAKGTTSAGACDYSESYVDA |
| | (96) | (97) | (98) |
| R4A10 | DYAVG | GIDTGGSTGYNPGLKS | VVLCYYNRVVRRNNCGGLGYDYGFDHF |
| | (99) | (100) | YVDA |
| | | | (101) |
| R4E5 | NYAVG | GIDTGGSTGYNPGLES | VVLCYFNYVVRRYNCGGLGYGHGFNSF |
| | (96) | (102) | YVDA |
| | | | (103) |
| R4G3 | DYAVG | GIDTGGSTGYNPGLES | VVLCYFNYVVRRYNCGGLGYGHGFNSF |
| | (99) | (102) | YVDA |
| | | | (104) |
| R4G11 | SYGIT | SISSGGTTDYNPALKS | WNLEYTWGGVGCASFADEDTHVDA |
| | (105) | (106) | (107) |
| R5A3 | DKAVG | SIDTGGSTGYNPGLKS | WGQGLLVTVSS |
| | (108) | (109) | (110) |
| R4C1 | DKAVG | SIDTAENTGYNPGLKS | ATVHQKTRKEKNCPDGYIYSSNTASGYD |
| | (108) | (111) | CGVWICRRVGSAFCSRTGDYTSPSEFDIY |
| | | | EFYVEG |
| | | | (112) |
| R2C3 | DKPVG | SIDTAENTGYNPGLKS | ATVHQKTRKEKSCPDGYLYSSNTGRGYD |
| | (113) | (111) | CGVWTCRRVGGEFCSATGDWTSPSEEDF |
| | | | YEFYVDT |
| | | | (114) |
| SKD | DKAVO | SIDTGGNTGYNPGLKS | TTVHQRTSEKRSCPGGSSRRYPSGASCDV |
| | (108) | (115) | SGGACACYVSNCRGVLCPTLNEIVAYTY |
| | | | EWHVDA |
| | | | (116) |
| SKM | DKAVG | SIDTGGNTGYNPGLKS | TTVHQETLRSCPDGYIDNSGCTADWGCA |
| | (108) | (115) | ALDCWRRRFGYHSTDPSHYTGATYIYTY |
| | | | SLHIDAW |
| | | | (117) |
| R2G3 | DKAVG | SIDTGGSTGYNPGLKS | ATVHQKTAEGDKTCPDGYEHTCGCIGGC |
| | (108) | (118) | GCKRSACIGALCCQASLGGWLSDGETYT |
| | | | YEFHVDT |
| | | | (119) |
| R2F12 | DKAVG | SIDTGGMTGYNPGLKS | ATVDQKTKNACPDDFDYRCSCIGGCGCA |
| | (108) | (120) | RKGCVGPLCCRSDLGGYLTDSPAYIYEW |
| | | | YIDL |
| | | | (121) |
| SR3A3 | SYAVL | SIDTAENTGYNPGLKS | ATVHQKTRKEKNCPDGYIYSSNITSGFDC |
| | (122) | (111) | GVWICRRVGSAFCSRTGDYTSPTELDIYE |
| | | | FYVEG |
| | | | (123) |
| R2D9 | DKAIG | SIDTRGNTGYNPGLKS | AIVQQITHKTCPNGYNWFDRCCSWDGTC |
| | (124) | (125) | GDGCCSNRAWPSGNGRADSSIGETYGYE |
| | | | FHVAA |
| | | | (126) |

In some embodiments, a variable heavy chain of a provided antibody or antigen-binding fragment has a CDR-H3 set forth in any one of SEQ ID NOS: 86, 88, 89, 92, 95, 98, 101, 103, 104, 107, 110, 112, 114, 116, 117, 119, 121, 123, or 126.

In some embodiments, a variable heavy chain of a provided antibody or antigen-binding fragment contains a CDR-H1, a CDR-H2 and CDR-H3 as set forth in Table 4. In some embodiments, a variable heavy chain of a provided antibody or antigen-binding fragment contains a CDR-H1 as set forth in any one of SEQ ID NOS: 84, 90, 93, 96, 99, 105, 108, 113, 122 or 124. In some embodiments, a variable heavy chain of a provided antibody or antigen-binding fragment contains a CDR-H2 as set forth in any one of SEQ ID NOS: 85, 87, 91, 94, 97, 100, 102, 106, 109, 111, 115, 118, 120 or 125. In some embodiments, a variable heavy chain of a provided antibody or antigen-binding fragment contains a CDR-H3 set forth in any one of SEQ ID NOS: 86, 88, 89, 92, 95, 98, 101, 103, 104, 107, 110, 112, 114, 116, 117,119, 121, 123, or 126.

In some embodiments, a variable heavy chain of a provided antibody or antigen-binding fragment has a CDR-H1, a CDR-H2 and CDR-H3 set forth in SEQ ID NOS: 84, 85 and 86, respectively. In some embodiments, a variable heavy chain of a provided antibody or antigen-binding fragment has a CDR-H1, a CDR-H2 and CDR-H3 set forth in SEQ ID NOS: 84, 87 and 88, respectively. In some embodiments, a variable heavy chain of a provided antibody or antigen-binding fragment has a CDR-H1, a CDR-H2 and CDR-H3 set forth in SEQ ID NOS: 84, 85 and 89, respectively. In some embodiments, a variable heavy chain of a provided antibody or antigen-binding fragment has a CDR-H1, a CDR-H2 and CDR-H3 set forth in SEQ ID NOS: 90, 91 and 92, respectively. In some embodiments, a variable heavy chain of a provided antibody or antigen-binding fragment has a CDR-H1, a CDR-H2 and CDR-H3 set forth in SEQ ID NOS: 93, 94 and 95, respectively. In some embodiments, a variable heavy chain of a provided antibody or antigen-binding fragment has a CDR-H1, a CDR-H2 and CDR-H3 set forth in SEQ ID NOS: 96, 97 and 98, respectively. In some embodiments, a variable heavy chain of a provided antibody or antigen-binding fragment has a CDR-H1, a CDR-H2 and CDR-H3 set forth in SEQ ID NOS: 99, 100 and 101, respectively. In some embodiments, a variable heavy chain of a provided antibody or antigen-binding fragment has a CDR-H1, a CDR-H2 and CDR-H3 set forth in SEQ ID NOS: 96, 102 and 103, respectively. In some embodiments, a variable heavy chain of a provided antibody or antigen-binding fragment has a CDR-H1, a CDR-H2 and CDR-H3 set forth in SEQ ID NOS: 99, 102 and 104, respectively. In some embodiments, a variable heavy chain of a provided antibody or antigen-binding fragment has a CDR-H1, a CDR-H2 and CDR-H3 set forth in SEQ ID NOS: 105, 106 and 107, respectively. In some embodiments, a variable heavy chain of a provided antibody or antigen-binding fragment has a CDR-H1, a CDR-H2 and CDR-H3 set forth in SEQ ID NOS: 108, 109 and 110, respectively. In some embodiments, a variable heavy chain of a provided antibody or antigen-binding fragment has a CDR-H1, a CDR-H2 and CDR-H3 set forth in SEQ ID NOS: 108, 111 and 112, respectively. In some embodiments, a variable heavy chain of a provided antibody or antigen-binding fragment has a CDR-H1, a CDR-H2 and CDR-H3 set forth in SEQ ID NOS: 113, 111 and 114, respectively. In some embodiments, a variable heavy chain of a provided antibody or antigen-binding fragment has a CDR-H1, a CDR-H2 and CDR-H3 set forth in SEQ ID NOS: 108, 115 and 116, respectively. In some embodiments, a variable heavy chain of a provided antibody or antigen-binding fragment has a CDR-H1, a CDR-H2 and CDR-H3 set forth in SEQ ID NOS: 108, 115 and 117, respectively. In some embodiments, a variable heavy chain of a provided antibody or antigen-binding fragment has a CDR-H1, a CDR-H2 and CDR-H3 set forth in SEQ ID NOS: 108, 118 and 119, respectively. In some embodiments, a variable heavy chain of a provided antibody or antigen-binding fragment has a CDR-H1, a CDR-H2 and CDR-H3 set forth in SEQ ID NOS: 108, 120 and 121, respectively. In some embodiments, a variable heavy chain of a provided antibody or antigen-binding fragment has a CDR-H1, a CDR-H2 and CDR-H3 set forth in SEQ ID NOS: 122, 111 and 123, respectively. In some embodiments, a variable heavy chain of a provided antibody or antigen-binding fragment has a CDR-H1, a CDR-H2 and CDR-H3 set forth in SEQ ID NOS: 124, 125 and 126, respectively.

In some embodiments, a variable heavy chain of a provided antibody or antigen-binding fragment has a sequence set forth in any one of SEQ ID NOS: 33-51, or a sequence of amino acids that exhibits at least 85%, 86%, 87%, 88%, 89%, 90%, 91%,92%, 93%, 94%, 95%, 96%, 97%, 98%, or 99% sequence identity to any one of SEQ ID NOS: 33-51. In some embodiments, a variable heavy chain of a provided antibody or antigen-binding fragment has a sequence set forth in any one of SEQ ID NOS: 33-51.

In some embodiments, a variable heavy chain of a provided antibody or antigen-binding fragment contains an ultralong CDR-H3. In cow antibodies, the ultralong CDR3 sequences form a structure where a subdomain with an unusual architecture is formed from a “stalk”, composed of two 12-residue, anti-parallel β-strands (ascending and descending strands), and a longer, e.g. 39-residue, disulfide-rich “knob” that sits atop the stalk, far from the canonical antibody paratope. The long anti-parallel β-ribbon serves as a bridge to link the knob domain with the main antibody scaffold. The unique “stalk and knob” structure of the ultralong CDR3 results in the two antiparallel s-strands (an ascending and descending stalk strand) supporting a disulfide bonded knob protruding out of the antibody surface to form a mini antigen binding domain. In some embodiments, the ultralong CDR3 antibodies comprise, in order, an ascending stalk region, a knob region, and a descending stalk region.

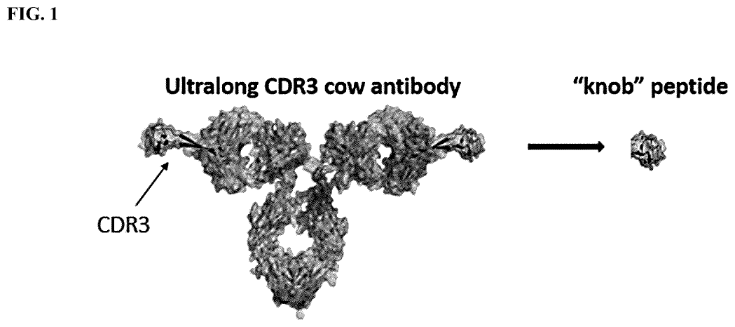

In some embodiments, a variable heavy chain of a provided antibody or antigen-binding fragment contains an ultralong CDR-H3 of between 25 and 70 amino acids in length. In some embodiments, the antibody or antigen-binding fragment contains an ultralong CDR-H3 of between 40 and 70 amino acids in length. In some embodiments, the ultralong CDR-H3 includes an ascending stalk domain (Stalk A), a disulfide-rich knob region, and a descending stalk domain (Stalk B), in which the knob region is positioned between the ascending and descending stalk domains. In some embodiments, the sequence of the ultralong CDR-H3 of a provided antibody or antigen-binding fragment provides a structure of an anti-parallel β-strands that protrude away from the antibody, in which the disulfide-rich knob region is positioned at the tip of the antibody (FIG. 1). Stalk A comprises mainly hydrophobic side chains and a relatively conserved motif at the base, which initiates the ascending strand. This conserved motif is typically found following the first cysteine residue in variable region sequences of the various bovine or cow sequences. In some embodiments, the base of Stalk A contains residues CTTVHQ (SEQ ID NO:127), CATVHQ (SEQ ID NO:128), CAIVQQ (SEQ ID NO:129), CATVDQ (SEQ ID NO:130) that stabilizes the base by interacting with residues of the CDR-H1. The Stalk A is connected by a variable number of residues, e.g. 2 to 8 amino acid residues, before a first conserved cysteine residue that forms part of the disulfide-bonded knob region. In some embodiments, the knob region includes a first conserved amino acid motif Cys-Pro (CP), in which the initial cysteine residue forms the first disulfide bond with another cysteine residue in the knob. The knob may include 2-12 cysteine residues that are able to form 2-6 disulfide bonds. The stalk can be of variable length, and Stalk B may comprise alternating aromatics that form a ladder through stacking interactions, that may contribute to the stability of the long solvent-exposed, two stranded β-ribbon (Wang et al. Cell. 2013, 153 (6): 1379-1393). In some embodiments, the Stalk B contains a conserved pattern of alternating tyrosines, sometimes with the motif YX₁YX₂Y (SEQ ID NO:131), that support the knob structure.

In some embodiments, a variable heavy chain provided herein containing an ultralong CDR-H3 exhibits high sequence identity to bovine germline segments IgHV1-7 (SEQ ID NO:81), IGHD8-2 (SEQ ID NO: 82) and IGHJ2-4 (SEQ ID NO:83). In some embodiments, the V, D and J portions of a provided ultralong CDR-H3 variable heavy chain exhibits at least at or about 80%, at least at or about 85% or a least at or about 90% sequence identity to any of SEQ ID NOS: 81, 82 and 83, respectively.

In some embodiments, a variable heavy chain of a provided antibody or antigen-binding fragment contains an ultralong CDR-H3 of any of antibodies R4C1, R2C3, SKD, SKM, R2G3, R2F12, SR3A3 or R2D9. In some embodiments, a variable heavy chain of a provided antibody or antigen-binding fragment contains an ultralong CDR-H3 set forth in any one of SEQ ID NOS: 112, 114, 116, 117, 119, 121, 123, or 126. In some embodiments, a variable heavy chain of a provided antibody or antigen-binding fragment has the sequence set forth in any one of SEQ ID NOS: 33, 34, 35, 40, 45, 46, 50 or 51, or a sequence of amino acids that exhibits 85%, 86%, 87%, 88%, 89%, 90%, 91%,92%, 93%, 94%, 95%, 96%, 97%, 98%, or 990% sequence identity to any one of SEQ ID NOS: 33, 34, 35, 40, 45, 46, 50 or 51. In some embodiments, a variable heavy chain of a provided antibody or antigen-binding fragment has the sequence set forth in any one of SEQ ID NOS: 33, 34, 35, 40, 45, 46, 50 or 51.

In some embodiments, a provided antibody or antigen-binding fragment thereof is humanized.

Methods for engineering, humanizing or resurfacing non-human or human antibodies can also be used and are well known in the art. A humanized, resurfaced or similarly engineered antibody can have one or more amino acid residues from a source that is non-human, e.g., but not limited to, cow, bovine, mouse, rat, rabbit, non-human primate or other mammal. These non-human amino acid residues are replaced by residues that are often referred to as “import” residues, which are typically taken from an “import” variable, constant or other domain of a known human sequence.

Such imported sequences can be used to reduce immunogenicity or reduce, enhance or modify binding, affinity, on-rate, off-rate, avidity, specificity, half-life, or any other suitable characteristic, as known in the art. In general, the CDR residues are directly and most substantially involved in influencing antibody binding. Accordingly, part or all of the non-human or human CDR sequences are maintained while the non-human sequences of the variable and constant regions can be replaced with human or other amino acids.

Antibodies can also optionally be humanized, resurfaced, engineered or human antibodies engineered with retention of high affinity for the antigen and other favorable biological properties. To achieve this goal, humanized (or human) or engineered antibodies and resurfaced antibodies can be optionally prepared by a process of analysis of the parental sequences and various conceptual humanized and engineered products using three-dimensional models of the parental, engineered, and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, framework (FR) residues can be selected and combined from the consensus and import sequences so that the desired antibody characteristic, such as increased affinity for the target antigen(s), is achieved.

Humanization, resurfacing or engineering of antibodies described herein can be performed using any known method, such as but not limited to those described in, Winter (Jones et al., Nature 321:522 (1986); Riechmann et al, Nature 332:323 (1988); Verhoeyen et al., Science 239: 1534 (1988)), Sims et al., J. Immunol. 151: 2296 (1993); Chothia and Lesk, J. Mol. Biol. 196:901 (1987), Carter et al., Proc. Natl. Acad. Sci. U.S.A. 89:4285 (1992); Presta et al, J. Immunol. 151:2623 (1993), U.S. Pat. Nos. 5,639,641, 5,723,323; 5,976,862; 5,824,514; 5,817,483; 5,814,476; 5,763, 192; 5,723,323; 5,766,886; 5,714,352; 6,204,023; 6, 180,370; 5,693,762; 5,530, 101; 5,585,089; 5,225,539; 4,816,567; PCT/: US98/16280; US96/18978; US91/09630; US91/05939; US94/01234; GB89/01334; GB91/01134; GB92/01755; WO90/14443; WO90/14424; WO90/14430; EP 229246; 7,557,189; 7,538, 195; and 7,342,110, each of which is entirely incorporated herein by reference, including the references cited therein.

In some embodiments, the provided humanized antibody or binding fragment thereof comprises a heavy chain variable region comprising a sequence of the formula V1-X-V2, wherein the V1 region of the heavy chain comprises a heavy chain sequence portion containing three framework regions (e.g., FR-1, FR-2, and FR-3) separating two CDR regions (CDR1 and CDR2) of a human V gene or a humanized bovine V gene; the X region comprises any of the provided ultralong CDR3 sequence derived from a bovine ultralong CDR3, such as any described above; and the V2 region comprises a portion of the heavy chain including FR-4 of a human J gene segment or a humanized bovine J gene.

In some embodiments, the variable heavy chain of a provided antibody or antigen binding fragment is based on or derived from a humanized heavy chain framework sequence that is humanized compared to a bovine or cow sequence. In some embodiments, the V1 region is humanized variant of the V region of a bovine antibody, e.g., BLV1H12 (V region set forth in SEQ ID NO:140). In some embodiments, the V1 region comprises a sequence that exhibits at least at or about 65%, at least at or about 70%, at least at or about 75%, at least at or about 80%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, at least at or about 99% sequence identity to the amino acid sequence set forth in SEQ ID NO:140. In some embodiments, the humanized V1 region comprises the sequence set forth in SEQ ID NO: 141. In some embodiments, a V1 region of the heavy chain variable region of the sequence of the formula V1-X-V2 is set forth in SEQ ID NO:141.

In some embodiments, the a humanized antibody or antigen binding fragment provided herein has a chimeric heavy chain that is based on or derived from a human heavy chain framework sequence that exhibits sequence or structural similarities to a bovine or cow sequence, and that incorporates a provided ultralong CDR3. Thus, it is understood that in some cases, humanization can include engineering any of the provided ultralong CDR3 sequence derived from a bovine ultralong CDR3, such as any described above, into a human framework. The human framework may be of germline origin, or may be derived from non-germline (e.g., mutated or affinity matured) sequences. Genetic engineering techniques well known to those in the art, including as disclosed herein, may be used to generate a hybrid DNA sequence containing a human framework and a non-human ultralong CDR3.

Unlike human antibodies which may be encoded by V region genes derived from one of seven families, bovine antibodies which produce ultralong CDR3 sequences appear to utilize a single V region family which may be considered to be most homologous to the human VH4 family. In particular embodiments where ultralong CDR3 sequences derived from cattle are to be humanized to produce an antibody comprising an ultralong CDR3, human V region sequences derived from the VH4 family may be genetically fused to a bovine-derived ultralong CDR3 sequence. Exemplary VH4 germline gene sequences in the human antibody locus include VH4-39, VH4-59*03, VH4-34*02, and VH4-34*09 human heavy chain germline sequences. In some embodiments, the human heavy chain germline sequence is a sequence set forth in any one of SEQ ID NOs: 132-135. In some embodiments, the human heavy chain germline sequence is a sequence encoded by the sequence set forth in any one of SEQ ID NOs: 136-139.

In some embodiments, the provided humanized antibodies or antigen-binding fragments include a fusion of a human VH4 framework sequence (e.g. H4-39, VH4-59*03, VH4-34*02, and VH4-34*09, such as set forth in any of SEQ ID NOS: 132-135) to a bovine-derived ultralong CDR3, such as any described above. In some aspects, such fusions can be generated through the following steps. First, the second cysteine of a V region genetic sequence is identified along with the nucleotide sequence encoding the second cysteine. Generally, the second cysteine marks the boundary of the framework and CDR3 two residues upstream (N-terminal) of the CDR3. Second, the second cysteine in a bovine-derived V region sequence is identified which similarly marks 2 residues upstream (N-terminal) of the CDR3. In some embodiments, the bovine-derived V region sequence is the V gene of the VH sequence set forth in any one of SEQ ID NOS: 33, 34, 35, 40, 45, 46, 50 and 51. For instance, the portion of the VH sequence depicting the second cysteine of the V region sequence is shown as the first residue in sequences in FIG. 4. Third, the genetic material encoding the human V region is combined with the genetic sequence encoding the ultralong CDR3. Thus, a genetic fusion may be made, wherein the ultralong CDR3 sequence is placed in frame of the human V region sequence.

In some embodiments, the V2 region of a heavy chain variable region comprising a sequence of the formula V1-X-V2, as described is a J region sequence. In some aspects, a humanized antibody comprising an ultralong CDR3 is as near to human in amino acid composition as possible. In some embodiments, the V2 region of the heavy chain variable region comprises an amino acid sequence selected from the group consisting of (i) WGHGTAVTVSS (SEQ ID NO: 142), (ii) WGKGTTVTVSS (SEQ ID NO: 143), (iii) WGKGTTVTVSS (SEQ ID NO: 144), (iv) WGRGTLVTVSS (SEQ ID NO: 145), (v) WGKGTTVTVSS (SEQ ID NO: 146), and (vi) WGQGLLVTVSS (SEQ ID NO: 147). In some embodiments, the V2 region of the heavy chain comprises the sequence set forth in SEQ ID NO: 147. In some embodiments, a J region sequence may be mutated from a bovine-derived sequence to a human sequence.

In some embodiments, a provided heavy chain variable region comprises a sequence of the formula V1-X-V2, wherein V1 is set forth in SEQ ID NO:141, X is an ultralong CDR-H3 set forth in SEQ ID NOS: 112, and V2 is set forth in any of SEQ ID NOS: 142-147. In some embodiments, a provided heavy chain variable region comprises a sequence of the formula V1-X-V2, wherein V1 is set forth in SEQ ID NO:141, X is an ultralong CDR-H3 set forth SEQ ID NOS: 114, and V2 is set forth in any of SEQ ID NOS: 142-147. In some embodiments, a provided heavy chain variable region comprises a sequence of the formula V1-X-V2, wherein V1 is set forth in SEQ ID NO:141, X is an ultralong CDR-H3 set forth in SEQ ID NO: 116, and V2 is set forth in any of SEQ ID NOS: 142-147. In some embodiments, a provided heavy chain variable region comprises a sequence of the formula V1-X-V2, wherein V1 is set forth in SEQ ID NO:141, X is an ultralong CDR-H3 set forth in SEQ ID NO: 117, and V2 is set forth in any of SEQ ID NOS: 142-147. In some embodiments, a provided heavy chain variable region comprises a sequence of the formula V1-X-V2, wherein V1 is set forth in SEQ ID NO:141, X is an ultralong CDR-H3 set forth in SEQ ID NO: 119, and V2 is set forth in any of SEQ ID NOS: 142-147. In some embodiments, a provided heavy chain variable region comprises a sequence of the formula V1-X-V2, wherein V1 is set forth in SEQ ID NO:141, X is an ultralong CDR-H3 set forth in SEQ ID NO: 121, and V2 is set forth in any of SEQ ID NOS: 142-147. In some embodiments, a provided heavy chain variable region comprises a sequence of the formula V1-X-V2, wherein V1 is set forth in SEQ ID NO:141, X is an ultralong CDR-H3 set forth in SEQ ID NO: 121, and V2 is set forth in any of SEQ ID NOS: 142-147. In some embodiments, a provided heavy chain variable region comprises a sequence of the formula V1-X-V2, wherein V1 is set forth in SEQ ID NO:141, X is an ultralong CDR-H3 set forth in SEQ ID NO: 123, and V2 is set forth in any of SEQ ID NOS: 142-147. In some embodiments, a provided heavy chain variable region comprises a sequence of the formula V1-X-V2, wherein V1 is set forth in SEQ ID NO:141, X is an ultralong CDR-H3 set forth in SEQ ID NO: 126, and V2 is set forth in any of SEQ ID NOS: 142-147.

In some embodiments, a provided antibody includes a heavy chain in which any of the provided variable heavy chain sequences as described is joined to a constant region of an immunoglobulin. In particular embodiments, the constant region is from a human immunoglobulin. In some embodiments, the constant region is a constant region of human IgG1. In some embodiments, the heavy chain includes a variable heavy chain as described that is joined to a human constant region. In some embodiments, the human constant region includes the CH1-CH2-CH3 constant domains. In some embodiments, the human constant region is of human IgG1 (e.g., with the sequence set forth in SEQ ID NO: 196, or a naturally occurring variant thereof, for instance with the K97R, D239E, or L241M mutation).

Alterations to the variable region notwithstanding, those skilled in the art will appreciate that the antibodies described herein will comprise antibodies (e.g., antibodies or antigen binding fragments thereof) in which at least a fraction of one or more of the constant region domains has been deleted or otherwise altered so as to provide desired biochemical characteristics such as increased serum half-life when compared with an antibody of approximately the same immunogenicity comprising a native or unaltered constant region. In some embodiments, the constant region of the modified antibodies will comprise a human constant region. Modifications to the constant region compatible with this invention comprise additions, deletions or substitutions of one or more amino acids in one or more domains. That is, the modified antibodies described herein can comprise alterations or modifications to one or more of the three heavy chain constant domains (CH1, CH2 or CH3). In some embodiments, modified constant regions wherein one or more domains are partially or entirely deleted are contemplated. In some embodiments, the omitted constant region domain will be replaced by a short amino acid spacer (e.g., 10 residues) that provides some of the molecular flexibility typically imparted by the absent constant region.

It will be noted that in certain embodiments, the modified antibodies can be engineered to fuse the CH3 domain directly to the hinge region of the respective modified antibodies. In other constructs it may be desirable to provide a peptide spacer between the hinge region and the CH2 and/or CH3 domains. Such a spacer can be added, for instance, to ensure that the regulatory elements of the constant domain remain free and accessible or that the hinge region remains flexible. However, it should be noted that amino acid spacers can, in some cases, prove to be immunogenic and elicit an unwanted immune response against the construct. Accordingly, in certain embodiments, any spacer added to the construct will be relatively non-immunogenic, or even omitted altogether, so as to maintain the desired biochemical qualities of the modified antibodies.

Besides the deletion of whole constant region domains, it will be appreciated that the antibodies described herein can be provided by the partial deletion or substitution of a few or even a single amino acid. For example, it may be desirable to simply delete that part of one or more constant region domains that control the effector function (e.g., complement C1Q binding) to be modulated. Such partial deletions of the constant regions can improve selected characteristics of the antibody (serum half-life) while leaving other desirable functions associated with the subject constant region domain intact. Moreover, as alluded to above, the constant regions of the disclosed antibodies can be modified through the mutation or substitution of one or more amino acids that enhances the profile of the resulting construct. In this respect it may be possible to disrupt the activity provided by a conserved binding site (e.g., Fc binding) while substantially maintaining the configuration and immunogenic profile of the modified antibody. Certain embodiments can comprise the addition of one or more amino acids to the constant region to enhance desirable characteristics such as decreasing or increasing effector function or provide for more cytotoxin or carbohydrate attachment. In such embodiments it can be desirable to insert or replicate specific sequences derived from selected constant region domains.

In some embodiments, any of the provided antibody or antigen binding fragment further comprises a light chain variable region. In some embodiments, the antibody variable heavy region or heavy chain is based on a bovine sequence and is paired with a variable light region or light chain of a bovine antibody. In some embodiments, the antibody variable heavy region or heavy chain is based on a humanized sequence and is paired with a variable light region or light chain of a bovine antibody. In some embodiments, the antibody variable heavy region or heavy chain is based on a humanized sequence and is paired with a humanized variable light region or light chain of a bovine antibody. In some embodiments, the antibody variable heavy region or heavy chain is based on a humanized sequence and is paired with a variable light region or light chain of a human antibody. In some embodiments, the light chain is a lambda light chain.

In some embodiments, the light chain variable region of a provided antibody or antigen binding fragment comprises a CDR L1, CDR L2, and CDR L3 of a light chain variable region of an antibody RBDA2, RBD C6, RBD F4, R2B1, R2D6, R2G1, R4A10, R4E5, R4G3, R4G11, R5A3, R4C1, R2C3, SKD, SKM, R2G3, R2F12, R2F12, SR3A3, or R2D9.

In some embodiments, a light chain variable region of a provided antibody or antigen binding fragment comprises a CDR L1, CDR L2, and CDR L3 of a light chain variable region of a bovine antibody, such as a variable light region of BLVH12, BLV5D3, BLV8C11, BF1H1, BLV5B8 and/or F18. In some embodiments, a light chain variable region of a provided antibody or antigen binding fragment comprises a CDR L1, CDR L2, and CDR L3 of the light chain variable region set forth in SEQ ID NO:2

Table 5 sets forth exemplary CDRs of a variable light chain of a provided antibody.

TABLE 5
Exemplary variable light chain complementarity determining region (CDR)
variable light chain
(SEQ ID NO)

| CDR L1 | CDR L2 | CDR L3 |
|---|---|---|
| SGSSSNVGNGYVS | GDTSRAS | ASAEDSSSNAV |
| (SEQ ID NO: 151) | (SEQ ID NO:152) | (SEQ ID NO: 153) |

In some embodiments, a variable heavy chain and a variable light chain variable region of a provided antibody or antigen binding fragment comprises a CDR H1, CDR H2, CDR H3, CDR L1, CDR L2, and CDR L3, respectively, of an antibody RBDA2, RBD C6, RBD F4, R2B1, R2D6, R2G1, R4A10, R4E5, R4G3, R4G11, R5A3, R4C1, R2C3, SKD, SKM, R2G3, R2F12, R2F12, SR3A3, or R2D9.

In some embodiments, a provided antibody or antigen binding fragment contains a variable heavy chain that has a CDR-H1, a CDR-H2 and CDR-H3 set forth in SEQ ID NOS: 84, 85 and 86, respectively, and a variable light chain that has a CDR-L1, a CDR-L2 and CDR-L3 set forth in SEQ ID NOS: 151, 152 and 153, respectively. In some embodiments, a provided antibody or antigen binding fragment contains a variable heavy chain that has a CDR-H1, a CDR-H2 and CDR-H3 set forth in SEQ ID NOS: 84, 87 and 88, respectively, and a variable light chain that has a CDR-L1, a CDR-L2 and CDR-L3 set forth in SEQ ID NOS: 151, 152 and 153, respectively. In some embodiments, a provided antibody or antigen binding fragment contains a variable heavy chain that has a CDR-H1, a CDR-H2 and CDR-H3 set forth in SEQ ID NOS: 84, 85 and 89, respectively, and a variable light chain that has a CDR-L1, a CDR-L2 and CDR-L3 set forth in SEQ ID NOS: 151, 152 and 153, respectively. In some embodiments, a provided antibody or antigen binding fragment contains a variable heavy chain that has a CDR-H1, a CDR-H2 and CDR-H3 set forth in SEQ ID NOS: 90, 91 and 92, respectively, and a variable light chain that has a CDR-L1, a CDR-L2 and CDR-L3 set forth in SEQ ID NOS: 151, 152 and 153, respectively. In some embodiments, a provided antibody or antigen binding fragment contains a variable heavy chain that has a CDR-H1, a CDR-H2 and CDR-H3 set forth in SEQ ID NOS: 93, 94 and 95, respectively, and a variable light chain that has a CDR-L1, a CDR-L2 and CDR-L3 set forth in SEQ ID NOS: 151, 152 and 153, respectively. In some embodiments, a provided antibody or antigen binding fragment contains a variable heavy chain that has a CDR-H1, a CDR-H2 and CDR-H3 set forth in SEQ ID NOS: 96, 97 and 98, respectively, and a variable light chain that has a CDR-L1, a CDR-L2 and CDR-L3 set forth in SEQ ID NOS: 151, 152 and 153, respectively. In some embodiments, a provided antibody or antigen binding fragment contains a variable heavy chain that has a CDR-H1, a CDR-H2 and CDR-H3 set forth in SEQ ID NOS: 99, 100 and 101, respectively, and a variable light chain that has a CDR-L1, a CDR-L2 and CDR-L3 set forth in SEQ ID NOS: 151, 152 and 153, respectively. In some embodiments, a provided antibody or antigen binding fragment contains a variable heavy chain that has a CDR-H1, a CDR-H2 and CDR-H3 set forth in SEQ ID NOS: 96, 102 and 103, respectively, and a variable light chain that has a CDR-L1, a CDR-L2 and CDR-L3 set forth in SEQ ID NOS: 151, 152 and 153, respectively. In some embodiments, a provided antibody or antigen binding fragment contains a variable heavy chain that has a CDR-H1, a CDR-H2 and CDR-H3 set forth in SEQ ID NOS: 99, 102 and 104, respectively, and a variable light chain that has a CDR-L1, a CDR-L2 and CDR-L3 set forth in SEQ ID NOS: 151, 152 and 153, respectively. In some embodiments, a provided antibody or antigen binding fragment contains a variable heavy chain that has a CDR-H1, a CDR-H2 and CDR-H3 set forth in SEQ ID NOS: 105, 106 and 107, respectively, and a variable light chain that has a CDR-L1, a CDR-L2 and CDR-L3 set forth in SEQ ID NOS: 151, 152 and 153, respectively. In some embodiments, a provided antibody or antigen binding fragment contains a variable heavy chain that has a CDR-H1, a CDR-H2 and CDR-H3 set forth in SEQ ID NOS: 108, 109 and 110, respectively, and a variable light chain that has a CDR-L1, a CDR-L2 and CDR-L3 set forth in SEQ ID NOS: 151, 152 and 153, respectively. In some embodiments, a provided antibody or antigen binding fragment contains a variable heavy chain that has a CDR-H1, a CDR-H2 and CDR-H3 set forth in SEQ ID NOS: 108, 111 and 112, respectively, and a variable light chain that has a CDR-L1, a CDR-L2 and CDR-L3 set forth in SEQ ID NOS: 151, 152 and 153, respectively. In some embodiments, a provided antibody or antigen binding fragment contains a variable heavy chain that has a CDR-H1, a CDR-H2 and CDR-H3 set forth in SEQ ID NOS: 113, 111 and 114, respectively, and a variable light chain that has a CDR-L1, a CDR-L2 and CDR-L3 set forth in SEQ ID NOS: 151, 152 and 153, respectively. In some embodiments, a provided antibody or antigen binding fragment contains a variable heavy chain that has a CDR-H1, a CDR-H2 and CDR-H3 set forth in SEQ ID NOS: 108, 115 and 116, respectively, and a variable light chain that has a CDR-L1, a CDR-L2 and CDR-L3 set forth in SEQ ID NOS: 151, 152 and 153, respectively. In some embodiments, a provided antibody or antigen binding fragment contains a variable heavy chain that has a CDR-H1, a CDR-H2 and CDR-H3 set forth in SEQ ID NOS: 108, 115 and 117, respectively, and a variable light chain that has a CDR-L1, a CDR-L2 and CDR-L3 set forth in SEQ ID NOS: 151, 152 and 153, respectively. In some embodiments, a provided antibody or antigen binding fragment contains a variable heavy chain that has a CDR-H1, a CDR-H2 and CDR-H3 set forth in SEQ ID NOS: 108, 118 and 119, respectively, and a variable light chain that has a CDR-L1, a CDR-L2 and CDR-L3 set forth in SEQ ID NOS: 151, 152 and 153, respectively. In some embodiments, a provided antibody or antigen binding fragment contains a variable heavy chain that has a CDR-H1, a CDR-H2 and CDR-H3 set forth in SEQ ID NOS: 108, 120 and 121, respectively, and a variable light chain that has a CDR-L1, a CDR-L2 and CDR-L3 set forth in SEQ ID NOS: 151, 152 and 153, respectively. In some embodiments, a provided antibody or antigen binding fragment contains a variable heavy chain that has a CDR-H1, a CDR-H2 and CDR-H3 set forth in SEQ ID NOS: 122, 111 and 123, respectively, and a variable light chain that has a CDR-L1, a CDR-L2 and CDR-L3 set forth in SEQ ID NOS: 151, 152 and 153, respectively. In some embodiments, a provided antibody or antigen binding fragment contains a variable heavy chain that has a CDR-H1, a CDR-H2 and CDR-H3 set forth in SEQ ID NOS: 124, 125 and 126, respectively, and a variable light chain that has a CDR-L1, a CDR-L2 and CDR-L3 set forth in SEQ ID NOS: 151, 152 and 153, respectively.

In some embodiments, the variable light region is a variable light region of a bovine antibody, such as a variable light region of BLVH12, BLV5D3, BLV8C11, BF1H1, BLV5B8 and/or F18. In some embodiments, the light chain variable region may comprise the polypeptide sequence of SEQ ID NO: 2 or a sequence of amino acids that exhibits at least 85%, 86%, 87%, 88%, 89%, 90%, 91%,92%, 93%, 94%, 95%, 96%, 97%, 98%, or 99% sequence identity to SEQ ID NO:2. In some embodiments, the light chain variable region may comprise the polypeptide sequence of SEQ ID NO: 2.

In some embodiments, a variable heavy chain and a variable light chain variable region of a provided antibody or antigen binding fragment comprises the sequences set forth in SEQ ID NO:33 and SEQ ID NO:2, respectively. In some embodiments, a variable heavy chain and a variable light chain variable region of a provided antibody or antigen binding fragment comprises the sequences set forth in SEQ ID NO:34 and SEQ ID NO:2, respectively. In some embodiments, a variable heavy chain and a variable light chain variable region of a provided antibody or antigen binding fragment comprises the sequences set forth in SEQ ID NO:35 and SEQ ID NO:2, respectively. In some embodiments, a variable heavy chain and a variable light chain variable region of a provided antibody or antigen binding fragment comprises the sequences set forth in SEQ ID NO:36 and SEQ ID NO:2, respectively. In some embodiments, a variable heavy chain and a variable light chain variable region of a provided antibody or antigen binding fragment comprises the sequences set forth in SEQ ID NO:37 and SEQ ID NO:2, respectively. In some embodiments, a variable heavy chain and a variable light chain variable region of a provided antibody or antigen binding fragment comprises the sequences set forth in SEQ ID NO:38 and SEQ ID NO:2, respectively. In some embodiments, a variable heavy chain and a variable light chain variable region of a provided antibody or antigen binding fragment comprises the sequences set forth in SEQ ID NO:39 and SEQ ID NO:2, respectively. In some embodiments, a variable heavy chain and a variable light chain variable region of a provided antibody or antigen binding fragment comprises the sequences set forth in SEQ ID NO:40 and SEQ ID NO:2, respectively. In some embodiments, a variable heavy chain and a variable light chain variable region of a provided antibody or antigen binding fragment comprises the sequences set forth in SEQ ID NO:41 and SEQ ID NO:2, respectively. In some embodiments, a variable heavy chain and a variable light chain variable region of a provided antibody or antigen binding fragment comprises the sequences set forth in SEQ ID NO:42 and SEQ ID NO:2, respectively. In some embodiments, a variable heavy chain and a variable light chain variable region of a provided antibody or antigen binding fragment comprises the sequences set forth in SEQ ID NO:43 and SEQ ID NO:2, respectively. In some embodiments, a variable heavy chain and a variable light chain variable region of a provided antibody or antigen binding fragment comprises the sequences set forth in SEQ ID NO:44 and SEQ ID NO:2, respectively. In some embodiments, a variable heavy chain and a variable light chain variable region of a provided antibody or antigen binding fragment comprises the sequences set forth in SEQ ID NO:45 and SEQ ID NO:2, respectively. In some embodiments, a variable heavy chain and a variable light chain variable region of a provided antibody or antigen binding fragment comprises the sequences set forth in SEQ ID NO:46 and SEQ ID NO:2, respectively. In some embodiments, a variable heavy chain and a variable light chain variable region of a provided antibody or antigen binding fragment comprises the sequences set forth in SEQ ID NO:47 and SEQ ID NO:2, respectively. In some embodiments, a variable heavy chain and a variable light chain variable region of a provided antibody or antigen binding fragment comprises the sequences set forth in SEQ ID NO:48 and SEQ ID NO:2, respectively. In some embodiments, a variable heavy chain and a variable light chain variable region of a provided antibody or antigen binding fragment comprises the sequences set forth in SEQ ID NO:48 and SEQ ID NO:2, respectively. In some embodiments, a variable heavy chain and a variable light chain variable region of a provided antibody or antigen binding fragment comprises the sequences set forth in SEQ ID NO:49 and SEQ ID NO:2, respectively. In some embodiments, a variable heavy chain and a variable light chain variable region of a provided antibody or antigen binding fragment comprises the sequences set forth in SEQ ID NO:50 and SEQ ID NO:2, respectively. In some embodiments, a variable heavy chain and a variable light chain variable region of a provided antibody or antigen binding fragment comprises the sequences set forth in SEQ ID NO:51 and SEQ ID NO:2, respectively.

In some embodiments, provided is an ultralong CDR3 antibody or antigen binding fragment containing a variable heavy chain and a variable light chain variable region that comprises the sequences set forth in SEQ ID NO:33 and SEQ ID NO:2, respectively. In some embodiments, provided is an ultralong CDR3 antibody or antigen binding fragment containing a variable heavy chain and a variable light chain variable region that comprises the sequences set forth in SEQ ID NO:34 and SEQ ID NO:2, respectively. In some embodiments, provided is an ultralong CDR3 antibody or antigen binding fragment containing a variable heavy chain and a variable light chain variable region that comprises the sequences set forth in SEQ ID NO:35 and SEQ ID NO:2, respectively. In some embodiments, provided is an ultralong CDR3 antibody or antigen binding fragment containing a variable heavy chain and a variable light chain variable region that comprises the sequences set forth in SEQ ID NO:40 and SEQ ID NO:2, respectively. In some embodiments, provided is an ultralong CDR3 antibody or antigen binding fragment containing a variable heavy chain and a variable light chain variable region that comprises the sequences set forth in SEQ ID NO:45 and SEQ ID NO:2, respectively. In some embodiments, provided is an ultralong CDR3 antibody or antigen binding fragment containing a variable heavy chain and a variable light chain variable region that comprises the sequences set forth in SEQ ID NO:46 and SEQ ID NO:2, respectively. In some embodiments, provided is an ultralong CDR3 antibody or antigen binding fragment containing a variable heavy chain and a variable light chain variable region that comprises the sequences set forth in SEQ ID NO:50 and SEQ ID NO:2, respectively. In some embodiments, provided is an ultralong CDR3 antibody or antigen binding fragment containing a variable heavy chain and a variable light chain variable region that comprises the sequences set forth in SEQ ID NO:51 and SEQ ID NO:2, respectively.

In some embodiments, the antibody is a full-length antibody containing any of the provided heavy chains (e.g. containing any of the provided variable heavy regions, such as set forth in any of SEQ ID NOS: 33-51, joined to a human IgG1 constant region) and a full length light chain.

In some embodiments, the light region is a light chain of a bovine antibody, such as a light chain of BLVH12, BLV5D3, BLV8C11, BF1H1, BLV5B8 and/or F18. In some embodiments, the light chain may comprise a sequence set forth in SEQ ID NO: 149. In some embodiments, the light chain may comprise a sequence set forth in SEQ ID NO: 150.

In some embodiments, a humanized heavy chain variable region or heavy chain may be paired with a humanized light chain.

In some embodiments, a humanized heavy chain variable region or heavy chain may be paired with a human light chain. In some embodiments, the light chain is homologous to a bovine light chain known to pair with a bovine ultralong CDR3 heavy chain. Several human VL sequences can be used to paired with the sequences above, including VL1-47, VL1-40, VL1-51, and VL2-18, which are homologous to the lambda region derived from Bos Taurus. In some embodiments, the light chain variable region is a sequence set forth in any one of SEQ ID NOS: 155-158. In some embodiments, the light chain variable region comprises a variable region of the VL1-51 germline sequence set forth in SEQ ID NO: 154. In some embodiments, the light chain is the VL1-51 germline sequence set forth in SEQ ID NO: 154.

In some embodiments, the light chain variable region is a human germline light chain sequence, such as any described above, that contains one or more amino acid modifications. Such modifications may include the substitution of certain amino acid residues in the human light chain to those residues at corresponding positions in a bovine light chain sequence. The modified light chains may improve the yield of the antibody comprising the ultralong CDR3 and/or increase its binding specificity. In some embodiments, the modifications include one or more of amino acid replacements S2A, T5N, P8S, A12G, A13S, and P14L based on Kabat numbering. In some embodiments, the modifications include amino acid replacements S2A, T5N, P8S, A12G, A13S, and P14L based on Kabat numbering. In some embodiments, the modifications are in the CDR1 and include amino acid replacements I29V and N32G. In some embodiments, the modifications are in the CDR2 and include substitution of DNN to GDT. In some embodiments, the modifications are inn CDR2 and include a substitution DNNKRP to GDTSRA. In some embodiments, the modifications include a combination of any of the foregoing. For example, provided modifications of a human germline light chain sequence include amino acid replacements S2A, T5N, P8S, A12G, A13S, and P14L based on Kabat numbering and substitution of DNN to GDT in CDR2.

Also provided are antibodies and antigen-binding fragments thereof having sequences at least at or about at least 90%, 91%, 92%, 93%, 94%, 95%, 96%, 97%, 98%, or 99% identical to such sequences. Thus, it is understood that the present disclosure also encompasses variants and equivalents which are substantially homologous to the provided antibodies, including chimeric, humanized and human antibodies, or antibody fragments thereof, set forth herein. These can contain, for example, conservative substitution mutations, i.e., the substitution of one or more amino acids by similar amino acids. For example, conservative substitution refers to the substitution of an amino acid with another within the same general class such as, for example, one acidic amino acid with another acidic amino acid, one basic amino acid with another basic amino acid or one neutral amino acid by another neutral amino acid. What is intended by a conservative amino acid substitution is well known in the art.

In some embodiments, the antibody is a full length antibody containing the heavy chain and the light chain.

In some embodiments, among the provided antibodies are antibody fragments. An “antibody fragment” or “antigen-binding fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; heavy chain variable (V_{H}) regions, single-chain antibody molecules such as scFvs and single-domain antibodies comprising only the V_{H} region; and multispecific antibodies formed from antibody fragments. In some embodiments, the antibody is or comprises an antibody fragment comprising a variable heavy chain (Vii) and a variable light chain (V_{L}) region. In particular embodiments, the antibodies are single-chain antibody fragments comprising a heavy chain variable (V_{H}) region and a light chain variable (V_{L}) region, such as scFvs.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In certain embodiments, antibody fragments are produced recombinantly. Fab, Fv, and scFv antibody fragments can all be expressed in and secreted from E. coli or other host cells, thus allowing the production of large amounts of these fragments. Such antibody fragments can also be isolated from antibody phage libraries. The antibody fragment can also be linear antibodies as described in U.S. Pat. No. 5,641,870, for example, and can be monospecific or bispecific. Other techniques for the production of antibody fragments will be apparent to the skilled practitioner.

In some aspects, the antibody fragments are scFvs. In other aspects, the antibody fragments are Fabs. In some embodiments, the antibody fragment is an F(ab) or F(ab′)₂.

In some embodiments, the antibody is a polyclonal antibody. In some embodiments, the antibody is a monoclonal antibody. In some embodiments, antibody is a recombinant antibody, a chimeric antibody, a humanized antibody, an antibody fragment, a bispecific antibody, or a trispecific antibody.

In some embodiments, the provided antibodies are monoclonal antibodies, including monoclonal antibody fragments. The term “monoclonal antibody” as used herein refers to an antibody obtained from or within a population of substantially homogeneous antibodies, i.e., the individual antibodies comprising the population are identical, except for possible variants containing naturally occurring mutations or arising during production of a monoclonal antibody preparation, such variants generally being present in minor amounts. In contrast to polyclonal antibody preparations, which typically include different antibodies directed against different epitopes, each monoclonal antibody of a monoclonal antibody preparation is directed against a single epitope on an antigen. The term is not to be construed as requiring production of the antibody by any particular method. A monoclonal antibody may be made by a variety of techniques, including but not limited to generation from a hybridoma, recombinant DNA methods, phage-display and other antibody display methods.

In some embodiments, the antibodies or antigen binding fragments are isolated. The antibodies or antigen binding fragments provided herein can be recombinant polypeptides, natural polypeptides, or synthetic polypeptides comprising an antibody, or fragment thereof. It will be recognized in the art that some amino acid sequences described herein can be varied without significant effect of the structure or function of the protein. Such mutants include deletions, insertions, inversions, repeats, and type substitutions.

The polypeptides and analogs can be further modified to contain additional chemical moieties not normally part of the protein. Those derivatized moieties can improve the solubility, the biological half-life or absorption of the protein. The moieties can also reduce or eliminate any desirable side effects of the proteins and the like. An overview for those moieties can be found in REMINGTON'S PHARMACEUTICAL SCIENCES, 21th ed., Mack Publishing Co., Easton, PA (2005).

Also provided are nucleic acid molecules encoding any of the antibodies or antigen binding fragments. The nucleic acid may encode an amino acid sequence comprising the V_{L} region and/or an amino acid sequence comprising the V_{H} region of the antibody (e.g., the light and/or heavy chains of the antibody). The nucleic acid may encode one or more amino acid sequence comprising the V_{L} region and/or an amino acid sequence comprising the V_{H} region of the antibody (e.g., the light and/or heavy chains of the antibody). In some embodiments, the nucleic acid, e.g., polynucleotide encodes one or more V_{H} region and/or one or more V_{L} region of the antibody, in any order or orientation. In some embodiments, the nucleic acid, e.g., polynucleotide encodes a V_{H} region and a V_{L} region, and the coding sequence for the V_{H} region is upstream of the coding sequence for the V_{L} region. In some embodiments, the nucleic acid, e.g., polynucleotide encodes a V_{H} region and a V_{L} region, and the coding sequence for the V_{L} region is upstream of the coding sequence for the V_{H} region.

Also provided hare methods for making the antibodies or antigen binding fragments, including vectors and host cells for producing the same. For instance, for production of antibodies, in addition to prokaryotes, eukaryotic microbes such as filamentous fungi or yeast are suitable cloning or expression hosts for antibody-encoding vectors, including fungi and yeast strains whose glycosylation pathways have been modified to mimic or approximate those in human cells, resulting in the production of an antibody with a partially or fully human glycosylation pattern. See Gerngross, Nat. Biotech. 22:1409-1414 (2004), and Li et al., Nat. Biotech. 24:210-215 (2006).

Exemplary eukaryotic cells that may be used to express polypeptides include, but are not limited to, COS cells, including COS 7 cells; 293 cells, including 293-6E cells; CHO cells, including CHO-S, DG44. Lec13 CHO cells, and FUT8 CHO cells; PER.C6® cells; and NSO cells. In some embodiments, the antibody heavy chains and/or light chains (e.g., V_{H} region and/or V_{L} region) may be expressed in yeast. See, e.g., U.S. Publication No. US 2006/0270045 A1. In some embodiments, a particular eukaryotic host cell is selected based on its ability to make desired post-translational modifications to the heavy chains and/or light chains (e.g., V_{H} region and/or V_{L} region). For example, in some embodiments, CHO cells produce polypeptides that have a higher level of sialylation than the same polypeptide produced in 293 cells.

In some embodiments, the antibody or antigen-binding fragment provided herein is produced in a cell-free system. Exemplary cell-free systems are described, e.g., in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

In some embodiments, any of the provided In some of any embodiments, an antibody or antigen-binding fragment containing the variable heavy chain binds to a spike protein of a coronavirus, e.g. SARs-CoV2. Exemplary features of provided antibodies are described, e.g. in Section II.C.

### B. Knob Peptides

Provided herein is an isolated knob peptide of over 30 amino acids in length directed against SARS-CoV-2, wherein the knob peptide is isolated or derived from an ultralong CDR3 of a bovine antibody. Also provided herein are knob peptides that are independently produced, such as synthetically or by recombinant DNA methods. In some embodiments, any of the provided knob peptides are derived from or based on an ultralong CDR3 of a bovine antibody as described above.

Provided herein are knob peptides that are 25-70 amino acids in length and contain 2 to 6 disulfide bonds. In some embodiments, the knob peptides are derived from an ultralong CDR3 of any of antibodies R4C1, R2C3, SKD, SKM, R2G3, R2F12, SR3A3 or R2D9. In some embodiments, the knob peptide is a contiguous sequence of amino acids of an ultralong CDR3 as contained in the heavy chain variable region set forth in any of SEQ ID NOS: 33, 34, 35, 40, 45, 46, 50 or 51. In some embodiments, the knob peptide may include a contiguous portion of amino acids of the ascending (Stalk A) or descending (Stalk B) of as contained in the ultralong CDR3 of the heavy chain variable region set forth in any of SEQ ID NOS: 33, 34, 35, 40, 45, 46, 50 or 51.

In some embodiments, the knob peptide includes a contiguous sequence of amino acids within an ultralong CDR3 set forth in any of SEQ ID NOS: 112, 114, 116, 117, 119, 121, 123 or 126. In some embodiments, the knob peptide lacks all or a portion of the ascending (Stalk A) or descending (Stalk B) domain of an ultralong CDR3. In some embodiments, the contiguous sequence of amino acids includes at least 40 amino acids within the sequence set forth in any one of SEQ ID NOS: 112, 114, 116, 117, 119, 121, 123, or 126. In some of any of the provided embodiments, the knob peptide is up to 70 amino acids in length and contains 2-6 disulfide bonds.

In some embodiments, a contiguous sequence of amino acids of a provided knob peptide includes 6-9 C-terminal amino acids after the last cysteine (Cys) residue of the respective sequence of amino acids set forth in any one of SEQ ID NOS: 112, 114, 116, 117, 119, 121, 123, or 126. In some embodiments, the knob peptide comprises at or about 6 C-terminal amino acids after the last cysteine (Cys) residue of the respective sequence of amino acids set forth in any one of SEQ ID NOS: 112, 114, 116, 117, 119, 121, 123, or 126. In some embodiments, the knob peptide comprises at or about 7 C-terminal amino acids after the last cysteine (Cys) residue of the respective sequence of amino acids set forth in any one of SEQ ID NOS: 112, 114, 116, 117, 119, 121, 123, or 126. In some embodiments, the knob peptide comprises at or about 8 C-terminal amino acids after the last cysteine (Cys) residue of the respective sequence of amino acids set forth in any one of SEQ ID NOS: 112, 114, 116, 117, 119, 121, 123, or 126. In some embodiments, the knob peptide comprises at or about 9 C-terminal amino acids after the last cysteine (Cys) residue of the respective sequence of amino acids set forth in any one of SEQ ID NOS: 112, 114, 116, 117, 119, 121, 123, or 126.

In some embodiments, a contiguous sequence of amino acids of a provided knob peptide begins 3-6 amino acids before the N-terminal Cys residue of the respective sequence of amino acids set forth in any one of SEQ ID NOS: 112, 114, 116, 117, 119, 121, 123, or 126. In some embodiments, a contiguous sequence of amino acids of a provided knob peptide begins at or about 3 amino acids before the N-terminal Cys residue of the respective sequence of amino acids set forth in any one of SEQ ID NOS: 112, 114, 116, 117, 119, 121, 123, or 126. In some embodiments, a contiguous sequence of amino acids of a provided knob peptide begins at or about 4 amino acids before the N-terminal Cys residue of the respective sequence of amino acids set forth in any one of SEQ ID NOS: 112, 114, 116, 117, 119, 121, 123, or 126. In some embodiments, a contiguous sequence of amino acids of a provided knob peptide begins at or about 5 amino acids before the N-terminal Cys residue of the respective sequence of amino acids set forth in any one of SEQ ID NOS: 112, 114, 116, 117, 119, 121, 123, or 126. In some embodiments, a contiguous sequence of amino acids of a provided knob peptide begins at or about 6 amino acids before the N-terminal Cys residue of the respective sequence of amino acids set forth in any one of SEQ ID NOS: 112, 114, 116, 117, 119, 121, 123, or 126.

In some embodiments, the knob peptide is 40 to 60 amino acids in length. In some embodiments, the knob peptide is at least 42 amino acids in length. In some embodiments, the knob peptide is 42 amino acids, 43 amino acids, 44 amino acids, 45 amino acids, 46 amino acids, 47 amino acids, 48 amino acids, 49 amino acids, 50 amino acids, 51 amino acids, 52 amino acids, 53 amino acids, 54 amino acids, 55 amino acids, 56 amino acids, 57 amino acids, 58 amino acids, 59 amino acids or 60 amino acids in length.

In some embodiments, the knob peptide has 2-12 cysteine (Cys) residues. In some embodiments, the knob peptide has 2, 4, 6, 8, 10 or 12 cysteine residues. In some embodiments, the knob peptide comprises at least 4 Cys residues. In some embodiments, the knob peptide contains 4 Cys residues.

In some embodiments, the knob peptide has 2 to 6 disulfide bonds. In some embodiments, the knob peptide has 2, 3, 4, 5 or 6 disulfide bonds. In some embodiments, the knob peptide has at least 2 disulfide bonds. In some embodiments, the knob peptide has 2 disulfide bonds.

Exemplary knob peptides provided herein include any set forth in Table 6. In some embodiments, the knob peptide comprises an amino acid sequence set forth in any one of SEQ ID NOS: 160, 169, 179, 189, 198, 208, 215 or 224. In some embodiments, the knob peptide is at or about 42 amino acids in length, 43 amino acids, 44 amino acids, 45 amino acids, 46 amino acids, 47 amino acids, 48 amino acids, 49 amino acids, 50 amino acids, 51 amino acids, 52 amino acids, 53 amino acids, 54 amino acids or 55 amino acids.

In some embodiments, the knob peptide comprises the amino acid sequence set forth in SEQ ID NO:160. In some embodiments, the knob peptide is set forth in any one of SEQ ID NOS: 63 or 159-168.

In some embodiments, the knob peptide comprises the amino acid sequence set forth in SEQ ID NO: 169. In some embodiments, the knob peptide is set forth in any one of SEQ ID NOS: 66 or 169-177.

In some embodiments, the knob peptide comprises the amino acid sequence set forth in SEQ ID NO:179. In some embodiments, the knob peptide is set forth in any one of SEQ ID NOS: 65 or 178-187.

In some embodiments, the knob peptide comprises the amino acid sequence set forth in SEQ ID NO:189. In some embodiments, the knob peptide is set forth in any one of SEQ ID NOS: 64 or 188-197.

In some embodiments, the knob peptide comprise the amino acid sequence set forth in SEQ ID NO:198. In some embodiments, the knob peptide is set forth in any one of SEQ ID NOS: 60 or 198-206.

In some embodiments, the knob peptide comprises the amino acid sequence set forth in SEQ ID NO:208. In some embodiments, the knob peptide is set forth in any one of SEQ ID NOS: 62 or 207-214.

In some embodiments, the knob peptide comprises the amino acid sequence set forth in SEQ ID NO:215. In some embodiments, the knob peptide is set forth in any of SEQ ID NOS: 61 and 215-223.

In some embodiments, the knob peptide comprises the amino acid sequence set forth in SEQ ID NO:224. In some embodiments, the knob peptide is set forth in any one of SEQ ID NOS: 68 or 224-232.

TABLE 6
Exemplary CDR H3-Knob Sequences

| | SEQ | |
|---|---|---|
| Name | ID NO | Knob Sequence |
| R4C1 | 159 | RKEKNCPDGYIYSSNTASGYDCGVWICRRVGSAFCSRTGDYTSPSEFDIYEFY |
| knob | 160 | RKEKNCPDGYIYSSNTASGYDCGVWICRRVGSAFCSRTGDYTS |
| | 161 | RKEKNCPDGYIYSSNTASGYDCGVWICRRVGSAFCSRTGDYTSP |
| | 162 | RKEKNCPDGYIYSSNTASGYDCGVWICRRVGSAFCSRTGDYTSPS |
| | 163 | RKEKNCPDGYIYSSNTASGYDCGVWICRRVGSAFCSRTGDYTSPSE |
| | 164 | RKEKNCPDGYIYSSNTASGYDCGVWICRRVGSAFCSRTGDYTSPSEF |
| | 165 | RKEKNCPDGYIYSSNTASGYDCGVWICRRVGSAFCSRTGDYTSPSEFD |
| | 166 | RKEKNCPDGYIYSSNTASGYDCGVWICRRVGSAFCSRTGDYTSPSEFDI |
| | 63 | RKEKNCPDGYIYSSNTASGYDCGVWICRRVGSAFCSRTGDYTSPSEFDIY |
| | 167 | RKEKNCPDGYIYSSNTASGYDCGVWICRRVGSAFCSRTGDYTSPSEFDIYE |
| | 168 | RKEKNCPDGYIYSSNTASGYDCGVWICRRVGSAFCSRTGDYTSPSEFDIYEF |
| R2C3 | 66 | RKEKSCPDGYLYSSNTGRGYDCGVWTCRRVGGEFCSATGDWTSPSEEDFYEF |
| knob | 169 | RKEKSCPDGYLYSSNTGRGYDCGVWTCRRVGGEFCSATGDWTS |
| | 170 | RKEKSCPDGYLYSSNTGRGYDCGVWTCRRVGGEFCSATGDWTSP |
| | 171 | RKEKSCPDGYLYSSNTGRGYDCGVWTCRRVGGEFCSATGDWTSPS |
| | 172 | RKEKSCPDGYLYSSNTGRGYDCGVWTCRRVGGEFCSATGDWTSPSE |
| | 173 | RKEKSCPDGYLYSSNTGRGYDCGVWTCRRVGGEFCSATGDWTSPSEE |
| | 174 | RKEKSCPDGYLYSSNTGRGYDCGVWTCRRVGGEFCSATGDWTSPSEED |
| | 175 | RKEKSCPDGYLYSSNTGRGYDCGVWTCRRVGGEFCSATGDWTSPSEEDF |
| | 176 | RKEKSCPDGYLYSSNTGRGYDCGVWTCRRVGGEFCSATGDWTSPSEEDFY |
| | 177 | RKEKSCPDGYLYSSNTGRGYDCGVWTCRRVGGEFCSATGDWTSPSEEDFYE |
| SKD | 178 | SEKRSCPGGSSRRYPSGASCDVSGGACACYVSNCRGVLCPTLNEIVAYTYEWH |
| knob | | VD |
| | 179 | SEKRSCPGGSSRRYPSGASCDVSGGACACYVSNCRGVLCPTLNEI |
| | 180 | SEKRSCPGGSSRRYPSGASCDVSGGACACYVSNCRGVLCPTLNEIV |
| | 181 | SEKRSCPGGSSRRYPSGASCDVSGGACACYVSNCRGVLCPTLNEIVA |
| | 182 | SEKRSCPGGSSRRYPSGASCDVSGGACACYVSNCRGVLCPTLNEIVAY |
| | 183 | SEKRSCPGGSSRRYPSGASCDVSGGACACYVSNCRGVLCPTLNEIVAYT |
| | 65 | SEKRSCPGGSSRRYPSGASCDVSGGACACYVSNCRGVLCPTLNEIVAYTY |
| | 184 | SEKRSCPGGSSRRYPSGASCDVSGGACACYVSNCRGVLCPTLNEIVAYTYE |
| | 185 | SEKRSCPGGSSRRYPSGASCDVSGGACACYVSNCRGVLCPTLNEIVAYTYEW |
| | 186 | SEKRSCPGGSSRRYPSGASCDVSGGACACYVSNCRGVLCPTLNEIVAYTYEWH |
| | 187 | SEKRSCPGGSSRRYPSGASCDVSGGACACYVSNCRGVLCPTLNEIVAYTYEWH |
| | | V |
| SKM | 188 | LRSCPDGYIDNSGCTADWGCAALDCWRRRFGYHSTDPSHYTGATYIYTYSHI |
| knob | | D |
| | 189 | LRSCPDGYIDNSGCTADWGCAALDCWRRRFGYHSTDPSHYTGAT |
| | 190 | LRSCPDGYIDNSGCTADWGCAALDCWRRRFGYHSTDPSHYTGATY |
| | 191 | LRSCPDGYIDNSGCTADWGCAALDCWRRRFGYHSTDPSHYTGATYI |
| | 192 | LRSCPDGYIDNSGCTADWGCAALDCWRRRFGYHSTDPSHYTGATYIY |
| | 193 | LRSCPDGYIDNSGCTADWGCAALDCWRRRFGYHSTDPSHYTGATYIYT |
| | 64 | LRSCPDGYIDNSGCTADWGCAALDCWRRRFGYHSTDPSHYTGATYIYTY |
| | 194 | LRSCPDGYIDNSGCTADWGCAALDCWRRRFGYHSTDPSHYTGATYIYTYS |
| | 195 | LRSCPDGYIDNSGCTADWGCAALDCWRRRFGYHSTDPSHYTGATYIYTYS |
| | 196 | LRSCPDGYIDNSGCTADWGCAALDCWRRRFGYHSTDPSHYTGATYIYTYSH |
| | 197 | LRSCPDGYIDNSGCTADWGCAALDCWRRRFGYHSTDPSHYTGATYIYTYSHI |
| R2G3 | 60 | EGDKTCPDGYEHTCGCIGGCGCKRSACIGALCCQASLGGWLSDGETYTYEF |
| knob | 198 | EGDKTCPDGYEHTCGCIGGCGCKRSACIGALCCQASLGGWLS |
| | 199 | EGDKTCPDGYEHTCGCIGGCGCKRSACIGALCCQASLGGWLSD |
| | 200 | EGDKTCPDGYEHTCGCIGGCGCKRSACIGALCCQASLGGWLSDG |
| | 201 | EGDKTCPDGYEHTCGCIGGCGCKRSACIGALCCQASLGGWLSDGE |
| | 202 | EGDKTCPDGYEHTCGCIGGCGCKRSACIGALCCQASLGGWLSDGET |
| | 203 | EGDKTCPDGYEHTCGCIGGCGCKRSACIGALCCQASLGGWLSDGETY |
| | 204 | EGDKTCPDGYEHTCGCIGGCGCKRSACIGALCCQASLGGWLSDGETYT |
| | 205 | EGDKTCPDGYEHTCGCIGGCGCKRSACIGALCCQASLGGWLSDGETYTY |
| | 206 | EGDKTCPDGYEHTCGCIGGCGCKRSACIGALCCQASLGGWLSDGETYTYE |
| R2F12 | 207 | KTKNACPDDFDYRCSCIGGCGCARKGCVGPLCCRSDLGGYLTDSPAYIYEWY |
| knob | 208 | KTKNACPDDFDYRCSCIGGCGCARKGCVGPLCCRSDLGGYLTDS |
| | 209 | KTKNACPDDFDYRCSCIGGCGCARKGCVGPLCCRSDLGGYLTDSP |
| | 210 | KTKNACPDDFDYRCSCIGGCGCARKGCVGPLCCRSDLGGYLTDSPA |
| | 211 | KTKNACPDDFDYRCSCIGGCGCARKGCVGPLCCRSDLGGYLTDSPAY |
| | 212 | KTKNACPDDFDYRCSCIGGCGCARKGCVGPLCCRSDLGGYLTDSPAYI |
| | 213 | KTKNACPDDFDYRCSCIGGCGCARKGCVGPLCCRSDLGGYLTDSPAYIY |
| | 62 | KTKNACPDDFDYRCSCIGGCGCARKGCVGPLCCRSDLGGYLTDSPAYIYE |
| | 214 | KTKNACPDDFDYRCSCIGGCGCARKGCVGPLCCRSDLGGYLTDSPAYIYEW |
| SR3A3 | 61 | KEKNCPDGYIYSSNITSGFDCGVWICRRVGSAFCSRTGDYTSPTELDIYEF |
| knob | 215 | KEKNCPDGYIYSSNITSGFDCGVWICRRVGSAFCSRTGDYTS |
| | 216 | KEKNCPDGYIYSSNITSGFDCGVWICRRVGSAFCSRTGDYTSP |
| | 217 | KEKNCPDGYIYSSNITSGFDCGVWICRRVGSAFCSRTGDYTSPT |
| | 218 | KEKNCPDGYIYSSNITSGFDCGVWICRRVGSAFCSRTGDYTSPTE |
| | 219 | KEKNCPDGYIYSSNITSGFDCGVWICRRVGSAFCSRTGDYTSPTEL |
| | 220 | KEKNCPDGYIYSSNITSGFDCGVWICRRVGSAFCSRTGDYTSPTELD |
| | 221 | KEKNCPDGYIYSSNITSGFDCGVWICRRVGSAFCSRTGDYTSPTELDI |
| | 222 | KEKNCPDGYIYSSNITSGFDCGVWICRRVGSAFCSRTGDYTSPTELDIY |
| | 223 | KEKNCPDGYIYSSNITSGFDCGVWICRRVGSAFCSRTGDYTSPTELDIYE |
| R2D9 | 68 | ITHKTCPNGYNWFDRCCSWDGTCGDGCCSNRAWPSGNGRADSSIGETYGYEF |
| knob | 224 | ITHKTCPNGYNWFDRCCSWDGTCGDGCCSNRAWPSGNGRADSS |
| | 225 | ITHKTCPNGYNWFDRCCSWDGTCGDGCCSNRAWPSGNGRADSSI |
| | 226 | ITHKTCPNGYNWFDRCCSWDGTCGDGCCSNRAWPSGNGRADSSIG |
| | 227 | ITHKTCPNGYNWFDRCCSWDGTCGDGCCSNRAWPSGNGRADSSIGE |
| | 228 | ITHKTCPNGYNWFDRCCSWDGTCGDGCCSNRAWPSGNGRADSSIGET |
| | 229 | ITHKTCPNGYNWFDRCCSWDGTCGDGCCSNRAWPSGNGRADSSIGETY |
| | 230 | ITHKTCPNGYNWFDRCCSWDGTCGDGCCSNRAWPSGNGRADSSIGETYG |
| | 231 | ITHKTCPNGYNWFDRCCSWDGTCGDGCCSNRAWPSGNGRADSSIGETYGY |
| | 232 | ITHKTCPNGYNWFDRCCSWDGTCGDGCCSNRAWPSGNGRADSSIGETYGYE |

In some embodiments, the knob peptide is recombinantly produced. In some embodiments, the knob peptide is purified.

In some embodiments, also provided are methods for producing any of the provided knob peptides. In some embodiments, the methods include methods in which a provided peptide that contains 2 or more cysteine residues is able to be produced and purified as a disulfide-bonded soluble protein. In some embodiments, the provided methods include transforming a host cell, e.g., E. coli, with an expression vector encoding the knob peptide. In some embodiments, the expression vector encodes a fusion protein that includes the soluble peptide and a chaperone, e.g., a bacterial chaperone. In some embodiments, the knob peptide and the chaperone, e.g., bacterial chaperone, are joined by a linker. In some embodiments, the linker is a cleavable linker.

Techniques for manipulating nucleic acids, such as those for generating mutation in sequences, subcloning, labeling, probing, sequencing, hybridization and so forth, are described in detail in scientific publications and patent documents. See, for example, Sambrook J, Russell D W (2001) Molecular Cloning: a Laboratory Manual, 3rd ed. Cold Spring Harbor Laboratory Press, New York; Current Protocols in Molecular Biology, Ausubel ed., John Wiley & Sons, Inc., New York (1997); Laboratory Techniques in Biochemistry and Molecular Biology; Hybridization With Nucleic Acid Probes, Part I, Theory and Nucleic Acid Preparation, Tijssen ed., Elsevier, N.Y. (1993).

In some embodiments, the fusion protein has increased solubility relative to the knob peptide alone. In some aspects, this increased solubility is conferred at least in part by the inclusion of the chaperone, e.g., bacterial chaperone. In some aspects, the inclusion of the chaperone, e.g., bacterial chaperone, promotes solubility of the fusion protein while permitting disulfide bond formation in the soluble peptide, including in host cell environments that have been engineered or modified to promote disulfide bond formation. In some embodiments, the chaperone, e.g., bacterial chaperone, is TrxA.

In some embodiments, the provided methods further include culturing the host cell, e.g., the bacteria, such as E. coli, under conditions permissive of expression of the fusion protein. In some embodiments, the provided methods further include, following the culturing, isolating the expressed fusion protein from supernatant of a lysate of the host cell, e.g., the bacteria, such as E. coli. In some embodiments, the provided methods further include cleaving the cleavable linker, thereby producing the soluble peptide that is free of the bacterial chaperone.

In some embodiments, the cleavable linker is an enterokinase cleavage tag. In some embodiments, the cleavable linker includes the amino acid sequence DDDDK (SEQ ID NO: 235). In some embodiments, the knob peptide includes a flexible linker peptide (e.g. GS) between the knob peptide and the cleavable linker, such as to allow protease access to the substrate. In some embodiments, the cleaving of the cleavable linker includes adding enterokinase. In some embodiments, enterokinase is added to the supernatant of the host cell lysate. In some embodiments, the provided methods further include, following cleaving the cleavable linker, removing the enterokinase and/or the bacterial chaperone from the solution containing the knob peptide. In some embodiments the flexible linker (GS) remains fused to the knob peptide, e.g. to the N-terminus of the knob peptide.

In some embodiments, the provided methods further include separating the knob peptide from any soluble aggregates present in solution, including soluble aggregates of the knob peptide. In some embodiments, the separating involves separating the active knob peptide from the larger, inactive or less active soluble aggregates thereof. In some embodiments, the separating is achieved using chromatographic methods. In some embodiments, the separating is by size exclusion chromatography. In some embodiments, the separating involves collecting one or more elution fractions containing the knob peptide, e.g., that is smaller in size than the soluble aggregates thereof, thereby producing a purified composition of knob peptides.

In some embodiments, the expression vector further includes an inducible promoter sequence to control the expression of the fusion protein. The term “promoter sequence” as used herein refers to a DNA sequence, which is generally located upstream of a gene present in a DNA polymer, and provides a site for initiation of the transcription of said gene into mRNA. Promoter sequences suitable for use in this invention may be derived from viruses, bacteriophages, prokaryotic cells or eukaryotic cells, and may be a constitutive promoter or an inducible promoter.

In some embodiments, the inducible promoter sequence is operably linked to the sequence encoding the fusion protein. The term “operatively linked” as used herein means that a first sequence is disposed sufficiently close to a second sequence such that the first sequence can influence the second sequence or regions under the control of the second sequence. For instance, a promoter sequence may be operatively linked to a gene sequence, and is normally located at the 5′-terminus of the gene sequence such that the expression of the gene sequence is under the control of the promoter sequence. In addition, a regulatory sequence may be operatively linked to a promoter sequence so as to enhance the ability of the promoter sequence in promoting transcription. In such case, the regulatory sequence is generally located at the 5′-terminus of the promoter sequence.

Promoter sequences suitable for use in this invention are preferably derived from any one of the following: viruses, bacterial cells, yeast cells, fungal cells, algal cells, plant cells, insect cells, animal cells, and human cells. For example, a promoter useful in bacterial cells includes, but is not limited to, tac promoter, T7 promoter, T7 A1 promoter, lac promoter, trp promoter, trc promoter, araBAD promoter, and XPRPL promoter. A promoter useful in plant cells includes, e.g., 35S CaMV promoter, actin promoter, ubiquitin promoter, etc. Regulatory elements suitable for use in mammalian cells include CMV-HSV thymidine kinase promoters, SV40, RSV-promoters, CMV enhancers, or SV40 enhancers.

Vectors suitable for use in this invention include those commonly used in genetic engineering technology, such as bacteriophages, plasmids, cosmids, viruses, or retroviruses. Vectors suitable for use in this invention may include other expression control elements, such as a transcription starting site, a transcription termination site, a ribosome binding site, a RNA splicing site, a polyadenylation site, a translation termination site, etc. Vectors suitable for use in this invention may further include additional regulatory elements, such as transcription/translation enhancer sequences, and at least a marker gene or reporter gene allowing for the screening of the vectors under suitable conditions. Marker genes suitable for use in this invention include, for instance, dihydrofolate reductase gene and G418 or neomycin resistance gene useful in eukaryotic cell cultures, and ampicillin, streptomycin, tetracycline or kanamycin resistance gene useful in E. coli and other bacterial cultures. Vectors suitable for use in this invention may further include a nucleic acid sequence encoding a secretion signal. These sequences are well known to those skilled in the art.

Depending on the vector and host cell system used, the recombinant gene product (protein) produced according to this invention may either remain within the recombinant cell, be secreted into the culture medium, be secreted into periplasm, or be retained on the outer surface of a cell membrane. The recombinant gene product (protein) produced by the method of this invention can be purified by using a variety of standard protein purification techniques, including, but not limited to, affinity chromatography, ion exchange chromatography, gel filtration, electrophoresis, reverse phase chromatography, chromatofocusing and the like. The recombinant gene product (protein) produced by the method of this invention is preferably recovered in “substantially pure” form. As used herein, the term “substantially pure” refers to a purity of a purified protein that allows for the effective use of said purified protein as a commercial product.

In some embodiments, the provided methods for producing a fusion protein containing the knob peptide and a chaperone, e.g., bacterial chaperone, can be performed using any host organism which is capable of expressing heterologous polypeptides, and is capable of being genetically modified. A host organism is preferably a unicellular host organism, however, the use of multicellular organisms is also encompassed by the provided methods, provided the organism can be modified as described herein and a polypeptide of interest expressed therein. For purposes of clarity, the term “host cell” will be used herein throughout, but it should be understood, that a host organism can be substituted for the host cell, unless unfeasible for technical reasons.

In some embodiments, the host cell is a prokaryotic cell, such as a bacterial cell. The host cell may be a gram positive bacterial cells, such as Bacillus or gram negative bacteria such as E. coli. The host organisms may be aerobic or anaerobic organisms. In some embodiments, host cells are those which have characteristics which are favorable for expressing polypeptides, such as host cells having fewer proteases than other types of cells. Suitable bacteria for this purpose include archaebacteria and eubacteria, for example, Enterobacteriaceae. Other examples of useful bacteria include Escherichia, Enterobacter, Azotobacter, Erwinia, Bacillus, Pseudomonas, Klebsiella, Proteus, Salmonella, Serratia, Shigella, Rhizobia, Vitreoscilla, and Paracoccus. Additional examples of useful bacteria include Corynebacterium, Lactococcus, Lactobacillus, and Streptomyces species, in particular Corynebacterium glutamicum, Lactococcus lactis, Lactobacillus plantarum, Streptomyces coelicolor, Streptomyces lividans. Suitable E. coli hosts include E. coli DHB4, E. coli BL-21 (which are deficient in both lon (Phillips et al. J. Bacteriol. 159: 283, 1984) and ompT proteases), E. coli AD494, E. coli W3110 (ATCC 27,325), E. coli 294 (ATCC 31,446), E. coli B, and E. coli X1776 (ATCC 31,537). Other strains include E. coli B834 which are methionine deficient and, therefore, enables high specific activity labeling of target proteins with ³⁵S-methionine or selenomethionine (Leahy et al. Science 258: 987, 1992). Yet other strains of interest include the BLR strain, and the K-12 strains HMS174 and NovaBlue, which are recA-derivative that improve plasmid monomer yields and may help stabilize target plasmids containing repetitive sequences.

In some embodiments, the E. coli host cell used in the provided methods is engineered or modified to improve soluble expression of disulfide-bonded proteins in the E. coli cytosol. In some embodiments, the cytoplasmic thiol-redox equilibrium environment is changed via alteration in reducing pathways, such as thioredoxin reductase. Various types of mutant strains, including SHuffle (New England Biolabs) and Origami™ (DE3) (Novagen, Germany), which lack glutathione reductase Agor, thioredoxin reductase, and/or glutathione biosynthesis pathways, are commercially available. In some embodiments, the E. coli strain transformed as part of the provided methods is the Origami™ (DE3) (Novagen, Germany) mutant strain.

In some embodiments, the purified knob peptide retains an N-terminal or C-terminal linker flexible linker. In some embodiments, the flexible linker is a GS linker.

In some embodiments, the knob peptide further comprises a linker for cyclization of the peptide. For instance, the linker is GGGGAMGS (SEQ ID NO: 234). In some embodiments, the peptide is cyclized.

In some of any embodiments, a provided knob peptide binds to a spike protein of a coronavirus, e.g. SARs-CoV2. Exemplary features of provided antibodies are described, e.g. in Section II.C. In some embodiments, knob peptide binds to the spike protein of SARS-CoV2 or a binding portion or epitope thereof with a binding affinity dissociation constant of less than at or about 10⁻⁹ M, less than at or about 10⁻¹⁰ M, less than at or about 10⁻¹¹ M or less than at or about 10⁻¹² M. In some embodiments, the knob peptide neutralizes SARS-CoV2 infection of a human cell. In some embodiments, the knob peptide neutralizes SARS-CoV2 with an EC50 of less than about 100 ng/mL, 10 ng/mL, 1 ng/mL, 0.1 ng/mL, 0.01 ng/mL, 0.001 ng/mL. In some embodiments, the knob peptide neutralizes SARS-COV2 with an EC50 of less than about 100 nM, 10 nM, 1 nM, 0.1 nM, 0.01 nM or 0.001 nM. In some embodiments, the knob peptide neutralizes SARS-COV2 with an EC50 of less than at or about 500 pM, 250 pM, 100 pM, 50 pM, 25 pM, 10 pM, 5 pM, 2.5 pM, 1 pM, 0.5 pM or less. In some embodiments, the SARS-CoV2 is selected from Wuhan-Hu-1 strain, UK (B.1.1.7) strain or South African (B.1.351) strain. In some embodiments, the SARS-CoV2 is Wuhan-Hu-1 strain.

Also provided is a multispecific binding protein, comprising a plurality of any of the provided knob peptides. In some embodiments, the plurality of knob peptides are paratopes. In some embodiments, the plurality of knob peptides are 2, 3, or 4 peptides. Exemplary formats for generating a multispecific polypeptide are depicted in FIG. 12.

In some embodiments, one or more knobs are linked in tandem in a single polypeptide chain separated with a flexible linker (e.g. GGGS or other similar flexible linker, including longer linkers of (GGGS)n where n is 1-3). In some embodiments, the tandem single polypeptide may include 2, 3, 4 or more knob peptides to produce a bivalent, trivalent, tetravalent or other multivalent molecule.

In some embodiments, the knob peptides are re-formatted by replacement of a knob region of an ultralong CDR-H3 scaffold, including any of the humanized ultralong heavy chain molecules described herein. The heavy chain can be complexed with a light chain, such as any of the light chain molecules described herein. In some embodiment, when produced in a cell, a two chain polypeptide is formed by dimerization resulting from disulfide formation between two heavy chain molecules. In some embodiments, the modified immunoglobulin containing a knob peptide is a homodimer containing the knob peptide. In other embodiments, two different heavy chains may be co-expressed in a cell using knobs-into-hole engineering strategy or other strategy to produce a heterodimer in which two different heavy chains, each carrying a different knob peptide, may interact to form a heterodimer. In some embodiments, residues of the constant chain are modified by amino acid substitution to promote the heterodimer formation. In some of any embodiments, the one more amino acid modifications are selected from a knob-into-hole modification and a charge mutation to reduce or prevent self-association due to charge repulsion. The heterodimer can be formed by transforming into a cell both a first nucleic acid molecule encoding a first polypeptide subunit and a second nucleic acid molecule encoding a second different polypeptide subunit. In some aspects, the heterodimer is produced upon expression and secretion from a cell as a result of covalent or non-covalent interaction between residues of the two polypeptide subunits to mediate formation of the dimer. In such processes, generally a mixture of dimeric molecules is formed, including homodimers and heterodimers. For the generation of heterodimers, additional steps for purification can be necessary. For example, the first and second polypeptide can be engineered to include a tag with metal chelates or other epitope, where the tags are different. The tagged domains can be used for rapid purification by metal-chelate chromatography, and/or by antibodies, to allow for detection by western blots, immunoprecipitation, or activity depletion/blocking in bioassays. Methods include those described in U.S. Pat. No. 10,995,127. In some embodiments, a human IgG1 includes a T22Y amino acid substitution in the CH3 domain and a second IgG1 heavy chain includes a Y86T amino acid substitution in the heavy chain.

Also provided herein are methods for identifying an ultralong CDR H3 knob, such as a bovine CDR H3 knob, by amino acid sequence, including from a sequence library. In some aspects, methods for identifying an ultralong CDR H3 knob include defining the region of the knob domain, such as by reference to the formula described herein, e.g. set forth below.

In some embodiments, a method for identifying an ultralong CDR H3 knob, includes defining the knob region N-terminal boundary as the first DH cysteine in the “CPDG” motif. In some embodiments, the method further includes defining the C-terminal boundary as the position located by subtracting number of ascending stalk residues from the framework 4 tryptophan position. In some aspects, the method can be used for identifying an ultralong CDR H3 knob from any antibody sequence. In particular embodiments, the antibody sequence is a bovine antibody, such as any of the antibodies described herein.

An expression of this embodiment of the method is shown below:
Knob boundary position (C-terminal end)=Position of conserved framework 4 tryptophan−X; wherein X=number of amino acids, starting at the framework 3 canonical cysteine that defines the ascending stalk, and ending at the amino acid preceding the conserved first D region cysteine in the “CPDG” motif;
Number of residues in the knob (K)=L−2X; wherein L=number of amino acids encompassing stalk and knob domains, starting at canonical framework 3 cysteine and ending at canonical framework 4 tryptophan;
K position=(X+1) to (X+K)

Also provided is a composition comprising any of the provided knob peptide. Also provided is a composition comprising a plurality of any of the provided knob peptides. In some embodiments, the plurality of knob peptides are paratopes. In some the plurality of knob peptides are 2, 3 or 4 peptides. In some embodiments, the composition is a pharmaceutical composition and may contain a pharmaceutical carrier. In some embodiments, the composition is formulated for inhalation, such as described herein.

### C. Exemplary Features

In some embodiments, any of the binding polypeptides provided herein, including an antibody or antigen binding fragment or a knob peptide, binds to a coronavirus. In some embodiments, the binding polypeptides, including antibodies or antigen-binding fragments or knob peptides provided herein, can bind to and/or neutralize an alphacoronavirus, a betacoronavirus, a gammacoronavirus, and/or a deltacoronavirus. In certain embodiments, this binding and/or neutralization can be specific for a particular genus of coronavirus or for a particular subgroup of a genus. In some embodiments, the coronavirus is SARS CoV-2. In some embodiments, any of the binding polypeptides provided herein, including an antibody or antigen binding fragment or a knob peptide, binds to a SARS CoV-2 spike (S) protein.

In some embodiments, the binding polypeptide, including an antibody or antigen binding fragment or a knob peptide, binds to an epitope within SARS-CoV-2 spike protein. In some embodiments, the binding polypeptide, including an antibody or antigen binding fragment or a knob peptide binds to the receptor binding domain (RBD) of SARS-CoV-2 spike protein. In some embodiments, the epitope recognized by a provided binding polypeptide, including an antibody or antigen binding fragment or a knob peptide is within RBD of SARS-CoV-2 spike protein.

In another aspect, provided herein are binding polypeptides, including antibodies or antigen binding fragments or knob peptides, that bind the same or an overlapping epitope of S (e.g., an epitope of SARS CoV-2 S) as an antibody or antigen binding fragment or knob peptide described herein. In some embodiments, provided herein are binding polypeptides, including antibodies or antigen binding fragments or knob peptides, that bind the same or an overlapping epitope of S (e.g., an epitope of SARS CoV-2 S) as an antibody or antigen binding fragment RBDA2, RBD C6, RBD F4, R2B1, R2D6, R2G1, R4A10, R4E5, R4G3, R4G11, R5A3, R4C1, R2C3, SKD, SKM, R2G3, R2F12, R2F12, SR3A3, or R2D9. In some embodiments, provided herein are binding polypeptides, including antibodies or antigen binding fragments or knob peptides, that bind the same or an overlapping epitope of (e.g., an epitope of SARS CoV-2 S) as an ultralong CDR3 antibody or antigen binding fragment R4C1, R2C3, SKD, SKM, R2G3, R2F12, SR3A3, or R2D9. In some embodiments, provided herein are binding polypeptides, including antibodies or antigen binding fragments or knob peptides, that bind the same or an overlapping epitope of S (e.g., an epitope of SARS CoV-2 S) as a knob peptide R4C1 knob peptide, R2C3 knob peptide, SKD knob peptide, SKM knob peptide, R2G3 knob peptide, R2F12 knob peptide, SR3A3 knob peptide, or R2D9 knob peptide.

In some embodiments, the epitope of an antibody can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization may be accomplished using any of the known methods in the art (e.g., Giege R et al, (1994) Acta Crystallogr D Biol Crystallogr 50(Pt 4): 339-350; McPherson A (1990) Eur J Biochem 189: 1-23; Chayen N E (1997) Structure 5: 1269-1274; McPherson A (1976) J Biol Chem 251: 6300-6303). Antibody: antigen crystals may be studied using well known X-ray diffraction techniques and may be refined using computer software such as X-PLOR (Yale University, 1992, distributed by Molecular Simulations, Inc.; see, e.g., Meth Enzymol (1985) volumes 114 & 1 15, eds Wyckoff H W et al; U.S. Patent Application No. 2004/0014194), and BUSTER (Bricogne G (1993) Acta Crystallogr D Biol Crystallogr 49(Pt 1): 37-60; Bricogne G (1997) Meth Enzymol 276A: 361-423, ed Carter C W; Roversi P et al, (2000) Acta Crystallogr D Biol Crystallogr 56(Pt 10): 1316-1323). Mutagenesis mapping studies may be accomplished using any method known to one of skill in the art. See, e.g., Champe M et al, (1995) supra and Cunningham B C & Wells J A (1989) supra for a description of mutagenesis techniques, including alanine scanning mutagenesis techniques.

In some embodiments, the epitope of an antibody is determined using alanine scanning mutagenesis studies. Usually, binding to the antigen is reduced or disrupted when a residue within the epitope is substituted to alanine. In one embodiment, the Kd of binding to the antigen is increased by about 5-fold, 10-fold, 20-fold, 10-fold or more when a residue within the epitope is substituted for alanine. In one embodiment, binding affinity is determined by ELISA. In addition, antibodies that recognize and bind to the same or overlapping epitopes of S (e.g., an epitope of SASRS CoV-2 S) can be identified using routine techniques such as an immunoassay, for example, by showing the ability of one antibody to block the binding of another antibody to a target antigen, i.e., a competitive binding assay.

In some embodiments, the binding polypeptides, including antibodies or antigen binding fragments or knob peptides, provided herein neutralize a coronavirus. In some embodiments, the binding polypeptides, including antibodies or antigen binding fragments or knob peptides, neutralize SARS-COV-2 including isolates or variants thereof. In some embodiments, the binding polypeptides, including antibodies or antigen binding fragments or knob peptides, are pan-neutralizing to two or more coronaviruses.

In some embodiments, the provided binding molecules, such as antibodies or antigen binding fragments, are capable of binding CoV-2 spike protein, such as SARS-CoV-2 spike protein, with at least a certain affinity, as measured by any of a number of known methods. In some embodiments, the affinity is represented by an equilibrium dissociation constant (K_{D}); in some embodiments, the affinity is represented by EC₅₀.

A variety of assays are known for assessing binding affinity and/or determining whether a binding molecule (e.g., an antibody or fragment thereof or knob peptide) specifically binds to a particular antigen (e.g., spike protein). It is within the level of a skilled artisan to determine the binding affinity of a binding molecule, such as by using any of a number of binding assays that are well known in the art. For example, in some embodiments, a BIAcore® instrument can be used to determine the binding kinetics and constants of a complex between two proteins, using surface plasmon resonance (SPR) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent).

SPR measures changes in the concentration of molecules at a sensor surface as molecules bind to or dissociate from the surface. The change in the SPR signal is directly proportional to the change in mass concentration close to the surface, thereby allowing measurement of binding kinetics between two molecules. The dissociation constant for the complex can be determined by monitoring changes in the refractive index with respect to time as buffer is passed over the chip. Other suitable assays for measuring the binding of one protein to another include, for example, immunoassays such as enzyme linked immunosorbent assays (ELISA) and radioimmunoassays (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, ELISA, analytical ultracentrifugation, spectroscopy, flow cytometry, sequencing and other methods for detection of expressed nucleic acids or binding of proteins.

In some embodiments, the binding molecule, e.g., antibody or fragment thereof or knob peptide, binds, such as specifically binds, to an antigen, e.g., a spike protein or receptor binding domain or an epitope therein, with an affinity or K_{A} (i.e., an equilibrium association constant of a particular binding interaction with units of 1/M; equal to the ratio of the on-rate [kₒₙ or kₐ] to the off-rate [k_{off} or k_{d}] for this association reaction, assuming bimolecular interaction) equal to or greater than 10⁸ M⁻¹. In some embodiments, the antibody or fragment thereof or knob peptide exhibits a binding affinity for the peptide epitope with a K_{D} (i.e., an equilibrium dissociation constant of a particular binding interaction with units of M: equal to the ratio of the off-rate [k_{off} or k_{d}] to the on-rate [kₒₙ or kₐ] for this association reaction, assuming bimolecular interaction) of equal to or less than 10⁻⁸ M. For example, the equilibrium dissociation constant K_{D} ranges from 10⁻⁸ M to 10⁻¹³ M, such as 10⁻⁹ M to 10⁻¹ M, 10⁻⁹ M to 10⁻¹² M, or 10⁻⁹ M to 10⁻¹ M, 10⁻⁹ M to 10⁻¹⁰ M, 10⁻¹⁰ M to 10⁻¹³ M, 10⁻¹⁰ M to 10⁻¹² M, or 10⁻¹⁰ M to 10⁻¹¹ M, 10⁻¹¹ M to 10⁻¹³ M, 10⁻¹¹ M to 10⁻¹² M or 10⁻¹¹ M to 10⁻³ M. In some embodiments, the K_{D} is less than at or about 10⁻⁹ M, less than at or about 10⁻¹⁰ M, less than at or about 10⁻¹¹ M or less than at or about 10⁻¹² M. Among provided binding molecules, including antibodies or antigen binding fragments or knob peptides, the K_{D} is less than at or about 10⁻¹² M. The on-rate (association rate constant; kₒₙ or kₐ; units of 1/Ms) and the off-rate (dissociation rate constant; k_{off} or k_{d}; units of 1/s) can be determined using any of the assay methods known in the art, for example, surface plasmon resonance (SPR).

In some embodiments, a provided binding polypeptide, including an antibody or antigen binding fragment or a knob peptide, is a neutralizing antibody. A “neutralizing antibody” refers to an antibody that reduces at least one activity of a polypeptide comprising the epitope to which the antibody specifically binds. In some aspects, a neutralizing antibody prevents a structure (i.e., organism, virus, particle, etc.) comprising the epitope to which it binds from entering a cell, thus protecting that cell from infection. In certain embodiments, a neutralizing antibody reduces an activity in vitro and/or in vivo, such as viral entry into a host cell. In some aspects, a neutralizing antibody can target an epitope of an infectious agent, such as a virus. In some aspects, a neutralizing antibody can target an epitope of a host cell to which an infectious agent binds, such as a viral receptor glycoprotein.

In some aspects, the epitope bound by a neutralizing antibody can be any protein that includes a receptor binding domain (RBD) that mediates binding to a cognate receptor on a target host cell that can be infected by the virus. In some embodiments, the first step in any viral life cycle is contact and attachment with a target host cell, which can be mediated by structural proteins or viral membrane proteins via a surface exposed receptor binding domain (RBD). Antibodies or other binding domains which target the RBD or subvert its function are among the class of antibodies known as neutralizing, as their binding occludes virus-host receptor interaction and therefore “neutralizes” the ability to gain entry into a host cell.

In some aspects, the RBD is located in an exposed surface of the viral membrane fusion glycoprotein. Viral fusion glycoproteins are characterized into classes based on structure, as most enveloped viruses function in the same membrane-fusion pathway. Class I viral membrane fusion proteins project away from the membrane (i.e., typical “spiked” appearance) and feature an alpha helix coiled coil. Native Class I proteins on the surface of virions are trimeric in both their pre and post fusion conformations, with each monomer requiring individual proteolytic cleavage prior to fusion in order to maintain function. The C terminal fragment as a result of this cleavage is anchored in the viral membrane, while the fusion peptide is relocated at or near the N terminus. In some aspects, Class I proteins include the Spike (S) protein of coronaviruses. In some aspects, the RBD is located within a surface exposed viral spike protein, or S protein. In some aspects, the binding domain specifically binds the S (spike) glycoprotein of a SARS virus, or RBD thereof.

During the interaction of the SARS CoV-2 S protein RBD with its' native ACE-2 receptor, the RBD presents a concave surface for the N terminus of the receptor peptidase on which amino acids 445-460 anchor the entire receptor-binding loop of the RBD core (Du et al., Nat Rev Microbiol. 7(3):226-236, 2009). A fragment that is located in the S1 S protein subunit and spans amino acids 318-510 is considered the likely minimal receptor-binding domain (RBD) needed for interaction with angiotensin-converting enzyme 2 (ACE-2) receptor.

Provided herein are antibodies and binding domains thereof that have specificity for the SARS CoV-2 S protein. In some aspects, these S specific antibodies were observed to block S protein interaction with the ACE2 receptor and therefore neutralize infection in vitro. In some embodiments, methods that calculate the neutralizing ability of an antibody include the plaque assay or plaque reduction and neutralization test (PRNT). Titrations of the virus are grown on cell monolayers that are overlaid with agarose in the presence of serial dilatation concentration of antibody or antigen binding fragment thereof. After incubation for a time period to achieve a cytopathic effect, such as for about 3 to 28 days, generally 7 to 10 days, the cells can be fixed and foci of absent cells visualized as plaques are determined. The effect of neutralizing antibody can be quantified via measurement of plaque number, plaque size, and plaque morphology. Percent neutralization, such as Percent Maximal Neutralization (PMN), can be calculated. Antibody efficacy can be reported as 50%, 60%, 70%, 80%, or 90% neutralization, which represent the last antibody dilution concentration capable of inhibiting 50%, 60%, 70%, 80%/6, or 90% of total plaques.

In some embodiments, the binding polypeptides, including antibodies or antigen binding fragments or knob peptides, is capable of neutralizing a coronavirus, such as SARS-CoV-2, with an EC50 equal to or less than about 100 ng/mL, 10 ng/mL, 1 ng/mL, 0.1 ng/mL, 0.01 ng/mL, 0.001 ng/mL, or less. In some embodiments, the EC50 is less than about 100 nM, 10 nM, 1 nM, 0.1 nM, 0.01 nM or 0.001 nM or less. In some embodiments, the neutralizing activity is subnanomolar, such as picomolar. In some embodiments, the neutralizing activity is less than 1 nM. In some embodiments, the neutralizing activity is less than at or about 500 pM, 250 pM, 100 pM, 50 pM, 25 pM, 10 pM, 5 pM, 2.5 pM, 1 pM, 0.5 pM or less.

In some embodiments, the binding polypeptides, including antibodies or antigen binding fragments or knob peptides, is capable of neutralizing at least two cross-lineage SARS CoV-2 isolates. In some embodiments, a broadly neutralizing antibody or binding polypeptide (e.g. knob peptide) described herein specifically binds to S and is capable of neutralizing at least two isolates of SARS CoV-2. In some embodiments, the two isolates are two cross-lineage isolates. In some embodiments, the antibody or binding polypeptide (e.g. knob peptide) is capable of neutralizing at least about 50%, 60%, 70%, 80%, 90%, or 100% of cross-lineage SARS CoV-2 isolates. In one embodiment, the antibody or binding polypeptide (e.g. knob peptide) is capable of neutralizing at least about 90% of cross-lineage SARS CoV-2 isolates. In some embodiments, the antibody or binding polypeptide (e.g. knob peptide) is capable of neutralizing the cross-lineage SARS CoV-2 isolates with a median IC50 equal to or less than about 100 ng/mL, 10 ng/mL, 1 ng/mL, 0.1 ng/mL, 0.01 ng/mL, 0.001 ng/mL, or less.

In another aspect, provided herein are antibodies or binding polypeptides (e.g. knob peptides) that compete (e.g., in a dose dependent manner) for binding to S (e.g., an epitope of SARS CoV-2 S) with an antibody or binding polypeptide (e.g. knob peptide) described herein, as determined using assays known to one of skill in the art or described herein (e.g., ELISA competitive assays or surface plasmon resonance). In another aspect, provided herein are antibodies or binding polypeptides (e.g. knob peptides) that competitively inhibit (e.g., in a dose dependent manner) an antibody described herein from binding to S (e.g., an epitope of SARS CoV-2 S), as determined using assays known to one of skill in the art or described herein (e.g., ELISA competitive assays, or suspension array or surface plasmon resonance assay).

In certain embodiments, the epitope of an antibody described herein is used as an immunogen to produce antibodies.

The affinity or avidity of an antibody or fusion polypeptide for an antigen can be determined experimentally using any suitable method well known in the art, e.g., flow cytometry, enzyme-linked immunosorbent assay (ELISA), or radioimmunoassay (RIA), or kinetics (e.g., BTACORE™ analysis). Direct binding assays as well as competitive binding assay formats can be readily employed. (See, for example, Berzofsky, et al., “Antibody-Antigen Interactions,” In Fundamental Immunology, Paul, W. E., Ed., Raven Press: New York, N.Y. (1984); Kuby, Janis Immunology, W. H. Freeman and Company: New York, N.Y. (1992); and methods described herein. The measured affinity of a particular antibody-antigen interaction can vary if measured under different conditions (e.g., salt concentration, pH, temperature). Thus, measurements of affinity and other antigen-binding parameters (e.g., KD or Kd, Kon, Koff) are made with standardized solutions of antibody and antigen, and a standardized buffer, as known in the art and such as the buffer described herein.

### III. Methods of Producing an Bovine-Derived Antibody or Antigen-Binding Fragment Against SARS-Cov-2

In one aspect, provided herein are methods for generating a broadly neutralizing anti-Spike (S) antibody directed to the Spike protein of SARS-CoV-2. In one embodiment, the antibody is polyclonal. In some embodiments, the antibody is monoclonal (e.g., a chimeric or humanized)

In some embodiments, a method of producing a broadly neutralizing anti-S antibody described herein comprises immunizing a bovine by administering at least one dose of an antigenic composition comprising a SARS CoV-2 specific antigen to produce a broadly neutralizing anti-S antibody. In some embodiments, the SARS CoV-specific antigen comprises a S trimer polypeptide. In some embodiments, the SARS CoV-specific antigen comprises a S monomer polypeptide. In some embodiments, the SARS CoV-specific antigen comprises a polynucleotide encoding a S trimer or monomer polypeptide.

In some embodiments, method of producing a broadly neutralizing anti-S antibody, comprises immunizing a bovine by administering at least one dose of an antigenic composition comprising a SARS CoV-2 specific antigen to produce a broadly neutralizing anti-S antibody, wherein the SARS CoV-2 specific antigen comprises a S trimer polypeptide or a polynucleotide encoding a S trimer polypeptide, and wherein the bovine produces the broadly neutralizing anti-S antibody. In some embodiments, method of producing a broadly neutralizing anti-S antibody, comprises immunizing a bovine by administering at least one dose of an antigenic composition comprising a SARS CoV-2 specific antigen to produce a broadly neutralizing anti-S antibody, wherein the SARS CoV-2 specific antigen comprises a S monomer polypeptide or a polynucleotide encoding a S monomer polypeptide, and wherein the bovine produces the broadly neutralizing anti-S antibody.

In some embodiments, the bovine is domestic cattle, bison, African buffalo, water buffalo, or yak. In some embodiments, the bovine is domestic cattle. In one embodiment, the domestic cattle is a dairy cow. In some embodiments, the cow is pregnant.

In some embodiments, the SARS CoV-2 specific antigen comprises a virus, pseudovirus, or virus-like particle comprising a S trimer polypeptide. In some embodiments, the SARS CoV-2 specific antigen comprises an isolated S trimer polypeptide. A S trimer polypeptide may be produced by a number of different means, for example, as described in US Patent Appl. Pub. No. 2014/0212458, Sanders, R. W. et al, PLoS Pathog. 9, e1003618 (2013) and Guenaga J., et al, Immunity 46(5:792-803.e3 (2017), each of which is incorporated by reference herein in its entirety. For example, HEK293F cells can be co-transfected with a pg140 encoding plasmid and a furin encoding plasmid. Supernatants comprising the antigen are purified using a lectin column. The affinity-purified antigen can be further purified to size homogeneity using size exclusion chromatography.

In some embodiments, a trimer is formed from a wildtype SARS CoV-2 S protein. In some embodiments, a trimer is formed from a variant SARS CoV-2 S protein.

In some embodiments, the antigenic composition further comprises an adjuvant. The skilled person is familiar with many potentially useful adjuvants, such as Freund's complete adjuvant, alum, and squalene. See, e.g., US Patent Appl. Pub. No. 20150361160, which is incorporated by reference herein in its entirety for all purposes. Adjuvants which may be used in compositions of the invention include, but are not limited to oil emulsion compositions (oil-in-water emulsions and water-in-oil emulsions), complete Freund's adjuvant (CFA) and incomplete Freund's adjuvant (IFA). In one embodiment, the adjuvant comprises RIBI, Iscomatrix, or ENABL CI (VaxLiant). Adjuvants suitable for use in the invention include bacterial or microbial derivatives such as derivatives of enterobacterial lipopolysaccharide (LPS), Lipid A derivatives, immunostimulatory oligonucleotides and ADP-ribosylating toxins and detoxified derivatives thereof.

Methods for immunizing a bovine, such as a cattle, to produce, for example, high titer colostrum, milk, serum, or immune tissues (e.g., PBMC), are known in in the art. Such methods are disclosed, for example, in US Patent Appl. Pub. Nos US20070053917 and US20130022619, each of which is incorporated by reference herein in its entirety for all purposes.

In some embodiments, the immunizing comprises administering a priming dose and at least one booster dose of the antigenic composition. In some embodiments, the immunizing comprises administering more than one booster doses of the antigenic composition. In one embodiment, the priming dose and at least one booster dose comprise the same antigenic composition. In some embodiments, the more than one booster doses comprise the same antigenic composition. The animal may be dosed with the immunogenic composition at intervals over a period of days, weeks or months. At the conclusion of the immunization regime, the hyperimmune material such as blood, milk or colostrum is harvested. In one embodiment, the hyperimmune material is collected less than 2 months, less than 3 months, less than 4 months, less than 5 months, less than 6 months, less than 9 months, or less than 12 months after administering the priming dose. In one embodiment, the hyperimmune material is collected between about 3 months and about 6 months after administering the priming dose. In one embodiment, the hyperimmune material is collected between about 3 months and about 9 months after administering the priming dose. In some embodiments, the hyperimmune material is collected between about 3 months and about 12 months after administering the priming dose. In one embodiment, the hyperimmune material is collected between about 6 months and about 12 months after administering the priming dose.

In some embodiments, the immunogenic composition comprises an SARS CoV-2 specific antigen derived from a single SARS CoV-2 isolate. In some embodiments, the immunogenic composition comprises an SARS CoV-2 specific antigen derived from more than one the SARS CoV-2 isolates. In some embodiments, the more than one SARS CoV-2 isolates belong to the same lineage. In some embodiments, the more than one SARS CoV-2 isolates belong to different lineages. One or more of the HIV isolates can be a circulating recombinant form SARS CoV-2.

In some embodiments, a method of producing a broadly neutralizing anti-S antibody further comprises isolating from the bovine a biological sample comprising the broadly neutralizing anti-S antibody. In some embodiments, the biological sample is milk, blood, serum, colostrum, or peripheral blood mononuclear cells (PBMC). In one embodiment, the biological sample is collected less than 2 months, less than 3 months, less than 4 months, less than 5 months, less than 6 months, less than 9 months, or less than 12 months after administering the priming dose. In one embodiment, the biological sample is collected between about 3 months and about 6 months after administering the priming dose. In some embodiments, the biological sample is collected between about 3 months and about 9 months after administering the priming dose. In some embodiments, the biological sample is collected between about 3 months and about 12 months after administering the priming dose. In some embodiments, the biological sample is collected between about 6 months and about 12 months after administering the priming dose.

In some embodiments, a method of producing a broadly neutralizing anti-S antibody further comprises purifying the broadly neutralizing anti-S antibody. In some embodiments, the antibody is a polyclonal antibody.

In some embodiments, a method of producing a broadly neutralizing anti-S antibody further comprises isolating from the bovine a biological sample comprising the broadly neutralizing anti-S antibody; purifying the broadly neutralizing anti-S antibody; processing the broadly neutralizing anti-S antibody to prepare an F(ab) or F(ab′)₂ fragment; and recovering the F(ab) or F(ab′)₂ fragment. In one embodiment, the antibody is a polyclonal antibody. In some embodiments, the antibody is a polyclonal F(ab) or F(ab′)₂ fragment. Methods for producing an F(ab′)₂ fragment and compositions thereof are known in in the art, for example, as disclosed in U.S. Pat. No. 6,709,655, which is incorporated by reference herein in its entirety for all purposes.

In some embodiments, a method of producing a broadly neutralizing anti-S antibody further comprises isolating a peripheral blood mononuclear cell (PMBCs) from the bovine, and cloning a polynucleotide that encodes a broadly neutralizing anti-S antibody. In one embodiment, the cloning the polynucleotide comprises performing single-cell RT-PCR amplification.

### IV. Pharmaceutical Compositions and Formulations

Also provided are compositions comprising the binding polypeptides, such as antibodies or antigen-binding fragments or knob peptides, described herein, including pharmaceutical compositions and formulations. In one embodiment, a composition comprises a broadly neutralizing anti-S antibody (e.g., bovine antibody or humanized bovine antibody) or antigen-binding fragment described herein. In some embodiments, a composition comprises a knob peptide as described, such as derived from an ultralong CDR3. The pharmaceutical compositions and formulations generally include one or more optional pharmaceutically acceptable carrier or excipient.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell, binding molecule, and/or antibody, and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

Formulations of the antibodies described herein can include lyophilized formulations and aqueous solutions.

In some embodiments, an antibody described herein may be administered within a pharmaceutically-acceptable diluent, carrier, or excipient, in unit dose form. Conventional pharmaceutical practice may be employed to provide suitable formulations or compositions to administer to individuals being treated for SARS CoV-2 infection. In some embodiments, the administration is prophylactic. Any appropriate route of administration may be employed, for example, administration may be parenteral, intravenous, intra-arterial, subcutaneous, intramuscular, intraperitoneal, intranasal, aerosol, suppository, oral administration, or via inhalation.

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, intracranial, intrathoracic, and intraperitoneal administration.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the binding molecule in a solvent, such as in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

Pharmaceutical compositions according to the invention may be, for example, in unit dose form, such as in the form of ampoules, vials, suppositories, tablets, pills, or capsules. The formulations can be administered to human individuals in therapeutically or prophylactic effective amounts (e.g., amounts which prevent, eliminate, or reduce a pathological condition) to provide therapy for a disease or condition. The preferred dosage of therapeutic agent to be administered is likely to depend on such variables as the type and extent of the disorder, the overall health status of the particular patient, the formulation of the compound excipients, and its route of administration.

In certain embodiments, the compositions described herein can be formulated for pneumonal administration, and in certain embodiments the composition is formulated for administration via inhalation (e.g., intrabronchial, intranasal or oral inhalation, intranasal drops). The composition may be administered with the use of a nebulizer, inhaler, atomizer, aerosolizer, mister, dry powder inhaler, metered dose inhaler, metered dose sprayer, metered dose mister, metered dose atomizer, or other suitable delivery device.

In some embodiments, the composition is a lyophilized composition. In some embodiments, the composition is formulated for aerosol administration, and in certain embodiments the composition is formulated for oral administration or administration via inhalation.

The pharmaceutical compositions described herein are prepared in a manner known per se, for example, by means of conventional dissolving, lyophilizing, mixing, granulating or confectioning processes. The pharmaceutical compositions may be formulated according to conventional pharmaceutical practice (see for example, in Remington: The Science and Practice of Pharmacy (21st ed.), ed. A. R. Gennaro, 2005, Lippincott Williams & Wilkins, Philadelphia, PA, and Encyclopedia of Pharmaceutical Technology, eds. J. Swarbrick and J. C. Boylan, 2013, Marcel Dekker, New York, NY).

In instances where aerosol administration is appropriate, the squalamine or a derivative thereof can be formulated as aerosols using standard procedures. The term “aerosol” includes any gas-borne suspended phase of a squalamine or a derivative thereof which is capable of being inhaled into the bronchioles or nasal passages, and includes dry powder and aqueous aerosol, and pulmonary and nasal aerosols. Specifically, aerosol includes a gas-bome suspension of droplets of squalamine or a derivative thereof, as may be produced in a metered dose inhaler or nebulizer, or in a mist sprayer. Aerosol also includes a dry powder composition of a compound of the invention suspended in air or other carrier gas, which may be delivered by insufflation from an inhaler device, for example. See Ganderton & Jones, Drug Delivery to the Respiratory Tract (Ellis Horwood, 1987); Gonda, Critical Reviews in therapeutic Drug Carrier Systems, 6:273-313 (1990); and Raeburn et al. Pharmacol. Toxicol. Methods, 27:143-159 (1992).

The formulations to be used for in vivo administration are generally sterile. The injection compositions are prepared in customary manner under sterile conditions; the same applies also to introducing the compositions into ampoules or vials and sealing the containers. Sterility may be readily accomplished, e.g., by filtration through sterile filtration membranes.

The pharmaceutical composition in some aspects can employ time-released, delayed release, and sustained release delivery systems such that the delivery of the composition occurs prior to, and with sufficient time to cause, sensitization of the site to be treated. Many types of release delivery systems are available and known. Such systems can avoid repeated administrations of the composition, thereby increasing convenience to the subject and the physician.

The pharmaceutical composition in some embodiments contains the binding polypeptides, such as antibodies or antigen binding fragments, in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

### V. Methods of Treatment and Uses

Provided herein are methods of treatment and uses for treating a coronavirus infection. In some embodiments, provided embodiments relate to methods for treating or preventing a coronavirus infection in a subject. In some embodiments, the methods are for prophylactic treatment of a viral infection in a subject at risk of a viral infection. In some embodiments, the methods are for treating a subject known or suspected of having a viral infection. In some embodiments, the methods may prevent a viral infection, such as a coronavirus infection, in a subject. In some embodiments, the methods may reduce signs of symptoms of the coronavirus infection in the subject, such as mitigate the presence or severity of one or more signs or symptoms. In some embodiments, the binding molecules, such as antibodies or antigen binding fragments or knob peptides, are administered to a subject in an effective amount to effect treatment of the infection. Also provided herein are uses of the binding polypeptides, such as antibodies or antigen binding fragments or knob peptides, in such methods and treatments, and in the preparation of a medicament in order to carry out such therapeutic methods. In some embodiments, the methods are carried out by administering the binding polypeptides, or compositions comprising the same, to the subject having, having had, or suspected of having the disease or condition. In some embodiments, the methods thereby treat the disease or condition or disorder in the subject. Also provided herein are of use of any of the compositions, such as pharmaceutical compositions provided herein, for the treatment of a disease or disorder associated with a coronavirus infection, for example, due to SARS-CoV-2.

The coronavirus infection typically involves respiratory tract infections, often in the lower respiratory tract. Symptoms can include high fever, dry cough, shortness of breath, pneumonia, gastro-intestinal symptoms such as diarrhea, organ failure (kidney failure and renal dysfunction), septic shock, and death in severe cases. The coronavirus infection may include any virus infection in the body of a subject that is treatable or preventable by administration of a binding polypeptide directed against a coronavirus spike protein wherein infectivity of the virus is at least partially dependent on the coronavirus spike protein.

In particular, the virus is a virus that infects the respiratory tissue of a subject (e.g., upper and/or lower respiratory tract, trachea, bronchi, lungs) and is treatable or preventable by administration of an binding polypeptide against the coronavirus spike protein. Coronaviruses can include the genera of alphacoronaviruses, betacoronaviruses, gammacoronaviruses, and deltacoronaviruses. For example, the virus includes coronavirus, such as SARS-CoV-2 (severe acute respiratory syndrome coronavirus 2), SARS-CoV (severe acute respiratory syndrome coronavirus), and MERS-CoV (Middle East respiratory syndrome (MERS) coronavirus). In some embodiments, the coronavirus infection is an infection of a subject with a coronavirus such as SARS-CoV-2, MERS-CoV, or SARS-CoV. In some embodiments, the coronavirus infection is an infection of a subject with SARS-CoV-2.

In some embodiments, the methods and uses include administering a provided binding polypeptide, such as an antibody or antigen binding fragment or knob peptide, into a subject (e.g. a human). In some embodiments, the binding polypeptide or a composition containing same is administered to the subject by a parenteral administration. In some embodiments, the binding polypeptide or a composition containing same is administered by intramuscularly, subcutaneously, intravenously, topically, orally or by inhalation. In particular embodiments, particularly for delivery of a knob peptide, the administration is by inhalation. In some embodiments, a provided binding polypeptide, such as a knob peptide, may be administered by aerosol administration, such as by delivery using an inhaler or nebulizer or a mist sprayer.

In some embodiments, a provided binding polypeptide, such as an antibody or antigen binding fragment or a knob peptide, is administered to the subject in an effective or therapeutically effective amount. An effective or therapeutically effective dose of a provided binding polypeptide, such as an antibody or antigen binding fragment or knob peptide, for treating or preventing a viral infection is an amount sufficient to alleviate one or more signs and/or symptoms of the infection in the treated subject, whether by inducing the regression or elimination of such signs and/or symptoms or by inhibiting the progression of such signs and/or symptoms. The dose amount may vary depending upon the age and the size of a subject to be administered, target disease, conditions, route of administration, and the like. In an embodiment, an effective or therapeutically effective dose of a provided binding polypeptide, such as an antibody or antigen-binding fragment thereof or a knob peptide, for treating or preventing viral infection, e.g., in an adult human subject, is about 0.001 mg/kg to about 200 mg/kg, such as 0.01 mg/kg to 200 mg/kg or 0.1 mg/kg to 200 mg/kg. Depending on the severity of the infection, the frequency and the duration of the treatment can be adjusted.

In some embodiments, the subject may have a viral infection, e.g., an influenza infection, or be predisposed to developing an infection. Subjects predisposed to developing an infection, or subjects who may be at elevated risk for contracting an infection (e.g., of coronavirus or influenza virus), include subjects with compromised immune systems because of autoimmune disease, subjects receiving immunosuppressive therapy (for example, following organ transplant), subjects afflicted with human immunodeficiency syndrome (HIV) or acquired immune deficiency syndrome (AIDS), subjects with forms of anemia that deplete or destroy white blood cells, subjects receiving radiation or chemotherapy, or subjects afflicted with an inflammatory disorder. Additionally, subjects of very young (e.g., 5 years of age or younger) or old age (e.g., 65 years of age or older) are at increased risk. Moreover, a subject may be at risk of contracting a viral infection due to proximity to an outbreak of the disease, e.g. subject resides in a densely-populated city or in close proximity to subjects having confirmed or suspected infections of a virus, or choice of employment, e.g. hospital worker, pharmaceutical researcher, traveler to infected area, or frequent flier.

The provided methods and uses include methods and uses for treating a viral infection in a subject. For instance, methods of treating include administering a provided binding polypeptide, such as an antibody or antigen-binding fragment or a knob peptide, to a subject having one or more signs or symptoms of a disease or infection, e.g., viral infection, at an effective or therapeutically effective amount or dose.

In some embodiments, the provided methods and uses include prophylactic methods and uses. In some embodiments, provided herein are methods for prophylactically administering a provided binding polypeptide, such as an antibody or antigen-binding fragment or a knob peptide, to a subject having who is at risk of viral infection so as to prevent such infection. In some embodiments, the amount administered is an effective or therapeutically effective amount or dose. In some embodiments, the provided methods and uses prevent a viral infection in the subject. In some embodiments, preventing a viral infection by a provided methods involves administering a provided binding polypeptide, such as an antibody or antigen binding fragment or knob peptide, to a subject to inhibit the manifestation of a disease or infection (e.g., viral infection) in the body of a subject. In some embodiments, the methods reduce one or more sign or symptom of a viral infection.

In aspects of any of the provided embodiments, the methods include a method for treating or preventing viral infection (e.g., coronavirus infection) or for inducing the regression or elimination or inhibiting the progression of at least one sign or symptom of viral infection.

In aspects of the provided methods or uses, a sign or symptom of a viral infection in a subject is survival or proliferation of virus in the body of the subject, e.g., as determined by viral titer assay (e.g., coronavirus propagation in embryonated chicken eggs or coronavirus spike protein assay). In some embodiments, a sign or symptom of viral infection includes, for example, fever or feeling feverish/chills; cough; sore throat; runny or stuffy nose; sneezing; muscle or body aches; headaches; fatigue (tiredness); vomiting; diarrhea; respiratory tract infection; chest discomfort; shortness of breath; bronchitis; and/or pneumonia.

In further embodiments of the present disclosure, a composition comprising a binding polypeptide described herein, such as an antibody or antigen binding fragment or a knob peptide, can additionally be combined with other compositions for the treatment of a coronavirus infection, such as SARS CoV-2 infection or the prevention of SARS CoV-2 transmission.

### VI. Exemplary Embodiments

Among the provided embodiments are:
1. An isolated knob peptide of over 30 amino acids in length directed against SARS-CoV-2, wherein the knob peptide is isolated or derived from an ultralong CDR3 of a bovine antibody.
2. The isolated knob peptide of exemplary embodiment 1, wherein the knob peptide comprises a contiguous sequence of amino acids within the sequence set forth in any one of SEQ ID NOS: 112, 114, 116, 117, 119, 121, 123, or 126, wherein the knob peptide is up to 70 amino acids in length and contains 2-6 disulfide bonds.
3. A knob peptide directed against SARS-CoV-2 spike protein, comprising a contiguous sequence of amino acids of at least 40 amino acids within the sequence set forth in any one of SEQ ID NOS: 112, 114, 116, 117, 119, 121, 123, or 126, wherein the knob peptide is up to 70 amino acids in length and contains 2-6 disulfide bonds.
4. The knob peptide of any of exemplary embodiments 1-3, wherein the knob peptide comprises 6-9 C-terminal amino acids after the last cysteine (Cys) residue of the respective sequence of amino acids set forth in any one of SEQ ID NOS: 112, 114, 116, 117, 119, 121, 123, or 126, optionally at or about 9 C-terminal amino acids after the last cysteine (Cys) residue of the respective sequence of amino acids set forth in any one of SEQ ID NOS: 112, 114, 116, 117, 119, 121, 123, or 126.
5. The knob peptide of any of exemplary embodiments 1-4, wherein the sequence of the knob peptide begins 3-6 amino acids before the N-terminal Cys residue of the respective sequence of amino acids set forth in any one of SEQ ID NOS: 112, 114, 116, 117, 119, 121, 123, or 126.
6. The knob peptide of any of exemplary embodiments 1-5, wherein the knob peptide comprises at or about 9 C-terminal amino acids after the last cysteine (Cys) residue of the respective sequence of amino acids set forth in any one of SEQ ID NOS: 112, 114, 116, 117, 119, 121, 123, or 126.
7. The knob peptide of any of exemplary embodiments 1-6, wherein the knob peptide is 40 to 60 amino acids in length, optionally wherein the knob peptide is at least 42 amino acids in length.
8. The knob peptide of any of exemplary embodiments 1-7, wherein the knob peptide is 42 amino acids, 43 amino acids, 44 amino acids, 45 amino acids, 46 amino acids, 47 amino acids, 48 amino acids, 49 amino acids, 50 amino acids, 51 amino acids, 52 amino acids, 53 amino acids, 54 amino acids, 55 amino acids, 56 amino acids, 57 amino acids, 58 amino acids, 59 amino acids or 60 amino acids in length.
9. The knob peptide of any of exemplary embodiments 1-8, wherein the knob peptide comprises at least 4 Cys residues, optionally wherein the knob peptide contains 4 Cys residues.
10. The knob peptide of any of exemplary embodiments 1-8, wherein the knob peptide contains 6, 8, 10 or 12 Cys residues.
11. The knob peptide of any of exemplary embodiments 1-10, wherein the knob peptide has at least 2 disulfide bonds.
12. The knob peptide of any of exemplary embodiments 1-11, wherein the knob peptide has 2 disulfide bonds.
13. The knob peptide of any of exemplary embodiments 1-11, wherein the knob peptide has 3, 4 or 5 disulfide bonds.
14. The knob peptide of any of exemplary embodiments 1-13, wherein the knob peptide comprises an amino acid sequence set forth in any one of SEQ ID NOS: 160, 169, 179, 189, 198, 208, 215 or 224.
15. The knob peptide of any of exemplary embodiments 1-14, wherein the knob peptide is at or about 42 amino acids, 43 amino acids, 44 amino acids, 45 amino acids, 46 amino acids, 47 amino acids, 48 amino acids, 49 amino acids, 50 amino acids, 51 amino acids, 52 amino acids, 53 amino acids, 54 amino acids, or 55 amino acids.
16. The knob peptide of any of exemplary embodiments 1-14, wherein the knob peptide comprises the amino acid sequence set forth in SEQ ID NO:160.
17. The knob peptide of exemplary embodiment 16, wherein the knob peptide is set forth in any one of SEQ ID NOS: 63 or 159-168.
18. The knob peptide of any of exemplary embodiments 1-14, wherein the knob peptide comprises the amino acid sequence set forth in SEQ ID NO: 169.
19. The knob peptide of any of exemplary embodiments 1-14 and 18, wherein the knob peptide is set forth in any one of SEQ ID NOS: 66 or 169-177.
20. The knob peptide of any of exemplary embodiments 1-14, wherein the knob peptide comprises the amino acid sequence set forth in SEQ ID NO:179.
21. The knob peptide of any of exemplary embodiments 1-14 and 20, wherein the knob peptide is set forth in any one of SEQ ID NOS: 65 or 178-187.
22. The knob peptide of any of exemplary embodiments 1-14, wherein the knob peptide comprises the amino acid sequence set forth in SEQ ID NO:189.
23. The knob peptide of any of exemplary embodiments 1-14 and 22, wherein the knob peptide is set forth in any one of SEQ ID NOS: 64 or 188-197.
24. The knob peptide of any of exemplary embodiments 1-14, wherein the knob peptide comprise the amino acid sequence set forth in SEQ ID NO:198.
25. The knob peptide of any of exemplary embodiments 1-14 and 24, wherein the knob peptide is set forth in any one of SEQ ID NOS: 60 or 198-206.
26. The knob peptide of any of exemplary embodiments 1-14, wherein the knob peptide comprises the amino acid sequence set forth in SEQ ID NO:208.
27. The knob peptide of any of exemplary embodiments 1-14 and 26, wherein the knob peptide is set forth in any one of SEQ ID NOS: 62 or 207-214.
28. The knob peptide of any of exemplary embodiments 1-14, wherein the knob peptide comprises the amino acid sequence set forth in SEQ ID NO:215.
29. The knob peptide of any of exemplary embodiments 1-14 and 28, wherein the knob peptide is set forth in any of SEQ ID NOS: 61 and 215-223.
30. The knob peptide of any of exemplary embodiments 1-14, wherein the knob peptide comprises the amino acid sequence set forth in SEQ ID NO:224.
31. The knob peptide of any of exemplary embodiments 1-14 and 30, wherein the knob peptide is set forth in any one of SEQ ID NOS: 68 or 224-232.
32. The knob peptide of any of exemplary embodiments 1-31, wherein the knob peptide further comprises an N-terminal or C-terminal linker, optionally a GS linker.
33. The knob peptide of any of exemplary embodiments 1-32, wherein the knob peptide further comprises a linker for cyclization of the peptide.
34. The knob peptide of exemplary embodiment 33, wherein the linker is GGGGAMGS (SEQ ID NO: 234).
35. The knob peptide of any of exemplary embodiments 1-34, wherein the peptide is cyclized.
36. The knob peptide of any of exemplary embodiments 1-35, wherein the knob peptide binds to the spike protein of SARS-CoV2 or a binding portion or epitope thereof with a binding affinity dissociation constant of less than at or about 10⁻⁹ M, less than at or about 10⁻¹⁰ M, less than at or about 10⁻¹¹ M or less than at or about 10⁻² M.
37. The knob peptide of any of exemplary embodiments 1-36, wherein the knob peptide neutralizes SARS-CoV2 infection of a human cell.
38. The knob peptide of any of exemplary embodiments 1-37, wherein the knob peptide neutralizes SARS-CoV2 with an EC50 of less than about 100 ng/mL, 10 ng/mL, 1 ng/mL, 0.1 ng/mL, 0.01 ng/mL, 0.001 ng/mL.
39. The knob peptide of any of exemplary embodiments 1-37, wherein the knob peptide neutralizes SARS-COV2 with an EC50 of less than about 100 nM, 10 nM, 1 nM, 0.1 nM, 0.01 nM or 0.001 nM.
40. The knob peptide of any of exemplary embodiments 1-39, wherein the knob peptide neutralizes SARS-COV2 with an EC50 of less than at or about 500 pM, 250 pM, 100 pM, 50 pM, 25 pM, 10 pM, 5 pM, 2.5 pM, 1 pM, 0.5 pM or less.
41. The knob peptide of any of exemplary embodiments 1-40, wherein the SARS CoV-2 is selected from Wuhan-Hu-1 strain, UK (B.1.1.7) strain or South African (B.1.351) strain.
42. The knob peptide of any of exemplary embodiments 1-40, wherein the SARS CoV-2 is Wuhan-Hu-1 strain.
43. The knob peptide of any of exemplary embodiments 1-42, wherein the knob peptide is recombinantly produced.
44. The knob peptide of any of exemplary embodiments 1-43, wherein the knob peptide is purified.
45. A multispecific binding protein, comprising a plurality of knob peptides of any of exemplary embodiments 1-44.
46. The multispecific binding protein, wherein the plurality of knob peptides are paratopes.
47. The multispecific binding protein of exemplary embodiment 45 or exemplary embodiment 46, wherein the plurality of knob peptides are 2, 3, or 4 peptides.
48. A composition comprising a knob peptide of any of exemplary embodiments 1-44.
49. A composition comprising a plurality of knob peptides of any of exemplary embodiments 1-44.
50. The composition of exemplary embodiment 49, wherein the plurality of knob peptides are paratopes.
51. The composition of exemplary embodiment 49 or exemplary embodiment 50, wherein the plurality of knob peptides are 2, 3 or 4 peptides.
52. The composition of any of exemplary embodiments 48-51 that is a pharmaceutical composition, optionally comprising a pharmaceutical carrier.
53. The composition of any of exemplary embodiments 48-52, wherein the composition is formulated for inhalation.
54. An antibody or antigen binding fragment directed against the spike protein of SARS-CoV2, wherein the antibody or antigen binding fragment comprises a heavy chain variable region (VH) and a light chain variable region (VL), wherein the VH comprises a CDR3 H3 set forth in any one of SEQ ID NOS: 86, 88, 89, 92, 95, 98, 101, 103, 104, 107, 110, 112, 114, 116, 117, 119, 121, 123, or 126.
55. The antibody or antigen binding fragment, wherein the VH further comprises a CDR-H1 set forth in any one of SEQ ID NOS: 84, 90, 93, 96, 99, 105, 108, 113, 122 or 124 and a CDR-H2 set forth in any one of SEQ ID NOS: 85, 87, 91, 94, 97, 100, 102, 106, 109, 111, 115, 118, 120 or 125.
56. The antibody or antigen binding fragment, wherein the VH comprises:
   (a) a CDR-H1, a CDR-H2 and CDR-H3 set forth in SEQ ID NOS: 84, 85 and 86, respectively;
   (b) a CDR-H1, a CDR-H2 and CDR-H3 set forth in SEQ ID NOS: 84, 87 and 88, respectively;
   (c) a CDR-H1, a CDR-H2 and CDR-H3 set forth in SEQ ID NOS: 84, 85 and 89, respectively;
   (d) a CDR-H1, a CDR-H2 and CDR-H3 set forth in SEQ ID NOS: 90, 91 and 92, respectively;
   (e) a CDR-H1, a CDR-H2 and CDR-H3 set forth in SEQ ID NOS: 93, 94 and 95, respectively;
   (f) a CDR-H1, a CDR-H2 and CDR-H3 set forth in SEQ ID NOS: 96, 97 and 98, respectively;
   (g) a CDR-H1, a CDR-H2 and CDR-H3 set forth in SEQ ID NOS: 99, 100 and 101, respectively;
   (h) a CDR-H1, a CDR-H2 and CDR-H3 set forth in SEQ ID NOS: 96, 102 and 103, respectively;
   (i) a CDR-H1, a CDR-H2 and CDR-H3 set forth in SEQ ID NOS: 99, 102 and 104, respectively;
   (j) a CDR-H1, a CDR-H2 and CDR-H3 set forth in SEQ ID NOS: 105, 106 and 107, respectively;
   (k) a CDR-H1, a CDR-H2 and CDR-H3 set forth in SEQ ID NOS: 108, 109 and 110, respectively;
   (l) a CDR-H1, a CDR-H2 and CDR-H3 set forth in SEQ ID NOS: 108, 111 and 112, respectively;
   (m) a CDR-H1, a CDR-H2 and CDR-H3 set forth in SEQ ID NOS: 113, 111 and 114, respectively;
   (n) a CDR-H1, a CDR-H2 and CDR-H3 set forth in SEQ ID NOS: 108, 115 and 116, respectively;
   (o) a CDR-H1, a CDR-H2 and CDR-H3 set forth in SEQ ID NOS: 108, 115 and 117, respectively;
   (p) a CDR-H1, a CDR-H2 and CDR-H3 set forth in SEQ ID NOS: 108, 118 and 119, respectively;
   (q) a CDR-H1, a CDR-H2 and CDR-H3 set forth in SEQ ID NOS: 108, 120 and 121, respectively;
   (r) a CDR-H1, a CDR-H2 and CDR-H3 set forth in SEQ ID NOS: 122, 111 and 123, respectively; or
   (s) a CDR-H1, a CDR-H2 and CDR-H3 set forth in SEQ ID NOS: 124, 125 and 126, respectively.
57. The antibody or antigen binding fragment of any of exemplary embodiments 54-56, wherein the VH comprises the sequence set forth in any one of SEQ ID NOS: 33-51.
58. The antibody or antigen binding fragment of any of exemplary embodiments 54-56, wherein the VH is humanized
59. The antibody or antigen binding fragment of any of exemplary embodiments 54-56 and 58, wherein the VH has the formula V1-X-V2, wherein the V1 region is a humanized variant of the V region of a bovine antibody, optionally wherein the bovine antibody is BLV1H12, the X is the CDR3 and X2 is a portion of the heavy chain including FR-4 of a human J gene segment or a humanized bovine J gene.
60. The antibody or antigen binding fragment of exemplary embodiment 59, wherein V1 is set forth in SEQ ID NO:141, X is set forth in any one of SEQ ID NOS: 86, 88, 89, 92, 95, 98, 101, 103, 104, 107, 110, 112, 114, 116, 117, 119, 121, 123, or 126, and V2 is set forth in any of SEQ ID NOS: 142-147.
61. An ultralong CDR3 antibody or antigen binding fragment directed against the spike protein of SARS-CoV2, wherein the antibody or antigen binding fragment comprises a heavy chain variable region (VH) and a light chain variable region (VL), wherein the VH comprises a CDR3 H3 set forth in any one of SEQ ID NOS: 112, 114, 116, 117, 119, 121, 123, or 126.
62. The ultralong CDR3 antibody or antigen binding fragment of exemplary embodiment 61, wherein the VH further comprises a CDR-H1 set forth in any one of SEQ ID NOS: 108, 113, 122 or 124 and a CDR-H2 set forth in any one of SEQ ID NOS: 111, 115, 118, 120 or 125.
63. The ultralong CDR3 antibody or antigen binding fragment of exemplary embodiment 61 or exemplary embodiment 62, wherein the VH comprises:
   (a) a CDR-H1, a CDR-H2 and CDR-H3 set forth in SEQ ID NOS: 108, 111 and 112, respectively;
   (b) a CDR-H1, a CDR-H2 and CDR-H3 set forth in SEQ ID NOS: 113, 111 and 114, respectively;
   (c) a CDR-H1, a CDR-H2 and CDR-H3 set forth in SEQ ID NOS: 108, 115 and 116, respectively;
   (d) a CDR-H1, a CDR-H2 and CDR-H3 set forth in SEQ ID NOS: 108, 115 and 117, respectively;
   (e) a CDR-H1, a CDR-H2 and CDR-H3 set forth in SEQ ID NOS: 108, 118 and 119, respectively;
   (f) a CDR-H1, a CDR-H2 and CDR-H3 set forth in SEQ ID NOS: 108, 120 and 121, respectively;
   (g) a CDR-H1, a CDR-H2 and CDR-H3 set forth in SEQ ID NOS: 122, 111 and 123, respectively; or
   (h) a CDR-H1, a CDR-H2 and CDR-H3 set forth in SEQ ID NOS: 124, 125 and 126, respectively.
64. The ultralong CDR3 antibody or antigen binding fragment of any of exemplary embodiments 61-63, wherein the VH comprises the sequence set forth in any one of SEQ ID NOS: 33, 34, 35, 40,45, 46, 50 or 51.
65. The ultralong CDR3 antibody or antigen binding fragment of any of exemplary embodiments 61-63, wherein the VH is humanized.
66. The ultralong CDR3 antibody or antigen binding fragment of any of exemplary embodiments 61-63 and 65, wherein the VH has the formula V1-X-V2, wherein the V1 region is a humanized variant of the V region of a bovine antibody, optionally wherein the bovine antibody is BLV1 H12, the X is the CDR3 and X2 is a portion of the heavy chain including FR-4 of a human J gene segment or a humanized bovine J gene.
67. The ultralong CDR3 antibody or antigen binding fragment of exemplary embodiment 66, wherein the V1 is set forth in SEQ ID NO:141, X is set forth in any one of SEQ ID NOS. 112, 114, 116, 117, 119, 121, 123, or 126, and V2 is set forth in any of SEQ ID NOS: 142-147.
68. The antibody or antigen binding fragment of any of exemplary embodiments 54-67, wherein the VH further comprises a constant region.
69. The antibody or antigen binding fragment of exemplary embodiment 68, wherein the constant region is a constant region of an immunoglobulin IgG1.
70. The antibody or antigen binding fragment of any of exemplary embodiments 54-69, wherein the VL is a bovine light chain variable region or is a humanized variant thereof.
71. The antibody or antigen binding fragment of any of exemplary embodiments 54-70, wherein the VL is a light chain variable region of BLVH12, BLV5D3, BLV8C11, BF1H1, BLV5B8 or F18.
72. The antibody or antigen binding fragment of any of exemplary embodiments 54-71, wherein the VL comprises a CDR-L1, CDR-L2 and CDR-L3 set forth in SEQ ID NO: 151, 152 and 153, respectively.
73. The antibody or antigen binding fragment of any of exemplary embodiments 54-72, wherein the VL is set forth in SEQ ID NO:2.
74. The antibody or antigen binding fragment of any of exemplary embodiments 54-70, wherein the VL is a humanized light chain.
75. The antibody or antigen binding fragment of any of exemplary embodiments 54-69, wherein the VL is a human light chain variable region.
76. The antibody or antigen binding fragment of any of exemplary embodiments 54-69 and 75, wherein the VL is a light chain variable region of VL1-47, VL1-40, VL1-51, and VL2-18, or a variant thereof comprising substitution of amino acids to residues at corresponding positions in a bovine light chain sequence.
77. The antibody or antigen binding fragment of exemplary embodiment 76, wherein the amino acid substitutions are selected from S2A, T5N, P8S, A12G, A13S, and P14L and/or substitution of DNN to GDT, optionally substitution DNNKRP to GDTSRA.
78. The antibody or antigen binding fragment of any of exemplary embodiments 54-69 and 76, wherein the VL comprises the sequence set forth in any of SEQ ID NOS. 155-158.
79. The antibody or antigen binding fragment of any of exemplary embodiments 54-78, wherein the light chain further comprises a constant region.
80. The antibody or antigen binding fragment of any of exemplary embodiments 54-79, wherein the light chain is set forth in SEQ ID NO:154.
81. The antibody or antigen binding fragment of any of exemplary embodiments 54-80 that is a full length antibody.
82. The antibody or antigen binding fragment of any of exemplary embodiments 54-80 that is an antigen binding fragment selected from a Fab or a single chain variable fragment (scFv).
83. The antibody or antigen binding fragment of any of exemplary embodiments 54-82 that is isolated or recombinant.
84. The antibody or antigen binding fragment of any of exemplary embodiments 54-83 that is a monoclonal antibody.
85. A composition comprising an antibody or antigen binding fragment of any of exemplary embodiments 54-84.
86. The composition of exemplary embodiment 85 that is a pharmaceutical composition, optionally comprising a pharmaceutical carrier.
87. A polynucleotide encoding the knob peptide of any of exemplary embodiments 1-44.
88. A polynucleotide encoding the multispecific polypeptide of any of exemplary embodiments 45-47.
89. A polynucleotide encoding the antibody or antigen binding fragment of any of exemplary embodiments 54-84.
90. The polynucleotide of any of exemplary embodiments 87-89 that is a synthetic nucleic acid.
91. The polynucleotide d of any of exemplary embodiments 87-90 that is cDNA.
92. A vector comprising the polynucleotide of any of exemplary embodiments 87-91.
93. The vector of exemplary embodiment 92 that is an expression vector.
94. A method of treating a coronavirus infection in a subject in need, the method comprising administering to a subject the knob peptide of any of exemplary embodiments 1-44, the multispecific polypeptide of any of exemplary embodiments 45-47 or the antibody or antigen binding fragment of any of exemplary embodiments 54-84.
95. A method of prophylactically treating a subject at risk for developing a coronavirus infection, the method comprising administering to a subject the knob peptide of any of exemplary embodiments 1-44, the multispecific polypeptide of any of exemplary embodiments 45-47 or the antibody or antigen binding fragment of any of exemplary embodiments 54-84.
96. A method of preventing a coronavirus in aa subject at risk for developing a coronavirus infection, the method comprising administering to a subject the knob peptide of any of exemplary embodiments 1-44, the multispecific polypeptide of any of exemplary embodiments 45-47 or the antibody or antigen binding fragment of any of exemplary embodiments 54-84.
97. The method of any of exemplary embodiments 94-96, wherein the coronavirus infection is caused by SARS-CoV, SARS-CoV-2, or MERS-CoV.
98. The method of any of exemplary embodiments 94-97, wherein the coronavirus infection is COVID-19.
99. The method of any of exemplary embodiments 94-98, wherein the administration is a parenteral administration.
100. The method of any of exemplary embodiments 94-99, wherein the administration is a subcutaneous or intravenous administration.
101. The method of any of exemplary embodiments 94-98, wherein the administration is by inhalation.
102. The method of exemplary embodiment 101, wherein the administration is by use of a nebulizer, inhaler, atomizer, aerosolizser, mister, dry powdered inhaler, metered dose inhaler, metered dose sprayer, metered dose mister, or metered dose atomizer.

### VII. Examples

The following examples are included for illustrative purposes only and are not intended to limit the scope of the invention.

### Example 1 Generation of Anti-SARS CoV-2 Antibodies

Cows were immunized with SARS CoV-2 Spike protein or receptor binding domain (RBD) portion thereof and sera was collected to assess binding activity.

### A. Spike Protein and Receptor Binding Domain Expression and Purification

SARS CoV-2 spike trimer protein from the parental Wuhan-Hu-1 isolate (NCBI YP_009724390.1) or the B.1.351 “South African” variant with the mutation E484K (and K417N and N501Y), or the parental receptor binding domain (RBD) protein (amino acids 319 to 541 of the spike protein), were produced by transfection of HEK293 cells. Approximately 120×10⁶ HEK293 Freestyle cells with 293fectin (Invitrogen) were combined with 120 μg of pCAGGS-based vector containing (1) the sequence encoding the extracellular domain of the Spike protein with furin-cleavage site removed and K986P and V987P stabilizing mutations, T4-fibritin trimerization domain and c-terminal 6× His-tag, or (2) spike RBD domain (amino acids 319 to 541 of the spike protein) with c-terminal 6× His-tag.

Cells were shaken at 37° C. for 4 days with 8% C02 with 150 μl TCM-ProteaseArrest tissue culture protease inhibitor (G-Biosciences) added on day 3. The supernatant containing secreted spike or RBD protein was clarified from the supernatant by centrifugation at 4000 RPM for 5 minutes followed by filtration through a 0.45 μm PES filter. The supernatant was concentrated and buffer-exchanged into PBS using Amicon Ultra Centrifugal Filter units (MWCO=50,000 for S protein preparation and 10,000 for the RBD protein) (EMD-Millipore) at 4° C. The concentrated supernatant was then purified using TALON cobalt metal affinity resin (Takara Bio) following the manufacturer's protocol, except that 50 mM, 100 mM, 200 mM, 300 mM and 400 mM imidazole gradient elution fractions (1 column volume of each) collected. Each elution fraction was resolved on an SDS-PAGE gel stained with InstantBlue Coomassie Protein Stain (Abcam). Fractions containing a single spike protein band or a single RBD band were pooled, buffer-exchanged into PBS as described above, and the concentration of protein quantified using Nanodrop One (Thermo Scientific) based on the extinction coefficient and molecular weight of the spike or RBD protein, respectively.

### B. Immunization Protocol

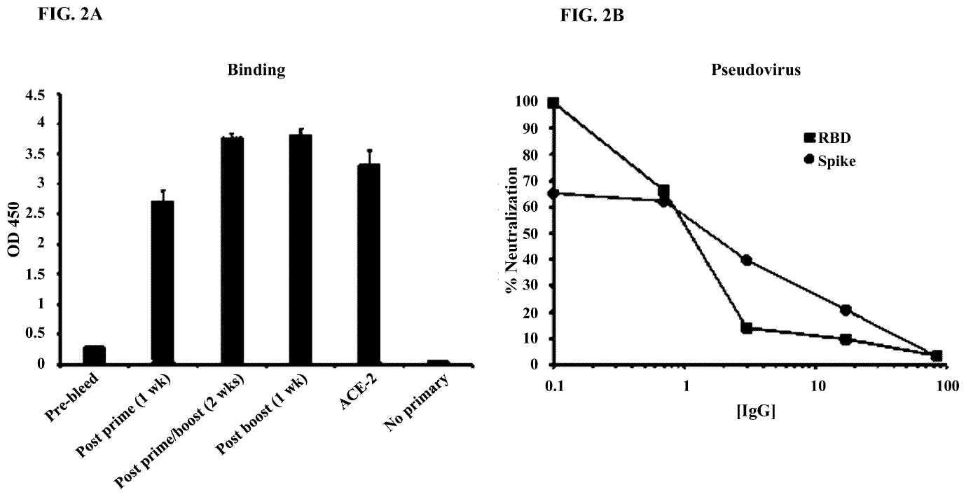

Two calves were immunized with purified Wuhan-Hu-1 spike protein or RBD protein variant with 200 μg/dose spread over 5 neck locations and boosted according to published methods (Sok et al. Nature 2017, 548(7665):108-111; Wang et al. Cell 2013, 153(6):1379-1393). Serum was collected and IgG ELISAs performed against the RBD domain of the SARS-CoV-2 spike on serum from the RBD immunized calf at a serum dilution range from 1:100 to 1:10,000. Spike protein reactivity was observed 7-21 days post-immunizations. As shown in FIG. 2A, binding activity for the RBD domain was significant after the first immunization.

Serum IgG was also assessed for neutralization of Spike protein and virus using a plaque reduction and neutralization test (PRNT). In this in vitro assay, virus and serum IgG are pre-incubated together before being concomitantly applied to permissive cells such that virus successfully bound by antibody can no longer penetrate cells and/or can no longer further propagate infection. As a result, foci of infection and cell damage called “plaques” appear to be smaller in size and/or number when the cellular monolayer is stained.

A pseudovirus expressing the SARS CoV-2 Spike protein was used as a model virus to assay percent neutralization of serum IgG from both parental Spike protein and RBD immunized cows in Vero6 cells. Compared with natural virus, the pseudovirus can be handled with BSL-2 considerations at high titer and can only infect cells in a single round. As shown in FIG. 2B, IgG obtained from cows in either of the immunization protocols was able to successfully neutralize the pseudovirus in a dose dependent manner. At higher concentrations, serum IgG (ng/mL) from cows immunized with the RBD alone was observed to neutralize 100% of pseudovirus.

Taken together, these results support that immunized cow serum, and antibodies contained therein, can neutralize SARS-CoV-2.

### Example 2 Generation of Ultralong CDR3 scFv Antibody or CDR3-Knob Only Phage Display Libraries for Antibody Discovery

Peripheral Blood Mononuclear cells (PBMCs) were collected from the immunized cows described in Example 1 and RNA was extracted to use to generate two phage display libraries as described below. Specifically, approximately 1-5×10⁷ PBMCs were collected after 14-64 days post-immunization and stored prior to RNA extraction and cDNA synthesis.

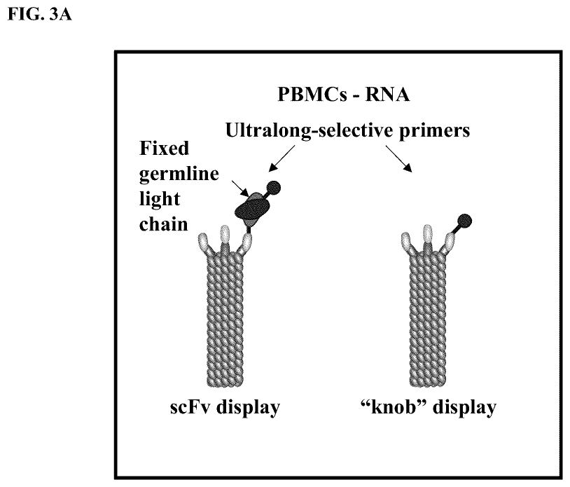

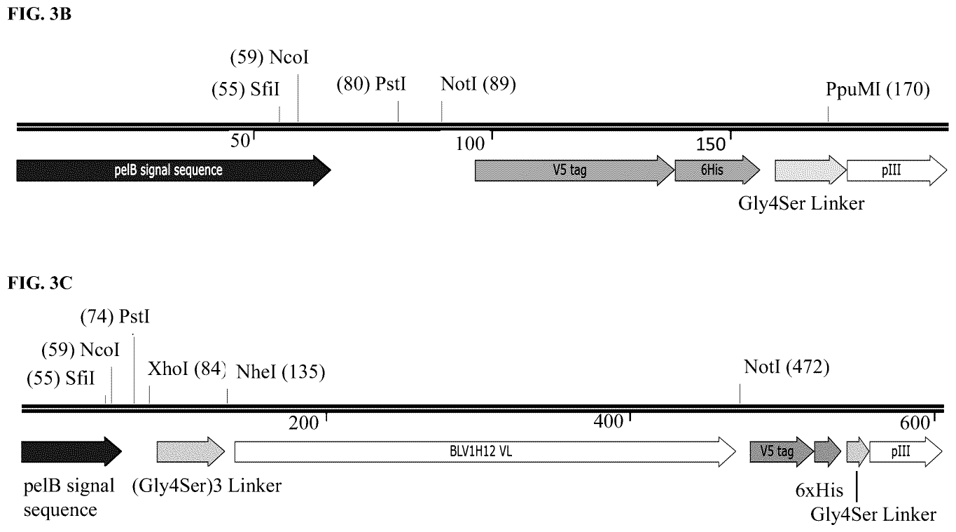

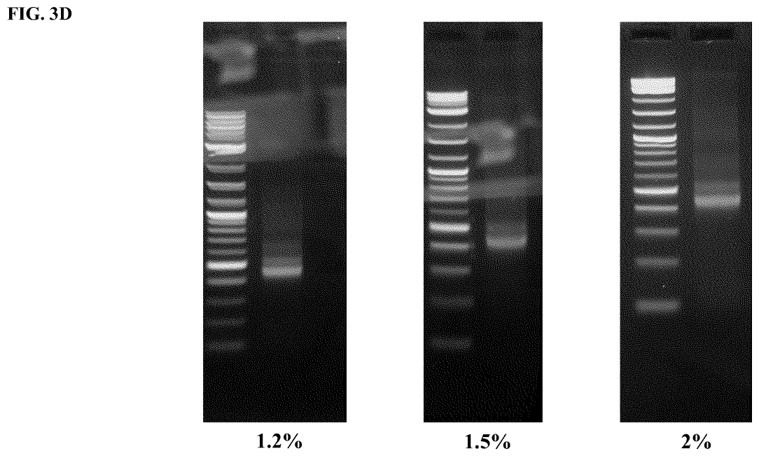

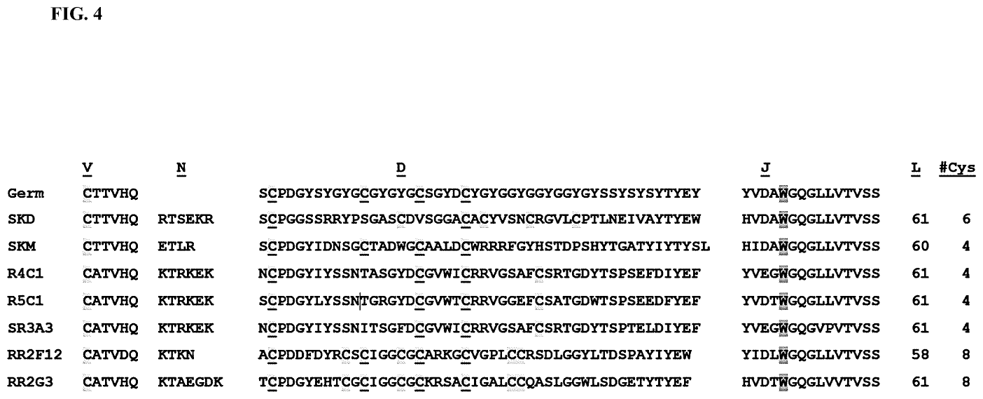

Two library strategies were employed, either using the antibodies in an scFv format with variable heavy chain (VH) and variable light chain (VL) fragments joined by a flexible linker peptide ((Gly4 Ser)3 15 amino acid linker), or using independent CDR3-knobs. In both approaches, the scFv or CDR3-knobs were fused to pIII via a flexible Gly4Ser linker. FIG. 3A depicts the pIII fusion constructs in each display library. The generation of the display libraries are summarized below.

### A. ScFv Library Construction

In the first strategy, immune cow derived VH DNA fragments were combined with a fixed light chain BLV1 H12 (Stanfield et al. Science immunology 2016, 1(1):aaf7962.). RBD and full length spike protein immune libraries were constructed for different immunization time points.

RNA was isolated from 5×10⁶-10⁷ bovine PBMC's using an RNAeasy kit (Qiagen). Immune cow antibody VH repertoires were obtained through cDNA synthesis from 5 μg total RNA using Superscript IV First-Strand cDNA synthesis kit (ThermoFisher, #18091050), followed by PCR amplification. To generate a VH template library, the cDNA template for VHs were synthesized using a pool of IgM (SEQ ID NO. 4), IgA (SEQ ID NO 5) and IgG-specific (SEQ ID NO. 3 and 6) primers.

In these hybrid libraries, full length donor ultra-long VHs were amplified from the VH template library with a VH family specific primer pair. Specifically, both VH regions were amplified with FR1 and FR4 primers specific for the bovine IgHV1-7 family (SEQ ID NO. 12 and 13, respectively) in order to enrich for VH regions with ultralong CDR3 regions. The amplified products were combined with Linker-BLV1H12 lambda light chain variable region (BLV1H12 light chain set forth in SEQ ID NOS 2 and encoded by a DNA sequence set forth in 1) by cloning into pre-cloned pTAU1 pIII fusion phage display vector (pTAU1-BLV1H12(-VH) (see FIG. 3C). The amplified products were subjected to 2 hours digestion with NcoI and XhoI (NEB) and subcloned into pTAU1-BLV1H12(-VH) as NcoI-XhoI fragments for separation of the VH and VL by the flexible linker peptide ((Gly4 Ser)3). In a further step, some ultra-long VH fragments were additionally enriched by separation from shorter VH fragments using agarose gel electrophoresis, prior to digestion with NcoI and XhoI restriction enzymes. As shown in FIG. 3D, a 2% agarose gel achieved the most separation between ultra-long VH fragments (-550 base pairs in length) and shorter VH fragments without ultralong CDR3 regions (-400 base pairs in length).

Next, this was ligated overnight with T4 DNA ligase at 16° C. Final libraries were obtained by electroporation of electrocompetent TG1 cells (Lucigen) with the purified ligation products. Each library was a minimum of 10⁷ clones with >90% with inserts.

### B. CDR3-Knob Library Construction

In a second strategy, a library of VH templates were generated substantially as described in the first strategy. Then, ultra-long VH only, immune cow derived CRD3-knob (also called “CDR3-knob only”) libraries were built by amplifying stalk-knob CDRs from the VH template library using conserved primers and cloning as pIII fusions into the pTAU1 phage display pIII fusion vector.

Specifically, RNA was isolated from 5×10′-10⁷ bovine PBMCs using an RNAeasy kit (Qiagen). Immune cow antibody CDR3-knob repertoires were obtained through cDNA synthesis from 5 μg total RNA using Superscript TV First-Strand cDNA synthesis kit (ThermoFisher), followed by PCR amplification. To generate the VH template library, the cDNA template for CDR3-knobs was synthesized using a pool of IgM (SEQ ID NO. 4), IgA (SEQ ID NO 5) and IgG-specific (SEQ ID NO. 3 and 6) primers.

Primary stalk-knob CDR3 were amplified from 1^{st} strand cDNA, with IgHV1-7 family specific primers specific for either side of the stalk domain of the CDR3 region (SEQ ID Nos: 7-11). These were then cloned into pTAU1 phage vector as NcoI-NotI fragments following 2 hours digestion with the NcoI and NotI (NEB), and ligated overnight with T4 DNA ligase at 16° C. (see FIG. 3B). Final libraries were obtained by electroporation of electrocompetent TG1 cells (Lucigen) with the purified ligation products. Each library was a minimum of 10⁷ clones with >90% with inserts.

### Example 3 Screening of Phage Display Libraries and Selection of Ultra-Lone VH or CDR3-Knob Domains Against SARS Cov-2

The VH ultra-long CDR3 scFv antibody or CDR-knob only libraries generated as described in Example 2 were subjected to two-five rounds of phage display selections against SARS CoV-2 target proteins (both parental Wuhan Hu-1 or “South African” B.1.351 variant Spike proteins or parental Wuhan Hu-1 RBD). Spike protein from either viral isolate or parental RBD were coated onto NUNC immunotubes with 1 mL of 10 μg/mL of target protein in PBS overnight at 4° C. Tubes were then blocked for 1 hour at room temperature on a blood mixer with 3-4 mL 2% Milk powder dissolved in PBS, and washed 3 times with PBS.

For each selection, approximately 10¹² phage particles from different immunized scFv or CDR3 knob libraries generated as described in Example 2 were added to 1 mL 4% milk powder dissolved in PBS, and made up to 2 mL total volume with PBS, and then added to the tubes with target protein and incubated on the blood mixer for 2 hours at room temperature. Tubes were then washed 10×PBS/0.1% Tween 20, and 10×PBS.

Bound phage were recovered with 1 mL fresh 0.1M triethylamine for 10 minutes on the blood mixer and neutralized with 0.5 mL 1M tris (pH 7.0) on ice. Log-phase TG1 Phage-Competent™ cells were infected with eluted phage for 1 hour at 37° C./200 rpm, and then grown at 30° C. overnight on 2×TY agar supplemented with 2% glucose/50 μg/mL carbenicillin.

After each round of selection described above, TG1 bacteria were scraped off the master plates into 20 mL 2×TY media supplemented with 20% glycerol/2% glucose/50 μg/mL carbenicillin. Approximately 4-5 mL of this solution was added to 20 mL of 2×TY media supplemented with 2% glucose/50 μg/mL carbenicillin containing 100 μl MI 3K07 helper phage (MOI=10). This suspension was incubated at 37° C./200 rpm for 1 hour, and added to 200 mL 2×TY/0.2M sucrose/50 μg/mL carbenicillin/25 μg/mL kanamycin/20 μm IPTG before incubating overnight at 30° C./200 rpm. Amplified phage were precipitated from cleared culture supernatants with 1/5 volume 2.5M NaCl, 20% PEG 8000 in a 250 mL Oakridge centrifuge tube after incubation on ice for 1 hour. The phage containing material was pelleted at 14,000 g in a Sorvall centrifuge for 20 minutes, resuspended in 2 mL PBS, and 1 mL reserved for use in the next round of selection. Between 2-5 rounds of selection were carried out for each library, with phage ELISA carried out for each round beginning at Round 2.

From each selection, individual colonies were picked into 600 μL 2×TY media supplemented with 50 μg/mL carbenicillin and 2% w/v glucose in 96-deepwell culture plates and incubated at 37° C. (with shaking) at 200 rpm overnight. For each culture, 50 μL was transferred to a fresh 96-deepwell plate containing 200 μL/well of the same medium and grown for 3 hours. Approximately 10⁸ kanamycin resistance units (k.r.u.) of M13K07 kanamycin-resistant helper phage was added to each well, and plates incubated at 37° C. for 1h. Expression medium (800 μL/well 2×TY media supplemented with 0.2M sucrose, 100 μg/mL carbenicillin, 25 μg/mL kanamycin, and 20 pM IPTG) was added to each well and amplification continued overnight at 30° C.

Culture plates were centrifuged at 2000 g for 10 mins at 4° C., and 25 μL of culture supernatant per well was used for ELISA. Half-area Costar ELISA plates were coated overnight at 4° C. with 50 μL/well RBD or Spike target protein at 1 μg/mL in PBS, blocked for 1 hour at room temperature with 100 μL/well of 2% milk powder dissolved in PBS, and then washed 2×100 μL/well PBS. Approximately 25 μL phage culture supernatant per well was added to each target plate or negative control plate containing 25 μL/well 4% milk powder/PBS, and allowed to bind for 1 hour at room temperature. Each plate was washed two times with 200 μL/well PBS with 0.1% Tween 20, then two times with 200 μL/well PBS. Bound phage were detected with 50μ:/well, 1:5000 diluted anti-M13-HRP conjugate (Sinobiologicals) in 2% milk powder/PBS for 1 hour at room temperature. The plates were washed and developed for 5-10 minutes at room temperature with 50 μL/well TMB (3,3′,5,5′-Tetramethylbenzidine) substrate buffer (Thermofisher). The reaction was stopped with 100 μL/well 0.5N H₂SO₄ per manufacture protocol and optical density read at 450 nm.

Positive clones from screening the scFv libraries were sequenced and both short and ultra-long VH sequences were transferred to the pFUSE human IgG1 Fc heavy chain expression vector for co-expression in mammalian HEK293 cells with chimeric BLV1H12 lambda light chain-human lambda light chain constant region. Positive clones from screening the knob-CDR3 only libraries were synthesized as full VH gene fragments and cloned into pFUSE human IgG1 Fc vector, and similarly expressed with the chimeric BLV1 H12 lambda light chain as described above. Specifically, each VH was PCR-amplified, from 10 ng phage plasmid miniprep (Qiagen), in a 50 μL reaction with 2X Phusion Hot Start II High-Fidelity PCR Master Mix (Thermo Scientific) and primers specific for V_{H} framework 1 (forward) and JH framework 4 (reverse). The PCR-generated insert was cloned into pFUSE mammalian expression vector at a 5′ EcoRI and 3′ NheI site on the 5′ end of a human IgG1 Fc gene. This was paired with a second pFUSE plasmid, containing bovine V_{L} (BLVIH12) and human λ C_{L} sequences, for transfection in HEK 293F cells. Cells were seeded at a density of 1×10⁶ cells/mL in 30-60 mL Freestyle 293 Expression Medium (Gibco), then incubated in a humidified environment at 37° C. and 8% C02. Heavy and light chain plasmids were combined 1:1 to a total amount of 1p g DNA per mL of 293F culture, then diluted in Opti MEM 1 media (Gibco) to a final volume of 1 mL per 30 mL of 293F culture. Approximately 60 μL 293fectin Transfection Reagent (Gibco) and 940 μL Opti MEM I were combined, for each 30 mL of 293F culture, then gently mixed and incubated for 5 minutes at room temperature before addition to diluted DNA. This mixture was incubated at room temperature for 30 minutes and then transferred to the 293F culture.

Medium was harvested 5 days after transfection and expressed chimeric bovine human IgG1 antibodies were purified by immobilized Protein A Sepharose (Cytiva Life Sciences) chromatography, then tested for antigen binding and neutralization of live and pseudovirus.

Selected candidate antibodies from the library screening were identified and sequenced (Table E1). A number of selected antibodies contained an ultralong CDR3 domain. Thus, despite ultralong CDR3 antibodies representing only about 10% of naturally occurring cow antibodies, candidate antibodies from the immunization described in Example 1 that were generated and screened by the above phage display approach were highly enriched for cow antibodies with an ultralong CDR3 (i.e., over 40% of candidates feature a CDR3 of at least 50 amino acids).

Exemplary antibodies SA-R2C3 and SA-R2D9 antibodies were derived from Ultra-long scFv library (immunization with parental Wuhan-Hu1 S protein), and identified by a screen involving selection on South African variant Spike protein. Exemplary SKM and SKD antibodies were identified from a screen from a phage library derived directly from CDR3-knob libraries as described.

Sequences alignments for exemplary ultralong antibodies SKD (SEQ ID NO:68), SKM (SEQ ID NO:69), R4C1 (SEQ ID NO:70), R5C1 (SEQ ID NO:71), SR3A3 (SEQ ID NO:72), R2F12 (SEQ ID NO:73), and R2G3 (SEQ ID NO: 74) are shown in FIG. 4 along with a germline reference sequence (SEQ ID NO. 75). The length of the CDR3 and number of cysteine residues are also shown for each.

TABLE E1
Exemplary Candidate SARS CoV-2 Antibodies

| | | VH Sequence | | CDR3 Sequence | |
|---|---|---|---|---|---|
| | | Amino | Nucleic | Amino | Nucleic |
| Name | CDR3 Length | Acid | Acid | Acid | Acid |

Antibody (VH) Candidates

| | | | | | |
|---|---|---|---|---|---|
| RBD A2 | 18 | 49 | 30 | — | — |
| RBD C6 | 18 | 48 | 29 | — | — |
| RBD F4 | 18 | 47 | 28 | — | — |
| R2B1 | 25 | 44 | 25 | — | — |
| R2D6 | 25 | 43 | 24 | — | — |
| R2G1 | 20 | 42 | 23 | — | — |
| R4A10 | 31 | 41 | 22 | — | — |
| R4E5 | 31 | 39 | 20 | — | — |
| R4G3 | — | 38 | 19 | — | — |
| R4G11 | 24 | 37 | 18 | — | — |
| R5A3 | 27 | 36 | 17 | — | — |

Ultralong CDR3 Antibody (VH) Candidates

| | | | | | |
|---|---|---|---|---|---|
| R4C1 | 61 | 40 | 21 | 63 | 55 |
| R2C3 | 61 | 50 | 31 | 66 | 58 |
| SKD | 61 | 46 | 27 | 65 | 57 |
| SKM | 60 | 45 | 26 | 64 | 56 |
| R2G3 | 61 | 33 | 14 | 60 | 52 |
| R2F12 | 58 | 35 | 16 | 62 | 54 |
| SR3A3 | 61 | 34 | 15 | 61 | 53 |
| R2D9 | 52 | 51 | 32 | 67 | 59 |

### Example 4 Assessment of Binding to Spike Protein and RBD

Selected clones, expressed and purified as chimeric bovine-human IgG1 antibodies as described in Example 3, were then assayed for their ability to bind RBD and Spike protein.

### A. SARS CoV-2

RBD and spike binding of chimeric bovine-human IgG1 antibodies was assessed by ELISA. Approximately 50 μL of RBD or Spike protein, at 1 μg/ml in PBS, was added to each well of a half-area Costar ELISA plate (Corning) and coated overnight at 4° C. The plate was blocked with 180 μL/well 2% milk powder/TBS/0.1% Tween20 at room temperature for 2 hours. Purified chimeric bovine-human IgG1 antibodies were diluted 5-fold from 20 nM-0.00129 nM in 2% milk powder/TBS/0.1% Tween20, and 50 μL/well of each dilution was added in duplicate to coated/uncoated wells. The plate was incubated at room temperature for 1 hour, then washed four times with 180 μL of TBS/0.1% Tween20, and bound IgG was detected with 50 μL/well of anti-human Fc-HRP (Jackson ImmunoResearch Laboratories, Inc.) diluted 1:5000 in 2% milk powder/TBS/0.1% Tween20 at room temperature for 30 minutes. The plate was then washed five times with 180 μL of TBS/0.1% Tween20 before 50 μL/well of TMB (3,3′,5,5′-Tetramethylbenzidine) substrate buffer (Thermo Scientific) was added. After 1-2 minutes at room temperature, the reaction was stopped with 50 L/well 1N H₂SO₄, and OD 450 nm values were recorded.

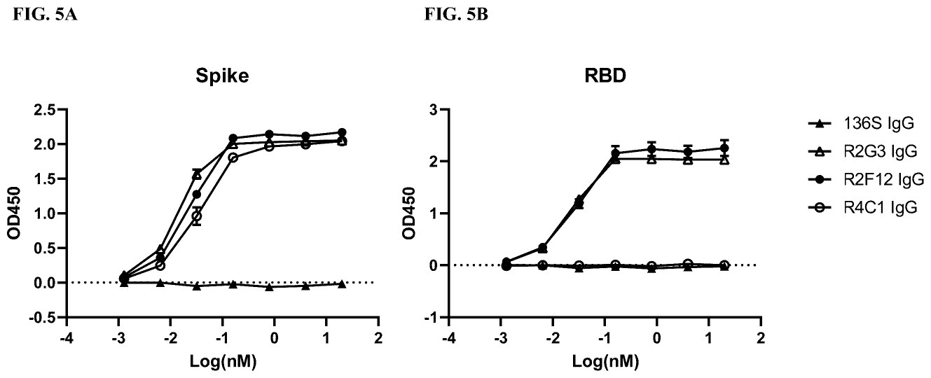

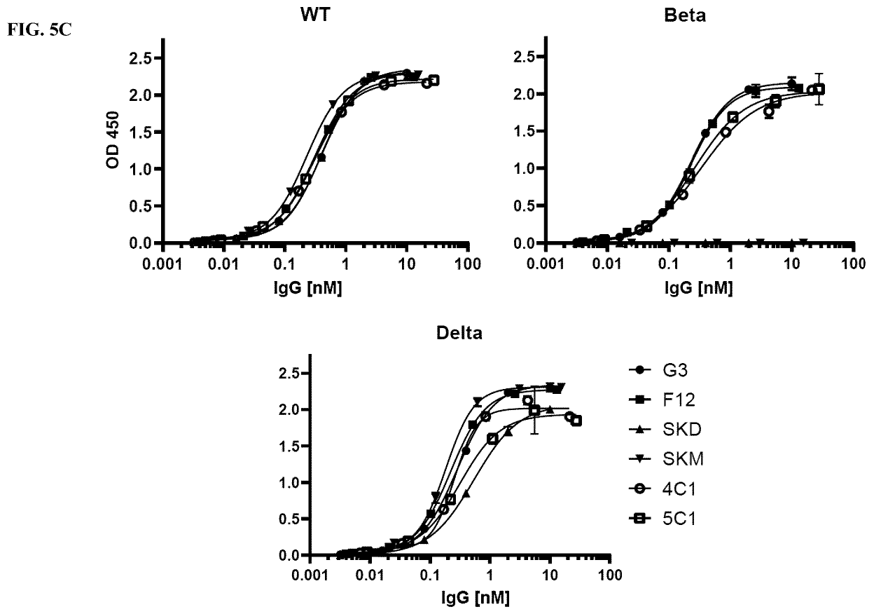

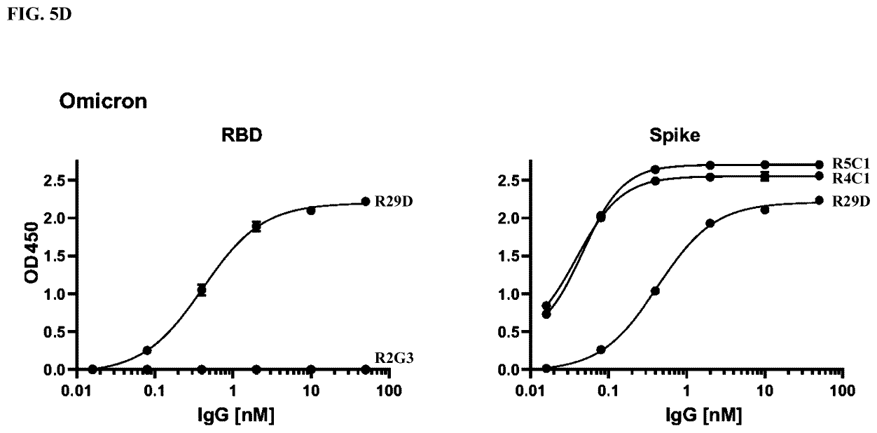

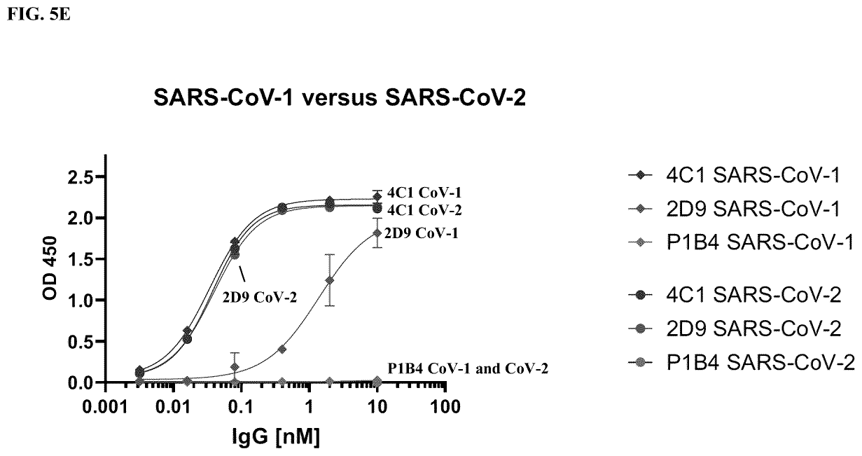

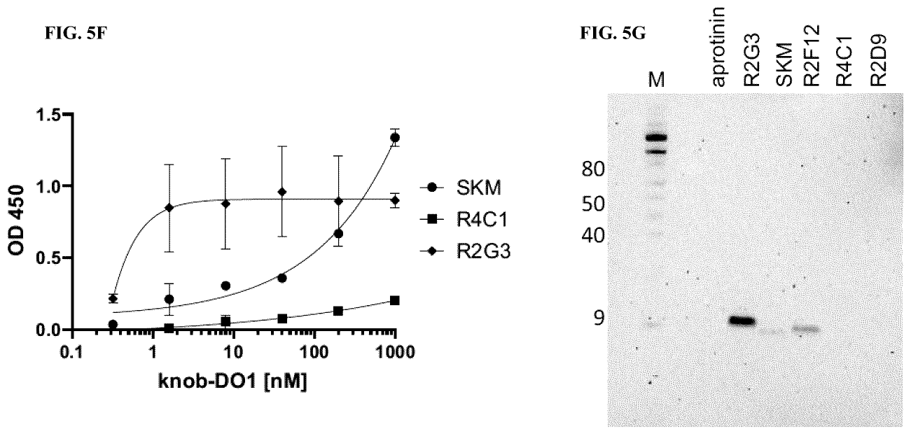

Representative results for three tested clones are shown in FIG. 5A and FIG. 5B. As shown in FIG. 5A, each of the purified chimeric bovine-human IgG1 antibodies (R2G3, R2F12, and R4C1) showed binding to the spike protein. An unrelated bovine-human IgG1 (136S IgG) did not show binding to the spike protein. As shown in FIG. 5B, purified chimeric bovine-human IgG1 antibodies with the V_{H} of clones R2G3 and R2F12 showed binding to the RBD. The unrelated bovine-human IgG1 (136S IgG), as well as the chimeric antibody with the V_{H} of clone R4C1, did not show binding to the RBD protein. These results are consistent with a finding that antibody R4C1 binds to a non-RBD epitope in the Spike protein, whereas R2G3 and R2F12 binding to a RBD epitope.

TABLE E2
Binding Activity of Exemplary Candidate SARS CoV-2 Antibodies

| | | | | |
|---|---|---|---|---|
| | | | | Spike |
| | | RBD | Bind | Protein |
| | Bind | Binding | Spike | Binding |
| Name | RBD | EC50 (nM) | Protein | EC50 (nM) |

Antibody (VH) Candidates

| | | | | |
|---|---|---|---|---|
| RBD A2 | Yes | 0.03 | Yes | 0.024 |
| RBD C6 | Yes | 0.03 | Yes | 0.03 |
| RBD F4 | Yes | 0.03 | Yes | 0.03 |
| R2B1 | Yes | 0.52 | Yes | 0.37 |
| R2D6 | Yes | 0.57 | Yes | 0.41 |

Ultralong CDR3 Antibody (VH) Candidates

| | | | | |
|---|---|---|---|---|
| R4C1 | Yes | — | Yes | 0.20 |
| R2C3 (R5C1) | Yes | — | Yes | 0.39 |
| SKD | Yes | 0.19 | Yes | 0.16 |
| SKM | Yes | 0.24 | Yes | 0.19 |
| R2G3 | Yes | 0.056 | Yes | 0.032 |
| R2F12 | Yes | 0.085 | Yes | 0.050 |
| SR3A3 | Yes | — | Yes | 0.037 |

### B. SARS CoV-2 Variants

RBD and spike binding of chimeric bovine-human IgG1 antibodies was assessed by ELISA against further isolates of SARS CoV-2, including variants from the beta, delta, and omicron lineages as well as a SARS CoV-1 virus. As described in Example 4, approximately 50 μL of RBD or Spike protein, at 1 g/mL in PBS, was added to each well and coated overnight at 4° C. The plate was blocked at room temperature for 2 hours. Purified chimeric bovine-human IgG1 antibodies were diluted 5-fold from 20 nM-0.00129 nM and 50 μL/well of each dilution was added in duplicate to coated/uncoated wells. The plate was incubated at room temperature for 1 hour, then washed four times, and bound IgG was detected with anti-human Fc-HRP (Jackson ImmunoResearch Laboratories, Inc.). The plate was then washed five times before TMB substrate buffer was added. After 1-2 minutes at room temperature, the reaction was stopped with H2SO4, and OD 450 nm values were recorded.

FIG. 5C. shows ELISA binding of IgG antibodies to recombinant stabilized spike proteins derived from the wild-type (WT) Wuhan-Hu-1 strain (left), beta strain (formerly described as the South African strain, middle), or delta strain (right). It was observed that exemplary antibodies SKD and SKM appear to lose detectable binding to beta, but maintain binding to WT and delta SARS CoV-2. The other antibodies are shown to bind across the range of concentrations tested for each S protein.

In a complementary set of experiments performed with RBD, FIG. 5D shows ELISA binding curves of select IgG antibodies against the omicron variant RBD (top left) or recombinant stabilized spike trimer (top right). Of the exemplary RBD binders tested, only R2D9 was observed to maintain binding to an omicron variant spike RBD. R4C1, R5C1 and R2D9 were also observed to bind to full-length omicron spike with EC50s in the subnanomolar range.

FIG. 5E reflects exemplary ELISA data of R4C1 and R2D9 on SARS-CoV-2 compared to SARS-CoV-1. P1B4, also known as NC-Cowl, was used as a negative control, see Sok, et.al. Nature 2017. These data show that R4C1 maintains complete binding activity to SARS-CoV-1, whereas alternative exemplary antibody R2D9 loses >10× binding. However it was observed that R2D9 still maintains some binding activity in the low nanomolar range to SARS-CoV-1.

Finally, FIG. 5F. shows ELISA binding activity (top) for three different exemplary antibody knob candidates against WT (Wuhan) SARS CoV-2 spike protein. For this experiment, each exemplary knob was expressed with a DO1 epitope tag, which was detected with an anti-DO1 antibody reflected on the X axis. FIG. 5G further depicts a modified western blot. Here, the indicated exemplary antibody knobs were heated to 70° C. in the presence of SDS, then resolved by SDS-PAGE before transferred to nitrocellulose membrane and detected with biotinylated RBD. RBD was biotinylated using EZ-Link NHS-LC-LC-biotin (Thermo Fisher). The NHS-LC-LC-biotin was reconstituted in DMF and combined with purified RBD at a 1:5 (RBD: biotin) molar ratio, then incubated at room temperature for 30 minutes. The reaction was then applied to a Pierce polyacrylamide spin desalting column 7K MWCO, equilibrated in PBS. Aprotonin was selected as a similar size control It was observed that the R2G3 knob maintained binding to RBD despite heat and SDS treatment.

### Example 5 Viral Neutralization

In some aspects, binding of an antibody to a viral antigenic protein is insufficient to mitigate cell entry or infectious propagation. Whereas some antibodies, known as neutralizing antibodies, have the ability to inhibit virus in vitro and/or in vivo and are thus considered more relevant for therapeutic applications. Therefore, candidate antibodies as described above were tested for their ability to neutralize infection of cells with a SARS CoV-3 pseudovirus, a model virus to assay neutralization capacity of candidate antibodies. Compared with natural occurring isolates of SARS virus, the pseudovirus can be handled with BSL-2 considerations at high titer and is therefore appropriate for screening, such as in a pseudovirus luciferase assay (PVLA).

A pseudovirus expressing the SARS CoV-2 S protein of the parental Wuhan-Hu-1 Spike protein sequence in its vial envelope was engineered such that the gene for luciferase expression was carried as its cargo. Upon successful penetration into the cell, luciferase is expressed such that the pseudovirus neutralization inhibition rate is inversely proportional to luciferase activity expressed as relative light units (RLUs). These pseudotyped viruses were used in a neutralizing assay performed in CRFK-hACE2 cells. As the receptor for SARS-CoV-2 entry, ACE2 overexpression is considered a mechanism by which cell lines can be produced that display “high infectability”. In the converse, a cell line with minimal or lower ACE2 expression can be considered to display “low infectability”.

Specifically, mock-medium or serially diluted (5-fold) antibody Fab was mixed with the same amount of the pseudotyped virus carrying SARS-CoV-2 wild-type (WT) and incubated at 37° C. for 1 h. Then, the mixtures were transduced into CRFK-hACE or CRFK-hDDP4 cells in the presence of polybrene (Santa Cruz Biotech, Santa Cruz, CA) (10 μg/mL). Following incubation of the transduced cells at 37° C. for 48 h, lysis buffer was added, and the RLU were measured.

A summary of pseudovirus neutralization of identified antibodies is set forth in Table E3. The cow ultralong CDR3 antibodies are highly potent and neutralize variant strains, with a half maximal inhibition at concentrations less than 1-5 ng/mL for some antibodies. In general, the ultralong CDR3 antibodies exhibited more potent neutralization than the antibodies with a standard CDR3 length.

TABLE E3
Pseudovirus Neutralization of Exemplary
Candidate SARS CoV-2 Antibodies

| Name | IC50 (ng/mL) |
|---|---|
| RBD A2 | 69 |
| RBD C6 | 278 |
| RBD F4 | 39.7 |
| R4C1 | 520 |
| R2C3 (R5C1) | 2.12-3.2 |
| SKD | 0.05-0.33 |
| SKM | 0.07-0.29 |
| R2G3 | >1 |
| R2F12 | 0.15-2 |
| SR3A3 | 1.45-160 |

### Example 6 Bacterial Expression and Purification of CDR3-Knob Only Antibodies

A system was developed to express and purify CDR3-knobs, which are small peptide sequences of 25-50 amino acids with 1-6 disulfide bonds derived from an ultralong CDR3 cow antibody as described above. The expression system included fusion with the bacterial chaperone TrxA. CDR3-knobs as well as trxA-CDR-knob fusions were tested for spike and RBD binding.

### A. TrxA-CDR3-Knob Fusion and CDR3-Knob Expression and Purification

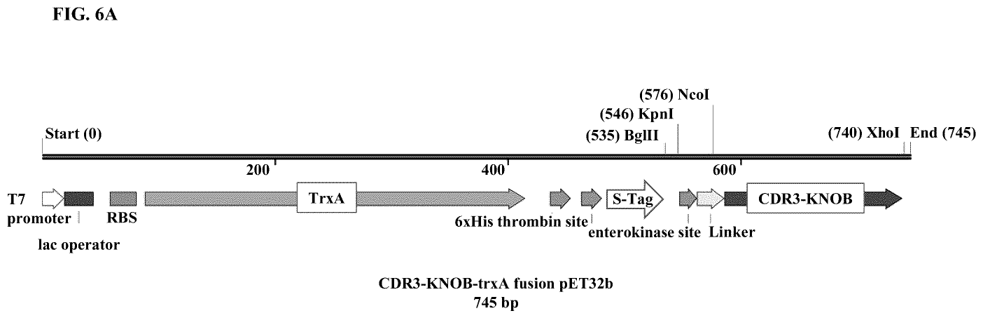

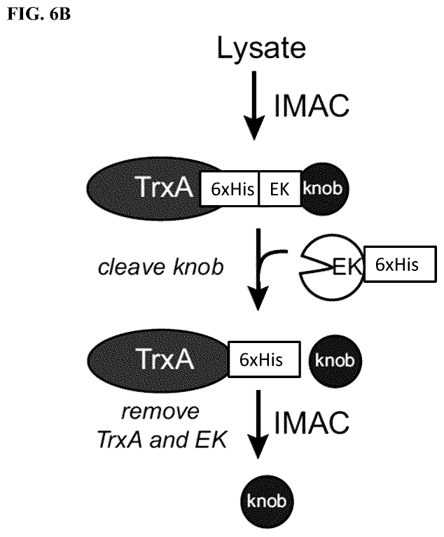

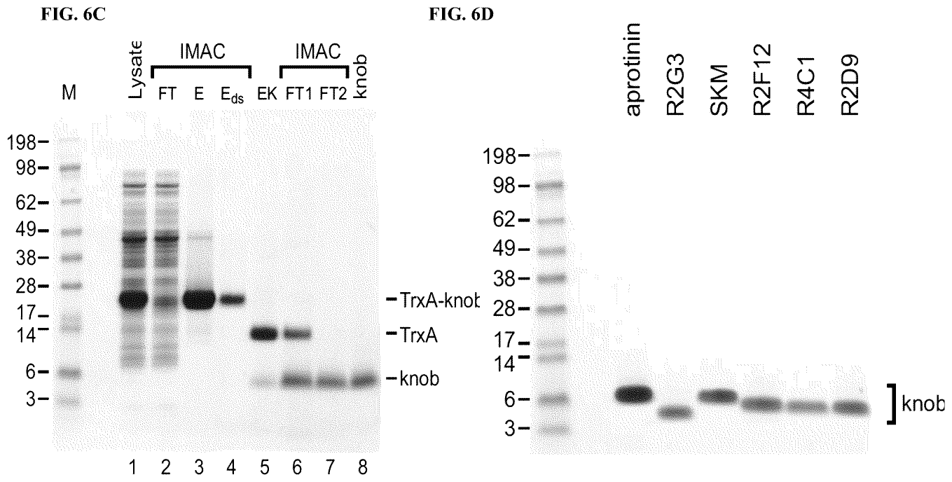

CDR3-knobs from candidate ultralong CDR3 antibodies described in Examples 2-5 were cloned into pET32b vectors (EMD-Millipore) as KpnI-XhoI (or NcoI-XhoI as appropriate) fragments (FIG. 6A), and transformed into Origami 2 DE3 bacteria, and expressed as described below. These CDR3-knobs had sequences set forth in SEQ ID NOs: 060-067, and encoded by a DNA sequence set forth in SEQ ID NOS: 052-060, respectively.

A trxA-CDR3-knob fusion clone was grown overnight at 37° C. in 20 mL of 2×TY/50 μg/mL carbenicillin/10 μg/mL tetracycline/2% glucose, transferred to 200 mL of the same medium, and grown at 37° C. to an OD600 nm of approximately 1.0, after which the bacteria were spun down and resuspended in 200 mL of 2×TY/50 μg/ml carbenicillin/0.5 mM IPTG and grown overnight at 22° C. The bacteria were again pelleted, resuspended in 10 mL of Bugbuster HT (EMD-Millipore), rotated for 30 minutes at room temperature, and debris pelleted for 20 minutes at 14,000 g at 4° C. The supernatant was added to an equilibrated Talon resin column (1 mL resin TaKaRa), rotated at 4° C. for 2 hours, washed with five column volumes wash buffer (5 mM imidazole), then 1 column volume wash buffer (10 mM imidazole), eluted with 2.5 mL of 300 mM imidazole elution buffer, and then buffer exchanged to PBS/saline with a PD10 spin column (GE Healthcare). The trxA-CDR3-knob was adjusted to 50 mM Tris pH 7.4, 150 mM NaCl, and 2.5 mM CaCl₂) (1× enterokinase (EK) reaction buffer), and 400u recombinant his-tagged Enterokinase (Genscript) was added and incubated overnight at room temperature. Digested trxA and enterokinase were removed by incubation on a fresh equilibrated Talon resin column (1.2 mL resin) for 2 hours at 4° C., and purified CDR-knob was collected in the flowthrough. Again, the sample was buffer exchanged to saline/PBS. In some cases, endotoxin removal may be carried out by anion exchange chromatography prior to use or testing, such as testing in a viral neutralization assay. CDR3-knobs cloned and expressed in E. coli as independent domains are set forth in SEQ ID NOS: 060-067.

The stepwise purification is depicted in FIG. 6B. As shown in FIG. 6C, stepwise purification, as monitored by SDS-PAGE, efficiently purified both trxA-CDR3-knob fusion proteins as well as soluble CDR3-knobs from E. coli lysates. FIG. 6D. depicts an exemplary SDS-PAGE gel of several purified ultralong CDR H3 knob peptides. The samples were treated with reducing agent DTT, which in some aspects is sufficient to break disulfide bonds. The similarly sized protein aprotinin was included as a size control.

IMAC-Purified trxA-CDR3-Knob Fusion Spike or RBD Binding

In order to assess CDR3-knob binding as trxA fusions, prior to enterokinase cleavage from trxA, half-area Costar ELISA plates were coated overnight at 4° C. with serial dilutions of IMAC purified trxA-knob fusions from 25 μL of trxA fusion in 50 μl/well PBS. RBD-binding clones R2G3, R2F12, SKM, and SKD (nucleic acid sequences set forth in SEQ ID NO: 052, SEQ ID NO: 054, SEQ ID NO: 056, and SEQ ID NO: 057, respectively; and amino acid sequences set forth in SEQ ID NO: 060, SEQ ID NO: 062, SEQ ID NO: 064, and SEQ ID NO: 065, respectively), and spike-binding clone R4C1 (nucleic acid sequence set forth in SEQ ID NO: 055, and amino acid sequence set forth in SEQ ID NO: 063), were tested.

Plates were then blocked for 1 hour at room temperature with 100 μL/well of 2% milk powder/PBS, and then washed twice with 100 μL/well of PBS. Approximately 50 μL/well of 1 μg/mL Wuhan-Hu-1 spike protein in 2% milk powder/PBS was incubated for 1 hour, and wells were then washed three times with 100 μl/well of PBS. To detect bound spike protein, 1 μg/mL of full length IgG chimeric ultralong CDR3 was added, either anti-RBD R2G3 IgG1 (for R4C1), or anti-R4C1 IgG1 antibody (for R2F12, R2G3, SKD and SKM fusions), in 2% milk powder/PBS, incubated for 1 hour, and then wells were washed three times with 100 μL/well of PBS. Bound IgG was then detected by incubation with 1:5000 diluted anti-human IgG-Fc-HRP conjugate in 2% milk powder/PBS for 1 hour, and wells were then washed three times with 100 μL/well of PBS. The plate was then washed and developed for 5-10 minutes at room temperature with 50 μL/well TMB (3,3′,5,5′-Tetramethylbenzidine) substrate buffer (Thermofisher). The reaction was stopped with 100 μL/well of 0.5N H₂SO₄ and read at 450 nm.

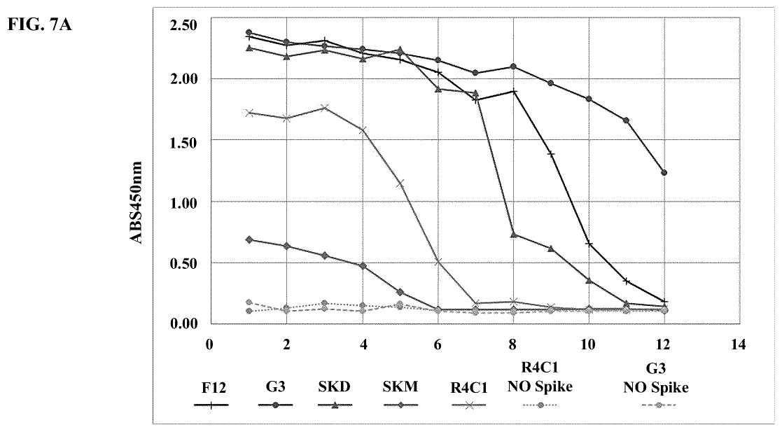

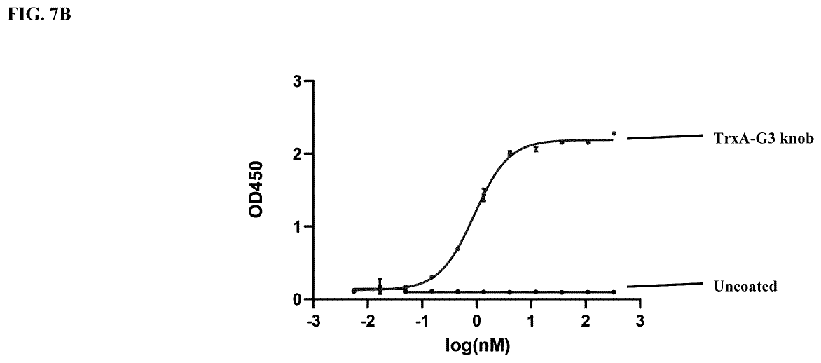

As shown in FIG. 7A (in which R2F12 is denoted as “F12”, and R2G3 is denoted as “G3”), the tested trxA-knob fusion proteins showed spike protein binding. Control conditions in which fusion proteins R3C1 and R2G3 were incubated in the absence of spike protein (denoted “R3C1 NO Spike” and “G3 NO Spike”) did not show binding. Binding for the TrxA-R2G3 fusion protein is also shown separately in FIG. 7B, relative to uncoated plates.

### B. Purified R2G3 CDR3 Knob Binding to Wuhan-Hu-1 RBD

Binding of purified R2G3 CDR3-knob (after enterokinase cleavage from trxA as described above) to RBD was evaluated by ELISA. The nucleic acid sequence encoding R2G3 CDR3-knob is set forth in SEQ ID NO: 052, and the amino acid sequence set forth in SEQ ID NO: 060.

Wells in a half-area Costar ELISA plate (Corning) were coated, in duplicate, with 50 μL/well of purified CDR3-knob diluted 2-fold from 84-0.082031 nM in PBS. The plate was incubated at 37° C. for 1 hour, then blocked with 180 μL/well of 2% milk powder/TBS, 0.1% Tween20 at room temperature for 2 hours. Next, biotinylated RBD was diluted to 0.5 ng/μL in 2% milk/TBS/0.1% Tween20, and 50 μL/well was added to coated/uncoated wells. After 1 hour at room temperature, wells were washed four times with 180 μL/well of TBS/0.1% Tween20, and bound biotinylated RBD was detected with 50 μL/well of streptavidin-HRP (Invitrogen) diluted 1:5000 in 2% milk/TBS/0.1% Tween20 for 30 minutes at room temperature. The wells were then washed five times with 180 μL/well TBS/0.1% Tween20 before addition of 50 μL/well TMB (3,3′,5,5′-Tetramethylbenzidine) substrate buffer (Thermo Scientific). After 1-2 minutes at room temperature, the reaction was stopped with 50 μL/well 1N H₂SO₄, and OD 450 nm values were recorded. The average OD450 of uncoated wells was subtracted from the OD450 in each coated well. Background-subtracted OD450 values were plotted in GraphPad Prism (GraphPad Software LLC) against Log(CDR3-knob nM).

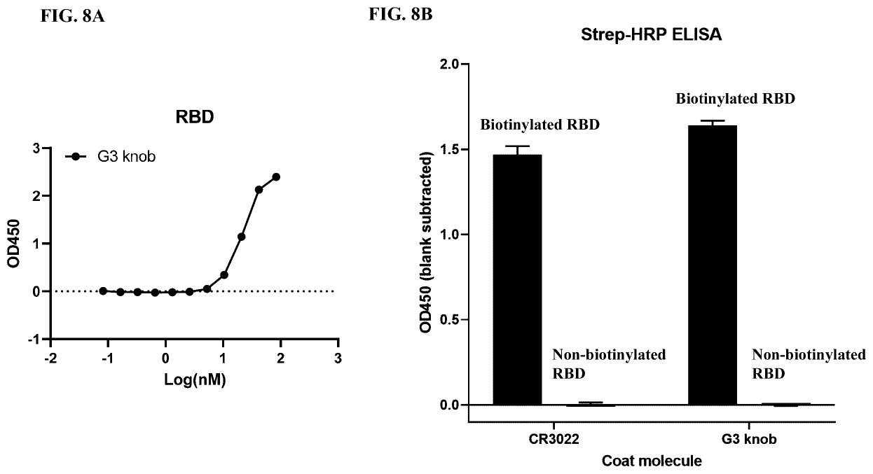

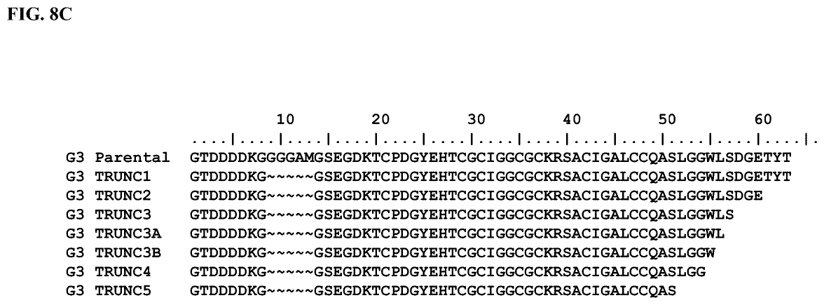

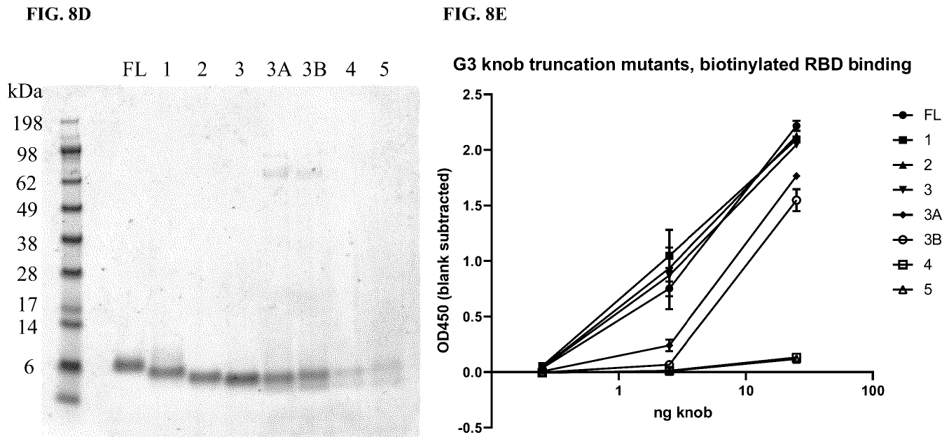

As shown in FIG. 8A, the soluble R2G3 knob showed binding to the RBD. As shown in FIG. 8B, soluble R2G3 knob binding was increased relative to that of a reference anti-spike protein antibody, CR3022.

### C. Binding of Truncated R2G3 CDR3 Knobs to Wuhan-Hu-1 RBD

Truncated R2G3 CDR3-knobs were cloned and produced as described above using pET32b vectors encoding an R2G3 truncated mutant followed by an enterokinase cleavage site. Amino acid sequences of the truncated R2G3 mutants are shown in FIG. 8C. As shown in FIG. 8D, Truncations 1-3 showed compact bands following enterokinase cleavage and gel electrophoresis (0.75 μg of truncated knob protein per lane, 250 mM DTT).

The truncated R2G3 CDR3-knobs were also tested for RBD binding as described above. As shown in FIG. 8E, Truncations 1-3 had preserved RBD binding ability, whereas Truncations 4 and 5 lacked RBD binding.

### D. Defining the Minimal CDR3-Knob C-Terminal Requirement

In order to define the C-terminal requirements (i.e., C-terminal minimal sequence) of a prototypical CDR3-knob, a series of R2G3 truncations were cloned into pET32b and expressed and purified as described in Example 6 above. These truncations were as set forth in Table E4 below.

TABLE E4
Exemplary R2G3 Truncations

| CLONE | Mature amino acid sequence after Enterokinase cleavage |
|---|---|
| G3 Parental | GGGGAMGSEGDKTCPDGYEHTCGCIGGCGCKRSACIGALCCQASLGGWLSDGETYT |
| G3 TRUNC1 | ~~~~~GGSEGDKTCPDGYEHTCGCIGGCGCKRSACIGALCCQASLGGWLSDGETYT |
| G3 TRUNC2 | ~~~~~GGSEGDKTCPDGYEHTCGCIGGCGCKRSACIGALCCQASLGGWLSDGE |
| G3 TRUNC3 | ~~~~~GGSEGDKTCPDGYEHTCGCIGGCGCKRSACIGALCCQASLGGWLS |
| G3 TRUNC3A | ~~~~~GGSEGDKTCPDGYEHTCGCIGGCGCKRSACIGALCCQASLGGWL |
| G3 TRUNC3B | ~~~~~GGSEGDKTCPDGYEHTCGCIGGCGCKRSACIGALCCQASLGGW |
| G3 TRUNC4 | ~~~~~GGSEGDKTCPDGYEHTCGCIGGCGCKRSACIGALCCQASLGG |
| G3 TRUNC5 | ~~~~~GGSEGDKTCPDGYEHTCGCIGGCGCKRSACIGALCCQAS |

The quality of expressed material was assessed by SDS-PAGE and RBD ELISA as described in Example 6D above. Only Truncations 4 (G3 TRUNC4) and 5 (G3 TRUNC5) were observed to exhibit no RBD binding capability. Truncations 3A (G3 TRUNC3A) and 3B (G3 TRUNC3B) demonstrated reduced binding in an ELISA and increased band diffuseness in SDS-PAGE as depicted in FIG. 8A. ELISAs performed with truncations 1-3 yielded no observed loss in binding activity relative to parental R2G3 CDR3-knob as shown in FIG. 8B. These data support that a minimum of at least 9 amino acids is required after the last non-canonical Cys residue for R2G3 binding.

### E. CDR3 Knob Purification by Size Exclusion Chromatography

Size exclusion chromatography (SEC) was used to resolve if soluble CDR3-knobs that were purified following bacterial expression were present in multiple forms. Soluble R4C1 and R2G3 knobs were produced as described above and subjected to SEC.

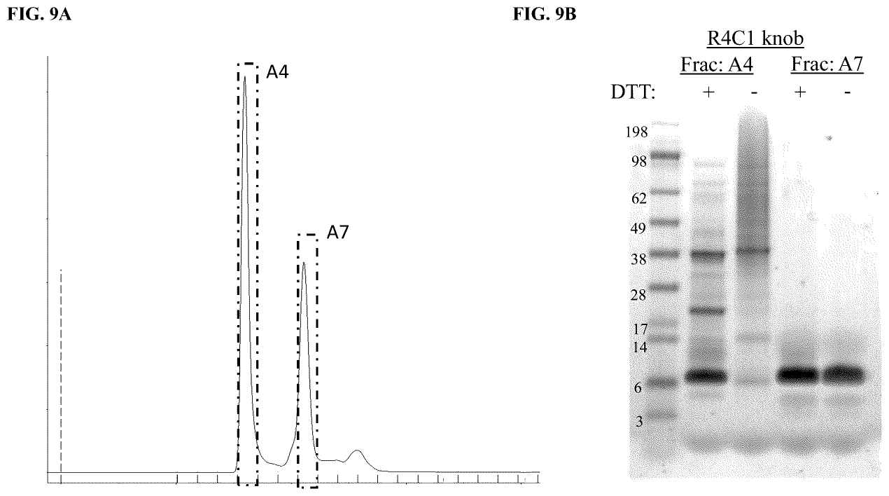

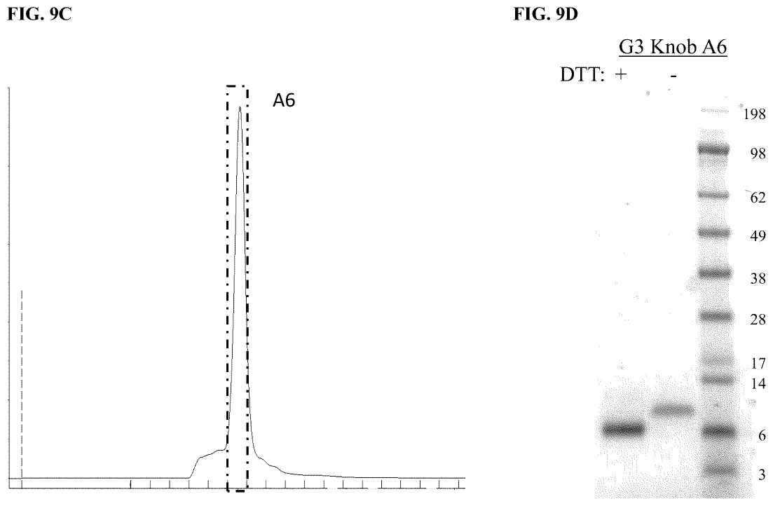

As shown in FIG. 9A, SEC revealed at least two distinct elution fractions (fractions A4 and A7) for purified R4C1 knobs, indicating that purified R4C1 knobs were present in multiple forms following bacterial expression. Gel electrophoresis was performed on fractions A4 and A7. As shown in FIG. 9B, fraction A4 contained a larger soluble aggregate as well as smaller, active soluble CDR3-knobs. Fraction A7 contained only the smaller, active soluble CDR3-knobs.

As shown in FIG. 9C, SEC revealed only one distinct elution fraction (fraction A6) for purified R2G3 knobs (fraction A6). This result was corroborated by gel electrophoresis performed on fraction A6 (FIG. 9D).

### Example 7 Comparison of SARS-CoV 2 Virus Neutralization of Chimeric Fab Ultralone CDR3 and CDR3 Knob

To assess virus neutralization of a CDR3-knob only antibody, assays to assess neutralization of pseudovirus or live WT SARS-CoV2 virus were carried out. In this example, purified R2G3 CDR3-knob (“G3-Knob”) or a Fab of the chimeric R2G3 ultralong CDR3 antibody (“G3-Fab”), or a full length TgG chimeric R2G3 ultralong CDR3 antibody (“G3”) were tested, as indicated.

A pseudovirus luciferase assay (PLSA) substantially as described in Example 5 was performed. Virus neutralization was assessed against pseudotyped virus carrying SARS-CoV-2 (Wuhan-Hu-1) wild-type (WT) spike protein, or the S variants (E484K/N507Y; B.1.1.7 or “UK” variant; and K417N/E484K/N501Y; B.1.351 or “SA” variant). Mock-medium or serially diluted (5-fold) antibody G3-Knob, G3-Fab or G3 was mixed with the same amount of the pseudotyped virus carrying SARS-CoV-2 wild-type (WT), the S variants (484K, B.1.1.7 and B.1.351) and incubated at 37° C. for 1 h. Then, the mixtures were transduced into CRFK-hACE or CRFK-hDDP4 cells in the presence of polybrene (Santa Cruz Biotech, Santa Cruz, CA) (10 μg/mL). As the receptor for SARS-CoV-2 entry, ACE2 overexpression is considered a mechanism by which cell lines can be produced that display “high infectability”. In the converse, a cell line with minimal or lower ACE2 expression can be considered to display “low infectability”.

Following incubation of the transduced cells at 37° C. for 48 h, lysis buffer was added, and the RLU were measured. Inhibition curves of serial dilutions of each antibody, G3-Fab or G3-Knob, against mock treatment were generated, and the 50% effective concentration (EC50) values were determined by GraphPad Prism software using a variable slope (GraphPad, La Jolla, CA). The results are summarized in Table E5.

To assess neutralizing activity against live SARS-CoV-2, selected antibodies of G3, G3-Fab or G3-Knob were investigated for their neutralizing activity against the replication of SARS-CoV-2 or B.1.17 or B.1.351 variants in Vero E6 cells. Briefly, 50-100 plaque forming units of SARS-CoV-2 hCoV/USA-WA1/2020 (wild type), SARS-CoV-2 hCoV-19/England/204820464/2020 (B.1.1.7 variant), or SARS-CoV-2 hCoV-19/South Africa/KRISP-EC-K005321/2020 (B.1.351 variants) were mixed with mock-medium or serially diluted (5-fold) G3-Fab or G3-Knob. Following incubation at 37° C. for 1 h, the mixtures were inoculated to confluent Vero E6 cells in 24 well plates. After 2 hr incubation, medium containing agar (1% final concentration) and neutral red was added to the cells. After 48-72 hr, plaques in each well were counted. The EC50 values were determined as described above and shown in Table E5 below.

Together, the results shown in Table E5 demonstrate that the exemplary cow ultralong CDR3 R2G3, in either a standard IgG Fab format or as a CDR3-knob only format, exhibited potent neutralizing activity against WT SARS-CoV-2 as well as the tested variants. The cow ultralong CDR3 antibody is highly potent and neutralizes variant strains, with a half maximal inhibition at concentrations less than 1-5 ng/mL, depending on the antibody format. Remarkably, despite being a short sequence of only 51 amino acids in length, the CDR3-knob only antibody retained subnanomolar potency. Due to the small size of the CDR3-knob antibodies, this examples supports utility of the CDR3-knob antibodies as novel therapeutic antibody candidates for an inhalation formulation for respiratory targets, including other viruses, bacteria, other infectious diseases, asthma or lung cancer.

TABLE E5
Neutralization pseudovirus and live virus for R2-G3 IgG, Fab and CDR3-knob

| | Pseudotype EC50 | | Pseudotype EC50 | | SARS-CoV-2 VeroE6 EC50 | | | SARS-CoV-2 VeroE6 EC50 | | |
|---|---|---|---|---|---|---|---|---|---|---|
| | (ng/mL) | | (pM) | | (ng/mL) | | | (pM) | | |
| | | SA | | SA | | UK | SA | | UK | SA |
| | WT | variant | WT | variant | WT | variant | variant | WT | variant | variant |
| G3 | 0.59 | 5.28 | 3.55 | 32.2 | 0.5 | 0.51 | 2.6 | 3.15 | 3.2 | 19.14 |
| G3 Fab | 1.23 | 1.19 | 22.8 | 22.39 | 3.67 | 1.35 | 5.03 | 72.58 | 26.19 | 103.31 |
| G3 Knob | 5.49 | 52.23 | 904.5 | 8830.1 | 0.92 | 1.94 | 333.28 | 712 | 535 | 49,968 |

### A. SARS CoV-2 Variants

In a further assessment of virus neutralization of ultralong CDR3 antibodies, assays to assess neutralization of live WT SARS-CoV2 virus or several variant SARS CoV-2 viruses were carried out. In this example, full length IgG chimeric ultralong CDR3 antibodies F12, G3, SKD, and SKM were tested, as indicated.

A pseudovirus luciferase assay (PLSA) substantially as described in Example 5 was performed. Virus neutralization was assessed against pseudotyped virus carrying SARS-CoV-2 (Wuhan-Hu-1) wild-type (WT) spike protein, the S variants (E484K/N507Y; B.1.1.7 or “UK” variant; and K417N/E484K/N501Y; B.1.351 or “SA” variant) or 484K. Mock-medium or serially diluted (5-fold) antibody was mixed with the same amount of the pseudotyped virus carrying SARS-CoV-2 wild-type (WT), the S variants (484K, B.1.1.7 and B.1.351) and incubated at 37° C. for 1 h. Then, the mixtures were transduced into Vero, CRFK-hACE or CRFK-hDDP4 cells in the presence of polybrene (Santa Cruz Biotech, Santa Cruz, CA) (10 μg/mL). As the receptor for SARS-CoV-2 entry, ACE2 overexpression is considered a mechanism by which cell lines can be produced that display “high infectability”. In the converse, a cell line with minimal or lower ACE2 expression can be considered to display “low infectability”.

Following incubation of the transduced cells at 37° C. for 48 h, lysis buffer was added, and the RLU were measured. As shown in FIGS. 10A-10D, each exemplary ultralong CDR3 antibody exhibited activity against more than one variant SARS CoV-2 S protein. Inhibition curves of serial dilutions of each antibody against mock treatment were generated, and the 50% effective concentration (EC50) values were determined by GraphPad Prism software using a variable slope (GraphPad, La Jolla, CA). The results are summarized in Table E6.

TABLE E6
Neutralization pseudovirus and live virus
for Exemplary Ultralong CDR3 Antibodies

EC₅₀ (pM)

| | WT | UK | 484K | SA |
|---|---|---|---|---|
| F12 | 6.15 | 4.64 | 24.19 | 200.85 |
| G3 | 4.03 | 3.79 | 10.60 | 80.235 |
| SKD | 4.47 | 7.71 | >1000 | >1000 |
| SKM | 5.77 | 9.74 | >1000 | >1000 |

Together, the results shown in Table E5 demonstrate that the exemplary cow ultralong CDR3 antibodies, F12, G3, SKD, and SKM, exhibited potent neutralizing activity against WT SARS-CoV-2 as well as the tested variants. The cow ultralong CDR3 antibody is highly potent and neutralizes variant strains, with a half maximal inhibition at concentrations less than 1-5 ng/mL, depending on the antibody format. Remarkably, despite being a short sequence of only 51 amino acids in length, the CDR3-knob only antibody retained subnanomolar potency. Due to the small size of the CDR3-knob antibodies, this examples supports utility of the CDR3-knob antibodies as novel therapeutic antibody candidates for an inhalation formulation for respiratory targets, including other viruses, bacteria, other infectious diseases, asthma or lung cancer.

### Example 8 SARS CoV-1 Cross Reactivity

To assess possible cross reactivity and broad neutralization of exemplary Ultralong CDR3 antibodies, assays to assess neutralization of pseudovirus were carried out. In this example, exemplary R4C1 and R2D9 ultralong CDR3 antibodies were tested, as indicated.

A pseudovirus luciferase assay (PLSA) substantially as described in Example 5 was performed. Virus neutralization was assessed against pseudotyped virus carrying SARS-CoV-2 (Wuhan-Hu-1) wild-type (WT) spike protein, the S protein of a SARS-CoV-1 virus, or a VSV-G control. Mock-medium or serially diluted (5-fold) antibody G3-Knob, G3-Fab or G3 was mixed with the same amount of the pseudotyped virus carrying SARS-CoV-2 wild-type (WT), SARS-CoV-1 wild-type, or VSV-G, and incubated at 37° C. for 1 h. Then, the mixtures were transduced into cells in the presence of polybrene (Santa Cruz Biotech, Santa Cruz, CA) (10 μg/mL).

Following incubation of the transduced cells at 37° C. for 48 h, lysis buffer was added, and percent neutralization were measured. Inhibition curves of serial dilutions of each antibody against mock treatment were generated, and the maximum percent neutralization (MPN), i.e. the percent at which the neutralization curve plateaus for those viruses neutralized, were determined by GraphPad Prism software using a variable slope (GraphPad, La Jolla, CA).

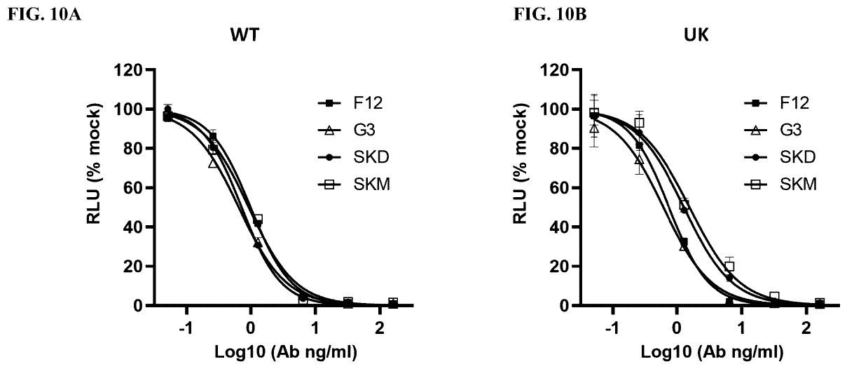

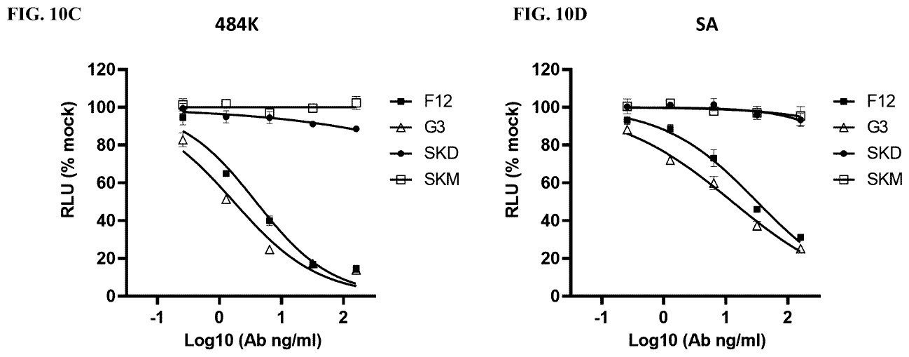

FIG. 11A. shows the IC50 values of different IgG antibodies against pseudoviruses from various coronavirus strains. Note that R4C1 and R2D9 maintain activity against the omicron variant of SARS-CoV-2. All of the antibodies exhibit subnanomolar potency, with several in the low picomolar range.

### Example 9 Neutralization of Live Variant Virus

To assess additional cross reactivity and potential broad neutralization of exemplary antibodies, assays to assess neutralization of pseudovirus in addition to live virus were carried out. In this example, exemplary SKM, SKD, R4C1 (IgG, Fab, and Knob), G3 (IgG, Fab, and Knob) and R2D9 (IgG and knob) as described above were tested as indicated.

A pseudovirus luciferase assay (PLSA) substantially as described in Example 5 was performed. Virus neutralization was assessed against pseudotyped virus carrying SARS-CoV-2 (Wuhan-Hu-1) wild-type (WT) spike protein, the S protein of a SARS-CoV-2 beta lineage virus, or a SARS-CoV-2 delta lineage virus. Mock-medium or serially diluted (5-fold) antibody, knob, or fab was mixed with the same amount of the pseudotyped virus carrying SARS-CoV-2 spike protein, and incubated at 37° C. for 1 h. Then, the mixtures were transduced into cells in the presence of polybrene (Santa Cruz Biotech, Santa Cruz, CA) (10 μg/mL). Following incubation of the transduced cells at 37° C. for 48 h, lysis buffer was added, and the RLU were measured.

Neutralization was also assayed using live virus in BSL-3 conditions. Similarly as described above, serially diluted (5-fold) antibody, knob, or fab was mixed with the same amount of wildtype SARS-CoV-2 virus (Wuhan-Hu-1), or either of an alpha (United Kingdom) or beta (South Africa) lineage variant, and incubated at 37° C. for 1 h. The cells were washed, and then plaque forming units (PFU) measured following incubation of the cells at 37° C. for 48 h.

In experiments with pseudo- or live virus, percent neutralization were measured. Inhibition curves of serial dilutions of each antibody against mock treatment were generated, and the maximum percent neutralization (MPN), i.e. the percent at which the neutralization curve plateaus for those viruses neutralized, were determined by GraphPad Prism software using a variable slope (GraphPad, La Jolla, CA). For example, results for exemplary antibody candidate R2G3 (IgG, Fab, and Knob) are shown in FIG. 11B The results are summarized in Table E7 in ng/mL, with standard deviations of three independent replicates to the right.

TABLE E7
Neutralization of Pseudovirus and live virus by ultralong CDR
H3 IgG, Fabs, and knobs against different SARS-CoV-2 strains

| | Pseudovirus | | | Live SARS CoV-2 | | |
|---|---|---|---|---|---|---|
| | WT | Beta | Delta | WT | Alpha | Beta |
| SKM (IgG) | 0.48 ± 0.02 | >1000 | 9.05 ± 1.20 | 3.07 ± 1.57 | | |
| SKD (IgG) | 0.41 ± 0.01 | >1000 | 9.85 ± 1.90 | 4.87 ± 3.65 | | |
| R4C1 (IgG) | 99.93 ± 31.08 | 375.15 ± 71.63 | 109.8 ± 43.3 | | | |
| R4C1 (Fab) | 184.25 ± 37.12 | 377.95 ± 120.1 | 244.5 ± 67.74 | | | |
| R4C1 (knob) | 641.95 ± 84.22 | 1024.9 ± 297.1 | 401.3 ± 97.30 | | | |
| G3 (IgG) | 0.20 ± 0.03 | 5.70 ± 0.14 | 0.56 ± 0.09 | 0.50 ± 0.05 | 0.51 ± 0.04 | 3.08 ± 1.73 |
| G3 (Fab) | 1.34 ± 0.20 | 2.20 ± 1.01 | 81.82 ± 49.9 | 3.77 ± 0.09 | 1.35 ± 0.07 | 5.34 ± 1.94 |
| G3 (knob) | 5.49 ± 1.29 | 66.2 ± 11.03 | 301.61 ± 157.19 | 0.96 ± 0.28 | 2.48 ± 1.64 | 374.07 ± 174.92 |
| R2D9 (IgG) | 0.26 ± 0.12 | 0.33 ± 0.09 | 0.32 ± 0.10 | | | |
| R2D9 (knob) | 24.18 ± 4.95 | 22.62 ± 6.80 | 66.76 ± 10.55 | | | |
| SKM (IgG) | 0.48 ± 0.02 | >1000 | 9.05 ± 1.20 | 3.07 ± 1.57 | | |

### Example 10 Bi- and Multispecific Antibodies with Ultralong CDR3s

Knobs derived from bovine ultralong CDRH3 antibodies are expressed as fusion proteins or as part of dimeric or multimeric molecules, creating bivalent, bispecific, multivalent, or multispecific proteins (FIG. 12). Two or more knobs are expressed as a fusion protein, for example with a flexible linker (e.g., Gly-Gly-Gly-Ser, or the like) between the C-terminus of one knob and the N-terminus of another knob. Additionally, bispecific molecules are made wherein one knob is in its wild-type conformation as a bovine, or humanized bovine, VH region and expressed with a light chain as an IgG, while a second knob is fused to the C-terminus of the heavy chain constant region. In this situation, the two VH regions are identical and have the specificity of knob 1, but the C-terminus has a new specificity as determined by knob 2.

In another approach, ‘knobs into holes’ technology is employed where two heavy chains are co-expressed where one heavy chain contains a VH region with one knob (knob 1) within its CDRH3 and a second heavy chain has a VH region with a second knob within its CDRH3 (knob 2). The two heavy chains also differ by having constant region mutations such that only the heterologous heavy chains effectively pair with one another to form a dimer. In this case, the homodimers are not formed to an appreciable extent. Such ‘knobs-into-holes’ mutations include T22Y (on one chain) and Y86T (on the other chain) in the CH3 domain of Fc.

DNA vectors encoding such molecules are generated by standard molecular biology techniques and expressed and purified as described above in previous Examples. Additionally, individual knobs are chemically covalently linked together using small molecule linkers, or polyethylene glycol (PEG) linkers, including heterobifunctional or heteromultifunctional linkers (e.g., Pierce). In this case, individual knobs are expressed and purified and then added together in the presence of linker and the appropriate reaction conditions to covalently couple the linkers to the knob proteins. Amine, carboxyl, maleimide, NHS ester, and hydrazide chemistries are commonly used in these cross-linking approaches. Furthermore, the knobs are used in the context of a nanoparticle to provide specificity or activity to the nanoparticle. In this regard, the nanoparticle can be a protein-based nanoparticle, including particles formed from viral proteins, albumin nanoparticles, and the like. The nanoparticles can also be derived from non-protein molecules including lipids (e.g., lipoparticles), carbohydrates, etc.

### Example 11 Bioinformatic Identification of Bovine Ultralong CDR H3 Knob Domain Ends

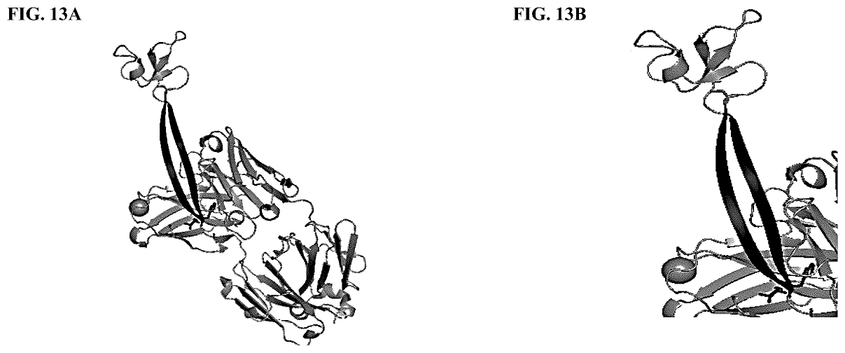

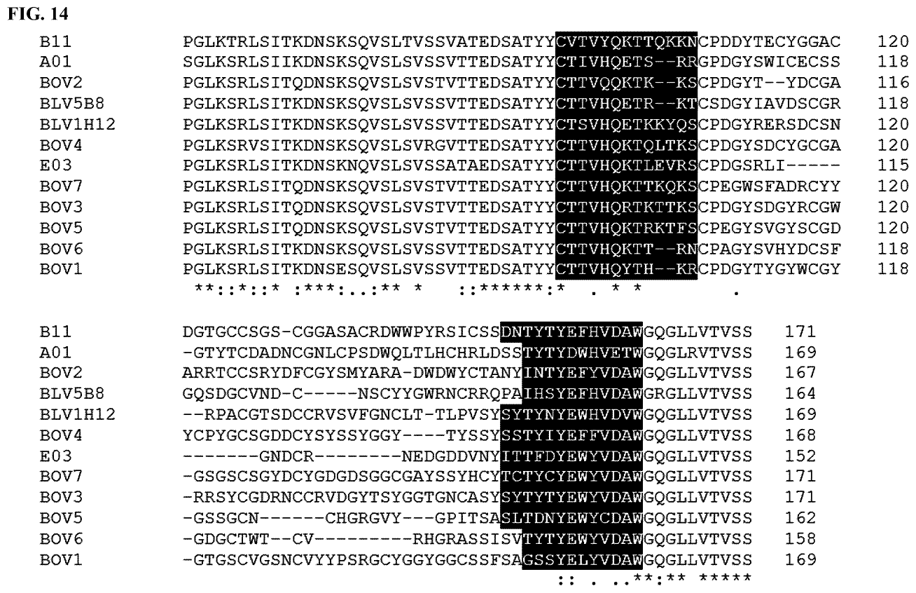

An algorithm was developed to identify bovine ultralong CDR H3 knob domain boundaries by amino acid sequence. By sequence, the bovine ultralong CDR H3 region ranges from “the third residue following the conserved cysteine in framework 3 to the residue immediately preceding the conserved tryptophan in framework 4” (Wang et al. Cell 2013, 153(6).1379-1393). Structurally, the knob domain is defined as the small disulfide-rich domain located upon the distal end of the anti-parallel β-ribbon stalk domain (FIGS. 13A and 13B).

Crystal structures of exemplary bovine ultralong antibodies (Table E8) were analyzed in conjunction with sequences (FIG. 14) to formulate a precise definition of the knob boundaries by both sequence and structure. In the analysis, the first residue of the knob domain was defined as the first conserved DH cysteine, or other residue at this position in rare exceptions such as A01, preceding the conserved “PDG” motif. For the purpose of locating the final knob domain residue, the stalk domain was then also defined. By crystal structure analysis, symmetry was observed in the length of the ascending and descending stalk 1-ribbon strands. The conserved framework 3 cysteine, preceding the first CDR H3 residue (Wang et al., 2013) by 3 amino acid positions, is located proximal to the base of the ascending stalk strand and is situated directly across from the conserved framework 4 tryptophan which is one residue downstream of the final CDR H3 residue (Wang et al., 2013). In the analysis, the first ascending stalk residue was defined as the conserved framework 3 cysteine and the final descending stalk residue was defined as the conserved framework 4 tryptophan. The C-terminal knob boundary position was located by subtracting the number of ascending stalk residues from the framework 4 tryptophan position (Table E8).

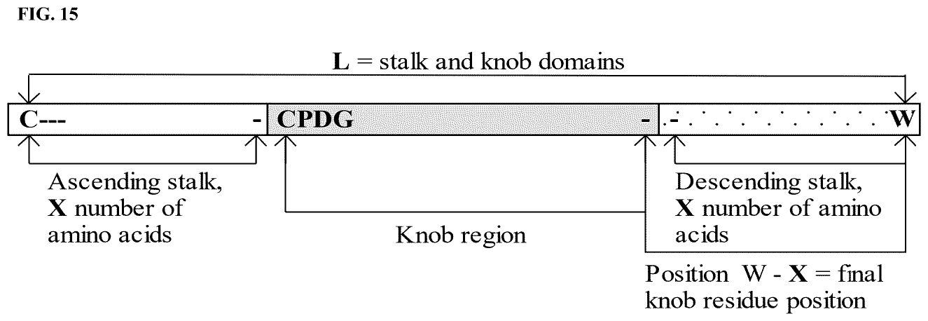

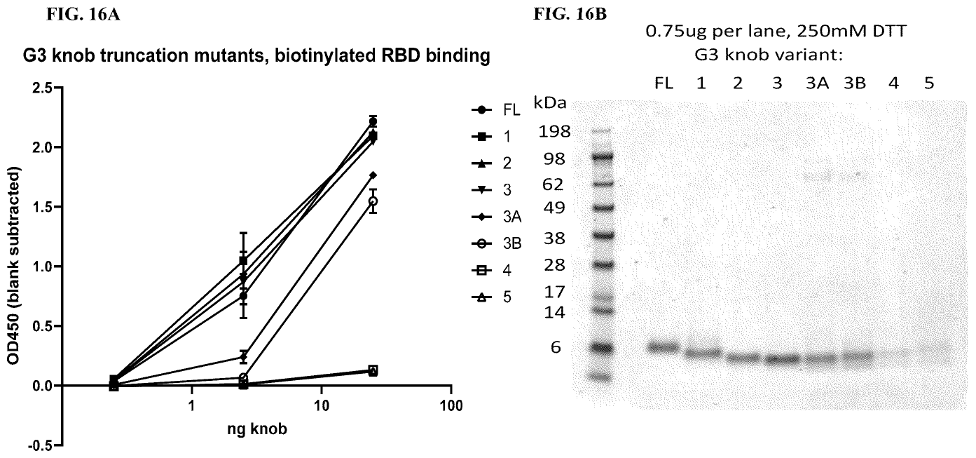

In summary, our algorithm (below) defines the knob region N-terminal boundary as the first DH cysteine in the “CPDG” motif and the C-terminal boundary as the position located by subtracting number of ascending stalk residues from the framework 4 tryptophan position (FIG. 15). The algorithm serves as a general rule that can be applied to bovine ultralong CDR H3 antibody sequences.

In summary, our algorithm (below) defines the knob region N-terminal boundary as the first DH cysteine in the “CPDG” motif and the C-terminal boundary as the position located by subtracting number of ascending stalk residues from the framework 4 tryptophan position (FIG. 15). The algorithm serves as a general rule that can be applied to bovine ultralong CDR H3 antibody sequences.

The algorithm is described as follows: L=number of amino acids encompassing stalk and knob domains, starting at canonical framework 3 cysteine and ending at canonical framework 4 tryptophan. X=number of amino acids, starting at the framework 3 canonical cysteine that defines the ascending stalk, and ending at the amino acid preceding the conserved first D region cysteine in the “CPDG” motif. Position of conserved framework 4 tryptophan−X=knob boundary position (C-terminal end); Number of residues in the knob (K)=L−2×; K position=(X+1) to (X+K)

TABLE E8

| | Length | Number of | Number of | |
|---|---|---|---|---|
| | encompassing | residues | amino acids | |
| | stalk and knob | in knob | in each stalk | PDB |
| Antibody | domains (L) | domain (K) | strand (X) | ID |
| A01 | 65 | 43 | 11 | 5ilt |
| B11 | 67 | 41 | 13 | 5ihu |
| BLV1H12 | 65 | 39 | 13 | 4k3d |
| BLV5B8 | 60 | 38 | 11 | 4k3e |
| E03 | 48 | 22 | 13 | 5ijv |
| BOV1 | 65 | 43 | 11 | 6e8v |
| BOV2 | 63 | 41 | 11 | 6e9g |
| BOV3 | 67 | 41 | 13 | 6e9h |
| BOV4 | 64 | 38 | 13 | 6e9i |
| BOV5 | 58 | 32 | 13 | 6e9k |
| BOV6 | 54 | 32 | 11 | 6e9q |
| BOV7 | 67 | 41 | 13 | 6e9u |

Bovine ultralong antibodies with published crystal structures that were analyzed, with X number of amino acids in the ascending and descending strands. Total number of amino acids comprising the stalk and knob domain (L) and knob domain alone (K) for each antibody are also noted.

### Example 12 Defining the Minimal CDR3-Knob C-Terminus and Minimal CDR3-Knob N-Terminus

The algorithm described in Example 11 was validated experimentally by expressing and testing C-terminal truncations (subsection A below) and N-terminal truncations (subsection B below) of a stalk and knob region from an antibody with an unknown structure. In some cases, 1, 2, 3, 4 or 5 amino acids may be added to the knob ends for improved expression or stability.

### A. Defining Minimal CDR3-Knob C-terminus

In order to define the C-terminal requirements of a prototypical CDR3-knob, a series of R2G3 truncations were cloned into pET32b and expressed as described in Example 6 above. The quality of expressed material was assessed by SDS-PAGE and RBD ELISA also as described in Example 6. Exemplary tested R2G3 truncations are set forth below in Table E9, each truncation was made with a reduced Terminal linker

TABLE E9
R2G3 CDR3-knob truncations-C-Terminus

| CLONE | Mature amino acid sequence after Enterokinase cleavage |
|---|---|
| G3 Parental | GGGGAMGSEGDKTCPDGYEHTCGCIGGCGCKRSACIGALCCQASLGGWLSDGETYT |
| G3 TRUNC1 | ~~~~~GGSEGDKTCPDGYEHTCGCIGGCGCKRSACIGALCCQASLGGWLSDGETYT |
| G3 TRUNC2 | ~~~~~GGSEGDKTCPDGYEHTCGCIGGCOCKRSACIGALCCQASLGGWLSDGE |
| G3 TRUNC3 | ~~~~~GGSEGDKTCPDGYEHTCGCIGGCGCKRSACIGALCCQASLGGWLS |
| G3 TRUNC3A | ~~~~~GGSEGDKTCPDGYEHTCGCIGGCGCKRSACIGALCCQASLGGWL |
| G3 TRUNC3B | ~~~~~GGSEGDKTCPDGYEHTCGCIGGCGCKRSACIGALCCQASLGGW |
| G3 TRUNC4 | ~~~~~GGSEGDKTCPDGYEHTCGCIGGCGCKRSACIGALCCQASLGG |
| G3 TRUNC5 | ~~~~~GGSEGDKTCPDGYEHTCGCIGGCGCKRSACIGALCCQAS |

As shown in FIG. 16A, only Truncations 4 and 5 resulted in no observed RBD binding. Truncations 3A and 3B demonstrated reduced binding in ELISA and increased band diffuseness in SDS-PAGE (FIG. 16B). Truncations 1-3 had no loss in binding activity relative to parental R2G3 CDR3-knob. Taken together, these results support a minimum of 9 amino acids after the last non-canonical Cys residue for R2G3 binding.

### B. Defining the Minimal CDR3-Knob N-Terminus

Similarly as described in Example 11, a series of R2G3 truncations were cloned into pET32b To define the N-terminal requirements of a prototypical CDR3-knob and expressed as described in Example 6 above. The quality of expressed material was assessed by SDS-PAGE and RBD ELISA as described in Example 6. Exemplary tested R2G3 truncations are set forth below in Table E10.

TABLE E10
R2G3 CDR3-knob truncations-N-Terminus

| CLONE | Mature amino acid sequence after Enterokinase cleavage |
|---|---|
| G3 Parental | GGSEGDKTCPDGYEHTCGCIGGCGCKRSACIGALCCQASLGGWLSD |
| G3 NTRUNC1 | GGS~GDKTCPDGYEHTCGCIGGCGCKRSACIGALCCQASLGGWLSD |
| G3 NTRUNC2 | GGS~~DKTCPDGYEHTCGCIGGCGCKRSACIGALCCQASLGGWLSD |
| G3 NTRUNC3 | GGS~~~KTCPDGYEHTCGCIGGCGCKRSACIGALCCQASLGGWLSD |
| G3 NTRUNC4 | GGS~~~~TCPDGYEHTCGCIGGCGCKRSACIGALCCQASLGGWLSD |
| G3 NTRUNC5 | GGS~~~~~CPDGYEHTCGCIGGCGCKRSACIGALCCQASLGGWLSD |

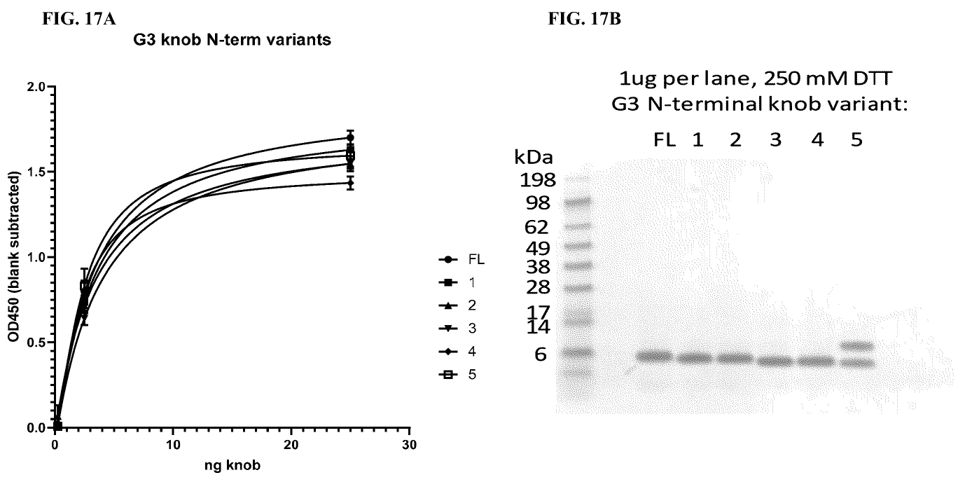

Each of the exemplary N-terminal truncation tested was observed to display similar binding profiles to biotinylated RBD by ELISA and band diffuseness in SDS-PAGE (FIGS. 17A and 17B, respectively). It was noted that truncation 5 resulted in two bands via SDS-PAGE, however this did not correlate with any reduction in binding activity. These results suggest that none of the amino acids deleted in these exemplary truncated R2G3 sequences are part of the knob domain.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

### VIII. Sequences

| SEQ ID | Name Sequence | |
|---|---|---|
| 001 | CAGGCCGTCCTGAACCAGCCAAGCAGCGTCTCCGGGTCTCTGG | BLVIH12 Light |
| | GGCAGCGGGTCTCAATCACCTGTAGCGGGTCTTCCTCCAATGTC | chain DNA |
| | GGCAACGGCTACGTGTCTTGGTATCAGCTGATCCCTGGCAGTG | |
| | CCCCACGAACCCTGATCTACGGCGACACATCCAGAGCTTCTGG | |
| | GGTCCCCGATCGGTTCTCAGGGAGCAGATCCGGAAACACAGCT | |
| | ACTCTGACCATCAGCTCCCTCCAGGCTGAGGACGAAGCAGATT | |
| | ATTTCTGCGCGTCTGCCGAGGACTCTAGTTCAAATGCCGTGTTT | |
| | GGAAGCGGCACCACACTGACAGTCCTA | |
| 002 | QAVLNQPSSVSGSLGQRVSITCSGSSSNVGNGYVSWYQLIPGSAPR | BLVIH12 variable |
| | TLIYGDTSRASGVPDRFSGSRSGNTATLTISSLQAEDEADYFCASA | Light chain |
| | EDSSSNAVFGSGTTLTVL | |
| 003 | CCGCTCTTCAGGGCACCCGAGTTCC | igGCDNA2REV |
| 004 | CTGACTGTGCTGTTGTTGAACTTCC | igMCDNA2REV |
| 005 | GACACGCTGTCGCCATTCTGGTTCC | igACDNA2REV |
| 006 | CGGGCACGGTCACCATGCTGCTGAGAGAGTAG | igGCDNA1.7REV |
| 007 | TTACCTGCGGCCGCTGAGGAGACGGTGACCAGGAGTCCAACTG | BOVVHFR4REV |
| | GAGCTCCATCAAG | |
| 008 | CAGCCGGCCATGGCCACATACTACAGTACTACTGTACACC | BOVSTALKFOR1 |
| 009 | CAGCCGGCCATGGCCACATACTACAGTACTACTGTATACC | BOVSTALKFOR2 |
| 010 | CAGCCGGCCATGGCCACATACTACAGTACTACTGTGCTCC | BOVSTALKFOR3 |
| 011 | CAGCCGGCCATGGCCACATACTACAGTGGTACTGTGCACC | BOVSTALKFOR4 |
| 012 | aaaaaGCCATGGTGCAGGTGCAGCTGCGGGAGTCGGG | BOVVHNCOFOR2 |
| | | NotI restriction |
| | | enzyme site |
| | | (bold/underline) |
| 013 | TTACCTCTCGAGTGAGGAGACGGTGACCAGGAGTCC | BOVVHFR4XHOREV |
| | | Xho I restriction |
| | | enzyme site |
| | | (bold/underline) |
| 014 | CAGGTGCAGCTGCGGGAGTCGGGCCCCAGCCTGGTGAAGCCCT | R2G3 |
| | CACAGACCCTCTCGCTCACCTGCGCGGCCTCTGGATTCTCATTG | |
| | AGCGACAAGGCTGTAGGCTGGGTCCGCCGGGCTCCAGGGAAG | |
| | GCGCTGGAGTGGCTCGGTAGTATAGACACTGGTGGAAGCACAG | |
| | GCTATAACCCAGGCCTGAAATCCCGGCTCAGCATCACCAAGGA | |
| | CAACTCCAAGAGCCAAGTCTCCCTGTCAATTAGCAGCGTAACG | |
| | TCTGAGGACTCGGCCACATACTACTGTGCAACTGTACACCAGA | |
| | AAACAGCTGAAGGAGACAAAACGTGTCCTGATGGTTACGAGC | |
| | ATACTTGTGGTTGCATTGGGGGTTGTGGTTGCAAAAGGTCTGCC | |
| | TGTATAGGTGCACTTTGTTGCCAAGCGTCGTTGGGTGGTTGGCT | |
| | TAGTGACGGTGAAACCTACACTTACGAGTTCCACGTCGATACC | |
| | TGGGGCCAAGGACTCGTGGTCACCGTCTCCTCA | |
| 015 | CAGGTGCAGCTGCGGGAGTCGGGCCCCAGCCTGGTGAAGCCCT | SR3A3 |
| | CACAGACCCTCTCCCTCACCTGCACAATCTCTGGATTCTCATTG | |
| | AGTAGCTATGCTGTACTCTGGGTCCGCCAGGCTCCAGGGAAGC | |
| | CGCTGGAGTGGCTCGGTAGTATAGACACTGCGGAAAACACAGG | |
| | CTATAACCCAGGCCTGAAATCCCGGCTCAGCATCACCAAGGAC | |
| | AACTCCAAGAGCCAAGTCTCTCTGTCAGTGAGCAGCGTGACAA | |
| | CCGAGGACTCGGCCACATACTACTGTGCTACTGTACACCAGAA | |
| | AACGCGAAAAGAAAAAAATTGTCCTGATGGCTATATCTATAGT | |
| | TCTAATATCACTAGCGGTTTTGATTGTGGTGTCTGGATTTGTCG | |
| | TCGCGTCGGTAGTGCCTTCTGTAGTCGTACTGGTGATTATACTA | |
| | GTCCTACTGAACTTGACATTTACGAGTTCTACGTCGAAGGGTG | |
| | GGGCCAGGGAGTCCCGGTCACCGTCTCCTCA | |
| 016 | CAGGTGCAGCTGCGGGAGTCGGGCCCCAGCCTGGTGAAGCCCT | R2F12 |
| | CACAGACCCTCTCCCTCACCTGCACGGTCTCTGGATTCTCATTG | |
| | AGCGACAAGGCTGTAGGCTGGGTCCGCCGGGCTCCAGGGAAG | |
| | GCGCTGGAGTGGCTCGGTAGTATAGACACTGGTGGAATGACAG | |
| | GCTATAACCCAGGCCTGAAATCCCGGCTCAGCATCACCAAGGA | |
| | CAACTCCAAAAGCCAAGTCTCTCTATCAGTGAATAGCGTGACA | |
| | ACTGAGGACTCGGCCACGTACTACTGTGCCACTGTAGACCAGA | |
| | AAACGAAAAATGCTTGCCCTGATGATTTCGATTATCGTTGTTCG | |
| | TGTATCGGTGGTTGTGGCTGCGCCCGTAAAGGATGCGTTGGTC | |
| | CTCTTTGTTGTCGTTCTGATTTGGGTGGCTATCTTACTGATAGTC | |
| | CTGCTTACATTTACGAATGGTATATTGATCTTTGGGGCCAAGGA | |
| | CTCCTGGTCACCGTCTCCTCA | |
| 017 | CAGGTGCAGCTGCGGGAGTCGGGCCCCAGCCTGGTGAAGCCGT | R5A3 |
| | CACAGACCCTCTCGCTCACCTGCACGGCCTCTGGATTCTCATTG | |
| | AGCGACAAGGCTGTAGGCTGGGTCCGCCAGGCTCCAGGGAAG | |
| | GCGCTGGAGTGGCTCGGTAGTATAGACACTGGTGGAAGCACAG | |
| | GCTATAACCCAGGCCTGAAATCCCGGCTCAGCATCACCAAGGA | |
| | CAACTCCAAGAGCCAAGTCTCTCTGTCAGTGAGCAGCGTGACA | |
| | ACTGAGGACTCGGCCACATACTACTGTACTACTGTGCACTGTA | |
| | GTGATGGTGGTTATGTTGAGGGGGGTTTTGGTTGTTGGCCTTGG | |
| | GATTATGGTTATCCTTACGTCGATGCCTGGGGCCAAGGACTCCT | |
| | GGTCACCGTCTCCTCA | |
| 018 | CAGGTGCAGCTGCGGGAGTCGGGCCCCAGCCTGGTGAAGCCCT | R4G11 |
| | CACAGACCCTCTCCCTCACCTGCACGGTCTCTGGATTCTCATTG | |
| | AGCAGCTATGGTATAACCTGGGTCCGCCAGGCTCCAGGGAAGG | |
| | CGCTGGAGTGCCTCGGTAGTATAAGCAGTGGTGGAACCACAGA | |
| | CTACAACCCAGCCCTGAAATCCCGGCTCAGCATCACCAAGGAC | |
| | AACTCCAAGAGCCAAGTCTCTCTGTCAGTGAGCAGCGTGACAC | |
| | CTGAGGACACGGCCACATACTACTGTTCGAAGTGGAATTTAGA | |
| | ATATACTTGGGGTGGTGTTGGTTGCGCTAGTTTTGCTGATGAGG | |
| | ACACCCACGTTGATGCCTGGGGCCAAGGACTCCTGGTCACCGT | |
| | CTCCTCA | |
| 019 | CAGGTGCAGCTGCGGGAGTCGGGCCCCAGCCTGATGAAGCCCT | R4G3 |
| | CACAGACCCTCTCCCTCACCTGCACGGTCTCTGGGTTCTCATTG | |
| | AGCGACTATGCTGTAGGCTGGGTCCGCCAGGCCCCAGGGAAGG | |
| | CGCTGGAGTGGCTCGGTGGTATAGACACTGGTGGAAGCACAGG | |
| | CTATAACCCAGGCCTGGAATCCCGGCTCAGCATCACCAAGGAC | |
| | AACTCCAAGAGCCAAGTCTCTCTGTCAGTGAGCAGCGTGACAA | |
| | CTGAGGACTCGGCCACATACTACTGTACTACTGTGGTCCTTTGT | |
| | TATTTTAATTATGTTGTTCGTCGTTATAATTGTGGTGGTCTTGGT | |
| | TATGGGCATGGCTTTAATAGTTTCTACGTCGATGCCTGGGGCCA | |
| | AGGACTCCTGGTCACCGTCTCCTCA | |
| 020 | CAGGTGCAGCTGCGGGAGTCGGGCCCCAGCCTGGTGAAGCCCT | R4E5 |
| | CACAGACCCTCTCCCTCACCTGCACGACCTCTGGATTCTCACTG | |
| | AGAAACTATGCTGTAGGCTGGGTCCGCCAGGCTCCGGGGAAGG | |
| | CGCTGGAGTGGCTCGGTGGTATAGACACTGGTGGAAGCACAGG | |
| | CTATAACCCAGGCCTGGAATCCCGGCTCAGCATCACCAAGGAC | |
| | AACTCCAAGAGCCAAGTCTCTCTGTCAGTGAGCAGCGTGACAA | |
| | CTGAGGACTCGGCCACATACTACTGTACTACTGTGGTCCTTTGT | |
| | TATTTTAATTATGTTGTTCGTCGTTATAATTGTGGTGGTCTTGGT | |
| | TATGGGCATGGCTTTAATAGTTTCTACGTCGATGCCTGGGGCCA | |
| | AGGACTCCTGGTCACCGTCTCCTCA | |
| 021 | CAGGTGCAGCTGCGGGAGTCGGGCCCCAGCCTGGTGAAGCCGT | R4C1 |
| | CACAGACCCTCTCGCTCACCTGCACGGCCTCTGGATTCTCATTG | |
| | AGCGATAAGGCTGTAGGCTGGGTCCGCCAGGCTCCAGGGAAGC | |
| | CGCTGGAGTGGCTCGGTAGTATAGACACTGCGGAAAACACAGG | |
| | CTATAACCCAGGCCTGAAATCTCGGCTCAGCATCACCAAGGAC | |
| | AACTCCAAGAGCCAAGTCTCTCTGTCAGTGAGCAGCGTGACAA | |
| | CTGAGGACTCGGCCACATACTACTGTGCTACTGTACACCAGAA | |
| | AACGCGAAAAGAAAAAAATTGTCCTGATGGCTATATCTATAGT | |
| | TCTAATACCGCCAGCGGTTATGATTGTGGTGTCTGGATTTGTCG | |
| | TCGCGTCGGTAGTGCCTTCTGTAGTCGTACTGGTGATTATACTA | |
| | GTCCTAGTGAATTTGACATTTACGAgTTCTACGTCGAAGGGTGG | |
| | GGCCAGGGAcTCCtGGTCACCGTCTCCTCA | |
| 022 | CAGGTGCAGCTGCGGGAGTCGGGCCCCAGCCTGGTGAAGCCCT | R4A10 |
| | CACAGACCCTCTCCCTCACCTGCACGACCTCTGGATTCTCATTG | |
| | AGCGACTATGCTGTAGGCTGGGTCCGCCAGGCTCCAGGGAAGG | |
| | CGCTGGAGTGGCTCGGTGGTATAGACACTGGTGGAAGCACAGG | |
| | CTATAACCCAGGCCTGAAATCCCGGCTCAGCATCACCAAGGAC | |
| | AACTCCAAGAGTCAAGTCTCTCTGTCAGTGAGCAGCGTGACAA | |
| | CTGAGGATTCGGCCACATACTACTGTACTGCCGTGGTCCTCTGT | |
| | TATTACAATCGGGTTGTGCGTCGTAATAATTGTGGTGGGCTTGG | |
| | TTATGATTATGGTTTTGATCATTTCTACGTCGATGCCTGGGGCC | |
| | AAGGACTCCTGGTCACCGTCTCCTCA | |
| 023 | CAGGTGCAGCTGCGGGAGTCGGGCCCCAGCCTGGTGAAGCCCT | R2G1 |
| | CACAGACCCTCTCCCTCACCTGCACGGTCTCTGGATTCTCATTG | |
| | AGCAACTATGCTGTAGGCTGGGTCCGCCAGGCTCCAGGGAAGG | |
| | CGCTGGAGTGCCTCGGTGATGTAGACAGTAGTGGAGGCACAGC | |
| | CTATAACCCAGCCCTGAAATCCCGGTTCATCATCGCCAAGGAC | |
| | AACTCCAAGAACCAAGTCTCTCTGTCAGTCCGCAGCGTGACAC | |
| | CTGAGGACACGGCCACATACTACTGTGCGAAGTTTGCTAAGGG | |
| | TACTACGAGTGCTGGTGCTTGTGATTATTCAGAAAGCTACGTCG | |
| | ATGCCTGGGGCCAGGGACTCCTGGTCACCGTCTCCTCA | |
| 024 | CAGGTGCAGCTGCGGGAGTCGGGCCCCAGCCTGGTGAAGCCCT | R2D6 |
| | CACAGACCCTCTCCCTCACCTGCACGACCTCTGGATTCTCACTG | |
| | AGCAGCTATGCTGTAGGCTGGGTCCGCCAGGCTCCGGGGAAGG | |
| | CGCTGGAGTGGGTTGGTGATATAGATTATGTCGGAAACACAGA | |
| | CTATAACCCAGCCCTGAAATCCCGGCTCAGCATCACCAAGGAC | |
| | AACTCCAAGAGCCAAGTCTCTCTGGTAGTGAGCAGCGTGACAG | |
| | CTGAGGACGCGGCCACATACTACTGTGCGAAATATTCCGGTGC | |
| | TTATGCTTATGCTGCTTGCAATTATTATGGTTGGCGTTGTGCTT | |
| | GGGAAAGCTACATCGATGCCTGGGGCCAAGGACTCCTGGTCAC | |
| | CGTCTCCTCA | |
| 025 | CAGGTGCAGCTGCGGGAGTCGGGCCCCAGCCTGGTGAAGCCCT | R2B1 |
| | CACAGACCCTCTCCCTCACCTGCACGGTCTCTGGATTTTCATTA | |
| | AGCGATAATAATGTAGGCTGGGTCCGCCAGGCTCCAGGAAAGG | |
| | CGCTGGAGTGGCTCGGTGTAATGCATAATGATGGGAACAAAGG | |
| | CTATAACCCAGCCCTGAAATCCCGGCTCAGCATCACCAAGGAC | |
| | AGCTCCAAGAGCCAAGTCTCTCTATCACTAAGCAGCGTGACAA | |
| | GTGAGGACACGGCCACATACTACTGTACAAGAGACAATGCACG | |
| | TTGTGATAGTTGGACGTATGACAGCTGTGATACTTGGTATCGCA | |
| | ATTCGTGGCACGTTGATGCCTGGGGCCAAGGACTCCTgGTCACC | |
| | GTCTCCTCA | |
| 026 | CAGGTGCAGCTGCGCGAGTCGGGCCCCAGCCTGGTGAAGCCGT | SKM-BLV1H12 |
| | CACAGACCCTCTCGCTCACCTGCACGGCCTCTGGATTCTCATTG | |
| | AGCGACAAGGCTGTAGGCTGGGTCCGCCAGGCTCCAGGGAAG | |
| | GCGCTGGAGTGGCTCGGTAGTATAGACACTGGTGGAAACACAG | |
| | GCTATAACCCAGGCCTGAAATCCCGGCTCAGCATCACCAAGGA | |
| | CAACTCCAAGAGTCAAGTCTCTCTGTCAGTGAGCAGCGTGACA | |
| | ACTGAGGACTCGGCCACATACTACTGTACTACTGTGCACCAAG | |
| | AGACCTTACGTAGTTGTCCTGATGGTTATATTGATAATTCTGGA | |
| | TGCACGGCTGATTGGGGTTGTGCAGCTCTTGATTGTTGGCGGCG | |
| | TCGTTTTGGTTACCACAGCACTGATCCTTCTCATTATACTGGTG | |
| | CGACGTATATTTACACGTACAGCTTGCACATCGATGCCTGGGG | |
| | CCAAGGACTCCTGGTCACCGTCTCCTCA | |
| 027 | CAGGTGCAGCTGCGCGAGTCGGGCCCCAGCCTGGTGAAGCCGT | SKD-BLV1H12 |
| | CACAGACCCTCTCGCTCACCTGCACGGCCTCTGGATTCTCATTG | |
| | AGCGACAAGGCTGTAGGCTGGGTCCGCCAGGCTCCAGGGAAG | |
| | GCGCTGGAGTGGCTCGGTAGTATAGACACTGGTGGAAACACAG | |
| | GCTATAACCCAGGCCTGAAATCCCGGCTCAGCATCACCAAGGA | |
| | CAACTCCAAGAGTCAAGTCTCTCTGTCAGTGAGCAGCGTGACA | |
| | ACTGAGGACTCGGCCACATACTACTGTACTACTGTGCACCAGC | |
| | GTACAAGCGAAAAAAGAAGTTGTCCTGGCGGTAGTAGTAGAC | |
| | GTTATCCTAGTGGCGCCAGTTGTGACGTTAGTGGGGGCGCTTGT | |
| | GCGTGTTATGTTTCTAATTGTAGAGGCGTTTTGTGTCCTACTCTT | |
| | AACGAAATCGTTGCTTATACCTACGAATGGCACGTCGACGCCT | |
| | GGGGCCAAGGACTCCTGGTCACCGTCTCCTCA | |
| 028 | CAGGTGCAGCTGCGGGAGTCGGGCCCCAGCCTGGTGAAGCCCT | RBD F4 |
| | CACAGACCCTCTCCCTCACCTGCACGGTCTCTGGATTCTCATTG | |
| | AGCAGCAATGGTGTGGTCTGGGTCCGCCAGGCTCCAGGGAAGG | |
| | CGCTGGAGTGGCTCGGTGATATATGCAGTACTGGAGGCACAAG | |
| | CTTTAACCCAGCCCTGAAATCCCGGCTCAGCATCGCCAAGGAC | |
| | AACTCCAAGAGCCAAGTCTCTCTGTCAGTGAGAAGCGTGACAC | |
| | CTGAGGACACGGCCACATATTACTGTGCAAGAAGTCGTGGTTA | |
| | TGATTGTTATGCTAATGTGGATGCTTTGGACTACGTCGATGCCT | |
| | GGGGCCAAGGACTCCTGGTCACCGTCTCCTCA | |
| 29 | CAGGTGCAGCTGCGGGAGTCGGGCCCCAGCCTGGTGAAGCCCT | RBD C6 |
| | CACAGACCCTCTCCCTCACCTGCACGGTCTCTGGATTCTCATTG | |
| | AGCAGCAATGGTGTAGTCTGGGTCCGCCAGGCTCCAGGGAGAC | |
| | CACTGGAGTGGCTCGGTGATATATGCAGTAATGGAGGCACAAG | |
| | CTTTAACCCAGCCCTGAAATCCCGGCTCAGCATCGCCAAGGAC | |
| | AACTCCGAGAGCCAAGTCTCTCTGACCGTGAGAAGCGTGACAC | |
| | CTGAGGACACAGCCACATATTACTGTGCAAGAAGTCGTGGTTA | |
| | TGATTGTTATGCTTATGTTTATGCTTTGGACACCGTCGATGCCT | |
| | GGGGCCAAGGACTCCTGGTCACCGTCTCCTCA | |
| 030 | CAGGTGCAGCTGCGGGAGTCGGGCCCCAGCCTGGTGAAGCCCC | RBD A2 |
| | TACAGATCCTCTCCCTCACCTGCACGGTCTCTGGATTCTCATTG | |
| | AGCAGCAATGGTGTGGTCTGGGTCCGCCAGGCTCCAGGGAAGG | |
| | CGCTGGAGTGGCTCGGTGATATATGCAGTACTGGAGGCACAAG | |
| | CTTTAACCCAGCCCTGAAATCCCGGCTCAGCATCGCCAAGGAC | |
| | AACTCCAAGAGCCAAGTCTCTCTGTCAGTGAGAAGCGTGACAC | |
| | CTGAGGACACGGCCACATATTACTGTGCAAGAAGTCGTGGTTA | |
| | TGATTGTTATGCTAATGTGGATGCTTTGGACTACGTCGATGCCT | |
| | GGGGCCAAGGACTCCTGGTCACCGTCTCCTCA | |
| 031 | CAGGTGCAGCTGCGGGAGTCGGGCCCCAGCCTGGTGAAGCCGT | SA-R2C3 |
| | CACAGACCCTCTCGCTCACCTGCACGGCCTCTGGATTCTCATTG | |
| | AGCGATAAGCCTGTAGGCTGGGTCCGCCAGGCTCCAGGGAAGC | |
| | CACTGGAGTGGCTCGGTAGTATAGACACTGCGGAAAACACAGG | |
| | CTATAACCCAGGCCTGAAATCTCGGCTCAGCATCACCAAGGAC | |
| | AACTCCAAGAGCCAAGTCTCTCTGTCACTGAGCAGCGTGACGA | |
| | CTGAGGACTCGGCCACATACTACTGTGCTACTGTACACCAGAA | |
| | AACGCGGAAGGAAAAAAGTTGTCCTGATGGCTATCTCTATAGT | |
| | TCTAATACCGGCCGCGGTTATGATTGTGGTGTCTGGACTTGTCG | |
| | TCGCGTCGGTGGTGAATTCTGTAGTGCTACTGGTGATTGGACTA | |
| | GTCCTAGTGAAGAAGACTTTTACGAATTCTACGTCGATACGTG | |
| | GGGCCAGGGAGCCCCGGTCACCGTCTCCTCA | |
| 032 | CAGGTGCAGCTGCGGGAGTCGGGCCCCAGCCTGGTGAAGCCGT | SA-R2D9 |
| | CACAGACCCTCTCGCTCACCTGCACGGCCTCTGGATTCTCATTA | |
| | AGCGACAAGGCTATTGGCTGGGTCCGCCAGGCTCCAGGGAAGG | |
| | CGCTGGAGTGGCTCGGTAGTATAGACACCCGTGGAAACACAGG | |
| | CTATAACCCAGGCCTGAAATCCCGACTCAGCATCACCAAGGAC | |
| | AGCTCCAAGAGCCAAGTCTCTCTGTCAGTGAACAGCGTGACAA | |
| | CTGAAGACTCGGCCACGTACCTCTGTGCTATTGTGCAGCAGAT | |
| | CACACACAAAACTTGTCCTAATGGTTACAATTGGTTTGATCGTT | |
| | GTTGTTCTTGGGATGGTACCTGTGGTGATGGTTGTTGCAGTAAT | |
| | CGTGCTTGGCCTAGTGGTAATGGTAGAGCCGACAGTAGTATTG | |
| | GTGAAACTTATGGTTACGAATTTCACGTGGCTGCCTGGGGCCA | |
| | AGGACTCCTGGTCACCGTCTCCTCA | |
| 033 | QVQLRESGPSLVKPSQTLSLTCAASGFSLSDKAVGWVRRAPGKAL | R2G3 |
| | EWLGSIDTGGSTGYNPGLKSRLSITKDNSKSQVSLSISSVTSEDSAT | |
| | YYCATVHQKTAEGDKTCPDGYEHTCGCIGGCGCKRSACIGALCC | |
| | QASLGGWLSDGETYTYEFHVDTWGQGLVVTVSS | |
| 034 | QVQLRESGPSLVKPSQTLSLTCTISGFSLSSYAVLWVRQAPGKPLE | SR3A3 |
| | WLGSIDTAENTGYNPGLKSRLSITKDNSKSQVSLSVSSVTTEDSAT | |
| | YYCATVHQKTRKEKNCPDGYIYSSNITSGFDCGVWICRRVGSAFC | |
| | SRTGDYTSPTELDIYEFYVEGWGQGVPVTVSS | |
| 035 | QVQLRESGPSLVKPSQTLSLTCTVSGFSLSDKAVGWVRRAPGKAL | R2F12 |
| | EWLGSIDTGGMTGYNPGLKSRLSITKDNSKSQVSLSVNSVTTEDS | |
| | ATYYCATVDQKTKNACPDDFDYRCSCIGGCGCARKGCVGPLCCR | |
| | SDLGGYLTDSPAYIYEWYIDLWGQGLLVTVSS | |
| 036 | QVQLRESGPSLVKPSQTLSLTCTASGFSLSDKAVGWVRQAPGKAL | R5A3 |
| | EWLGSIDTGGSTGYNPGLKSRLSITKDNSKSQVSLSVSSVTTEDSA | |
| | TYYCTTVHCSDGGYVEAGFGCWPWDYGYPYVDAWGQGLLVTV | |
| | SS | |
| 037 | QVQLRESGPSLVKPSQTLSLTCTVSGFSLSSYGITWVRQAPGKALE | R4G11 |
| | CLGSISSGGTTDYNPALKSRLSITKDNSKSQVSLSVSSVTPEDTATY | |
| | YCSKWNLEYTWGGVGCASFADEDTHVDAWGQGLLVTVSS | |
| 038 | QVQLRESGPSLMKPSQTLSLTCTVSGFSLSDYAVGWVRQAPGKAL | R4G3 |
| | EWLGGIDTGGSTGYNPGLESRLSITKDNSKSQVSLSVSSVTTEDSA | |
| | TYYCTTVVLCYFNYVVRRYNCGGLGYGHGFNSFYVDAWGQGLL | |
| | VTVSS | |
| 039 | QVQLRESGPSLVKPSQTLSLTCTTSGFSLRNYAVGWVRQAPGKAL | R4E5 |
| | EWLGGIDTGGSTGYNPGLESRLSITKDNSKSQVSLSVSSVTTEDSA | |
| | TYYCTTVVLCYFNYVVRRYNCGGLGYGHGFNSFYVDAWGQGLL | |
| | VTVSS | |
| 040 | QVQLRESGPSLVKPSQTLSLTCTASGFSLSDKAVGWVRQAPGKPL | R4C1 |
| | EWLGSIDTAENTGYNPGLKSRLSITKDNSKSQVSLSVSSVTTEDSA | |
| | TYYCATVHQKTRKEKNCPDGYIYSSNTASGYDCGVWICRRVGSA | |
| | FCSRTGDYTSPSEFDIYEFYVEGWGQGLLVTVSS | |
| 041 | QVQLRESGPSLVKPSQTLSLTCTTSGFSLSDYAVGWVRQAPGKAL | R4A10 |
| | EWLGGIDTGGSTGYNPGLKSRLSITKDNSKSQVSLSVSSVTTEDSA | |
| | TYYCTAVVLCYYNRVVRRNNCGGLGYDYGFDHFYVDAWGQGL | |
| | LVTVSS | |
| 042 | QVQLRESGPSLVKPSQTLSLTCTVSGFSLSNYAVGWVRQAPGKAL | R2G1 |
| | ECLGDVDSSGGTAYNPALKSRFIIAKDNSKNQVSLSVRSVTPEDTA | |
| | TYYCAKFAKGTTSAGACDYSESYVDAWGQGLLVTVSS | |
| 043 | QVQLRESGPSLVKPSQTLSLTCTTSGFSLSSYAVGWVRQAPGKAL | R2D6 |
| | EWVGDIDYVGNTDYNPALKSRLSITKDNSKSQVSLVVSSVTAEDA | |
| | ATYYCAKYSGAYAYAACNYYGWRCAWESYIDAWGQGLLVTVS | |
| | S | |
| 044 | QVQLRESGPSLVKPSQTLSLTCTVSGFSLSDNNVGWVRQAPGKAL | R2B1 |
| | EWLGVMHNDGNKGYNPALKSRLSITKDSSKSQVSLSLSSVTSEDT | |
| | ATYYCTRDNARCDSWTYDSCDTWYRNSWHVDAWGQGLLVTVS | |
| | S | |
| 045 | QVQLRESGPSLVKPSQTLSLTCTASGFSLSDKAVGWVRQAPGKAL | SKM-BLV1H12 |
| | EWLGSIDTGGNTGYNPGLKSRLSITKDNSKSQVSLSVSSVTTEDSA | |
| | TYYCTTVHQETLRSCPDGYIDNSGCTADWGCAALDCWRRRFGYH | |
| | STDPSHYTGATYIYTYSLHIDAWGQGLLVTVSS | |
| 046 | QVQLRESGPSLVKPSQTLSLTCTASGFSLSDKAVGWVRQAPGKAL | SKD-BLV1H12 |
| | EWLGSIDTGGNTGYNPGLKSRLSITKDNSKSQVSLSVSSVTTEDSA | |
| | TYYCTTVHQRTSEKRSCPGGSSRRYPSGASCDVSGGACACYVSNC | |
| | RGVLCPTLNEIVAYTYEWHVDAWGQGLLVTVSS | |
| 047 | QVQLRESGPSLVKPSQTLSLTCTVSGFSLSSNGVVWVRQAPGKAL | RBD F4 |
| | EWLGDICSTGGTSFNPALKSRLSIAKDNSKSQVSLSVRSVTPEDTA | |
| | TYYCARSRGYDCYANVDALDYVDAWGQGLLVTVSS | |
| 048 | QVQLRESGPSLVKPSQTLSLTCTVSGFSLSSNGVVWVRQAPGRPL | RBD C6 |
| | EWLGDICSNGGTSFNPALKSRLSIAKDNSESQVSLTVRSVTPEDTA | |
| | TYYCARSRGYDCYAYVYALDTVDAWGQGLLVTVSS | |
| 049 | QVQLRESGPSLVKPLQILSLTCTVSGFSLSSNGVVWVRQAPGKAL | RBD A2 |
| | EWLGDICSTGGTSFNPALKSRLSIAKDNSKSQVSLSVRSVTPEDTA | |
| | TYYCARSRGYDCYANVDALDYVDAWGQGLLVTVSS | |
| 050 | QVQLRESGPSLVKPSQTLSLTCTASGFSLSDKPVGWVRQAPGKPL | SA-R2C3 |
| | EWLGSIDTAENTGYNPGLKSRLSITKDNSKSQVSLSLSSVTTEDSA | |
| | TYYCATVHQKTRKEKSCPDGYLYSSNTGRGYDCGVWTCRRVGG | |
| | EFCSATGDWTSPSEEDFYEFYVDTWGQGAPVTVSS | |
| 051 | QVQLRESGPSLVKPSQTLSLTCTASGFSLSDKAIGWVRQAPGKAL | SA-R2D9 |
| | EWLGSIDTRGNTGYNPGLKSRLSITKDSSKSQVSLSVNSVTTEDSA | |
| | TYLCAIVQQITHKTCPNGYNWFDRCCSWDGTCGDGCCSNRAWPS | |
| | GNGRADSSIGETYGYEFHVAAWGQGLLVTVSS | |
| 052 | GAAGGAGACAAAACGTGTCCTGATGGTTACGAGCATACTTGTG | R2G3 |
| | GTTGCATTGGGGGTTGTGGTTGCAAAAGGTCTGCCTGTATAGG | |
| | TGCACTTTGTTGCCAAGCGTCGTTGGGTGGTTGGCTTAGTGACG | |
| | GTGAAACCTACACT | |
| 053 | AAAGAAAAAAATTGTCCTGATGGCTATATCTATAGTTCTAATA | SR3A3 |
| | TCACTAGCGGTTTTGATTGTGGTGTCTGGATTTGTCGTCGCGTC | |
| | GGTAGTGCCTTCTGTAGTCGTACTGGTGATTATACTAGTCCTAC | |
| | TGAACTTGACATTTACGAGTTC | |
| 054 | AAAACGAAAAATGCTTGCCCTGATGATTTCGATTATCGTTGTTC | R2F12 |
| | GTGTATCGGTGGTTGTGGCTGCGCCCGTAAAGGATGCGTTGGT | |
| | CCTCTTTGTTGTCGTTCTGATTTGGGTGGCTATCTTACTGATAGT | |
| | CCTGCTTACATTTACGAA | |
| 055 | AAAGAAAAAAATTGTCCTGATGGCTATATCTATAGTTCTAATA | R4C1 |
| | CCGCCAGCGGTTATGATTGTGGTGTCTGGATTTGTCGTCGCGTC | |
| | GGTAGTGCCTTCTGTAGTCGTACTGGTGATTATACTAGTCCTAG | |
| | TGAATTTGACATTTAC | |
| 056 | CTGCGTAGTTGTCCTGATGGTTATATTGATAATTCTGGATGCAC | SKM-BLV1H12 |
| | GGCTGATTGGGGTTGTGCAGCTCTTGATTGTTGGCGGCGTCGTT | |
| | TTGGTTACCACAGCACTGATCCTTCTCATTATACTGGTGCGACG | |
| | TATATTTACACGTAC | |
| 057 | AGCGAAAAAAGAAGTTGTCCTGGCGGTAGTAGTAGACGTTATC | SKD-BLV1H12 |
| | CTAGTGGCGCCAGTTGTGACGTTAGTGGGGGCGCTTGTGCGTG | |
| | TTATGTTTCTAATTGTAGAGGCGTTTTGTGTCCTACTCTTAACG | |
| | AAATCGTTGCTTATACCTAC | |
| 058 | CGGAAGGAAAAAAGTTGTCCTGATGGCTATCTCTATAGTTCTA | SA-R2C3 |
| | ATACCGGCCGCGGTTATGATTGTGGTGTCTGGACTTGTCGTCGC | |
| | GTCGGTGGTGAATTCTGTAGTGCTACTGGTGATTGGACTAGTCC | |
| | TAGTGAAGAAGACTTTTACGAATTC | |
| 059 | ATCACACACAAAACTTGTCCTAATGGTTACAATTGGTTTGATCG | SA-R2D9 |
| | TTGTTGTTCTTGGGATGGTACCTGTGGTGATGGTTGTTGCAGTA | |
| | ATCGTGCTTGGCCTAGTGGTAATGGTAGAGCCGACAGTAGTAT | |
| | TGGTGAAACTTATGGTTACGAATTT | |
| 060 | EGDKTCPDGYEHTCGCIGGCGCKRSACIGALCCQASLGGWLSDG | R2G3 |
| | ETYTYEF | |
| 061 | KEKNCPDGYIYSSNITSGFDCGVWICRRVGSAFCSRTGDYTSPTEL | SR3A3 |
| | DIYEF | |
| 062 | KTKNACPDDFDYRCSCIGGCGCARKGCVGPLCCRSDLGGYLTDSP | R2F12 |
| | AYIYE | |
| 063 | RKEKNCPDGYIYSSNTASGYDCGVWICRRVGSAFCSRTGDYTSPS | R4CI |
| | EFDIY | |
| 064 | LRSCPDGYIDNSGCTADWGCAALDCWRRRFGYHSTDPSHYTGAT | SKM-BLV1H12 |
| | YIYTY | |
| 065 | SEKRSCPGGSSRRYPSGASCDVSGGACACYVSNCRGVLCPTLNEI | SKD-BLV1H12 |
| | VAYTY | |
| 066 | RKEKSCPDGYLYSSNTGRGYDCGVWTCRRVGGEFCSATGDWTSP | SA-R2C3 |
| | SEEDFYEF | |
| 067 | ITHKTCPNGYNWFDRCCSWDGTCGDGCCSNRAWPSGNGRADSSI | SA-R2D9 |
| | GETYGYEF | |
| 68 | CTTVHQRTSEKRSCPGGSSRRYPSGASCDVSGGACACYVSNCRG | SKD |
| | VLCPTLNEIVAYTYEWHVDAWGQGLLVTVSS | |
| 69 | CTTVHQETLRSCPDGYIDNSGCTADWGCAALDCWRRRFGYHST | SKM |
| | DPSHYTGATYIYTYSLHIDAWGQGLLVTVSS | |
| 70 | CATVHQKTRKEKNCPDGYIYSSNTASGYDCGVWICRRVGSAFCS | R4C1 |
| | RTGDYTSPSEFDIYEFYVEGWGQGLLVTVSS | |
| 71 | CATVHQKTRKEKSCPDGYLYSSNTGRGYDCGVWTCRRVGGEFC | R5C1 |
| | SATGDWTSPSEEDFYEFYVDTWGQGLLVTVSS | |
| 72 | CATVHQKTRKEKNCPDGYIYSSNITSGFDCGVWICRRVGSAFCSR | SR3A3 |
| | TGDYTSPTELDIYEFYVEGWGQGVPVTVSS | |
| 73 | CATVDQKTKNACPDDFDYRCSCIGGCGCARKGCVGPLCCRSDLG | RR2F12 |
| | GYLTDSPAYIYEWYIDLWGQGLLVTVSS | |
| 74 | CATVHQKTAEGDKTCPDGYEHTCGCIGGCGCKRSACIGALCCQA | RR2G3 |
| | SLGGWLSDGETYTYEFHVDTWGQGLVVTVSS | |
| 75 | CTTVHQSCPDGYSYGYGCGYGYGCSGYDCYGYGGYGGYGGYG | Germ |
| | YSSYSYSYTYEYYVDAWGQGLLVTVSS | |
| 76 | MFVFLVLLPLVSSQCVNLTTRTQLPPAYTNSFTRGVYYPDKVFRSS | WT Wuhan-Hu-1 S |
| | VLHSTQDLFLPFFSNVTWFHAIHVSGTNGTKRFDNPVLPFNDGVY | protein |
| | FASTEKSNIIRGWIFGTTLDSKTQSLLIVNNATNVVIKVCEFQFQND | NCBI Reference |
| | PFLGVYYHKNNKSWMESEFRVYSSANNCTFEYVSQPFLMDLEGK | Sequence: |
| | QGNFKNLREFVFKNIDGYFKIYSKHTPINLVRDLPQGFSALEPLVD | YP_009724390.1 |
| | LPIGINITRFQTLLALHRSYLTPGDSSSGWTAGAAAYYVGYLQPRT | (RBD shown in bold, |
| | FLLKYNENGTITDAVDCALDPLSETKCTLKSFTVEKGIYQTSNFRV | intravirion |
| | QPTESIVRFPNITNLCPFGEVENATRFASVYAWNRKRISNCVAD | underline) |
| | YSVLYNSASFSTFKCYGVSPTKLNDLCFTNVYADSFVIRGDEVR | |
| | QIAPGQTGKIADYNYKLPDDFTGCVIAWNSNNLDSKVGGNYN | |
| | YLYRLFRKSNLKPFERDISTEIYQAGSTPCNGVEGENCYFPLQS | |
| | YGFQPTNGVGYQPYRVVVLSFELLHAPATVCGPKKSTNLVKN | |
| | KCVNFNFNGLTGTGVLTESNKKFLPFQQFGRDIADTTDAVRDPQT | |
| | LEILDITPCSFGGVSVITPGTNTSNQVAVLYQDVNCTEVPVAIHAD | |
| | QLTPTWRVYSTGSNVFQTRAGCLIGAEHVNNSYECDIPIGAGICAS | |
| | YQTQTNSPRRARSVASQSIIAYTMSLGAENSVAYSNNSIAIPTNFTI | |
| | SVTTEILPVSMTKTSVDCTMYICGDSTECSNLLLQYGSFCTQLNRA | |
| | LTGIAVEQDKNTQEVFAQVKQIYKTPPIKDFGGFNFSQILPDPSKPS | |
| | KRSFIEDLLFNKVTLADAGFIKQYGDCLGDIAARDLICAQKENGLT | |
| | VLPPLLTDEMIAQYTSALLAGTITSGWTFGAGAALQIPFAMQMAY | |
| | RFNGIGVTQNVLYENQKLIANQFNSAIGKIQDSLSSTASALGKLQD | |
| | VVNQNAQALNTLVKQLSSNFGAISSVLNDILSRLDKVEABVQIDR | |
| | LITGRLQSLQTYVTQQLIRAAEIRASANLAATKMSECVLGQSKRV | |
| | DFCGKGYHLMSFPQSAPHGVVFLHVTYVPAQEKNFTTAPAICHD | |
| | GKAHFPREGVFVSNGTHWFVTQRNFYEPQIITTDNTFVSGNCDVV | |
| | IGIVNNTVYDPLQPELDSFKEELDKYFKNHTSPDVDLGDISGINAS | |
| | VVNIQKEIDRLNEVAKNLNESLIDLQELKYEQYIKWPWYIWLGFIA | |
| | GLIAIVMVTIMLCCMTSCCSCLKGCCSCGSCCKFDEDDSEPVLKG | |
| | VKLHYT | |
| 77 | RVQPTESIVRFPNITNLCPFGEVENATRFASVYAWNRKRISNCVAD | Wuhan-Hu-1 S |
| | YSVLYNSASFSTFKCYGVSPTKLNDLCFTNVYADSFVIRGDEVRQI | protein |
| | APGQTGKIADYNYKLPDDFTGCVIAWNSNNLDSKVGGNYNYLYR | NCBI Reference |
| | LFRKSNLKPFERDISTEIYQAGSTPCNGVEGENCYFPLQSYGFQPTN | Sequence: |
| | GVGYQPYRVVVLSFELLHAPATVCGPKKSTNLVKNKCVNF | YP_009724390.1 |
| 78 | MFVFLVLLPLVSSQCVNLTTRTQLPPAYTNSFTRGVYYPDKVFRSS | RBD AA 319-541 |
| | VLHSTQDLFLPFFSNVTWFHAIHVSGTNGTKRFDNPVLPFNDGVY | Wuhan-Hu-1 S |
| | FASTEKSNIIRGWIFGTTLDSKTQSLLIVNNATNVVIKVCEFQFCND | protein with furin |
| | PFLGVYYHKNNKSWMESEFRVYSSANNCTFEYVSQPFLMDLEGK | site removed |
| | QGNFKNLREFVFKNIDGYFKIYSKHTPINLVRDLPQGFSALEPLVD | (AA685-686) and |
| | LPIGINITRFQTLLALHRSYLTPGDSSSGWTAGAAAYYVGYLQPRT | K986P and |
| | FLLKYNENGTITDAVDCALDPLSETKCTLKSFTVEKGIYQTSNFRV | V987P stabilizing |
| | QPTESIVRFPNITNLCPFGEVFNATRFASVYAWNRKRISNCVADYS | mutations (bold) |
| | VLYNSASFSTFKCYGVSPTKLNDLCFTNVYADSFVIRGDEVRQIAP | Extracellular |
| | GQTGKIADYNYKLPDDFTGCVIAWNSNNLDSKVGGNYNYLYRLF | domain only |
| | RKSNLKPFERDISTEIYQAGSTPCNGVEGENCYFPLQSYGFQPTNG | (AA1233-1273 |
| | VGYQPYRVVVLSFELLHAPATVCGPKKSTNLVKNKCVNFNFNGL | removed) NCBI |
| | TGTGVLTESNKKFLPFQQFGRDIADTTDAVRDPQTLEILDITPCSFG | Reference Sequence: |
| | GVSVITPGTNTSNQVAVLYQDVNCTEVPVAIHADQLTPTWRVYST | YP_009724390.1 |
| | GSNVFQTRAGCLIGAEHVNNSYECDIPIGAGICASYQTQTNSPRRA | |
| | VASQSIIAYTMSLGAENSVAYSNNSIAIPTNFTISVTTEILPVSMTKT | |
| | SVDCTMYICGDSTECSNLLLQYGSFCTQLNRALTGIAVEQDKNTQ | |
| | EVFAQVKQIYKTPPIKDFGGFNFSQILPDPSKPSKRSFIEDLLFNKVT | |
| | LADAGFIKQYGDCLGDIAARDLICAQKFNGLTVLPPLLTDEMIAQ | |
| | YTSALLAGTITSGWTFGAGAALQIPFAMQMAYRENGIGVTQNVL | |
| | YENQKLIANQFNSAIGKIQDSLSSTASALGKLQDVVNQNAQALNT | |
| | LVKQLSSNFGAISSVLNDILSRLDPPEAEVQIDRLITGRLQSLQTYV | |
| | TQQLIRAAEIRASANLAATKMSECVLGQSKRVDFCGKGYHLMSFP | |
| | QSAPHGVVFLHVTYVPAQEKNFTTAPAICHDGKAHFPREGVFVSN | |
| | GTHWFVTQRNFYEPQIITTDNTFVSGNCDVVIGIVNNTVYDPLQPE | |
| | LDSFKEELDKYFKNHTSPDVDLGDISGINASVVNIQKEIDRLNEVA | |
| | KNLNESLIDLQELKYEQYIKWPWYIWLGFIAGLIAIVMVTI | |
| 79 | MFVFLVLLPLVSSQCVNFTTRTQLPPAYTNSFTRGVYYPDKVFRSS | 7LYN |
| | VLHSTQDLFLPFFSNVTWFHAIHVSGTNGTKRFANPVLPFNDGVY | South African |
| | FASTEKSNIIRGWIFGTTLDSKTQSLLIVNNATNVVIKVCEFQFQND | (B.1.351) SARS- |
| | PFLGVYYHKNNKSWMESEFRVYSSANNCTFEYVSQPFLMDLEGK | CoV-2 spike |
| | QGNFKNLREFVFKNIDGYFKIYSKHTPINLVRGLPQGFSALEPLVD | protein variant |
| | LPIGINITRFQTLLALHISYLTPGDSSSGWTAGAAAYYVGYLQPRTF | (S-GSAS-B.1.351) |
| | LLKYNENGTITDAVDCALDPLSETKCTLKSFTVEKGIYQTSNFRVQ | |
| | PTESIVRFPNITNLCPFGEVENATRFASVYAWNRKRISNCVADYSV | |
| | LYNSASFSTFKCYGVSPTKLNDLCFTNVYADSFVIRGDEVRQIAPG | |
| | QTGNIADYNYKLPDDFTGCVIAWNSNNLDSKVGGNYNYLYRLFR | |
| | KSNLKPFERDISTEIYQAGSTPCNGVKGFNCYFPLQSYGFQPTYGV | |
| | GYQPYRVVVLSFELLHAPATVCGPKKSTNLVKNKCVNFNFNGLT | |
| | GTGVLTESNKKFLPFQQFGRDIADTTDAVRDPQTLEILDITPCSFGG | |
| | VSVITPGTNTSNQVAVLYQGVNCTEVPVAIHADQLTPTWRVYSTG | |
| | SNVFQTRAGCLIGAEHVNNSYECDIPIGAGICASYQTQTNSPGSAS | |
| | SVASQSIIAYTMSLGVENSVAYSNNSIAIPTNFTISVTTEILPVSMTK | |
| | TSVDCTMYICGDSTECSNLLLQYGSFCTQLNRALTGIAVEQDKNT | |
| | QEVFAQVKQIYKTPPIKDFGGFNFSQILPDPSKPSKRSFIEDLLENK | |
| | VTLADAGFIKQYGDCLGDIAARDLICAQKFNGLTVLPPLLTDEMIA | |
| | QYTSALLAGTITSGWTFGAGAALQIPFAMQMAYRENGIGVTQNV | |
| | LYENQKLIANQFNSAIGKIQDSLSSTASALGKLQDVVNQNAQALN | |
| | TLVKQLSSNFGAISSVLNDILSRLDKVEAEVQIDRLITGRLQSLQTY | |
| | VTQQLIRAAEIRASANLAATKMSECVLGQSKRVDFCGKGYHLMS | |
| | FPQSAPHGVVFLHVTYVPAQEKNFTTAPAICHDGKAHFPREGVFV | |
| | SNGTHWFVTQRNFYEPQIITTDNTFVSGNCDVVIGIVNNTVYDPLQ | |
| | PELDSFKEELDKYFKNHTSPDVDLGDISGINASVVNIQKEIDRLNE | |
| | VAKNLNESLIDLQELGKYEQGSGYIPEAPRDGQAYVRKDGEWVL | |
| | LSTFLGRSLEVLFQGPGHHHHHHHHSAWSHPQFEKGGGSGGGGS | |
| | GGSAWSHPQFEK | |
| 80 | MFVFLVLLPLVSSQCVNFTTRTQLPPAYTNSFTRGVYYPDKVFRSS | 7LYN |
| | VLHSTQDLFLPFFSNVTWFHAIHVSGTNGTKRFANPVLPENDGVY | South African |
| | FASTEKSNIIRGWIFGTTLDSKTQSLLIVNNATNVVIKVCEFQFCND | (B.1.351) SARS- |
| | PFLGVYYHKNNKSWMESEFRVYSSANNCTFEYVSQPFLMDLEGK | CoV-2 spike |
| | QGNFKNLREFVFKNIDGYFKIYSKHTPINLVRGLPQGFSALEPLVD | protein variant |
| | LPIGINITRFQTLLALHISYLTPGDSSSGWTAGAAAYYVGYLQPRTF | (S-GSAS-B.1.351) |
| | LLKYNENGTITDAVDCALDPLSETKCTLKSFTVEKGIYQTSNERVQ | with furin site |
| | PTESIVRFPNITNLCPFGEVENATRFASVYAWNRKRISNCVADYSV | removed and K986P |
| | LYNSASFSTFKCYGVSPTKLNDLCFTNVYADSFVIRGDEVRQIAPG | and V987P |
| | QTGNIADYNYKLPDDFTGCVIAWNSNNLDSKVGGNYNYLYRLFR | stabilizing |
| | KSNLKPFERDISTEIYQAGSTPCNGVKGENCYFPLQSYGFQPTYGV | mutations (bold) |
| | GYQPYRVVVLSFELLHAPATVCGPKKSTNLVKNKCVNFNFNGLT | Extracellular |
| | GTGVLTESNKKFLPFQQFGRDIADTTDAVRDPQTLEILDITPCSFGG | domain only |
| | VSVITPGTNTSNQVAVLYQGVNCTEVPVAIHADQLTPTWRVYSTG | |
| | SNVFQTRAGCLIGAEHVNNSYECDIPIGAGICASYQTQTNSPGSAS | |
| | SVASQSIIAYTMSLGVENSVAYSNNSIAIPTNFTISVTTEILPVSMTK | |
| | TSVDCTMYICGDSTECSNLLLQYGSFCTQLNRALTGIAVEQDKNT | |
| | QEVFAQVKQIYKTPPIKDFGGFNFSQILPDPSKPSKRSFIEDLLFNK | |
| | VTLADAGFIKQYGDCLGDIAARDLICAQKFNGLTVLPPLLTDEMIA | |
| | QYTSALLAGTITSGWTFGAGAALQIPFAMQMAYRENGIGVTQNV | |
| | LYENQKLIANQFNSAIGKIQDSLSSTASALGKLQDVVNQNAQALN | |
| | TLVKQLSSNFGAISSVLNDILSRLDPPEAEVQIDRLITGRLQSLQTY | |
| | VTQQLIRAAEIRASANLAATKMSECVLGQSKRVDFCGKGYHLMS | |
| | FPQSAPHGVVFLHVTYVPAQEKNFTTAPAICHDGKAHFPREGVFV | |
| | SNGTHWFVTQRNFYEPQIITTDNTFVSGNCDVVIGIVNNTVYDPLQ | |
| | PELDSFKEELDKYFKNHTSPDVDLGDISGINASVVNIQKEIDRLNE | |
| | VAKNLNESLIDLQELGKYEQGSGYIPEAPRDGQAYVRKDGEWVL | |
| | LSTFLGRSLEVLFQGPGHHHHHHHHSAWSHPQFEKGGGSGGGGS | |
| | GGSAWSHPQFEK | |
| 81 | QVQLRESGPS LVKPSQTLSL TCTVSGFSLS DKAVGWVRQA | IgHV1-7 V gene |
| | PGKALEWLGG IDTGGSTGYN PGLKSRLSIT KDNSKSQVSL | |
| | SVSSVTTEDS ATYYCTTVHQ | |
| 82 | SCPDGYSYGYGCGYGYGCSGYDCYGYGGYGGYGGYGYSSYSYS | IGHD8-2 |
| | YTYEY | |
| 83 | YVDAWGQGLLVTVSS | IGHJ2-4 |
| 84 | SNGVV | CDR H1 |
| 85 | DICSTGGTSFNPALKS | CDRH2 |
| 86 | SRGYDCYANVDALDYVDA | CDR H3 |
| 87 | DICSNGGTSFNPALKS | CDR H2 |
| 88 | SRGYDCYAYVYALDTVDA | CDR H3 |
| 89 | SRGYDCYANVDALDYVDA | CDRH3 |
| 90 | DNNVG | CDR H1 |
| 91 | VMHNDGNKGYNPALKS | CDR H2 |
| 92 | DNARCDSWTYDSCDTWYRNSWHVDA | CDR H3 |
| 93 | SYAVG | CDR H1 |
| 94 | DIDYVGNTDYNPALKS | CDR H2 |
| 95 | YSGAYAYAACNYYGWRCAWESYIDA | CDR H3 |
| 96 | NYAVG | CDR H1 |
| 97 | DVDSSGGTAYNPALKS | CDR H2 |
| 98 | FAKGTTSAGACDYSESYVDA | CDR H3 |
| 99 | DYAVG | CDR H1 |
| 100 | GIDTGGSTGYNPGLKS | CDR H2 |
| 101 | VVLCYYNRVVRRNNCGGLGYDYGFDHFYVDA | CDR H3 |
| 102 | GIDTGGSTGYNPGLES | CDR H2 |
| 103 | VVLCYFNYVVRRYNCGGLGYGHGENSFYVDA | CDR H3 |
| 104 | VVLCYFNYVVRRYNCGGLGYGHGENSFYVDA | CDR H3 |
| 105 | SYGIT | CDR H1 |
| 106 | SISSGGTTDYNPALKS | CDR H2 |
| 107 | WNLEYTWGGVGCASFADEDTHVDA | CDR H3 |
| 108 | DKAVG | CDR H1 |
| 109 | SIDTGGSTGYNPGLKS | CDR H2 |
| 110 | WGQGLLVTVSS | CDR H3 |
| 111 | SIDTAENTGYNPGLKS | CRD H2 |
| 112 | ATVHQKTRKEKNCPDGYIYSSNTASGYDCGVWICRRVGSAFCSRT | CDR H3 |
| | GDYTSPSEFDIYEFYVEG | |
| 113 | DKPVG | CDR H1 |
| 114 | ATVHQKTRKEKSCPDGYLYSSNTGRGYDCGVWTCRRVGGEFCSA | CDR H3 |
| | TGDWTSPSEEDFYEFYVDT | |
| 115 | SIDTGGNTGYNPGLKS | CDR H2 |
| 116 | TTVHQRTSEKRSCPGGSSRRYPSGASCDVSGGACACYVSNCRGVL | CDR H3 |
| | CPTLNEIVAYTYEWHVDA | |
| 117 | TTVHQETLRSCPDGYIDNSGCTADWGCAALDCWRRRFGYHSTDP | CDR H3 |
| | SHYTGATYIYTYSLHIDAW | |
| 118 | SIDTGGSTGYNPGLKS | CDR H2 |
| 119 | ATVHQKTAEGDKTCPDGYEHTCGCIGGCGCKRSACIGALCCQAS | CDR H3 |
| | LGGWLSDGETYTYEFHVDT | |
| 120 | SIDTGGMTGYNPGLKS | CDR H2 |
| 121 | ATVDQKTKNACPDDFDYRCSCIGGCGCARKGCVGPLCCRSDLGG | CDR H3 |
| | YLTDSPAYIYEWYIDL | |
| 122 | SYAVL | CDR H1 |
| 123 | ATVHQKTRKEKNCPDGYIYSSNITSGFDCGVWICRRVGSAFCSRT | CDR H3 |
| | GDYTSPTELDIYEFYVEG | |
| 124 | DKAIG | CDR H1 |
| 125 | SIDTRGNTGYNPGLKS | CDR H2 |
| 126 | AIVQQITHKTCPNGYNWFDRCCSWDGTCGDGCCSNRAWPSGNG | CDR H3 |
| | RADSSIGETYGYEFHVAA | |
| 127 | CTTVHQ | Stalk A |
| 128 | CATVHQ | Stalk A |
| 129 | CAIVQQ | Stalk A |
| 130 | CATVDQ | Stalk A |
| 131 | YX₁YX₂Y, where X₁ and X₂ are any amino acid | Tyrosine motif |
| 132 | QLQLQESGPGLVKPSETLSLTCTVSGGSISSSSYYWGWIRQP | Human heavy chain |
| | PGKGLEWIGSIYYSGSTYYNPSLKSRVTISVDTSKNQFSLKLS | variable region |
| | SVTAADTAVYYCAR | sequence VH4-39 |
| 133 | QVQLQESGPGLVKPSETLSLTCTVSGGSISSYYWSWIRQPPG | Human heavy chain |
| | KGLEWIGYIYYSGSTNYNPSLKSRVTISVDTSKNQFSLKLSS | variable region |
| | VTAADTAVYYCA | sequence |
| | | 4-59*03 |
| 134 | QVQLQQWGAGLLKPSETLSLTCAVYGGSFSGYYWSWIRQP | Human heavy chain |
| | PGKGLEWIGEINHSGSTNYNPSLKSRVTISVDTSKNQFSLKLS | variable region |
| | SVTAADTAVYYCAR | sequence |
| | | 4-34*02 |
| 135 | QVQLQESGPGLVKPSQTLSLTCAVYGGSFSGYYWSWIRQPP | Human heavy chain |
| | GKGLEWIGEINHSGSTNYNPSLKSRVTISVDTSKNQFSLKLSS | variable region |
| | VTAADTAVYYCAR | sequence |
| | | 4-34*09 |
| 136 | cagctgcagctgcaggagtcgggcccaggactggtgaagccttcggagaccctgtccctcac | Human heavy chain |
| | ctgcactgtctctggtggctccatcagcagtagtagttactactggggctggatccgccagcc | variable region |
| | cccagggaaggggctggagtggattgggagtatctattatagtgggagcacctactacaaccc | sequence 4-39 |
| | gtccctcaagagtcgagtcaccatatccgtagacacgtccaagaaccagttctccctgaagct | |
| | gagctctgtgaccgccgcagacacggctgtgtattactgtgcgagacacacagtgagggg | |
| 137 | caggtgcagctgcaggagtcgggcccaggactggtgaagccttcggagaccctgtccctcac | Human heavy chain |
| | ctgcactgtctctggtggctccatcagtagttactactggagctggatccggcagcccccagg | variable region |
| | gaagggactggagtggattgggtatatctattacagtgggagcaccaactacaacccctccct | sequence 4-59*03 |
| | caagagtcgagtcaccatatcagtagacacgtccaagaaccaattctccctgaagctgagctc | |
| | tgtgaccgctgcggacacggccgtgtattactgtgcg | |
| 138 | caggtgcagctgcaggagtcgggcccaggactggtgaagccttcacagaccctgtccctcac | Human heavy chain |
| | ctgcgctgtctatggtgggtccttcagtggttactactggagctggatccgccagcccccagg | variable region |
| | gaagggactggagtggattggggaaatcaatcatagtggaagcaccaactacaacccgtccct | sequence 4-34*09 |
| | caagagtcgagttaccatatcagtagacacgtctaagaaccagttctccctgaagctgagctc | |
| | tgtgactgccgcggacacggccgtgtattactgtgcgaga | |
| 139 | caggtgcagctacaacagtggggcgcaggactgttgaagccttcggagaccctgtccctcacc | Human heavy chain |
| | tgcgctgtctatggtgggtccttcagtggttactactggagctggatccgccagcccccaggga | variable region |
| | aggggctggagtggattggggaaatcaatcatagtggaagcaccaactacaacccgtccctca | sequence 4-34*02 |
| | agagtcgagtcaccatatcagtagacacgtccaagaaccagttctccctgaagctgagctctgt | |
| | gaccgccgcggacacggctgtgtattactgtgcgag | |
| 140 | QVQLRESGPSLVKPSQTLSLTCTASGFSLSDKAVGWVRQAP | BLV1H12 V1 |
| | GKALEWLGSIDTGGNTGYNPGLKSRLSITKDNSKSQVSLSVS | Region |
| | SVTTEDSATYY | |
| 141 | QVQLREWGAGLLKPSETLSLTCAVYGGSFSDKYWSWIRQPP | Humanized V |
| | GKGLEWIGSINHSGSTNYNPSLKSRVTISVDTSKNQFSLKLSS | region |
| | VTAADTAVYY | |
| 142 | WGHGTAVTVSS | V2 alternative |
| | | sequence |
| 143 | WGKGTTVTVSS | V2 alternative |
| | | sequence |
| 144 | WGKGTTVTVSS | V2 alternative |
| | | sequence |
| 145 | WGRGTLVTVSS | V2 alternative |
| | | sequence |
| 146 | WGKGTTVTVSS | V2 alternative |
| | | sequence |
| 147 | WGQGLLVTVSS | V2 alternative |
| | | sequence |
| 148 | ASTKGPSVFPLAPSSKSTSGGTAALGCLVKDYFPEPVTVSWN | Human IgGI Heavy |
| | SGALTSGVHTFPAVLQSSGLYSLSSVVTVPSSSLGTQTYICN | Constant Region |
| | VNHKPSNTKVDKKVEPKSCDKTHTCPPCPAPELLGGPSVFLF | |
| | PPKPKDTLMISRTPEVTCVVVDVSHEDPEVKFNWYVDGVEV | |
| | HNAKTKPREEQYNSTYRVVSVLTVLHQDWLNGKEYKCKVS | |
| | NKALPAPIEKTISKAKGQPREPQVYTLPPSRDELTKNQVSLT | |
| | CLVKGFYPSDIAVEWESNGQPENNYKTTPPVLDSDGSFFLYS | |
| | KLTVDKSRWQQGNVFSCSVMHEALHNHYTQKSLSLSPGK | |
| 149 | QAVLNQPSSVSGSLGQRVSITCSGSSSNVGNGYVSWYQLIPG | BLV1H12 Light |
| | SAPRTLIYGDTSRASGVPDRFSGSRSGNTATLTISSLQAEDEA | Chain |
| | DYFCASAEDSSSNAVFGSGTTLTVLGQPKSPPSVTLFPPSTEE | |
| | LNGNKATLVCLISDFYPGSVTVVWKADGSTITRNVETTRAS | |
| | KQSNSKYAASSYLSLTSSDWKSKGSYSCEVTHEGSTVTKTV | |
| | KPSECS | |
| 150 | QVQLRESGPSLVQPSQTLSLTCTASGFSLSDKAVGWVRQAP | BLV5B8 heavy |
| | GKALEWLGSIDTGGSTGYNPGLKSRLSITKDNSKSQVSLSVS | chain |
| | SVTTEDSATYYCTTVHQETRKTCSDGYIAVDSCGRGQSDGC | |
| | VNDCNSCYYGWRNCRRQPAIHSYEFHVDAWGRGLLVTVSS | |
| | ASTTAPKVYPLSSCCGDKSSSTVTLGCLVSSYMPEPVTVTW | |
| | NSGALKSGVHTFPAVLQSSGLYSLSSMVTVPGSTS | |
| | GQTFTCNVAHPASSTKVDKAVEPKSCDGS | |
| 151 | SGSSSNVGNGYVS | CDR L1 |
| 152 | GDTSRAS | CDR L2 |
| 153 | ASAEDSSSNAV | CDR L3 |
| 154 | QSVLTQPPSVSAAPGQKVTISCSGSSSNIGNNYVSWYQQLPG | Human VL 1-51 |
| | TAPKLLIYDNNKRPSGIPDRFSGSKSGTSATLGITGLQTGDEA | |
| | DYYCASAEDSSSNAVFGSGTTLTVLGQPKAAPSVTLFPPSSE | |
| | ELQANKATLVCLISDFYPGAVTVAWKADSSPVKAGVETTTP | |
| | SKQSNNKYAASSYLSLTPEQWKSHRSYSCQVTHEGSTVEKT | |
| | VAPTECS | |
| 155 | QSVLTQPPSASGTPGQRVTISCSGSSSNIGSNYVYWYQQLPG | Human germline |
| | TAPKLLIYRNNQRPSGVPDRFSGSKSGTSASLAISGLRSEDEA | light chain variable |
| | DYYCAAWDDSLSG | region sequence |
| | | VL1-47 |
| 156 | QSVLTQPPSVSGAPGQRVTISCTGSSSNIGAGYDVHWYQQLP | Human germline |
| | GTAPKLLIYGNSNRPSGVPDRFSGSKSGTSASLAITGLQAED | light chain variable |
| | EADYYCQSYDSSLSG | region sequence |
| | | VL1-40*1 |
| 157 | QSVLTQPPSVSAAPGQKVTISCSGSSSNIGNNYVSWYQQLPG | Human germline |
| | TAPKLLIYDNNKRPSGIPDRFSGSKSGTSATLGITGLQTGDEA | light chain variable |
| | DYYCGTWDSSLSA | region sequence |
| | | VL1-51*01 |
| 158 | QSALTQPPSVSGSPGQSVTISCTGTSSDVGSYNRVSWYQQPP | Human germline |
| | GTAPKLMIYEVSNRPSGVPDRFSGSKSGNTASLTISGLQAED | light chain variable |
| | EADYYCSSYTSSSTF | region sequence |
| | | VL2-18*02 |
| 159 | RKEKNCPDGYIYSSNTASGYDCGVWICRRVGSAFCSRTGDYTSPS | R4C1 knob |
| | EFDIYEFY | |
| 160 | RKEKNCPDGYIYSSNTASGYDCGVWICRRVGSAFCSRTGDYTS | R4C1 knob |
| 161 | RKEKNCPDGYIYSSNTASGYDCGVWICRRVGSAFCSRTGDYTSP | R4C1 knob |
| 162 | RKEKNCPDGYIYSSNTASGYDCGVWICRRVGSAFCSRTGDYTSPS | R4C1 knob |
| 163 | RKEKNCPDGYIYSSNTASGYDCGVWICRRVGSAFCSRTGDYTSPS | R4C1 knob |
| | E | |
| 164 | RKEKNCPDGYIYSSNTASGYDCGVWICRRVGSAFCSRTGDYTSPS | R4C1 knob |
| | EF | |
| 165 | RKEKNCPDGYIYSSNTASGYDCGVWICRRVGSAFCSRTGDYTSPS | R4C1 knob |
| | EFD | |
| 166 | RKEKNCPDGYIYSSNTASGYDCGVWICRRVGSAFCSRTGDYTSPS | R4C1 knob |
| | EFDI | |
| 167 | RKEKNCPDGYIYSSNTASGYDCGVWICRRVGSAFCSRTGDYTSPS | R4C1 knob |
| | EFDIYE | |
| 168 | RKEKNCPDGYIYSSNTASGYDCGVWICRRVGSAFCSRTGDYTSPS | R4C1 knob |
| | EFDIYEF | |
| 169 | RKEKSCPDGYLYSSNTGRGYDCGVWTCRRVGGEFCSATGDWTS | R2C3 knob |
| 170 | RKEKSCPDGYLYSSNTGRGYDCGVWTCRRVGGEFCSATGDWTSP | R2C3 knob |
| 171 | RKEKSCPDGYLYSSNTGRGYDCGVWTCRRVGGEFCSATGDWTSP | R2C3 knob |
| | S | |
| 172 | RKEKSCPDGYLYSSNTGRGYDCGVWTCRRVGGEFCSATGDWTSP | R2C3 knob |
| | SE | |
| 173 | RKEKSCPDGYLYSSNTGRGYDCGVWTCRRVGGEFCSATGDWTSP | R2C3 knob |
| | SEE | |
| 174 | RKEKSCPDGYLYSSNTGRGYDCGVWTCRRVGGEFCSATGDWTSP | R2C3 knob |
| | SEED | |
| 175 | RKEKSCPDGYLYSSNTGRGYDCGVWTCRRVGGEFCSATGDWTSP | R2C3 knob |
| | SEEDF | |
| 176 | RKEKSCPDGYLYSSNTGRGYDCGVWTCRRVGGEFCSATGDWTSP | R2C3 knob |
| | SEEDFY | |
| 177 | RKEKSCPDGYLYSSNTGRGYDCGVWTCRRVGGEFCSATGDWTSP | R2C3 knob |
| | SEEDFYE | |
| 178 | SEKRSCPGGSSRRYPSGASCDVSGGACACYVSNCRGVLCPTLNEI | SKD knob |
| | VAYTYEWHVD | |
| 179 | SEKRSCPGGSSRRYPSGASCDVSGGACACYVSNCRGVLCPTLNEI | SKD knob |
| 180 | SEKRSCPGGSSRRYPSGASCDVSGGACACYVSNCRGVLCPTLNEI | SKD knob |
| | V | |
| 181 | SEKRSCPGGSSRRYPSGASCDVSGGACACYVSNCRGVLCPTLNEI | SKD knob |
| | VA | |
| 182 | SEKRSCPGGSSRRYPSGASCDVSGGACACYVSNCRGVLCPTLNEI | SKD knob |
| | VAY | |
| 183 | SEKRSCPGGSSRRYPSGASCDVSGGACACYVSNCRGVLCPTLNEI | SKD knob |
| | VAYT | |
| 184 | SEKRSCPGGSSRRYPSGASCDVSGGACACYVSNCRGVLCPTLNEI | SKD knob |
| | VAYTYE | |
| 185 | SEKRSCPGGSSRRYPSGASCDVSGGACACYVSNCRGVLCPTLNEI | SKD knob |
| | VAYTYEW | |
| 186 | SEKRSCPGGSSRRYPSGASCDVSGGACACYVSNCRGVLCPTLNEI | SKD knob |
| | VAYTYEWH | |
| 187 | SEKRSCPGGSSRRYPSGASCDVSGGACACYVSNCRGVLCPTLNEI | SKD knob |
| | VAYTYEWHV | |
| 188 | LRSCPDGYIDNSGCTADWGCAALDCWRRRFGYHSTDPSHYTGAT | SKM knob |
| | YIYTYSHID | |
| 189 | LRSCPDGYIDNSGCTADWGCAALDCWRRRFGYHSTDPSHYTGAT | SKM knob |
| 190 | LRSCPDGYIDNSGCTADWGCAALDCWRRRFGYHSTDPSHYTGAT | SKM knob |
| | Y | |
| 191 | LRSCPDGYIDNSGCTADWGCAALDCWRRRFGYHSTDPSHYTGAT | SKM knob |
| | YI | |
| 192 | LRSCPDGYIDNSGCTADWGCAALDCWRRRFGYHSTDPSHYTGAT | SKM knob |
| | YIY | |
| 193 | LRSCPDGYIDNSGCTADWGCAALDCWRRRFGYHSTDPSHYTGAT | SKM knob |
| | YIYT | |
| 194 | LRSCPDGYIDNSGCTADWGCAALDCWRRRFGYHSTDPSHYTGAT | SKM knob |
| | YIYTYS | |
| 195 | LRSCPDGYIDNSGCTADWGCAALDCWRRRFGYHSTDPSHYTGAT | SKM knob |
| | YIYTYS | |
| 196 | LRSCPDGYIDNSGCTADWGCAALDCWRRRFGYHSTDPSHYTGAT | SKM knob |
| | YIYTYSH | |
| 197 | LRSCPDGYIDNSGCTADWGCAALDCWRRRFGYHSTDPSHYTGAT | SKM knob |
| | YIYTYSHI | |
| 198 | EGDKTCPDGYEHTCGCIGGCGCKRSACIGALCCQASLGGWLS | R2G3 knob |
| 199 | EGDKTCPDGYEHTCGCIGGCGCKRSACIGALCCQASLGGWLSD | R2G3 knob |
| 200 | EGDKTCPDGYEHTCGCIGGCGCKRSACIGALCCQASLGGWLSDG | R2G3 knob |
| 201 | EGDKTCPDGYEHTCGCIGGCGCKRSACIGALCCQASLGGWLSDG | R2G3 knob |
| | E | |
| 202 | EGDKTCPDGYEHTCGCIGGCGCKRSACIGALCCQASLGGWLSDG | R2G3 knob |
| | ET | |
| 203 | EGDKTCPDGYEHTCGCIGGCGCKRSACIGALCCQASLGGWLSDG | R2G3 knob |
| | ETY | |
| 204 | EGDKTCPDGYEHTCGCIGGCGCKRSACIGALCCQASLGGWLSDG | R2G3 knob |
| | ETYT | |
| 205 | EGDKTCPDGYEHTCGCIGGCGCKRSACIGALCCQASLGGWLSDG | R2G3 knob |
| | ETYTY | |
| 206 | EGDKTCPDGYEHTCGCIGGCGCKRSACIGALCCQASLGGWLSDG | R2G3 knob |
| | ETYTYE | |
| 207 | KTKNACPDDFDYRCSCIGGCGCARKGCVGPLCCRSDLGGYLTDSP | R2F12 knob |
| | AYIYEWY | |
| 208 | KTKNACPDDFDYRCSCIGGCGCARKGCVGPLCCRSDLGGYLTDS | R2F12 knob |
| 209 | KTKNACPDDFDYRCSCIGGCGCARKGCVGPLCCRSDLGGYLTDSP | R2F12 knob |
| 210 | KTKNACPDDFDYRCSCIGGCGCARKGCVGPLCCRSDLGGYLTDSP | R2F12 knob |
| | A | |
| 211 | KTKNACPDDFDYRCSCIGGCGCARKGCVGPLCCRSDLGGYLTDSP | R2F12 knob |
| | AY | |
| 212 | KTKNACPDDFDYRCSCIGGCGCARKGCVGPLCCRSDLGGYLTDSP | R2F12 knob |
| | AYI | |
| 213 | KTKNACPDDFDYRCSCIGGCGCARKGCVGPLCCRSDLGGYLTDSP | R2F12 knob |
| | AYIY | |
| 214 | KTKNACPDDFDYRCSCIGGCGCARKGCVGPLCCRSDLGGYLTDSP | R2F12 knob |
| | AYIYEW | |
| 215 | KEKNCPDGYIYSSNITSGFDCGVWICRRVGSAFCSRTGDYTS | SR3A3 knob |
| 216 | KEKNCPDGYIYSSNITSGFDCGVWICRRVGSAFCSRTGDYTSP | SR3A3 knob |
| 217 | KEKNCPDGYIYSSNITSGFDCGVWICRRVGSAFCSRTGDYTSPT | SR3A3 knob |
| 218 | KEKNCPDGYIYSSNITSGFDCGVWICRRVGSAFCSRTGDYTSPTE | SR3A3 knob |
| 219 | KEKNCPDGYIYSSNITSGFDCGVWICRRVGSAFCSRTGDYTSPTEL | SR3A3 knob |
| 220 | KEKNCPDGYIYSSNITSGFDCGVWICRRVGSAFCSRTGDYTSPTEL | SR3A3 knob |
| | D | |
| 221 | KEKNCPDGYIYSSNITSGFDCGVWICRRVGSAFCSRTGDYTSPTEL | SR3A3 knob |
| | DI | |
| 222 | KEKNCPDGYIYSSNITSGFDCGVWICRRVGSAFCSRTGDYTSPTEL | SR3A3 knob |
| | DIY | |
| 223 | KEKNCPDGYIYSSNITSGFDCGVWICRRVGSAFCSRTGDYTSPTEL | SR3A3 knob |
| | DIYE | |
| 224 | ITHKTCPNGYNWFDRCCSWDGTCGDGCCSNRAWPSGNGRADSS | R2D9 knob |
| 225 | ITHKTCPNGYNWFDRCCSWDGTCGDGCCSNRAWPSGNGRADSSI | R2D9 knob |
| 226 | ITHKTCPNGYNWFDRCCSWDGTCGDGCCSNRAWPSGNGRADSSI | R2D9 knob |
| | G | |
| 227 | ITHKTCPNGYNWFDRCCSWDGTCGDGCCSNRAWPSGNGRADSSI | R2D9 knob |
| | GE | |
| 228 | ITHKTCPNGYNWFDRCCSWDGTCGDGCCSNRAWPSGNGRADSSI | R2D9 knob |
| | GET | |
| 229 | ITHKTCPNGYNWFDRCCSWDGTCGDGCCSNRAWPSGNGRADSSI | R2D9 knob |
| | GETY | |
| 230 | ITHKTCPNGYNWFDRCCSWDGTCGDGCCSNRAWPSGNGRADSSI | R2D9 knob |
| | GETYG | |
| 231 | ITHKTCPNGYNWFDRCCSWDGTCGDGCCSNRAWPSGNGRADSSI | R2D9 knob |
| | GETYGY | |
| 232 | ITHKTCPNGYNWFDRCCSWDGTCGDGCCSNRAWPSGNGRADSSI | R2D9 knob |
| | GETYGYE | |
| 233 | MFVFLVLLPLVSSQCVNLTTRTQLPPAYTNSFTRGVYYPDKVFRSS | Spike protein |
| | VLHSTQDLFLPFFSNVTWFHAIHVSGTNGTKRFDNPVLPFNDGVY | |
| | FASTEKSNIIRGWIFGTTLDSKTQSLLIVNNATNVVIKVCEFQFCND | |
| | PFLGVYYHKNNKSWMESEFRVYSSANNCTFEYVSQPFLMDLEGK | |
| | QGNFKNLREFVFKNIDGYFKIYSKHTPINLVRDLPQGFSALEPLVD | |
| | LPIGINITRFQTLLALHRSYLTPGDSSSGWTAGAAAYYVGYLQPRT | |
| | FLLKYNENGTITDAVDCALDPLSETKCTLKSFTVEKGIYQTSNFRV | |
| | QPTESIVRFPNITNLCPFGEVENATRFASVYAWNRKRISNCVADYS | |
| | VLYNSASFSTFKCYGVSPTKLNDLCFTNVYADSFVIRGDEVRQIAP | |
| | GQTGKIADYNYKLPDDFTGCVIAWNSNNLDSKVGGNYNYLYRLF | |
| | RKSNLKPFERDISTEIYQAGSTPCNGVEGENCYFPLQSYGFQPTNG | |
| | VGYQPYRVVVLSFELLHAPATVCGPKKSTNLVKNKCVNFNFNGL | |
| | TGTGVLTESNKKFLPFQQFGRDIADTTDAVRDPQTLEILDITPCSFG | |
| | GVSVITPGTNTSNQVAVLYQDVNCTEVPVAIHADQLTPTWRVYST | |
| | GSNVFQTRAGCLIGAEHVNNSYECDIPIGAGICASYQTQTNSPRRA | |
| | RSVASQSIIAYTMSLGAENSVAYSNNSIAIPTNFTISVTTEILPVSMT | |
| | KTSVDCTMYICGDSTECSNLLLQYGSFCTQLNRALTGIAVEQDKN | |
| | TQEVFAQVKQIYKTPPIKDFGGFNFSQILPDPSKPSKRSFIEDLLENK | |
| | VTLADAGFIKQYGDCLGDIAARDLICAQKFNGLTVLPPLLTDEMIA | |
| | QYTSALLAGTITSGWTFGAGAALQIPFAMQMAYRENGIGVTQNV | |
| | LYENQKLIANQFNSAIGKIQDSLSSTASALGKLQDVVNQNAQALN | |
| | TLVKQLSSNFGAISSVLNDILSRLDKVEAEVQIDRLITGRLQSLQTY | |
| | VTQQLIRAAEIRASANLAATKMSECVLGQSKRVDFCGKGYHLMS | |
| | FPQSAPHGVVFLHVTYVPAQEKNFTTAPAICHDGKAHFPREGVFV | |
| | SNGTHWFVTQRNFYEPQIITTDNTFVSGNCDVVIGIVNNTVYDPLQ | |
| | PELDSFKEELDKYFKNHTSPDVDLGDISGINASVVNIQKEIDRLNE | |
| | VAKNLNESLIDLQELGKYEQYIKWPWYIWLGFIAGLIAIVMVTIML | |
| | CCMTSCCSCLKGCCSCGSCCKFDEDDSEPVLKGVKLHYT | |
| 234 | GGGGAMGS | Linker |
| 235 | DDDDK | Cleavable linker |
| 236 | GGSEGDKTCPDGYEHTCGCIGGCGCKRSACIGALCCQASLGGWL | G3 Parental |
| | SD | |
| 237 | GGSGDKTCPDGYEHTCGCIGGCGCKRSACIGALCCQASLGGWLS | G3 NTRUNC1 |
| | D | |
| 238 | GGSDKTCPDGYEHTCGCIGGCGCKRSACIGALCCQASLGGWLSD | G3 NTRUNC2 |
| 239 | GGSKTCPDGYEHTCGCIGGCGCKRSACIGALCCQASLGGWLSD | G3 NTRUNC3 |
| 240 | GGSTCPDGYEHTCGCIGGCGCKRSACIGALCCQASLGGWLSD | G3 NTRUNC4 |
| 241 | GGSCPDGYEHTCGCIGGCGCKRSACIGALCCQASLGGWLSD | G3 NTRUNCS |

What is claimed:

1. An isolated knob peptide of over 30 amino acids in length directed against SARS-CoV-2, wherein the knob peptide is isolated or derived from an ultralong CDR3 of a bovine antibody.

2. The isolated knob peptide of claim 1, wherein the knob peptide comprises a contiguous sequence of amino acids within the sequence set forth in any one of SEQ ID NOS: 112, 114, 116, 117, 119, 121, 123, or 126, wherein the knob peptide is up to 70 amino acids in length and contains 2-6 disulfide bonds.

3. A knob peptide directed against SARS-CoV-2 spike protein, comprising a contiguous sequence of amino acids of at least 40 amino acids within the sequence set forth in any one of SEQ ID NOS: 112, 114, 116, 117, 119, 121, 123, or 126, wherein the knob peptide is up to 70 amino acids in length and contains 2-6 disulfide bonds.

4. The knob peptide of any of claims 1-3, wherein the knob peptide comprises 6-9 C-terminal amino acids after the last cysteine (Cys) residue of the respective sequence of amino acids set forth in any one of SEQ ID NOS: 112, 114, 116, 117, 119, 121, 123, or 126, optionally at or about 9 C-terminal amino acids after the last cysteine (Cys) residue of the respective sequence of amino acids set forth in any one of SEQ ID NOS: 112, 114, 116, 117, 119, 121, 123, or 126.

5. The knob peptide of any of claims 1-4, wherein the sequence of the knob peptide begins 3-6 amino acids before the N-terminal Cys residue of the respective sequence of amino acids set forth in any one of SEQ ID NOS: 112, 114, 116, 117, 119, 121, 123, or 126.

6. The knob peptide of any of claims 1-5, wherein the knob peptide comprises at or about 9 C-terminal amino acids after the last cysteine (Cys) residue of the respective sequence of amino acids set forth in any one of SEQ ID NOS: 112, 114, 116, 117, 119, 121, 123, or 126.

7. The knob peptide of any of claims 1-6, wherein the knob peptide is 40 to 60 amino acids in length, optionally wherein the knob peptide is at least 42 amino acids in length.

8. The knob peptide of any of claims 1-7, wherein the knob peptide is 42 amino acids, 43 amino acids, 44 amino acids, 45 amino acids, 46 amino acids, 47 amino acids, 48 amino acids, 49 amino acids, 50 amino acids, 51 amino acids, 52 amino acids, 53 amino acids, 54 amino acids, 55 amino acids, 56 amino acids, 57 amino acids, 58 amino acids, 59 amino acids or 60 amino acids in length.

9. The knob peptide of any of claims 1-8, wherein the knob peptide comprises at least 4 Cys residues, optionally wherein the knob peptide contains 4 Cys residues.

10. The knob peptide of any of claims 1-8, wherein the knob peptide contains 6, 8, 10 or 12 Cys residues.

11. The knob peptide of any of claims 1-10, wherein the knob peptide has at least 2 disulfide bonds.

12. The knob peptide of any of claims 1-11, wherein the knob peptide has 2 disulfide bonds.

13. The knob peptide of any of claims 1-11, wherein the knob peptide has 3, 4 or 5 disulfide bonds.

14. The knob peptide of any of claims 1-13, wherein the knob peptide comprises an amino acid sequence set forth in any one of SEQ ID NOS: 160, 169, 179, 189, 198, 208, 215 or 224.

15. The knob peptide of any of claims 1-14, wherein the knob peptide is at or about 42 amino acids, 43 amino acids, 44 amino acids, 45 amino acids, 46 amino acids, 47 amino acids, 48 amino acids, 49 amino acids, 50 amino acids, 51 amino acids, 52 amino acids, 53 amino acids, 54 amino acids, or 55 amino acids.

16. The knob peptide of any of claims 1-14, wherein the knob peptide comprises the amino acid sequence set forth in SEQ ID NO:160.

17. The knob peptide of claim 16, wherein the knob peptide is set forth in any one of SEQ ID NOS: 63 or 159-168.

18. The knob peptide of any of claims 1-14, wherein the knob peptide comprises the amino acid sequence set forth in SEQ ID NO. 169.

19. The knob peptide of any of claims 1-14 and 18, wherein the knob peptide is set forth in any one of SEQ ID NOS: 66 or 169-177.

20. The knob peptide of any of claims 1-14, wherein the knob peptide comprises the amino acid sequence set forth in SEQ ID NO:179.

21. The knob peptide of any of claims 1-14 and 20, wherein the knob peptide is set forth in any one of SEQ ID NOS: 65 or 178-187.

22. The knob peptide of any of claims 1-14, wherein the knob peptide comprises the amino acid sequence set forth in SEQ ID NO:189.

23. The knob peptide of any of claims 1-14 and 22, wherein the knob peptide is set forth in any one of SEQ ID NOS: 64 or 188-197.

24. The knob peptide of any of claims 1-14, wherein the knob peptide comprise the amino acid sequence set forth in SEQ ID NO:198.

25. The knob peptide of any of claims 1-14 and 24, wherein the knob peptide is set forth in any one of SEQ ID NOS: 60 or 198-206.

26. The knob peptide of any of claims 1-14, wherein the knob peptide comprises the amino acid sequence set forth in SEQ ID NO:208.

27. The knob peptide of any of claims 1-14 and 26, wherein the knob peptide is set forth in any one of SEQ ID NOS: 62 or 207-214.

28. The knob peptide of any of claims 1-14, wherein the knob peptide comprises the amino acid sequence set forth in SEQ ID NO:215.

29. The knob peptide of any of claims 1-14 and 28, wherein the knob peptide is set forth in any of SEQ ID NOS: 61 and 215-223.

30. The knob peptide of any of claims 1-14, wherein the knob peptide comprises the amino acid sequence set forth in SEQ ID NO:224.

31. The knob peptide of any of claims 1-14 and 30, wherein the knob peptide is set forth in any one of SEQ ID NOS: 68 or 224-232.

32. The knob peptide of any of claims 1-31, wherein the knob peptide further comprises an N-terminal or C-terminal linker, optionally a GS linker.

33. The knob peptide of any of claims 1-32, wherein the knob peptide further comprises a linker for cyclization of the peptide.

34. The knob peptide of claim 33, wherein the linker is GGGGAMGS (SEQ ID NO: 234).

35. The knob peptide of any of claims 1-34, wherein the peptide is cyclized.

36. The knob peptide of any of claims 1-35, wherein the knob peptide binds to the spike protein of SARS-CoV2 or a binding portion or epitope thereof with a binding affinity dissociation constant of less than at or about 10⁻⁹ M, less than at or about 10⁻¹⁰ M, less than at or about 10⁻¹¹ M or less than at or about 10⁻¹² M.

37. The knob peptide of any of claims 1-36, wherein the knob peptide neutralizes SARS-CoV2 infection of a human cell.

38. The knob peptide of any of claims 1-37, wherein the knob peptide neutralizes SARS-CoV2 with an EC50 of less than about 100 ng/mL, 10 ng/mL, 1 ng/mL, 0.1 ng/mL, 0.01 ng/mL, 0.001 ng/mL.

39. The knob peptide of any of claims 1-37, wherein the knob peptide neutralizes SARS-COV2 with an EC50 of less than about 100 nM, 10 nM, 1 nM, 0.1 nM, 0.01 nM or 0.001 nM.

40. The knob peptide of any of claims 1-39, wherein the knob peptide neutralizes SARS-COV2 with an EC50 of less than at or about 500 pM, 250 pM, 100 pM, 50 pM, 25 pM, 10 pM, 5 pM, 2.5 pM, 1 pM, 0.5 pM or less.

41. The knob peptide of any of claims 1-40, wherein the SARS CoV-2 is selected from Wuhan-Hu-1 strain, UK (B.1.1.7) strain or South African (B.1.351) strain.

42. The knob peptide of any of claims 1-40, wherein the SARS CoV-2 is Wuhan-Hu-1 strain.

43. The knob peptide of any of claims 1-42, wherein the knob peptide is recombinantly produced.

44. The knob peptide of any of claims 1-43, wherein the knob peptide is purified.

45. A multispecific binding protein, comprising a plurality of knob peptides of any of claims 1-44 and 103-105.

46. The multispecific binding protein of claim 45, wherein the plurality of knob peptides are paratopes.

47. The multispecific binding protein of claim 45 or claim 46, wherein the plurality of knob peptides are 2, 3, or 4 peptides.

48. A composition comprising a knob peptide of any of claims 1-44 and 103-105.

49. A composition comprising a plurality of knob peptides of any of claims 1-44 and 103-105.

50. The composition of claim 49, wherein the plurality of knob peptides are paratopes.

51. The composition of claim 49 or claim 50, wherein the plurality of knob peptides are 2, 3 or 4 peptides.

52. The composition of any of claims 48-51 that is a pharmaceutical composition, optionally comprising a pharmaceutical carrier.

53. The composition of any of claims 48-52, wherein the composition is formulated for inhalation.

54. An antibody or antigen binding fragment directed against the spike protein of SARS-CoV2, wherein the antibody or antigen binding fragment comprises a heavy chain variable region (VH) and a light chain variable region (VL), wherein the VH comprises a CDR3 H3 set forth in any one of SEQ ID NOS: 86, 88, 89, 92, 95, 98, 101, 103, 104, 107, 110, 112, 114, 116, 117, 119, 121, 123, or 126.

55. The antibody or antigen binding fragment, wherein the VH further comprises a CDR-H1 set forth in any one of SEQ ID NOS: 84, 90, 93, 96, 99, 105, 108, 113, 122 or 124 and a CDR-H2 set forth in any one of SEQ ID NOS: 85, 87, 91, 94, 97, 100, 102, 106, 109, 111, 115, 118, 120 or 125.

56. The antibody or antigen binding fragment, wherein the VH comprises:
(a) a CDR-H1, a CDR-H2 and CDR-H3 set forth in SEQ ID NOS: 84, 85 and 86, respectively;
(b) a CDR-H1, a CDR-H2 and CDR-H3 set forth in SEQ ID NOS: 84, 87 and 88, respectively;
(c) a CDR-H1, a CDR-H2 and CDR-H3 set forth in SEQ ID NOS: 84, 85 and 89, respectively;
(d) a CDR-H1, a CDR-H2 and CDR-H3 set forth in SEQ ID NOS: 90, 91 and 92, respectively;
(e) a CDR-H1, a CDR-H2 and CDR-H3 set forth in SEQ ID NOS: 93, 94 and 95, respectively;
(f) a CDR-H1, a CDR-H2 and CDR-H3 set forth in SEQ ID NOS: 96, 97 and 98, respectively;
(g) a CDR-H1, a CDR-H2 and CDR-H3 set forth in SEQ ID NOS: 99, 100 and 101, respectively;
(h) a CDR-H1, a CDR-H2 and CDR-H3 set forth in SEQ ID NOS: 96, 102 and 103, respectively;
(i) a CDR-H1, a CDR-H2 and CDR-H3 set forth in SEQ ID NOS: 99, 102 and 104, respectively;
(j) a CDR-H1, a CDR-H2 and CDR-H3 set forth in SEQ ID NOS: 105, 106 and 107, respectively;
(k) a CDR-H1, a CDR-H2 and CDR-H3 set forth in SEQ ID NOS: 108, 109 and 110, respectively;
(l) a CDR-H1, a CDR-H2 and CDR-H3 set forth in SEQ ID NOS: 108, 111 and 112, respectively;
(m) a CDR-H1, a CDR-H2 and CDR-H3 set forth in SEQ ID NOS: 113, 111 and 114, respectively;
(n) a CDR-H1, a CDR-H2 and CDR-H3 set forth in SEQ ID NOS: 108, 115 and 116, respectively;
(o) a CDR-H1, a CDR-H2 and CDR-H3 set forth in SEQ ID NOS: 108, 115 and 117, respectively;
(p) a CDR-H1, a CDR-H2 and CDR-H3 set forth in SEQ ID NOS: 108, 118 and 119, respectively;
(q) a CDR-H1, a CDR-H2 and CDR-H3 set forth in SEQ ID NOS: 108, 120 and 121, respectively;
(r) a CDR-H1, a CDR-H2 and CDR-H3 set forth in SEQ ID NOS: 122, 111 and 123, respectively; or
(s) a CDR-H1, a CDR-H2 and CDR-H3 set forth in SEQ ID NOS: 124, 125 and 126, respectively.

57. The antibody or antigen binding fragment of any of claims 54-56, wherein the VH comprises the sequence set forth in any one of SEQ ID NOS: 33-51.

58. The antibody or antigen binding fragment of any of claims 54-56, wherein the VH is humanized

59. The antibody or antigen binding fragment of any of claims 54-56 and 58, wherein the VH has the formula V1-X-V2, wherein the V1 region is a humanized variant of the V region of a bovine antibody, optionally wherein the bovine antibody is BLV1H12, the X is the CDR3 and X2 is a portion of the heavy chain including FR4 of a human J gene segment or a humanized bovine J gene.

60. The antibody or antigen binding fragment of claim 59, wherein V1 is set forth in SEQ ID NO:141, X is set forth in any one of SEQ ID NOS: 86, 88, 89, 92, 95, 98, 101, 103, 104, 107, 110, 112, 114, 116, 117, 119, 121, 123, or 126, and V2 is set forth in any of SEQ ID NOS: 142-147.

61. An ultralong CDR3 antibody or antigen binding fragment directed against the spike protein of SARS-CoV2, wherein the antibody or antigen binding fragment comprises a heavy chain variable region (VH) and a light chain variable region (VL), wherein the VII comprises a CDR3 H3 set forth in any one of SEQ ID NOS: 112, 114, 116, 117, 119, 121, 123, or 126.

62. The ultralong CDR3 antibody or antigen binding fragment of claim 61, wherein the VH further comprises a CDR-H1 set forth in any one of SEQ ID NOS: 108, 113, 122 or 124 and a CDR-H2 set forth in any one of SEQ ID NOS: 111, 115, 118, 120 or 125.

63. The ultralong CDR3 antibody or antigen binding fragment of claim 61 or claim 62, wherein the VH comprises:
(a) a CDR-H1, a CDR-H2 and CDR-H3 set forth in SEQ ID NOS: 108, 111 and 112, respectively;
(b) a CDR-H1, a CDR-H2 and CDR-H3 set forth in SEQ ID NOS: 113, 111 and 114, respectively;
(c) a CDR-H1, a CDR-H2 and CDR-H3 set forth in SEQ ID NOS: 108, 115 and 116, respectively;
(d) a CDR-H1, a CDR-H2 and CDR-H3 set forth in SEQ ID NOS: 108, 115 and 117, respectively;
(e) a CDR-H1, a CDR-H2 and CDR-H3 set forth in SEQ ID NOS: 108, 118 and 119, respectively;
(f) a CDR-H1, a CDR-H2 and CDR-H3 set forth in SEQ ID NOS: 108, 120 and 121, respectively;
(g) a CDR-H1, a CDR-H2 and CDR-H3 set forth in SEQ ID NOS: 122, 111 and 123, respectively; or
(h) a CDR-H1, a CDR-H2 and CDR-H3 set forth in SEQ ID NOS: 124, 125 and 126, respectively.

64. The ultralong CDR3 antibody or antigen binding fragment of any of claims 61-63, wherein the VH comprises the sequence set forth in any one of SEQ ID NOS: 33, 34, 35, 40, 45, 46, 50 or 51.

65. The ultralong CDR3 antibody or antigen binding fragment of any of claims 61-63, wherein the VH is humanized.

66. The ultralong CDR3 antibody or antigen binding fragment of any of claims 61-63 and 65, wherein the VH has the formula V1-X-V2, wherein the V1 region is a humanized variant of the V region of a bovine antibody, optionally wherein the bovine antibody is BLV1H12, the X is the CDR3 and X2 is a portion of the heavy chain including FR-4 of a human J gene segment or a humanized bovine J gene.

67. The ultralong CDR3 antibody or antigen binding fragment of claim 66, wherein the V1 is set forth in SEQ ID NO:141, X is set forth in any one of SEQ ID NOS: 112, 114, 116, 117, 119, 121, 123, or 126, and V2 is set forth in any of SEQ ID NOS: 142-147.

68. The antibody or antigen binding fragment of any of claims 54-67, wherein the VH further comprises a constant region.

69. The antibody or antigen binding fragment of claim 68, wherein the constant region is a constant region of an immunoglobulin IgG1.

70. The antibody or antigen binding fragment of any of claims 54-69, wherein the VL is a bovine light chain variable region or is a humanized variant thereof.

71. The antibody or antigen binding fragment of any of claims 54-70, wherein the VL is a light chain variable region of BLVH12, BLV5D3, BLV8C11, BF1H1, BLV5B8 or F18.

72. The antibody or antigen binding fragment of any of claims 54-71, wherein the VL comprises a CDR-L1, CDR-L2 and CDR-L3 set forth in SEQ ID NO: 151, 152 and 153, respectively.

73. The antibody or antigen binding fragment of any of claims 54-72, wherein the VL is set forth in SEQ ID NO:2.

74. The antibody or antigen binding fragment of any of claims 54-70, wherein the VL is a humanized light chain.

75. The antibody or antigen binding fragment of any of claims 54-69, wherein the VL is a human light chain variable region.

76. The antibody or antigen binding fragment of any of claims 54-69 and 75, wherein the VL is a light chain variable region of VL1-47, VL1-40, VL1-51, and VL2-18, or a variant thereof comprising substitution of amino acids to residues at corresponding positions in a bovine light chain sequence.

77. The antibody or antigen binding fragment of claim 76, wherein the amino acid substitutions are selected from S2A, T5N, P8S, A12G, A13S, and P14L and/or substitution of DNN to GDT, optionally substitution DNNKRP to GDTSRA.

78. The antibody or antigen binding fragment of any of claims 54-69 and 76, wherein the VL comprises the sequence set forth in any of SEQ ID NOS: 155-158.

79. The antibody or antigen binding fragment of any of claims 54-78, wherein the light chain further comprises a constant region.

80. The antibody or antigen binding fragment of any of claims 54-79, wherein the light chain is set forth in SEQ ID NO:154.

81. The antibody or antigen binding fragment of any of claims 54-80 that is a full length antibody.

82. The antibody or antigen binding fragment of any of claims 54-80 that is an antigen binding fragment selected from a Fab or a single chain variable fragment (scFv).

83. The antibody or antigen binding fragment of any of claims 54-82 that is isolated or recombinant.

84. The antibody or antigen binding fragment of any of claims 54-83 that is a monoclonal antibody.

85. A composition comprising an antibody or antigen binding fragment of any of claims 54-84.

86. The composition of claim 85 that is a pharmaceutical composition, optionally comprising a pharmaceutical carrier.

87. A polynucleotide encoding the knob peptide of any of claims 1-44 and 103-105.

88. A polynucleotide encoding the multispecific polypeptide of any of claims 45-47.

89. A polynucleotide encoding the antibody or antigen binding fragment of any of claims 54-84.

90. The polynucleotide of any of claims 87-89 that is a synthetic nucleic acid.

91. The polynucleotide of any of claims 87-90 that is cDNA.

92. A vector comprising the polynucleotide of any of claims 87-91.

93. The vector of claim 92 that is an expression vector.

94. A method of treating a coronavirus infection in a subject in need, the method comprising administering to a subject the knob peptide of any of claims 1-44 and 103-105, the multispecific polypeptide of any of claims 45-47 or the antibody or antigen binding fragment of any of claims 54-84.

95. A method of prophylactically treating a subject at risk for developing a coronavirus infection, the method comprising administering to a subject the knob peptide of any of claims 1-4 and 103-105, the multispecific polypeptide of any of claims 45-47 or the antibody or antigen binding fragment of any of claims 54-84.

96. A method of preventing a coronavirus in aa subject at risk for developing a coronavirus infection, the method comprising administering to a subject the knob peptide of any of claims 1-44 and 103-105, the multispecific polypeptide of any of claims 45-47 or the antibody or antigen binding fragment of any of claims 54-84.

97. The method of any of claims 94-96, wherein the coronavirus infection is caused by SARS-CoV, SARS-CoV-2, or MERS-CoV.

98. The method of any of claims 94-97, wherein the coronavirus infection is COVID-19.

99. The method of any of claims 94-98, wherein the administration is a parenteral administration.

100. The method of any of claims 94-99, wherein the administration is a subcutaneous or intravenous administration.

101. The method of any of claims 94-98, wherein the administration is by inhalation.

102. The method of claim 101, wherein the administration is by use of a nebulizer, inhaler, atomizer, aerosolizser, mister, dry powdered inhaler, metered dose inhaler, metered dose sprayer, metered dose mister, or metered dose atomizer.

103. A knob peptide of any of claims 1-44, wherein the knob has an amino acid sequence length K;
the sequence begins at position X+1 and ends at X+K; and K=L−2X; and
wherein L is the number of amino acids in an amino acid sequence of an antibody starting at the conserved cysteine in framework 3 and ending at the conserved tryptophan in framework 4, and X is the number of amino acids from the first cysteine in framework 3 to the first conserved cysteine encoded by the DH region in CDR H3.

104. The knob peptide of claim 103, wherein the antibody sequence is a bovine antibody.

105. The knob peptide of claim 103 or claim 104, wherein the knob sequence has a sequence that is further extended by one, two, three, four, or five amino acids at the N and/or C termini.
